SEQUENCE LISTING (21 sequences)

SEQ ID NO: 1 - protein, 120 aa, synthetic construct [sequence omitted]

SEQ ID NO: 2 - protein, 5 aa, synthetic construct
DTYIH

SEQ ID NO: 3 - protein, 17 aa, synthetic construct
RIYPTNGYTRYADSVKG

SEQ ID NO: 4 - protein, 11 aa, synthetic construct
WGGDGFYAMDY

SEQ ID NO: 5 - protein, 107 aa, synthetic construct [sequence omitted]

SEQ ID NO: 6 - protein, 11 aa, synthetic construct
RASQDVNTAVA

SEQ ID NO: 7 - protein, 7 aa, synthetic construct
SASFLYS

SEQ ID NO: 8 - protein, 9 aa, synthetic construct
QQHYTTPPT

SEQ ID NO: 9 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSMHEALHNHYTQKSLSLSPGK

SEQ ID NO: 10 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 12 - protein, 5 aa, synthetic construct
DYYIT

SEQ ID NO: 13 - protein, 17 aa, synthetic construct
WIYPGSGNTKYNEKFKG

SEQ ID NO: 14 - protein, 8 aa, synthetic construct
YGNYWFAY

SEQ ID NO: 15 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 16 - protein, 15 aa, synthetic construct
KASQSVDFDGDSYMN

SEQ ID NO: 17 - protein, 7 aa, synthetic construct
AASNLES

SEQ ID NO: 18 - protein, 9 aa, synthetic construct
QQSNEDPWT

SEQ ID NO: 19 - protein, 447 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 218 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 4 aa, synthetic construct
GGFG

(19) United States
(12) Patent Application Publication
(10) Pub. No.: US 2026/0224720 A1
(43) Pub. Date: Aug. 6, 2026

(54) ECTEINASCIDIN DERIVATIVE ANTIBODY DRUG CONJUGATES

(71) Applicant: ADCYTHERIX SAS, Marseille (FR)

(72) Inventor: Julia GAVRILYUK, San Jose, CA (US)

(73) Assignee: ADCYTHERIX SAS, Marseille (FR)

(21) Appl. No.: 19/148,580

(22) PCT Filed: Jan. 16, 2024
(86) PCT No.: PCT/US2024/011578

§ 371 Date: Jul. 16, 2025

Related U.S. Application Data
(60) Provisional application No. 63/480,285, filed on Jan. 17, 2023.

(51) Int. Cl. A61K 47/68; C07D 515/22; C07F 9/6561

(52) U.S. Cl. CPC A61K 47/6803; A61K 47/6849; A61K 47/6889; C07D 515/22; C07F 9/6561

(57) ABSTRACT

Provided herein are compounds, methods, and pharmaceutical compositions for use in treatment of cancer. In certain embodiments, ecteinascidin derivative antibody drug conjugate compounds are provided which display remarkable efficacy and bioavailability for the treatment of, for example, cancer in a human.

### CROSS-REFERENCE

This application claims the benefit of U.S. Provisional Application Ser. No. 63/480,285 filed Jan. 17, 2023, which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 10, 2024, is named 65657-702_601_SL.xml and is 20,721 bytes in size.

### FIELD OF THE INVENTION

Provided herein are compounds, methods, and pharmaceutical compositions for use in the treatment of cancer. In certain embodiments, antibody drug conjugates that include ecteinascidin compounds are provided which display remarkable efficacy, pharmacokinetic parameters, selectivity, and safety for the treatment of, for example, cancer in a human.

### BACKGROUND OF THE INVENTION

Antibody drug conjugates (ADCs) are a developing class of biopharmaceutical drugs that are designed for targeted therapy for treating cancer. In contrast to chemotherapy that lacks selectivity with respect to cancer cells, ADCs are designed to target and attack cancer cells while sparing healthy cells. ADCs accomplish selectivity by attaching a cytotoxic payload or drug to an antibody via a linker. ADCs leverage the antibody's binding selectivity to target delivery of the cytotoxic drug to an abnormal cell.

Ecteinascidins such as trabectedin and lurbinectedin are known to be cancer therapeutics. For example, lurbinectedin has been used to treat small cell lung cancer and trabectedin has been used to treat soft-tissue sarcoma and ovarian cancer. However, trabectedin and lurbinectedin are toxic even at a low concentration.

Accordingly, there is a continuing need for targeted treatments that can optimize an effective dose of ecteinascidin therapeutics.

### SUMMARY OF THE INVENTION

Provided herein are compounds, compositions and methods useful for treating proliferative disease in a subject.

In one embodiment, provided herein are compounds according to Formula (XI), or a pharmaceutically acceptable salt, ester, stereoisomer, or tautomer thereof:

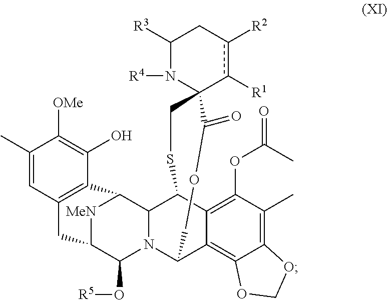

wherein:
R¹ and R² taken together to form an aromatic ring or bicyclic heteroaromatic ring; wherein the aromatic ring and bicyclic heteroaromatic ring are optionally substituted with one to six substituents independently selected from the group consisting of C₁₋₁₀-alkoxy, C₁₋₁₀-alkyl, amino, hydroxyl, halogen, nitro, —P(O)(OH)₂, —SH, and —SO₃H;
R³ is hydrogen or CH₂OR⁶;
R⁴⁰ is H, C₁₋₁₀-alkyl, C₆₋₁₀-aryl, C₃₋₁₀-heterocycle, C(O)R⁸, C(O)OR⁸, C(O)NHR⁸, SO₂R⁸, SO₂NHR⁸; wherein the C₁₋₁₀-alkyl or C₆₋₁₀-aryl is optionally substituted with 1-3 substituents independently selected from the group consisting of —OH, —F, —Cl, —Br, —C₁₋₆-alkyl, —C₃₋₆-cycloalkyl, and —C₃₋₆-heterocyclyl; or L¹
R⁵ and R⁶ are each independently selected from the group consisting of H, C₁₋₁₀-alkyl, C₆₋₁₀-aryl, phosphate, thiophosphate, phosphoramide, C(O)R⁹, C(O)OR⁹, and C(O)NHR⁹, SO₃H, SO₂R⁹, wherein the C₁₋₁₀-alkyl or C₆₋₁₀-aryl is optionally substituted with 1-3 substituents independently selected from the group consisting of —OH, —F, —Cl, —Br, optionally substituted —C₁₋₆-alkyl, optionally substituted —C₃₋₆-cycloalkyl, and optionally substituted —C₃₋₆-heterocyclyl; or L²
wherein the phosphate and thiophosphate of R⁵ or R⁶ are optionally substituted with one to six substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
   wherein the substituent on the phosphate and thiophosphate is optionally terminated with a protecting group;
wherein L¹ or L² is a reactive linker;
R⁸ is selected from the group consisting of C₁₋₆-alkyl, C₃₋₁₀-cycloalkyl, [PEG]_{y}, C₆₋₁₀-aryl, and C₆₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
R⁹ is selected from the group consisting of C₁₋₆-alkyl, C₃₋₁₀-cycloalkyl, [PEG]_{y}, C₆₋₁₀-aryl, and C₆₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
wherein one of R⁴, R⁵, and R⁶ is a linker to a reactive group R¹⁰; or is a linker to an antibody Ab;
wherein [symbol] is either a single or a double bond; and
wherein y is an integer selected from 0 to 32.

In certain embodiments, provided herein are compounds according to Formula (XII) or a pharmaceutically acceptable salt, ester, stereoisomer, or tautomer thereof, comprising:

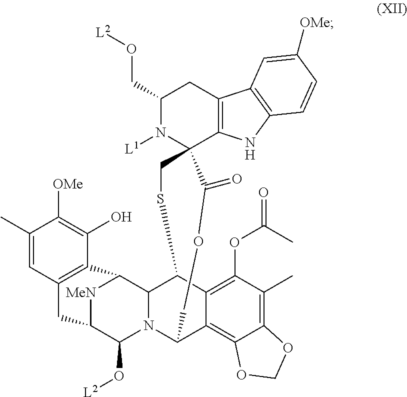

wherein L¹ or one instance of L² is a reactive linker.

In certain embodiments, provided herein are compounds according to Formula (XIII), or a pharmaceutically acceptable salt, ester, stereoisomer, or tautomer thereof, comprising:

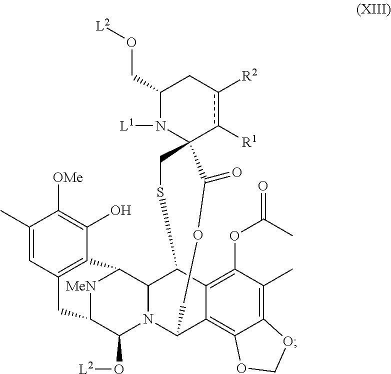

wherein R¹ and R² form at least one aromatic ring or bicyclic heteroaromatic ring, optionally substituted with one to six substituents, independently in each instance, selected from C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

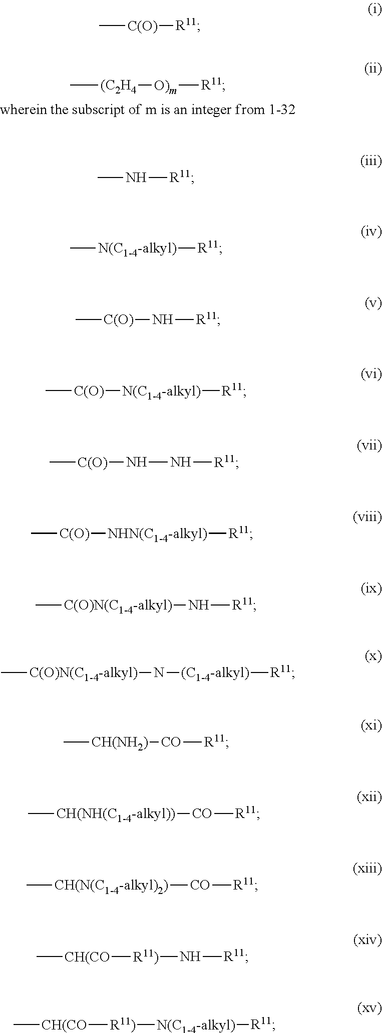

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH;
wherein L² comprises at least one of the following:
(a) —H, if L¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

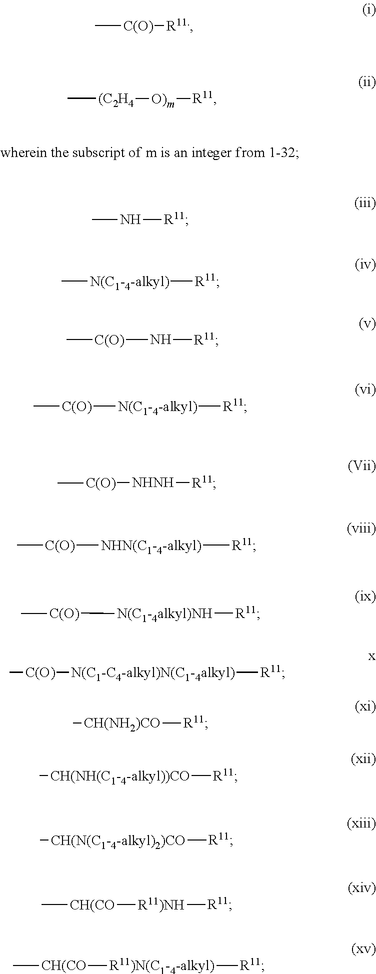

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
R^{11d} is selected from the group consisting of —O— and —NH.
wherein each L² independently comprises at least one of the following:
(a) —H, if L¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

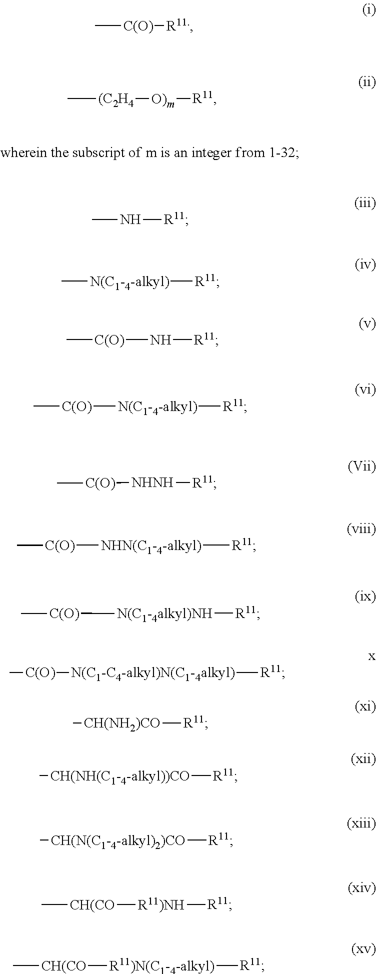

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
R^{11d} is selected from the group consisting of —O— and —NH.

In certain embodiments, provided herein are compounds according to Formula (XIV) or a pharmaceutically acceptable salt, ester, or tautomer thereof, comprising:

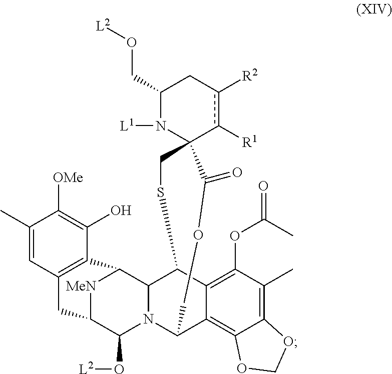

wherein R¹ and R² form at least one aromatic ring or bicyclic heteroaromatic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein L¹ or one instance of L² is a linker;
wherein L¹ is:

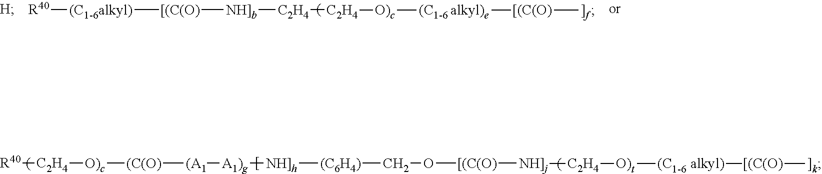

wherein L² is:

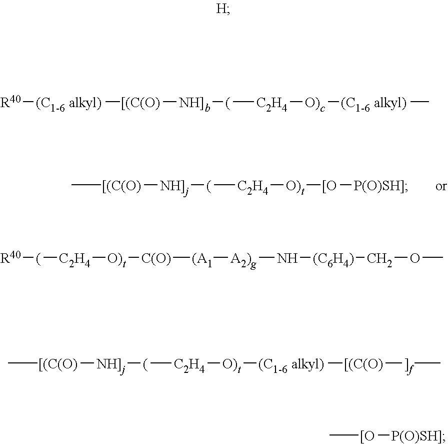

wherein one of L¹ or L² is not H;
wherein R⁴⁰ is a reactive group;
wherein A₁ and A₂ are independently in each instance an amino acid;
wherein subscript:
   b and j are, independently in each instance, selected from 0 to 4;
   c and t are, independently in each instance, selected from 0 to 32;
   e, f, g, h, and k, are, independently in each instance, 0 or 1.

In certain embodiments, a compound of Formula (XVa), (XVb), or (XVc), or a pharmaceutically acceptable salt, ester, stereoisomer, or tautomer thereof, comprising:

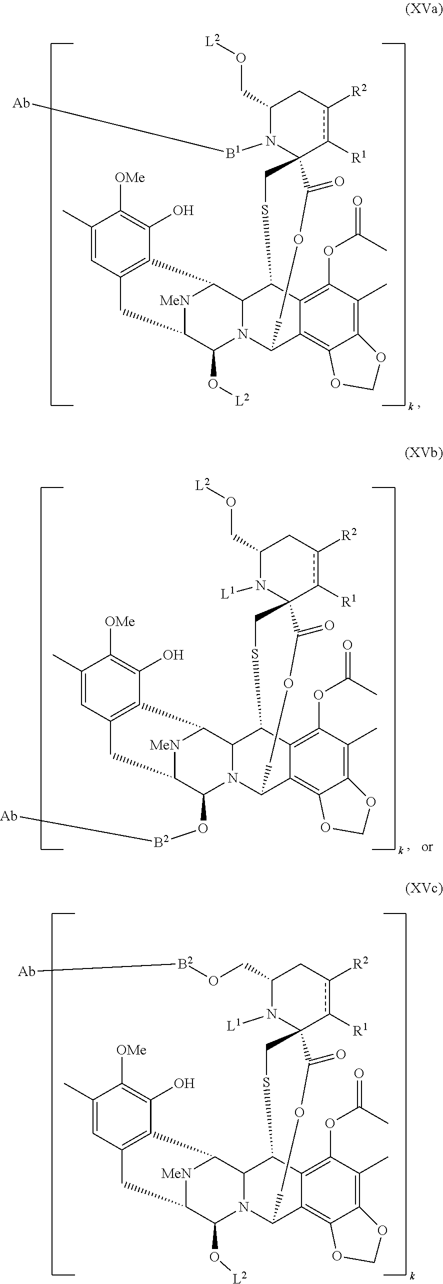

wherein Ab is targeting agent;
wherein subscript k is an integer from 1 to 10;
wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H; and
wherein B¹ is a linker;
wherein B² is a linker.

One embodiment provides a pharmaceutical composition comprising the compound of Formula (XI), (XII), (XIII), (XIV), (XVa), (XVb), (XVc), or a pharmaceutically acceptable salt, ester, stereoisomer, or tautomer thereof, and a pharmaceutically acceptable excipient.

One embodiment provides a method of treating a disease or condition comprising administering the compound of Formula (XI), (XII), (XIII), (XIV), (XVa), (XVb), (XVc), or a pharmaceutically acceptable salt, ester, stereoisomer, or tautomer thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides tumor volume measurements of female B-NDG mice subcutaneously injected with NCI-N87 cells and later dosed with either vehicle control (PBS) or 0.5 mg/kg bodyweight Trastuzumab-LD7.

FIG. 2 provides bodyweight measurements of female B-NDG mice subcutaneously injected with NCI-N87 cells and later dosed with either vehicle control (PBS) or 0.5 mg/kg bodyweight Trastuzumab-LD7.

FIG. 3 provides bodyweight measurements of male CD-1 mice intravenously injected with an isotype human IgG1 antibody conjugated to LD7 at dose rates of 6, 15 or 30 mg/kg body weight (mpk).

### DETAILED DESCRIPTION OF THE INVENTION

Provided herein are compounds, compositions and methods useful for treating proliferative diseases in a subject. Further provided are dosage forms useful for such methods.

### Definitions

When referring to the compounds provided herein, the following terms have the following meanings unless indicated otherwise. Unless defined otherwise, all technical and scientific terms used herein have the same meaning as is commonly understood by one of ordinary skill in the art. If a plurality of definitions exists for a term herein, those in this section prevail unless stated otherwise.

All publications, patent applications, patents, and other references mentioned herein are incorporated herein by reference in their entirety. In case of conflict, the present application will control.

The articles “a,” “an,” and “the” as used herein not only include certain embodiments with a single member, but also may include embodiments with more than one member. For example, an aspect “comprising a compound of Formula Ib and an excipient” should be understood as presenting certain embodiments with at least a second compound of Formula Ib, at least a second excipient, or both.

The term “or” as used herein similarly is a Boolean “or,” unless the alternatives cannot be combined without logical incompatibility. For example, an aspect “comprising an excipient selected from A, B, or C” should be understood as applying to embodiments comprising A and B; B and C; A and C; or A, B, and C.

The term “about” as used herein to modify a numerical value indicates a defined range around that value. If “X” were the value, “about X” would generally indicate a value from 0.95X to 1.05X. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and to provide written description support for a claim limitation of, e.g., “0.98X.” When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 7, 9, or 11%” is equivalent to “about 7%, about 9%, or about 11%.”

The terms “comprise”, “comprising”, “include”, and “including” when used in this specification and in the claims are intended to specify the presence of the stated features, integers, components, or steps, but they do not preclude the presence or addition of one or more additional features, integers, components, or steps thereof.

The term “conjugate” as used herein refers to compound having multimeric antigen-binding compound or a multimeric immunoglobulin, a linker, and a biologically active molecule.

The term “spacer” as used herein refers to chemical building blocks of the linker used to spatially separate the multimeric antigen-binding compound or a multimeric immunoglobulin from the biologically active molecule and to allow for acatabolism of the linker inside of cells.

The term “antibody” as used herein includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As with full antibody molecules, antigen-binding fragments may be monospecific or multispecific (e.g., bispectific). A multispecific antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antigen-binding fragment of an antibody of the present disclosure using routine techniques available in the art.

Biologically active molecules herein (also referred to herein as “drugs,” “toxins,” “cytotoxic agents,” “chemotherapeutic agents,” and the link) include any molecules that have a therapeutic use in mammal when targeted to a specific cell, cell type, or tissue. In typical embodiments the molecule is beneficially delivered to a target within the mammal and in particular is beneficially delivered to and then within a cell (e.g., endocytosis) as compared to molecules released into the vascular or lymphatic systems. In certain embodiments, biologically active molecules are compounds that result in the inhibition, retardation, reduction, and/or prevention of cell growth. Biologically active molecules can also result in cell death via necrosis or apoptosis.

The term “alkyl”, as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, secbutyl, t-butyl, pentyl, isopentyl, neopentyl, hexyl, isohexyl, 3-methylpentyl, 2,2-dimethylbutyl, and 2,3-dimethylbutyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, refers to a saturated straight or branched hydrocarbon having one to six carbon atoms, i.e., C₁ to C₆ alkyl. In certain embodiments, the lower alkyl group is a primary, secondary, or tertiary hydrocarbon. The term includes both substituted and unsubstituted moieties.

The term “cycloalkyl”, as used herein, unless otherwise specified, refers to a saturated cyclic hydrocarbon. In certain embodiments, the cycloalkyl group may be a saturated, and/or bridged, and/or non-bridged, and/or a fused bicyclic group. In certain embodiments, the cycloalkyl group includes three to ten carbon atoms, i.e., C₃ to C₁₀ cycloalkyl. In some embodiments, the cycloalkyl has from 3 to 15 (C₃₋₁₅), from 3 to 10 (C₃₋₁₀), or from 3 to 7 (C₃₋₇) carbon atoms. In certain embodiments, the cycloalkyl group is cyclopropyl, cyclobutyl, cyclopentyl, cyclohexyl, cyclohexylmethyl, cycloheptyl, bicyclo[2.1.1]hexyl, bicyclo[2.2.1]heptyl, decalinyl, or adamantyl.

The term “cycloalkenyl”, as used herein, unless otherwise specified, refers to an unsaturated cyclic hydrocarbon. In certain embodiments, cycloalkenyl refers to mono- or multicyclic ring systems that include at least one double bond. In certain embodiments, the cycloalkenyl group may be a bridged, non-bridged, and/or a fused bicyclic group. In certain embodiments, the cycloalkyl group includes three to ten carbon atoms, i.e., C₃ to C₁₀ cycloalkyl. In some embodiments, the cycloalkenyl has from 3 to 7 (C₃₋₁₀), or from 4 to 7 (C₃₋₇) carbon atoms.

“Alkylene” refers to divalent saturated aliphatic hydrocarbon groups particularly having from one to eleven carbon atoms which can be straight-chained or branched. In certain embodiments, the alkylene group contains 1 to 6 carbon atoms. The term includes both substituted and unsubstituted moieties. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” refers to monovalent olefinically unsaturated hydrocarbon groups, in certain embodiment, having up to about 11 carbon atoms, from 2 to 8 carbon atoms, or from 2 to 6 carbon atoms, which can be straight-chained or branched and having at least 1 or from 1 to 2 sites of olefinic unsaturation. The term includes both substituted and unsubstituted moieties. Exemplary alkenyl groups include ethenyl (i.e., vinyl, or —CH═CH₂), n-propenyl (—CH₂CH═CH₂), isopropenyl (—C(CH₃)═CH₂), and the like.

“Alkenylene” refers to divalent olefinically unsaturated hydrocarbon groups, in certain embodiments, having up to about 11 carbon atoms or from 2 to 6 carbon atoms which can be straight-chained or branched and having at least 1 or from 1 to 2 sites of olefinic unsaturation. This term is exemplified by groups such as ethenylene (—CH═CH—), the propenylene isomers (e.g., —CH═CHCH₂— and —C(CH₃)═CH— and —CH═C(CH₃)—) and the like.

“Alkynyl” refers to acetylenically unsaturated hydrocarbon groups, in certain embodiments, having up to about 11 carbon atoms or from 2 to 6 carbon atoms which can be straight-chained or branched and having at least 1 or from 1 to 2 sites of alkynyl unsaturation. Non-limiting examples of alkynyl groups include acetylenic, ethynyl (—C≡CH), propargyl, (—CH₂C≡CH), and the like.

The term “aryl”, as used herein, and unless otherwise specified, refers to phenyl, biphenyl, or naphthyl. The term includes both substituted and unsubstituted moieties. An aryl group can be substituted with any described moiety, including, but not limited to, one or more moieties selected from the group consisting of halogen (fluoro, chloro, bromo or iodo), alkyl, haloalkyl, hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

“Alkoxy” refers to the group —OR′ where R′ is alkyl or cycloalkyl. Alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkoxycarbonyl” refers to a radical —C(O)-alkoxy where alkoxy is as defined herein.

“Amino” refers to the radical —NH₂.

“Carboxyl” or “carboxy” refers to the radical —C(O)OH.

The term “alkylamino” or “arylamino” refers to an amino group that has one or two alkyl or aryl substituents, respectively. In certain embodiments, the alkyl substituent is lower alkyl. In another embodiment, the alkyl or lower alkyl is unsubstituted.

“Halogen” or “halo” refers to chloro, bromo, fluoro or iodo.

“Monoalkylamino” refers to the group alkyl-NR′—, wherein R′ is selected from hydrogen and alkyl or cycloalkyl.

“Thioalkoxy” refers to the group —SR′ where R′ is alkyl or cycloalkyl.

The term “heterocyclyl” or “heterocyclic” refers to a monovalent monocyclic non-aromatic ring system and/or multicyclic ring system that contains at least one non-aromatic ring, wherein one or more of the non-aromatic ring atoms are heteroatoms independently selected from O, S, or N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Heterocyclyl groups are bonded to the rest of the molecule through the non-aromatic ring. In certain embodiments, the heterocyclyl is a monocyclic, bicyclic, tricyclic, or tetracyclic ring system, which may include a fused or bridged ring system, and in which the nitrogen or sulfur atoms may be optionally oxidized, the nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic radicals include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, O-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted as described herein.

The term “heteroaryl” refers to refers to a monovalent monocyclic aromatic group and/or multicyclic aromatic group that contain at least one aromatic ring, wherein at least one aromatic ring contains one or more heteroatoms independently selected from O, S, and N in the ring. Heteroaryl groups are bonded to the rest of the molecule through the aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted as described herein.

In certain embodiments, a heteroaryl group refers to a monocyclic 5- or 6-membered heteroaryl group optionally substituted by a C₁₋₃-alkyl group in the carbon skeleton, where the 6-membered heteroaryl group contains one, two, or three nitrogen atoms and the 5-membered heteroaryl group contains an imino group optionally substituted by a C₁₋₃-alkyl or phenyl-C₁₋₃-alkyl group, an oxygen or sulfur atom, or an imino group optionally substituted by a C₁₋₃-alkyl or phenyl-C₁₋₃-alkyl group or an oxygen or sulfur atom and additionally a nitrogen atom or an imino group optionally substituted by a C₁₋₃-alkyl or phenyl-C₁₋₃-alkyl group and two nitrogen atoms, and where a phenyl ring may be fused to the abovementioned monocyclic heterocyclic group via two adjacent carbon atoms, in which the bonding takes place via a nitrogen atom or via a carbon atom of the heterocyclic moiety or a fused phenyl ring.

The term “alkylaryl” refers to an aryl group with an alkyl substituent. The term “aralkyl” or “arylalkyl” includes an alkyl group with an aryl substituent.

The term “alkylheterocyclyl” refers to a heterocyclyl group with an alkyl substituent. The term alkylheterocyclyl includes an alkyl group with a heterocyclyl substituent.

The term “alkylheteroaryl” refers to a heteroaryl group with an alkyl substituent. The term alkylheteroaryl includes an alkyl group with a heteroaryl substituent.

The term “protecting group” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

“Pharmaceutically acceptable salt” refers to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts further include, by way of example only and without limitation, sodium, potassium, calcium, magnesium, ammonium, tetraalkylammonium and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate and the like.

The term “purine” or “pyrimidine” base refers to, but is not limited to, adenine, N⁶-alkylpurines, N⁶-acylpurines (wherein acyl is C(O)(alkyl, aryl, alkylaryl, or arylalkyl), N⁶-benzylpurine, N⁶-halopurine, N⁶-vinylpurine, N⁶-acetylenic purine, N⁶-acyl purine, N⁶-hydroxyalkyl purine, N⁶-alkylaminopurine, N⁶-thioalkyl purine, N²-alkylpurines, N²-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C⁵-alkylpyrimidines, C⁵-benzylpyrimidines, C⁵-alopyrimidines, C⁵-vinylpyrimidine, C⁵-acetylenic pyrimidine, C⁵-acyl pyrimidine, C⁵-hydroxyalkyl purine, C⁵-amidopyrimidine, C⁵-cyanopyrimidine, C⁵-iodopyrimidine, C⁶-iodo-pyrimidine, C⁵—Br-vinyl pyrimidine, C⁵—Br-vinyl pyrimidine, C⁵-nitropyrimidine, C⁵-amino-pyrimidine, N²-alkylpurines, N²-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 7-deazaguanine, 7-deazaadenine, 2,6-diaminopurine, and 6-chloropurine. Functional oxygen and nitrogen groups on the base can be protected as necessary or desired. Suitable protecting groups are well known to those skilled in the art, and include trimethylsilyl, dimethylhexylsilyl, t-butyldimethylsilyl, and t-butyldiphenylsilyl, trityl, alkyl groups, and acyl groups such as acetyl and propionyl, methanesulfonyl, and p-toluenesulfonyl.

The term “acyl” or “O-linked ester” refers to a group of the formula C(O)R′, wherein R′ is alkyl or cycloalkyl (including lower alkyl), carboxylate reside of amino acid, aryl including phenyl, alkaryl, arylalkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or arylalkyl sulphonyl including methanesulfonyl, the mono, di or triphosphate ester, trityl or monomethoxy-trityl, substituted benzyl, alkaryl, arylalkyl including benzyl, alkoxyalkyl including methoxymethyl, aryloxyalkyl such as phenoxymethyl. Aryl groups in the esters optimally comprise a phenyl group. In particular, acyl groups include acetyl, trifluoroacetyl, methylacetyl, cyclpropylacetyl, propionyl, butyryl, hexanoyl, heptanoyl, octanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, 4-phenylbenzoyl.

The term “amino acid” refers to naturally occurring and synthetic α, β γ or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In certain embodiments, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

The term “substantially free of” or “substantially in the absence of” with respect to a composition refers to a composition that includes at least 85 or 90% by weight, in certain embodiments 95%, 98%, 99% or 100% by weight, of the designated enantiomer of that compound. In certain embodiments, in the methods and compounds provided herein, the compounds are substantially free of enantiomers.

Similarly, the term “isolated” with respect to a composition refers to a composition that includes at least 85, 90%, 95%, 98%, 99% to 100% by weight, of the compound, the remainder comprising other chemical species or enantiomers.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen”, the position is understood to have hydrogen at its natural isotopic composition.

“Isotopic enrichment” refers to the percentage of incorporation of an amount of a specific isotope at a given atom in a molecule in the place of that atom's natural isotopic abundance. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. The isotopic enrichment of the compounds provided herein can be determined using conventional analytical methods known to one of ordinary skill in the art, including mass spectrometry and nuclear magnetic resonance spectroscopy.

“Isotopically enriched” refers to an atom having an isotopic composition other than the natural isotopic composition of that atom. “Isotopically enriched” may also refer to a compound containing at least one atom having an isotopic composition other than the natural isotopic composition of that atom.

As used herein, “alkyl,” “cycloalkyl,” “alkenyl,” “cycloalkenyl,” “alkynyl,” “aryl,” “alkoxy,” “alkoxycarbonyl,” “amino,” “carboxyl,” “alkylamino,” “arylamino,” “thioalkyoxy,” “heterocyclyl,” “heteroaryl,” “alkylheterocyclyl,” “alkylheteroaryl,” “acyl,” “aralkyl,” “alkaryl,” “purine,” “pyrimidine,” “carboxyl” and “amino acid” groups optionally comprise deuterium at one or more positions where hydrogen atoms are present, and wherein the deuterium composition of the atom or atoms is other than the natural isotopic composition.

Also as used herein, “alkyl,” “cycloalkyl,” “alkenyl,” “cycloalkenyl,” “alkynyl,” “aryl,” “alkoxy,” “alkoxycarbonyl,” “carboxyl,” “alkylamino,” “arylamino,” “thioalkyoxy,” “heterocyclyl,” “heteroaryl,” “alkylheterocyclyl,” “alkylheteroaryl,” “acyl,” “aralkyl,” “alkaryl,” “purine,” “pyrimidine,” “carboxyl” and “amino acid” groups optionally comprise carbon-13 at an amount other than the natural isotopic composition.

As used herein, EC₅₀ refers to a dosage, concentration or amount of a particular test compound that elicits a dose-dependent response at 50% a of maximal expression of a particular response that is induced, provoked or potentiated by the particular test compound.

As used herein, the IC₅₀ refers to an amount, concentration or dosage of a particular test compound that achieves a 50% inhibition of a maximal response in an assay that measures such response.

The term “host”, as used herein, refers to any unicellular or multicellular organism in which the virus can replicate, including cell lines and animals, and in certain embodiments, a human. Alternatively, the host can be carrying a part of the Flaviviridae viral genome, whose replication or function can be altered by the compounds of the present invention. The term host specifically includes infected cells, cells transfected with all or part of the Flaviviridae genome and animals, in particular, primates (including chimpanzees) and humans. In most animal applications of the present invention, the host is a human patient. Veterinary applications, in certain indications, however, are clearly anticipated by the present invention (such as chimpanzees).

As used herein, the terms “subject” and “patient” are used interchangeably herein. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is refractory or non-responsive to current treatments for hepatitis C infection. In another embodiment, the subject is a farm animal (e.g., a horse, a cow, a pig, etc.) or a pet (e.g., a dog or a cat). In certain embodiments, the subject is a human.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent which is known to be useful for, or has been or is currently being used for the treatment or prevention of a disorder or one or more symptoms thereof.

“Therapeutically effective amount” refers to an amount of a compound or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

As used herein, the terms “prophylactic agent” and “prophylactic agents” as used refer to any agent(s) which can be used in the prevention of a disorder or one or more symptoms thereof. In certain embodiments, the term “prophylactic agent” includes a compound provided herein. In certain other embodiments, the term “prophylactic agent” does not refer a compound provided herein. For example, a prophylactic agent is an agent which is known to be useful for, or has been or is currently being used to prevent or impede the onset, development, progression and/or severity of a disorder.

As used herein, the phrase “prophylactically effective amount” refers to the amount of a therapy (e.g., prophylactic agent) which is sufficient to result in the prevention or reduction of the development, recurrence or onset of one or more symptoms associated with a disorder (or to enhance or improve the prophylactic effect(s) of another therapy (e.g., another prophylactic agent).

As used herein, the phrase “reactive linker group,” refers to functional groups used for conjugation, i.e., activated esters, haloacetamides, enzymatic conjugations, click-chemistry. Conjugation moiety can be known in the art. For example, the conjugation moieties in Signal Transduction and Targeted Therapy (2022) 7:93; https://doi.org/10.1038/s41392-022-00947-7, are herein incorporated by reference in their entirety for all purposes. Chemical Society Reviews (2019), 48(16), 4361-4374; and Current Topics in Medicinal Chemistry (Sharjah, United Arab Emirates) (2017), 17(32), 3393-3424; also set forth linkers and antibody drug conjugates which are herein incorporated by reference in their entirety for all purposes. As used herein, a reactive linker or reactive linker group may comprise an alkyl or heteroalkyl spacer segment, which is not reactive, wherein the spacer segment is capped with a reactive group (e.g., R⁴⁰). Examples of reactive groups include any group capable of forming a bond with an antibody. Specifically, examples of reactive groups include maleimides (configured to form a bond with a sulfur atom of a cysteine side-chain), N-hydroxysuccinimides (configured to form a bond with a nitrogen atom of a lysine side-chain), or primary amines (configured to form a bond with an amide of a glutamine side-chain, e.g., via a transglutaminase). Reactive linkers may further comprise a peptide group (e.g., a cleavable peptide, e.g., Val-Cit), a self-immolative group (e.g., a p-aminobenzyloxycarbonyl (“PABC”) group), a polymeric group (e.g., polyethylene glycol (PEG)), an alkyl group (e.g., C₁₋₁₂ alkyl), a heteroalkyl (e.g., a peptide) group, or a combination thereof. A linker may further comprise one or more cyclyl groups. For example, a cyclyl group may be a fused heteroaryl group formed from the reaction of a bicyclooctyne and a triazine (e.g., in a “Click” reaction), or a cyclyl group formed from a reaction between a sulfur atom and a maleimido group.

### Antibodies

In some embodiments, the term “antibody” is used in the broadest sense and includes polyclonal and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (for example, sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, for example, bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745. (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme). The CDRs of the antibodies described herein may be defined by the Kabat, IMGT, Chothia, AbM, Aho, contact numbering scheme, or any combination thereof.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three CDRs (See for example, Kindt et al. Kuby Immunology, 6th ed., W.H. Freeman and Co., page 91(2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively (See for example, Portolano et al., J. Immunol. 150:880-887 (1993); Clarkson et al., Nature 352:624-628 (1991)).

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

Among the provided antibodies are antibody fragments. An “antibody fragment,” “antigen-binding fragment,” “antigen-binding domain,” “antigen-binding region,” “antigen binding fragment,” “antigen binding domain,” “antigen binding region,” and similar terms refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabodies; linear antibodies; single-chain antibody molecules (for example, scFv or sFv); and multispecific antibodies formed from antibody fragments. In particular embodiments, the antibodies are single-chain antibody fragments comprising a variable heavy chain region and/or a variable light chain region, such as scFvs. Generally, an antibody fragment or antigen-binding fragment will comprise one or more CDRs from a parental antibody that are sufficient to confer binding specificity.

Generally, a humanized antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (for example, the antibody from which the CDR residues are derived), for example, to restore or improve antibody specificity or affinity. In some embodiments, a humanized antibody refers to forms of non-human (for example, murine) or not fully humanized antibodies having specific immunoglobulin chains, chimeric immunoglobulins, or fragments thereof that contain minimal non-human (for example, murine) sequences.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived or selected from human antibody libraries, including phage display and cell-free libraries, containing antibody-encoding sequences derived from a human repertoire. In certain embodiments, a human antibody can have sequence liabilities removed or its affinity increased by successive rounds of selection by a method such as phage display.

### Fc Constant Regions

Generally, the fragment crystallizable (Fc) region or domain of an antibody mediates downstream effector functions via its interaction with Fc-receptors on immune cells (for example, innate immune cells) or with complement protein C1q, the recognition molecule of the complement system. Furthermore, the interaction with Fc-receptors can lead to killing of targeted cells through a variety of immune effector mechanisms, including antibody-dependent cell-mediated cytotoxicity (ADCC) and antibody-dependent cellular phagocytosis (ADCP), and antibody-mediated complement activation may lead to complement-dependent cytotoxicity (CDC). In addition, both Fc-receptor interactions and complement activation can exert a broad range of immunomodulatory functions.

Accordingly, in certain instances, mutations within the Fc region that reduce, inhibit, ablate, and/or abrogate Fc-mediated function are advantageous for reducing immune activation resulting from the binding of an antibody to the target. In some embodiments, one or more amino acid modifications may be introduced into the Fc region of an antibody, thereby generating an Fc region variant. An Fc region may comprise a C-terminal region of an immunoglobulin heavy chain that comprises a hinge region, CH₂ domain, CH₃ domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions.

### Pharmaceutical Compositions

A pharmaceutical composition disclosed herein can be administered to a subject by any suitable administration route, including but not limited to, parenteral (intravenous, subcutaneous, intraperitoneal, intramuscular, intravascular, intrathecal, intravitreal, infusion, or local), topical, oral, or nasal administration.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one antibody of the present disclosure is administered.

### Compounds, Linker-Drug Conjugates, and Antibody-Drug Conjugates

Provided herein are ecteinascidin derivative compounds useful for the treatment, diagnosis, or detection of pathological conditions. In certain embodiments, provided herein are compounds or a pharmaceutically acceptable salt, solvate, stereoisomeric form, tautomeric form or polymorphic form thereof. The ecteinascidin derivative compounds can be useful as payloads in antibody drug conjugates (ADCs), payload-linker compounds, and ADC compounds.

In certain embodiments, the disclosure provides a compound comprising a linker covalently bonded to lurbinectedin via a secondary alcohol, or a secondary amine. In certain embodiments, the disclosure provides a compound comprising a linker covalently bonded to trabectedin via a secondary alcohol, or a secondary amine. In certain embodiments, provided herein is a compound comprising a linker covalently bonded to ecubectedin via a secondary alcohol, or a secondary amine.

In certain embodiments, provided herein are compounds according to Formula I:

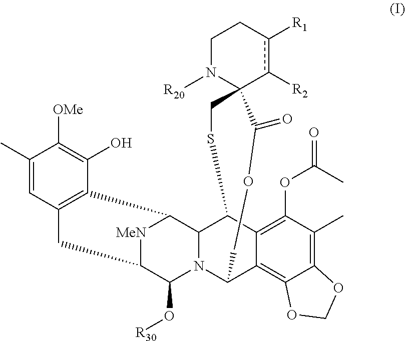

R¹ and R² can form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H. In an example, R¹ and R² can be taken together with the intervening atoms to which they are attached to form a monocyclic or bicyclic ring.
R²⁰ is either: (a) selected from the group consisting of H, C₁₋₁₀-alkyl, C₆₋₁₀-aryl, C₃₋₁₀-heterocycle, C(O)R₅, C(O)OR₅, C(O)NHR₅, SO₂R₅, and SO₂NHR₅, wherein the C₁₋₁₀-alkyl is substituted or unsubstituted, wherein the C₆₋₁₀-aryl is substituted or unsubstituted; or (b) L¹, wherein L¹ is a reactive linker group.
R²⁰ is optionally substituted by 1-3 substituents, independently in each instance, selected from the group consisting of OH, F, Cl, Br, C₁₋₅-alkyl, C₃₋₆-cycloalkyl, and C₃₋₆-heterocycle.
R³⁰ is either: (c) selected from the group consisting of H, C₁₋₁₀-alkyl, C₁₋₁₀-aryl, phosphate, thiophosphate, phosphoramide, C(O)R₆, C(O)OR₆, and C(O)NHR₆, sulfonyl, sulfonylamide, wherein the C₁₋₁₀-alkyl is substituted or unsubstituted, wherein the C₆₋₁₀-aryl is substituted or unsubstituted, or (d) L², wherein L² is a reactive linker group.

In certain embodiments, the phosphate and thiophosphate are optionally substituted with one to two substituents independently, in each instance, selected from the group consisting of H, C₁₋₆-alkyl, C₃₋₁₀-cycloalkyl, poly(ethylene) glycol (PEG), C₆₋₁₀-aryl, and C₅₋₁₀-heteroaryl, wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H. The substituent on the phosphate and thiophosphate is optionally terminated with a protecting group.

In certain embodiments, at least one of R²⁰ or R³⁰ is not H.

In certain embodiments, R⁵ and R⁶ are each, independently in each instance, selected from the group consisting of C₁₋₆-alkyl, C₃₋₁₀-cycloalkyl, (poly(ethylene) glycol)_{y} ([PEG]_{y}), C₆₋₁₀-aryl, and C₆₋₁₀-heteroaryl, wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H. In certain embodiments, subscript y is an integer selected from 1 to 32. In certain embodiments, when R⁵ and/or R⁶ include [PEG]_{y}, the terminal PEG group can be terminated with any suitable moiety, including, but not limited to —OH, —NH₂, and/or —OMe.

L¹ of formula (I) can be at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S.
R¹⁰ can be R^{10a}-R^{10b}, wherein R^{10a} is either absent or —(CH₂)ₑ—, wherein e is independently in each instance, selected from 1-4.
R^{10b} can be selected from the group consisting of:

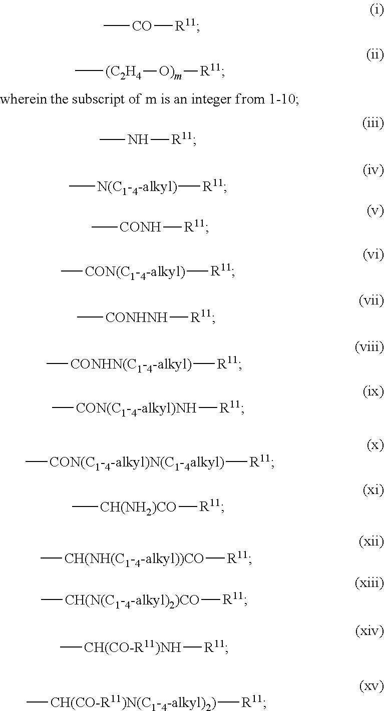

R¹¹ can be selected from the group consisting of R^{11a}, R^{11b}, R^{11c}, R^{11d}, and combinations thereof.
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—.
R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6.
R^{11c} is —(CH₂)ₓ—, wherein the subscript of x is an integer selected from 1 to 10.
R^{11d} is selected from the group consisting of —O— and —NH.
L² of formula (I) can be at least one of the following:
(a) —H, if L¹ is not H;
(b) —PO₃H—R¹¹ or —PO₃H₂;
(c) —PO₂SH—R¹¹ or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
R^{10a} is either absent or —(CH₂)ₑ—, wherein e is independently in each instance, selected from 1-4;
R^{10b} is selected from the group consisting of:

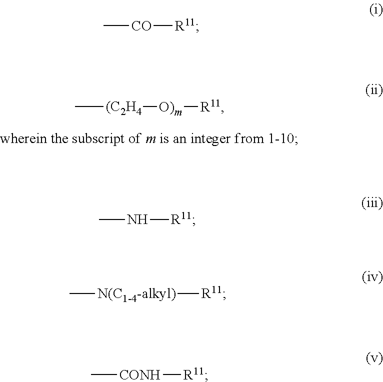

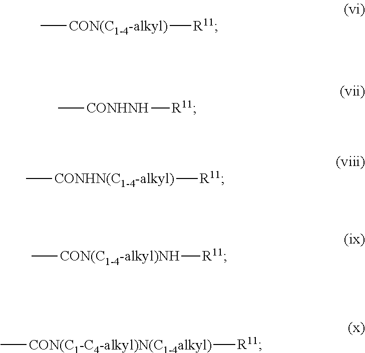

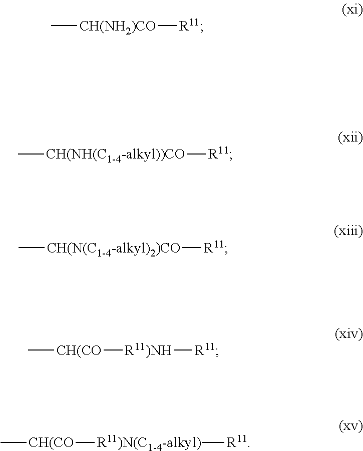

In certain embodiments, R¹¹ can be selected from the group consisting of R^{11a}, R^{11b}, R^{11c}, R^{11d}, and combinations thereof; wherein R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—; R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6; R^{11c} is —(CH₂)ₓ—, wherein the subscript of x is an integer selected from 1 to 10; and R^{11d} is selected from the group consisting of —O— and —NH.

In certain embodiments, R¹¹ is selected from R^{11a}-R^{11b}-R^{11c}-R^{11d}; R^{11b}-R^{11c}-R^{11d}; R^{11c}-R^{11d}; or R^{11a}-R^{11c}-R^{11d}.

In certain embodiments, L¹ and L² are independently at least one of the following bivalent structures:

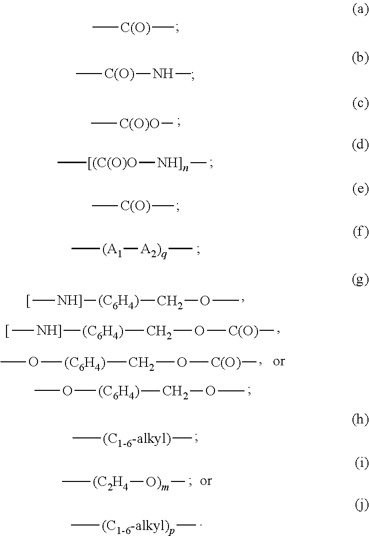

In some embodiments of Formula (I), L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—; wherein L¹ is terminated in R⁴⁰, wherein R⁴⁰ is a reactive group. In some embodiments, one or more occurrences of -(A₁)_{q}- is Val-Cit or Cit-Val. In some embodiments, L¹ is —C(O)—(C₁₋₆-alkyl)-R⁴⁰. In some embodiments, L¹ is —C(O)—(C₂H₄—O)ₘ—(C₁₋₆-alkyl)-NH—C(O)—(C₁₋₆-alkyl)-R⁴⁰. In some embodiments, L¹ is —C(O)—(C₂H₄—O)ₘ—(CH₂)₂—NH—C(O)—(CH₂)₂—R⁴⁰. In some embodiments, L¹ is —C(O)—(C₂H₄—O)ₘ—NH—C(O)—O—CH₂—(C₆H₄)—NH-(A₁)_{q}—C(O)—(C₂H₄—O)ₘ—(C₁₋₆-alkyl)-R⁴⁰. In some embodiments, L¹ is -(A₁)_{q}-C(O)—O—CH₂—(C₆H₄)—NH-(A₁)_{q}-C(O)—(C₁₋₆-alkyl)-R⁴⁰. In some embodiments, L¹ is di(pyrrolidine-1-yl)methyl. In some embodiments, L¹ is —C(O)(C₂H₄—O)₂—(CH₂)₂—NH—C(O)—(CH₂)₂—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Va)-C(O)—(C₂H₄—O)₂—(CH₂)₂—R⁴⁰. In some embodiments, L¹ is -(Gly)-C(O)—O—CH₂—(C₆H₄)—NH-(Cit-Val)-C(O)—(CH₂)₅—R⁴⁰.

In some embodiments of Formula (I), L² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² is —PO₂SH—(C₂H₄—O)ₘ—(C₁₋₆-alkyl)-R⁴⁰. In some embodiments, L² is —PO₂SH—(C₂H₄—O)ₘ—(C₁₋₆ alkyl)-NH—C(O)—O—CH₂—(C₆H₄)NH-(A₁)_{q}-C(O)—(C₂H₄—O)ₘ—(C₁₋₆-alkyl)-R⁴⁰. In some embodiments, L² is —PO₂SH—(C₂H₄—O)ₘ—NH—C(O)—(C₂H₄—O)ₘ—NH—C(O)—(C₁₋₆-alkyl)-R⁴⁰. In some embodiments, L² is —PO₂SH₂. In some embodiments, L² is —PO₃H₂. In some embodiments, L² is —PO₂SH—(C₂H₄—O)₃—(CH₂)₂—R⁴⁰. In some embodiments, L² is —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L² is —PO₂SH—(C₂H₄—O)₃—(CH₂)₂—NH—C(O)—(C₂H₄—O)₄—(CH₂)₂—NH—C(O)—(CH₂)₂—R⁴⁰.

In some embodiments of antibody drug conjugates derived from a compound of Formula (I), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—. In some embodiments, at least one occurrence of -(A₁)_{q}- is selected from: Val-Cit or Cit-Val. In some embodiments, B¹ comprises —C(O)—(C₁₋₆-alkyl)-. In some embodiments, B¹ comprises —C(O)—(C₂H₄—O)ₘ—(C₁₋₆-alkyl)-NH—C(O)—(C₁₋₆-alkyl)-. In some embodiments, B¹ comprises —C(O)—(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)₂—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)—(CH₂)₅—.

In some embodiments of antibody drug conjugates derived from a compound of Formula (I), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—.

In certain embodiments of a compound of Formula (I), L¹ or L² are terminated in R⁴⁰, wherein R⁴⁰ is a reactive group. In certain embodiments, R¹¹ is terminated in R⁴⁰, wherein R⁴⁰ is a reactive group. In some embodiments, the reactive group is -NHBoc

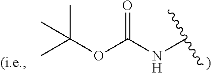 [SMILES: *NC(=O)OC(C)(C)C]

or —NH₂. In some embodiments, the reactive group is maleimide.

In some embodiments of a compound of Formula (I), when R²⁰ is H, then R³⁰ is L²; and when R³⁰ is H, then R²⁰ is L. In some embodiments of a compound of Formula (I), when L¹ is H, then at least one occurrence of L² is not H; and when all occurrences of L² are H, then L¹ is not H. In some embodiments, R²⁰ is di(pyrrolidin-1-yl)methyl or L¹, and R³⁰ is H. In some embodiments, R³⁰ is —PO₃H₂, —PO₂SH₂, or L²; and R²⁰ is H.

In some embodiments of an antibody drug conjugate derived from a compound of Formula (I), B¹ comprises the reaction product of R⁴⁰ as found in L¹ with Ab, such that a covalent linkage is formed therebetween. In some embodiments of an antibody drug conjugate derived from a compound of Formula (I), B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

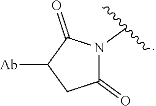

In certain embodiments of a compound of Formula (I), A₁ and A₂ are independent in each instance an amino acid (e.g., an amino acid residue), wherein the amino acid is selected from a natural amino acid or a non-natural amino acid.

In certain embodiments, subscript n is an integer selected from 0 to 4; subscript m is an integer selected from 0 to 20; subscript p is an integer 0 or 1; and subscript t is an integer selected from 0 to 10. In certain embodiments, each instance of subscript n is independently an integer selected from 0 to 4; each instance of subscript m is independently an integer selected from 1 to 20; each instance of subscript p is independently an integer 0 or 1; each instance of subscript q is independently an integer selected from 1 to 6; and each instance of subscript n is independently an integer selected from 0 to 10.

In certain embodiments, R²⁰ is a C₁-alkyl optionally substituted with at least one C₁₋₆-heterocycle. In certain embodiments, R²⁰ is a C₁-alkyl optionally substituted with a bipyrrolidinyl substituent. In certain embodiments, R²⁰ is a C₁-alkyl optionally substituted with 1 to 2 pyrrolidonyl substituents.

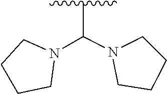 [SMILES: *C(N1CCCC1)N1CCCC1]

In certain embodiments, R²⁰ is 1,1′-(ethane-1,1-d)dipyrrolidine, wherein

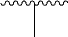

indicates a bond through which the illustrated substituent is bonded. In some embodiments, R²⁰ is di(pyrrolidin-1-yl)methyl.

In certain embodiments, R³⁰ is phosphate or thiophosphate. In certain embodiments, R³⁰ is substituted phosphate or substituted thiophosphate. In certain embodiments, R³⁰ is

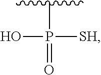 [SMILES: *P(=O)(O)S]

wherein

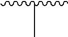

indicates the bond through which the thiophosphate is bonded.

In certain embodiments, the compound (e.g., the compound of Formula (I)) is selected from

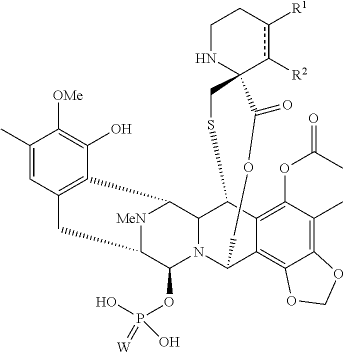

wherein W is O or S; wherein [symbol] is either a single or a double bond.

In certain embodiments, the compound (e.g., the compound of Formula (I)) can be at least one of the compounds listed in Table A.

TABLE A

| | |
|---|---|
| I-A | 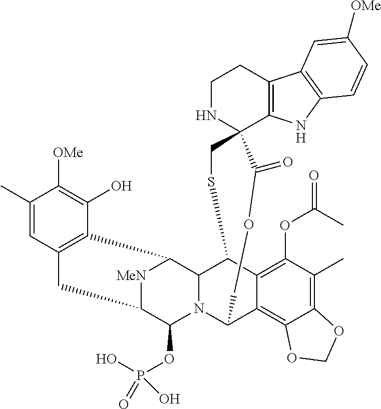 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)O)N2C)OCO4] |
| I-B | 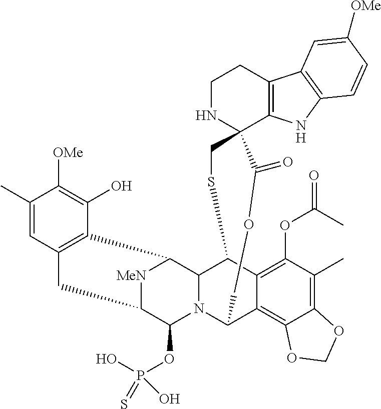 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(O)(O)=S)N2C)OCO4] |
| I-C | 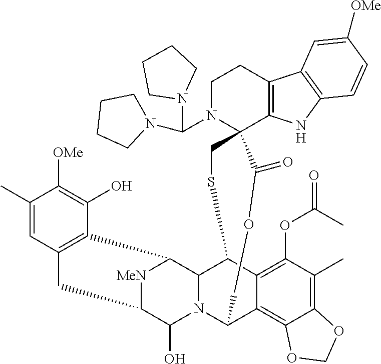 [SMILES: COc1ccc2[nH]c3c(c2c1)CCN(C(N1CCCC1)N1CCCC1)[C@]31CS[C@@H]2c3c(OC(C)=O)c(C)c4c(c3[C@H](COC1=O)N1C2[C@H]2c3c(cc(C)c(OC)c3O)C[C@@H](C1O)N2C)OCO4] |
| I-D | 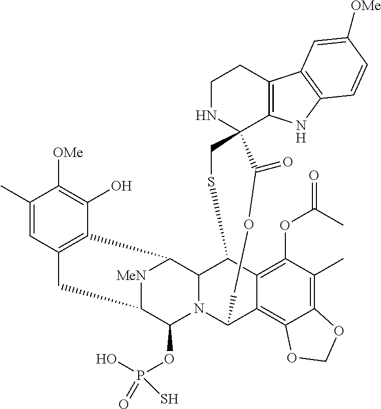 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4] |
| I-E | 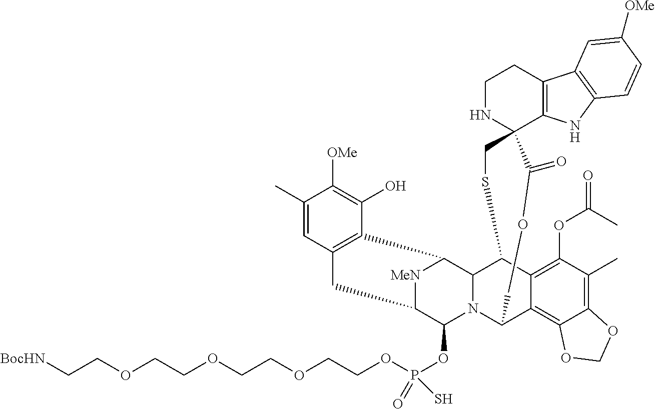 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4] |
| I-F | 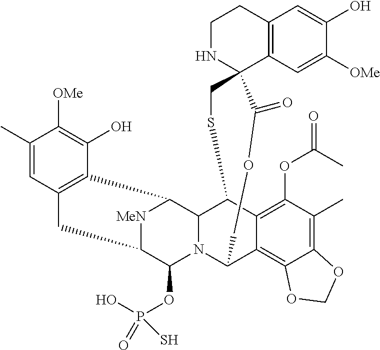 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4] |
| I-G | 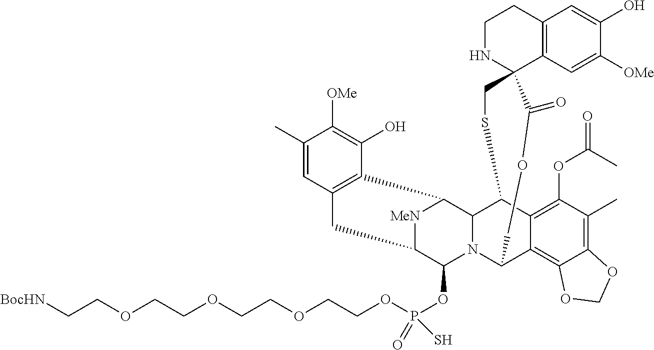 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4] |

The present disclosure provides a compound of Formula (II) or a pharmaceutically acceptable salt, ester, stereoisomer or tautomer thereof, comprising:

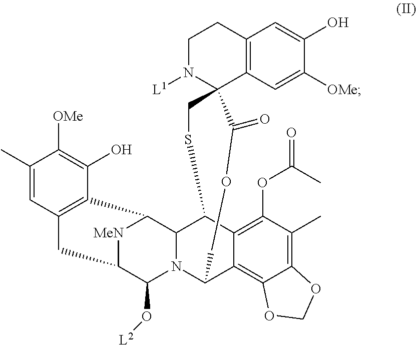

wherein one of L¹ or L² is a reactive linker.

In certain embodiments, the reactive linker comprises a nucleophilic group reactive with an electrophilic group on at least a portion of a targeting moiety.

In certain embodiments, L¹ comprises at least one of the following:
(a) —H, wherein at least one of L¹ or L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰,
The C₅₋₁₀-heteroaryl optionally comprises 1-3 heteroatoms independently selected from the group N, O, and S.
R¹⁰ is R^{10a}-R^{10b}, wherein R^{10a} is either absent or —(CH₂)ₑ—, wherein e is independently in each instance, selected from 1-4.
R^{10b} is selected from the group consisting of:

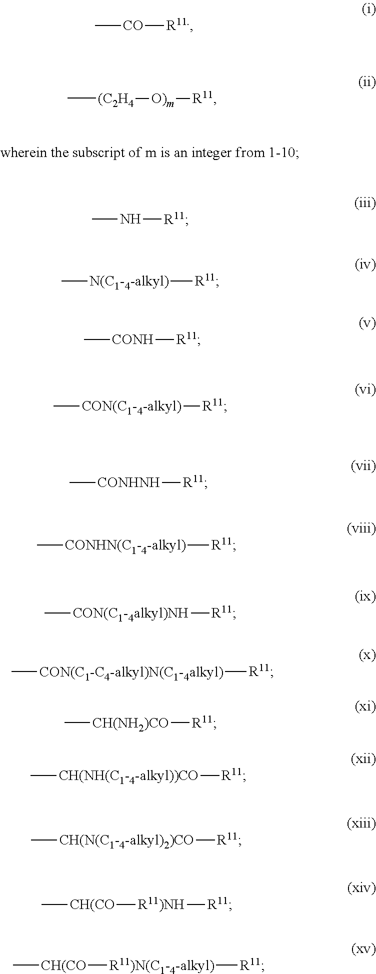

In certain embodiments, R¹¹ is selected from the group consisting of R^{11a}, R^{11b}, R^{11c}, R^{11d}, and combinations thereof.

In certain embodiments, R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—; R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6; R^{11c} is —(CH₂)ₓ—, wherein the subscript of x is an integer selected from 1 to 10; and R^{11d} is selected from the group consisting of —O— and —NH.

In some embodiments of a compound of Formula (II), L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L¹ is terminated in R⁴⁰, wherein R⁴⁰ is a reactive group. In some embodiments, at least one occurrence of -(A₁)_{q}- is selected from: Val-Cit or Cit-Val. In some embodiments, L¹ is —C(O)—(C₁₋₆-alkyl)-R⁴⁰. In some embodiments, L¹ is —C(O)—(C₂H₄—O)—(C₁₋₆-alkyl)-NH—C(O)—(C₁₋₆-alkyl)-R⁴⁰. In some embodiments, L¹ is —C(O)—(C₂H₄—O)ₘ—(CH₂)₂—NH—C(O)—(CH₂)₂—R⁴⁰. In some embodiments, L¹ is —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-R⁴⁰. In some embodiments, L¹ is -(A₁)_{q}-C(O)—O—CH₂—(C₆H₄)—NH-(A₁)_{q}-C(O)—(C₁₋₆-alkyl)-R⁴⁰. In some embodiments, L¹ is di(pyrrolidin-1-yl)methyl. In some embodiments, L¹ is —C(O)—(C₂H₄—O)₂—(CH₂)₂—NH—C(O)—(CH₂)₂—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L¹ is -(Gly)-C(O)—O—CH₂—(C₆H₄)—NH-(Cit-Val)-C(O)—(CH₂)₅—R⁴⁰.

In some embodiments of a compound of Formula (II), L² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)—; wherein L² is terminated in R⁴⁰, wherein R⁴⁰ is a reactive group. In some embodiments, L² is —PO₂SH—(C₂H₄—O)—(C₁₋₆-alkyl)-R⁴⁰. In some embodiments, L² is —PO₂SH—(C₂H₄—O)ₘ—(C₁₋₆ alkyl)-NH—C(O)—O—CH₂—(CH)—NH-(A₁)_{q}-C(O)—(C₂H₄—O)ₘ—(C₁₋₆-alkyl)-R⁴⁰. In some embodiments, L² is —PO₂SH—(C₂H₄—O)ₘ—NH—C(O)—(C₂H₄—)ₘ-NH—C(O)—(C₁₋₆-alkyl)-R⁴⁰. In some embodiments, L² is —PO₂SH₂. In some embodiments, L² is —PO₃H₂. In some embodiments, L² is —PO₂SH—(C₂H₄—O)₃—(CH₂)₂—R⁴⁰. In some embodiments, L² is —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L² is —PO₂SH—(C₂H₄—O)₃—(CH₂)₂—NH—C(O)—(C₂H₄—O)₄—(CH₂)₂—NH—C(O)—(CH₂)₂—R⁴⁰.

In some embodiments of an antibody drug conjugate derived from a compound of Formula (II), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, at least one occurrence of -(A₁)_{q}- is selected from: Val-Cit or Cit-Val. In some embodiments, B¹ comprises —C(O)—(C₁₋₆-alkyl)-. In some embodiments, B¹ comprises —C(O)—(C₂H₄—O)ₘ—(C₁₋₆-alkyl)-NH—C(O)—(C₁₋₆-alkyl)-. In some embodiments, B¹ comprises —C(O)—(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)₂—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)—(CH₂)₅—.

In some embodiments of an antibody drug conjugate derived from a compound of Formula (II), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 of instances —(C₂H₄—O)ₘ—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of -(C₁₋₆-alkyl)-, 0-2 instances of —O—CH₂—(C₆H₄)—NH—, and 0-3 instances of —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—.

In certain embodiments, L¹ or L² are terminated in R⁴⁰, wherein R⁴⁰ is a reactive group. In certain embodiments, R¹¹ is terminated in R⁴⁰, wherein R⁴⁰ is a reactive group. In some embodiments, the reactive group is -NHBoc

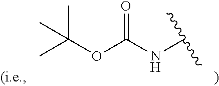 [SMILES: *NC(=O)OC(C)(C)C]

or —NH₂. In some embodiments, the reactive group is maleimide.

In some embodiments of a compound of Formula (II), when L¹ is H, then at least one occurrence of L² is not H; and when all occurrences of L² are H, then L¹ is not H.

In some embodiments of an antibody drug conjugate derived from a compound of Formula (II), B¹ comprises the reaction product of R⁴⁰ as found in L¹ with Ab, such that a covalent linkage is formed therebetween. In some embodiments, B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

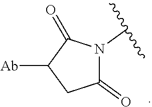

In certain embodiments of a compound of Formula (II) or an antibody drug conjugate derived therefrom, A₁ and A₂ are independent in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of a compound of Formula (II) or an antibody drug conjugate derived therefrom, subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently selected from an integer selected from 0 to 4; each instance of subscript m is independently selected from an integer selected from 0 to 20; each instance of subscript p is independently selected from an integer 0 or 1; each instance of subscript q is independently selected from an integer selected from 1 to 6; and each instance of subscript n is independently selected from an integer selected from 0 to 10.

Provided herein is a compound of Formula (III), or a pharmaceutically acceptable salt, ester, stereoisomer or tautomer thereof, comprising

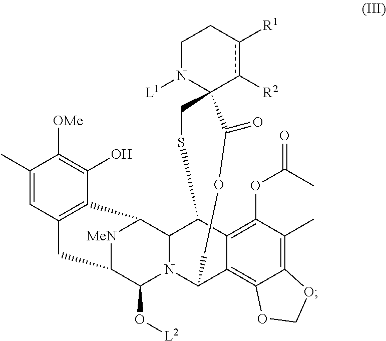

wherein R¹ and R² form at least one aromatic ring or bicyclic heterocyclic ring, optionally substituted with one to six substituents, independently in each instance, selected from C₁₋₁₀-alkoxy, C₁₋₁₀-alkyl, amino, hydroxyl, nitro, —P(O)(OH)₂, thiol, and —SO₃H; wherein [symbol] is either a single or a double bond.

In certain embodiments, L¹ comprises at least one of the following:
(a) —H, wherein at least one of L¹ or L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S.

In certain embodiments, R¹⁰ is R^{10a}-R^{10b}, wherein R^{10a} is either absent or —(CH₂)ₑ, wherein e is independently in each instance, selected from 1-4; and R^{10b} is selected from the group consisting of:

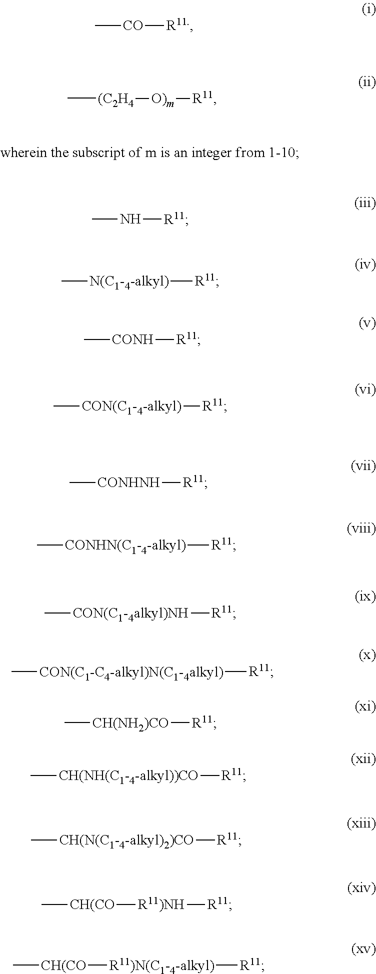

wherein R¹¹ is selected from the group consisting of R^{11a}, R^{11b}, R^{11c}, R^{11d}, and combinations thereof;
wherein R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
R^{11c} is —(CH₂)ₓ—, wherein the subscript of x is an integer selected from 1 to 10; and
R^{11d} is selected from the group consisting of —O— and —NH.

In certain embodiments, L² comprises at least one of the following:
(a) —H, wherein at least one of L¹ or L² is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S.
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
R^{10a} is either absent or —(CH₂)ₑ—, wherein e is independently in each instance, selected from 1-4;
R^{10b} is selected from the group consisting of:

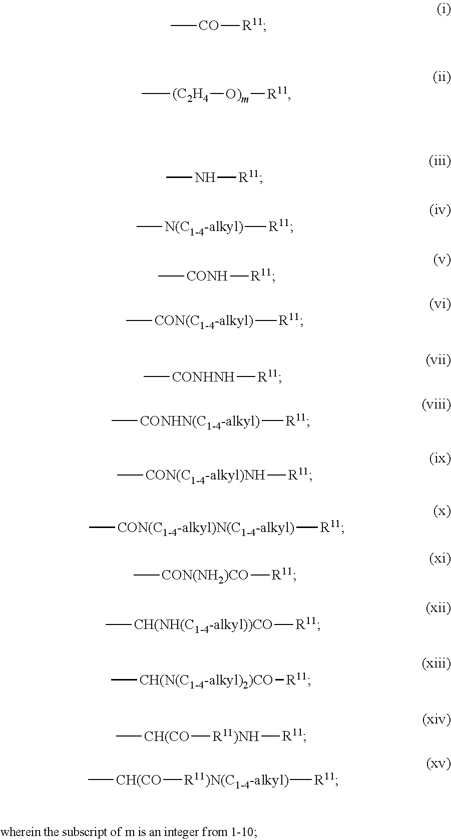

wherein R¹¹ is selected from the group consisting of R^{11a}, R^{11b}, R^{11c}, R^{11d}, and combinations thereof; wherein
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—; R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6; R^{11c} is —(CH₂)ₓ—, wherein the subscript of x is an integer selected from 1 to 10; and R^{11d} is selected from the group consisting of —O— and —NH.

In some embodiments of a compound of Formula (III), L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L¹ is terminated in R⁴⁰, wherein R⁴⁰ is a reactive group. In some embodiments, at least one occurrence of -(A₁)_{q}- is selected from: Val-Cit or Cit-Val. In some embodiments, L¹ is —C(O)—(C₁₋₆-alkyl)-R⁴⁰. In some embodiments, L¹ is —C(O)—(C₂H₄—O)ₘ—(C₁₋₆-alkyl)-NH—C(O)—(C₁₋₆-alkyl)-R⁴⁰. In some embodiments, L¹ is —C(O)—(C₂H₄—O)ₘ—(CH₂)₂—NH—C(O)—(CH₂)₂—R⁴⁰. In some embodiments, L¹ is —C(O)—(C₂H₄—O)ₘ—NH—C(O)—O—CH₂—(CH₄)—NH-(A₁)_{q}-C(O)—(C₂H₄—O)—C₁₋₆-alkyl)-R⁴⁰. In some embodiments, L¹ is -(A₁)_{q}-C(O)—O—CH₂—(C₆H₄)—NH-(A₁)_{q}-C(O)—(C₁₋₆-alkyl)-R⁴⁰. In some embodiments, L¹ is di(pyrrolidin-1-yl)methyl. In some embodiments, L¹ is —C(O)—(C₂H₄—O)₂—(CH₂)₂—NH—C(O)—(CH₂)₂—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L¹ is -(Gly)-C(O)—O—CH₂—(CH₄)—NH-(Cit-Val)-C(O)—(CH₂)₅—R⁴⁰.

In some embodiments of a compound of Formula (III), L² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² is —PO₂SH—(C₂H₄—O)ₘ—(C₁₋₆-alkyl)-R⁴⁰. In some embodiments, L² is —PO₂SH—(C₂H₄—O)ₘ—(C₁₋₆ alkyl)-NH—C(O)—O—CH₂—(CH₄)—NH-(A₁)_{q}-C(O)—(C₂H₄—O)ₘ—(C₁₋₆-alkyl)-R⁴⁰. In some embodiments, L2 is —PO₂SH—(C₂H₄—O)ₘ—NH—C(O)—(C₂H₄—O)ₘ—NH—C(O)—(C₁₋₆-alkyl)-R⁴⁰. In some embodiments, L² is —PO₂SH₂. In some embodiments, L² is —PO₃H₂. In some embodiments, L² is —PO₂SH—(C₂H₄—O)₃—(CH₂)₂—R⁴⁰. In some embodiments, L² is —PO₂SH—(C₂H₄—O)₃—(CH₂)₂—NH—C(O)—O—CH₂—(C₆H₄)—NH-(Cit-Val)-C(O)—(C₂H₄—O)₂(CH₂)₂—R⁴⁰. In some embodiments, L² is —PO₂SH—(C₂H₄—O)₃—(CH₂)₂—NH—C(O)—(C₂H₄—O)₄—(CH₂)₂—NH—C(O)—(CH₂)₂—R⁴⁰.

In some embodiments of an antibody drug conjugate derived from a compound of Formula (III), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, at least one occurrence of -(A₁)_{q}- is selected from: Val-Cit or Cit-Val. In some embodiments, B¹ comprises —C(O)—(C₁₋₆-alkyl)-. In some embodiments, B¹ comprises —C(O)—(C₂H₄—O)ₘ—(C₁₋₆-alkyl)-NH—C(O)—(C₁₋₆-alkyl)-. In some embodiments, B¹ comprises —C(O(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)₂—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)—(CH₂)₅—.

In some embodiments of an antibody drug conjugate derived from a compound of Formula (III), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)_{q}—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—.

In certain embodiments of a compound of Formula (III), L¹ or L2 are terminated in R⁴⁰, wherein R⁴⁰ is a reactive group. In certain embodiments, R¹¹ is terminated in R⁴⁰, wherein R⁴⁰ is a reactive group. In some embodiments, the reactive group is -NHBoc

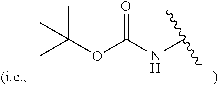 [SMILES: *NC(=O)OC(C)(C)C]

or —NH₂. In some embodiments, the reactive group is maleimide.

In some embodiments of a compound of Formula (III), when L¹ is H, then at least one occurrence of L² is not H; and when all occurrences of L² are H, then L¹ is not H.

In some embodiments of an antibody drug conjugate derived from a compound of Formula (I), B¹ comprises the reaction product of R as found in L¹ with Ab, such that a covalent linkage is formed therebetween. In some embodiments, B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

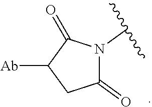

In certain embodiments of a compound of Formula (III) or an antibody drug conjugate derived therefrom, A₁ and A₂ are independent in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of a compound of Formula (III), subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently selected from an integer selected from 0 to 4; each instance of subscript m is independently selected from an integer selected from 0 to 20; each instance of subscript p is independently selected from an integer 0 or 1; each instance of subscript q is independently selected from an integer selected from 1 to 6; and each instance of subscript n is independently selected from an integer selected from 0 to 10.

In certain embodiments, the compound can be of Formula (IV) or a pharmaceutically acceptable salt, ester, or tautomer thereof, comprising:

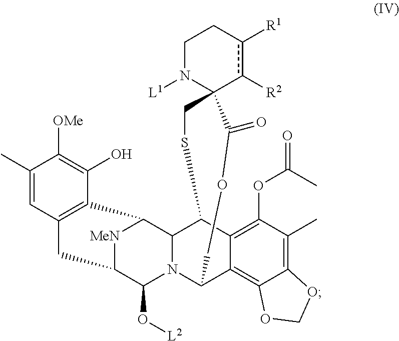

wherein R¹ and R² form at least one aromatic ring or bicyclic heterocyclic ring, optionally substituted with one to six substituents, independently in each instance, selected from C₁₋₁₀ alkoxy, C₁₋₁₀ alkyl, amino, hydroxyl, nitro, —P(O)(OH)₂, thiol, and —SO₃H; wherein [symbol] is either a single or a double bond.

In certain embodiments, L¹ is R⁴⁰—(C₁₋₆ alkyl)-[(C(O)—NH]ₙ—(—C₂H₄—O)ₘ—(C₁₋₆ alkyl)_{q}-[(C(O)—]ₚ; or R⁴⁰—(—C₂H₄—O)ₘ—C(O)-(A₁-A₂)_{q}-[—NH]ₙ—(C₆H₄)—CH₂—O—[(C(O)—NH]ₙ—(—C₂H₄—O)ₜ—(C₁₋₆ alkyl)-[(C(O)—]ₚ. L² is R⁴⁰—(C₁₋₆ alkyl)-[(C(O)—NH]ₙ—(—C₂H₄—O)ₘ—(C₁₋₆alkyl)-[(C(O)—NH]ₚ—(—C₂H₄—O)ₘ—[O—P(O)SH]; or R⁴⁰—(—C₂H₄—O)ₘ—C(O)-(A₁-A₂)_{q}-NH—(CH₄)—CH₂—O—[(C(O)—NH]ₙ—(—C₂H₄—O)ₜ—(C₁₋₆ alkyl)-[(C(O)—]ₚ-[O—P(O)SH]. In certain embodiments, L¹ is R⁴⁰—(C₁₋₆ alkyl)-C(O)-(A₁-A₂)_{g}-[—NH]ₕ—(C₆H₄)—CH₂—O—[(C(O)—NH]ⱼ—.

In certain embodiments, R⁴⁰ can be a reactive group; wherein A₁ and A₂ are independent in each instance an amino acid; wherein subscript n is, independently in each instance, selected from 0 to 4; wherein subscript m is, independently in each instance, selected from 0 to 20; wherein subscript p is, independently in each instance, 0 or 1; wherein subscript q is, independently in each instance, 0 or 1; and wherein subscript t is, independently in each instance, selected from 0 to 10. The linker can be cleavable or non-cleavable.

In certain embodiments, A₁-A₂ can be valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some embodiments of a compound of Formula (IV), L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L¹ is terminated in R⁴⁰, wherein R⁴⁰ is a reactive group. In some embodiments, at least one occurrence of -(A₁)_{q}- is selected from: Val-Cit or Cit-Val. In some embodiments, L¹ is —C(O)-(Cia-alkyl)-R⁴⁰. In some embodiments, L¹ is —C(O)—(C₂H₄—O)ₘ—(C₁₋₆-alkyl)-NH—C(O)—(C₁₋₆-alkyl)-R⁴⁰. In some embodiments, L¹ is —C(O)—(C₂H₄—O)ₘ—(CH₂)₂—NH—C(O)—(CH₂)₂—R⁴⁰. In some embodiments, L¹ is —C(O)—(C₂H₄—O)ₙ—NH—C(O)—O—CH₂—(C₆H₄)—NH-(A₁)-C(O)—(C₂H₄—O)ₘ—(C₁₋₆-alkyl)-R⁴⁰. In some embodiments, L¹ is -(A₁)_{q}-C(O)—O—CH₂—(C₆H₄)—NH-(A₁)_{q}-C(O)—(C₁₋₆-alkyl)-R⁴⁰. In some embodiments, L¹ is di(pyrrolidin-1-yl)methyl. In some embodiments, L¹ is —C(O)—(C₂H₄—O)₂—(CH₂)₂—NH—C(O)—(CH₂)₂—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L¹ is -(Gly)-C(O)—O—CH₂—(C₆H₄)—NH-(Cit-Val)-C(O)—(CH₂)₅—R⁴⁰.

In some embodiments of a compound of Formula (IV), L² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—; wherein L² is terminated in R⁴⁰, wherein R⁴⁰ is a reactive group. In some embodiments, L² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—; wherein L² is terminated in R⁴⁰, wherein R⁴⁰ is a reactive group. In some embodiments, L² is —PO₂SH—(C₂H₄—O)ₘ—(C₁₋₆-alkyl)-R⁴⁰. In some embodiments, L² is —PO₂SH—(C₂H₄—O)ₘ—(C₁₋₆ alkyl)-NH—C(O)—O—CH₂—(C₆H₄)—NH-(A₁)_{q}-C(O)—(C₂H₄—O)ₘ—(C₁₋₆-alkyl)-R⁴⁰. In some embodiments, L² is —PO₂SH—(C₂H₄—O)—NH—C(O)—(C₂H₄—O)ₘ—NH—C(O)—(C₁₋₆-alkyl)-R⁴⁰. In some embodiments, L² is —PO₂SH₂. In some embodiments, L² is —PO₃H₂. In some embodiments, L² is —PO₂SH—(C₂H₄—O)₃—(CH₂)₂—R⁴⁰. In some embodiments, L² is —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L² is —PO₂SH—(C₂H₄—O)₃—(CH₂)₂—NH—C(O)—(C₂H₄—O)₄—(CH₂)₂—NH—C(O)—(CH₂)₂—R⁴⁰.

In some embodiments of an antibody drug conjugate derived from a compound of Formula (IV), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—. In some embodiments, at least one occurrence of -(A₁)_{q}- is selected from: Val-Cit or Cit-Val. In some embodiments, B¹ comprises —C(O)—(C₁₋₆-alkyl)-. In some embodiments, B¹ comprises —C(O)—(C₂H₄—O)ₘ—(C₁₋₆-alkyl)-NH—C(O)—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)—(CH₂)₅—.

In some embodiments of an antibody drug conjugate derived from a compound of Formula (IV), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, and 0-3 instances —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—.

In certain embodiments of a compound of Formula (IV), L¹ or L² are terminated in R⁴⁰, wherein R⁴⁰ is a reactive group. In certain embodiments, R⁴⁰ is terminated in R⁴⁰, wherein R⁴⁰ is a reactive group. In some embodiments, the reactive group is -NHBoc

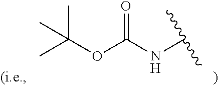 [SMILES: *NC(=O)OC(C)(C)C]

or —NH₂. In some embodiments, the reactive group is maleimide.

In some embodiments of a compound of Formula (IV), when L¹ is H, then at least one occurrence of L² is not H; and when all occurrences of L² are H, then L¹ is not H.

In some embodiments of an antibody drug conjugate derived from a compound of Formula (IV), B¹ comprises the reaction product of R⁴⁰ as found in L¹ with Ab, such that a covalent linkage is formed therebetween. In some embodiments, B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

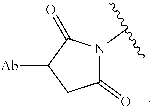

In certain embodiments of a compound of Formula (IV) or an antibody drug conjugate derived therefrom, A₁ and A₂ are independent in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of a compound of Formula (IV), subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently selected from an integer selected from 0 to 4; each instance of subscript m is independently selected from an integer selected from 0 to 20; each instance of subscript p is independently selected from an integer 0 or 1; each instance of subscript q is independently selected from an integer selected from 1 to 6; and each instance of subscript n is independently selected from an integer selected from 0 to 10.

In certain embodiments, the compound (e.g., a compound of Formula (IV), a compound of Formula (III)) is:

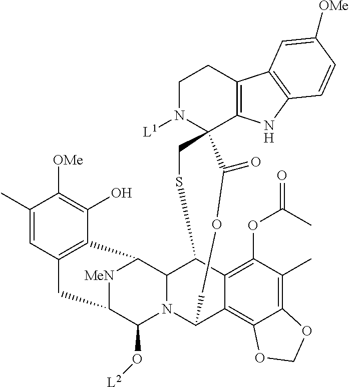

In certain embodiments, the compound (e.g., a compound of Formula (IV), a compound of Formula (III)) is:

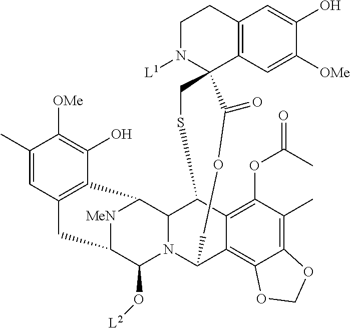

In certain embodiments, the linker L¹ or L² independently comprises:

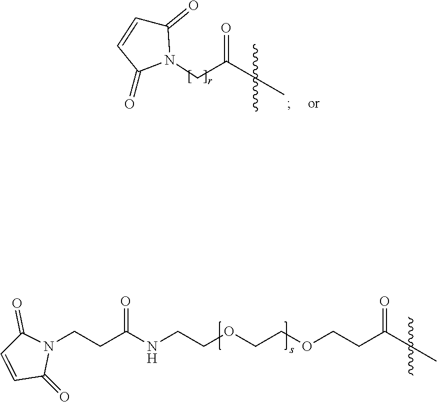

wherein

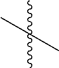

indicates the point of attachment to the compound of Formula (IV) or to a remaining portion of L¹ or L²; wherein r is an integer select from 1 to 12; and wherein s is an integer select from 1 to 32.

In an example, the compound (e.g., a compound of Formula (IV), a compound of Formula (III)) can be

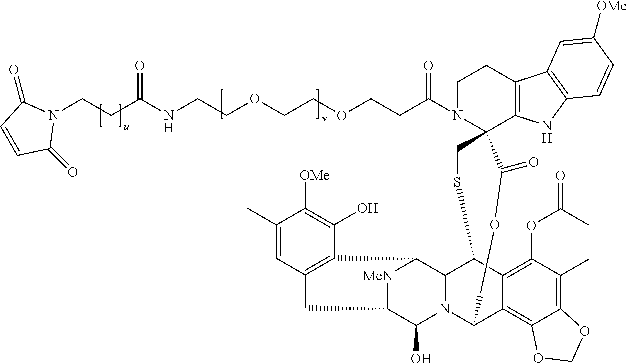

wherein subscript v is an integer from 1 to 32, 1 to 14, or 1 to 8; and wherein subscript u is an integer between 0 and 11.

In an example, the compound (e.g., a compound of Formula (III), a compound of Formula (IV)) can be

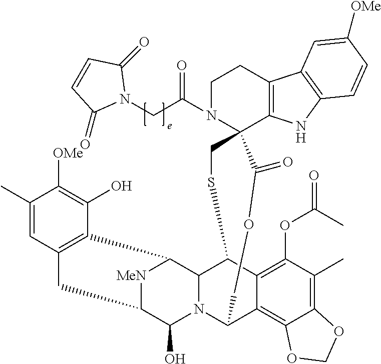

wherein subscript e is an integer selected from 1 to 6, 1 to 4, or 1 to 3.

In certain embodiments, the compound of Formula (III) or (IV) can be at least one of the compounds in Table B.

TABLE B

| | |
|---|---|
| II-A | 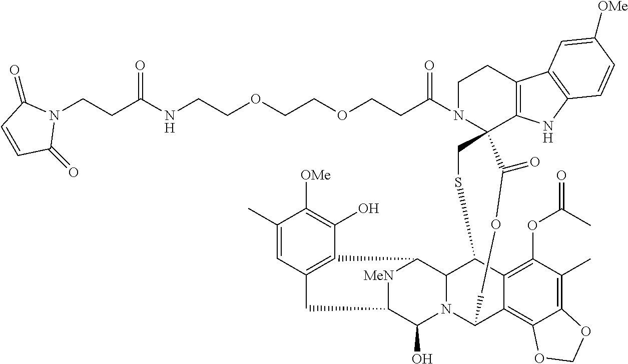 [SMILES: COc1ccc2[nH]c3c(c2c1)CCN(C(=O)CCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-B | 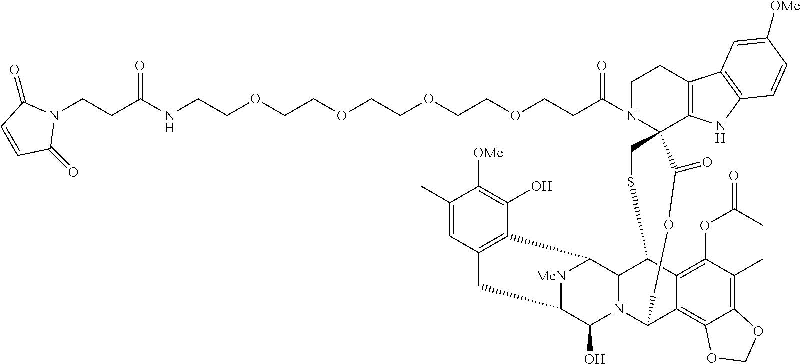 [SMILES: COc1ccc2[nH]c3c(c2c1)CCN(C(=O)CCOCCOCCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-C | 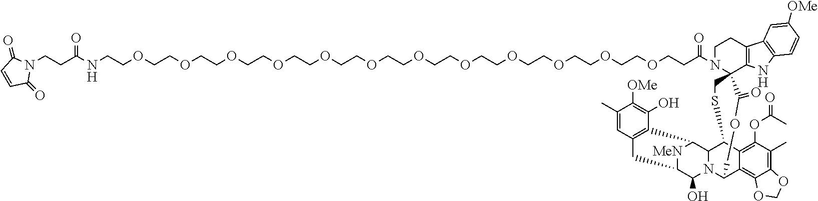 [SMILES: COc1ccc2[nH]c3c(c2c1)CCN(C(=O)CCOCCOCCOCCOCCOCCOCCOCCOCCOCCOCCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-D | 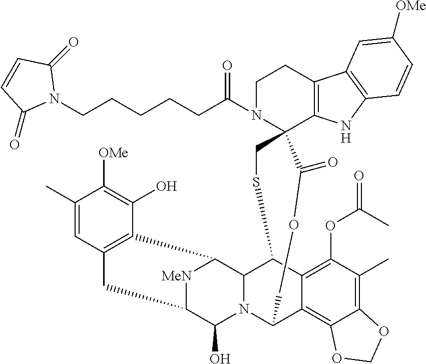 [SMILES: COc1ccc2[nH]c3c(c2c1)CCN(C(=O)CCCCCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-E | 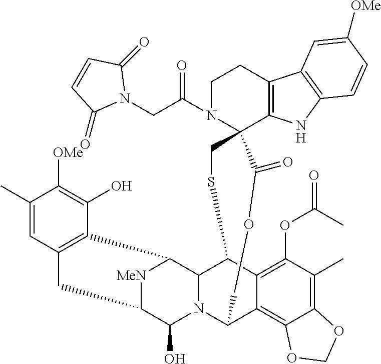 [SMILES: COc1ccc2[nH]c3c(c2c1)CCN(C(=O)CN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-F | 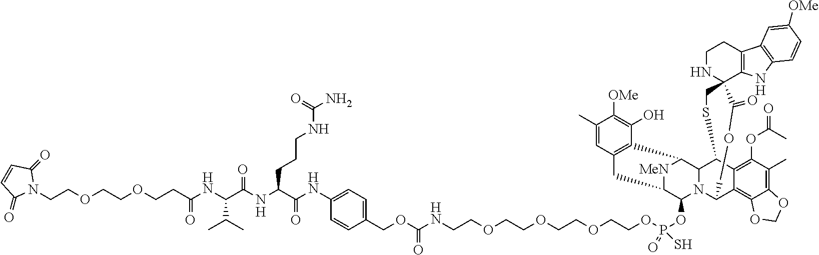 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4] |
| II-G | 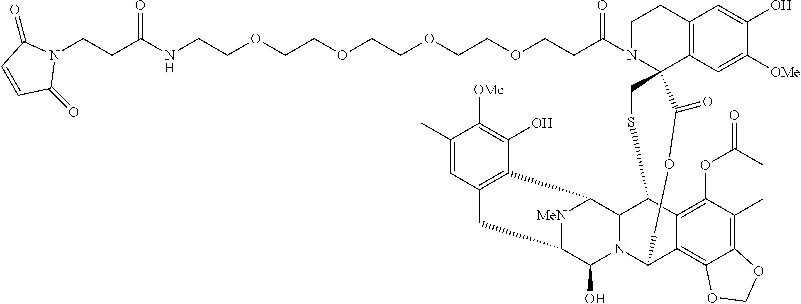 [SMILES: COc1cc2c(cc1O)CCN(C(=O)CCOCCOCCOCCOCCNC(=O)CCN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-H | 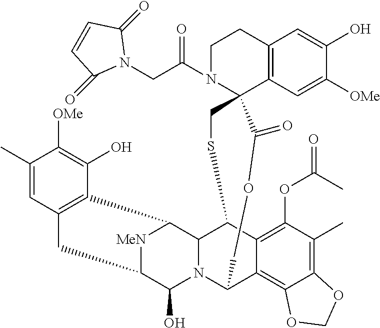 [SMILES: COc1cc2c(cc1O)CCN(C(=O)CN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-I | 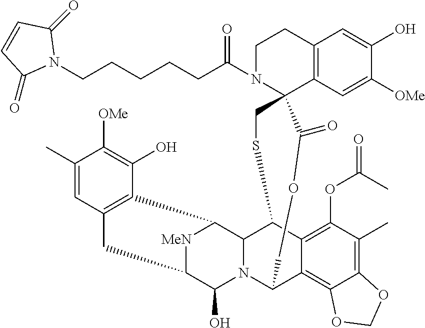 [SMILES: COc1cc2c(cc1O)CCN(C(=O)CCCCCN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-J | 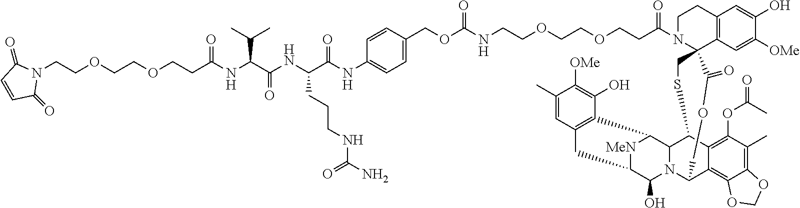 [SMILES: COc1cc2c(cc1O)CCN(C(=O)CCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |
| II-K | 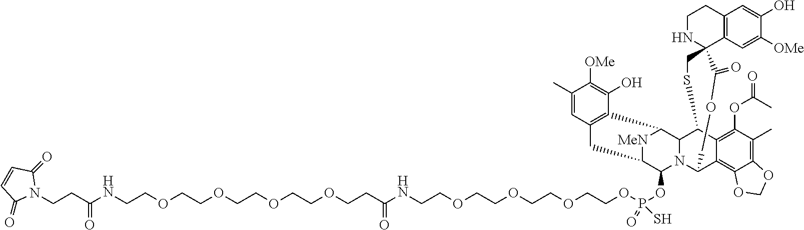 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)CCOCCOCCOCCOCCNC(=O)CCN1C(=O)C=CC1=O)N2C)OCO4] |
| II-L | 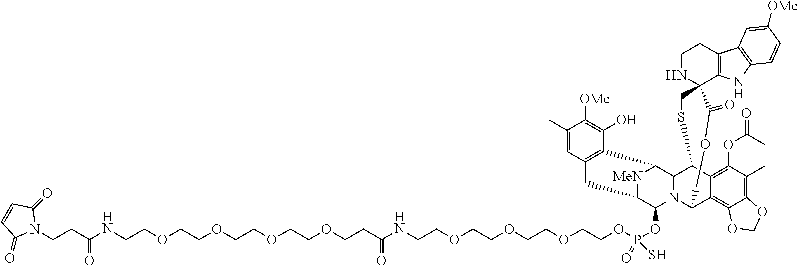 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)CCOCCOCCOCCOCCNC(=O)CCN1C(=O)C=CC1=O)N2C)OCO4] |
| II-M | 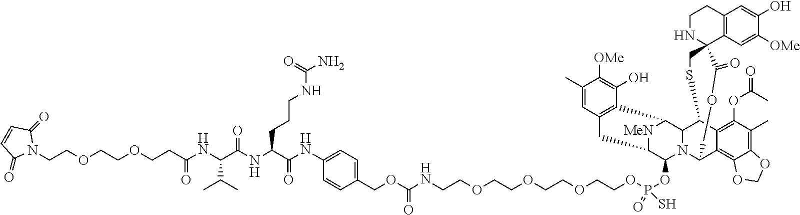 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4] |
| II-N | 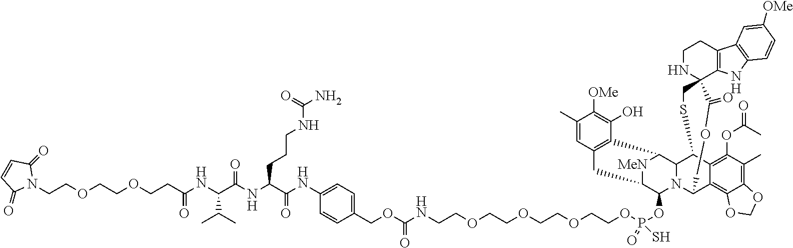 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4] |
| II-O | 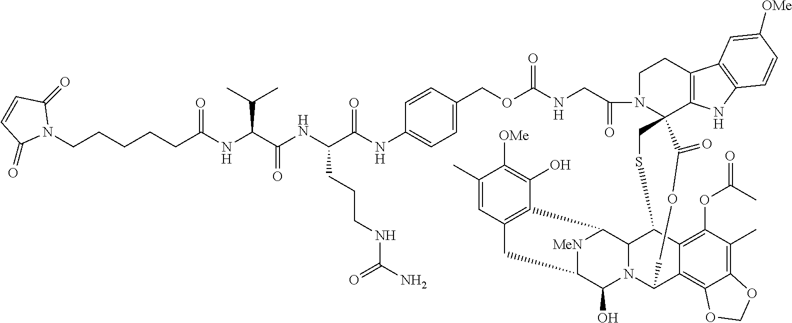 [SMILES: COc1ccc2[nH]c3c(c2c1)CCN(C(=O)CNC(=O)OCc1ccc(NC(=O)[C@H](CCCNC(N)=O)NC(=O)[C@@H](NC(=O)CCCCCN2C(=O)C=CC2=O)C(C)C)cc1)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4] |

Also provided herein is a compound of Formula (Va) or (Vb), or a pharmaceutically acceptable salt, ester, stereoisomer or tautomer thereof, comprising:

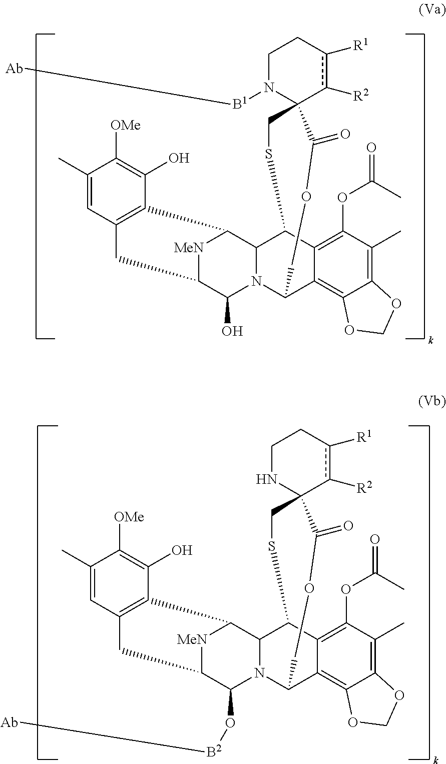

wherein Ab is targeting agent; wherein subscript k is an integer from 1 to 10;
wherein R¹ and R² form at least one aromatic ring or bicyclic heterocyclic ring, optionally substituted with one to six substituents, independently in each instance, selected from C₁₋₁₀ alkoxy, C₁₋₁₀ alkyl, amino, hydroxyl, nitro, —P(O)(OH)₂, thiol, and —SO₃H; and wherein one of B¹ or B² is a linker. Ab can be an antibody, antibody fragment, protein, or peptide.

In certain embodiments, B¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;

In certain embodiments, R¹⁰ is R^{10a}-R^{10b}, wherein R^{10a} is either absent or —(CH₂)ₑ, wherein e is independently in each instance, selected from 1-4.

In certain embodiments, R^{10b} is selected from the group consisting of:

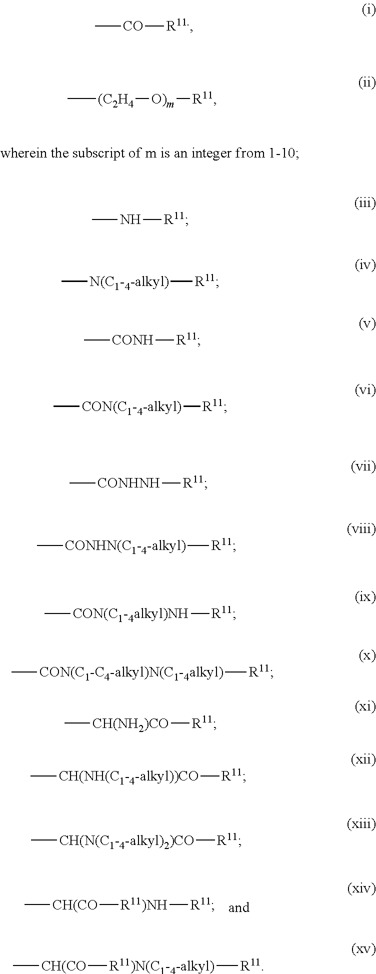

In certain embodiments, R⁴⁰ is selected from the group consisting of R^{11a}, R^{11b}, R^{11c}, R^{11d}, and combinations thereof; wherein R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—; R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6; R^{11c} is —(CH₂)ₓ—, wherein the subscript of x is an integer selected from 1 to 10; and R^{11d} is selected from the group consisting of —O— and —NH.

In certain embodiments, B² comprises at least one of the following:
(a) —H, wherein at least one of L¹ or L² is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S.

In certain embodiments, R¹⁰ is R^{10a}-R^{10b}, wherein
R^{10a} is either absent or —(CH₂)ₑ—, wherein e is independently in each instance, selected from 1-4;
R^{10b} is selected from the group consisting of:

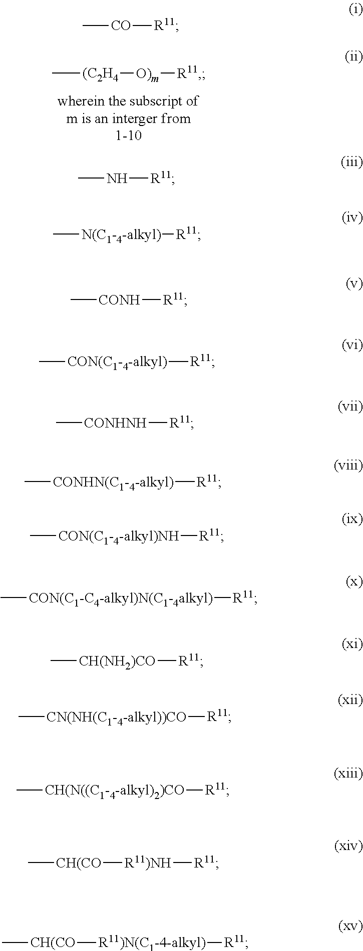

In certain embodiments, R¹¹ is selected from the group consisting of R^{11a}, R^{11b}, R^{11c}, R^{11d}, and combinations thereof, wherein R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—; R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6; R^{11c} is —(CH₂)ₓ, wherein the subscript of x is an integer selected from 1 to 10; and R¹Id is selected from the group consisting of —O— and —NH.

In certain embodiments, L¹ is R⁵⁰—(C₁₋₆ alkyl)-[(C(O)—NH]ₙ—(—C₂H₄—O)ₘ—(C₁₋₆ alkyl)_{q}-[(C(O)—]ₚ; or R⁴⁰—(—C₂H₄—O)ₘ—C(O)-(A₁-A₂)_{q}-[—NH]ₙ—(CH₄)—CH₂—O—[(C(O)—NH]ₙ—(—C₂H₄—O)—(C₁₋₆ alkyl)-[(C(O)—]ₚ. In certain embodiments, L² is R⁵⁰—(C₁₋₆alkyl)-[(C(O)—NH]ₙ—(—C₂H₄—O)ₘ—(C₁₋₆ alkyl)-[(C(O)—NH]ₚ—(—C₂H₄—O)ₘ—[O—P(O)SH]; or R⁴⁰—(—C₂H₄—O)ₘ—C(O)-(A₁-A₂)_{q}-NH—(C₆H₄)—CH₂—O—[(C(O)—NH]ₙ—(—C₂H₄—O)—(C₁₋₆alkyl)-[(C(O)—]ₚ-[O—P(O)SH]. R⁴⁰ can be a conjugation unit.

In some embodiments of a compound of Formula (Va) or Formula (Vb), Ab is an antibody, antibody fragment, protein, or peptide. In some embodiments, the antibody or antibody fragment includes one or more of SEQ ID NOs. 1 to 20 as listed in Table 6. Non-limiting examples of antibodies include trastuzumab and brentuximab.

In some embodiments, a compound of Formula (Va) or a compound of Formula (Vb) is an antibody drug conjugate derived from a compound of Formula (I). In some embodiments, a compound of Formula (Va) or a compound of Formula (Vb) is an antibody drug conjugate derived from a compound of Formula (II). In some embodiments, a compound of Formula (Va) or a compound of Formula (Vb) is an antibody drug conjugate derived from a compound of Formula (III). In some embodiments, a compound of Formula (Va) or a compound of Formula (Vb) is an antibody drug conjugate derived from a compound of Formula (IV).

In certain embodiments, the compound (e.g., a compound of Formula (Va) or (Vb)) can be

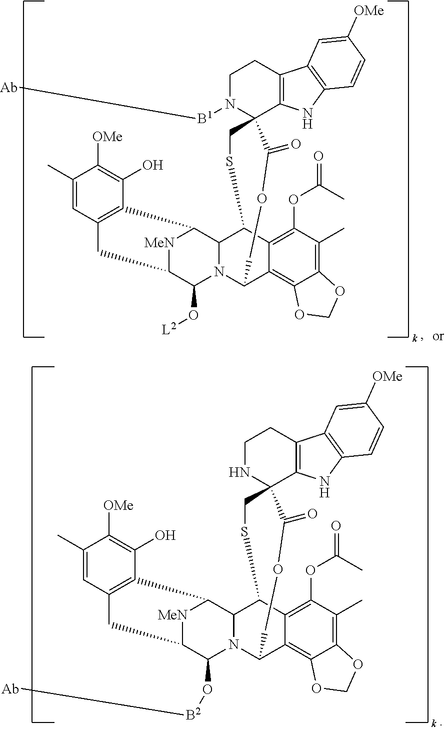

In certain embodiments, the compound (e.g., a compound of Formula (Va) or (Vb)) can be

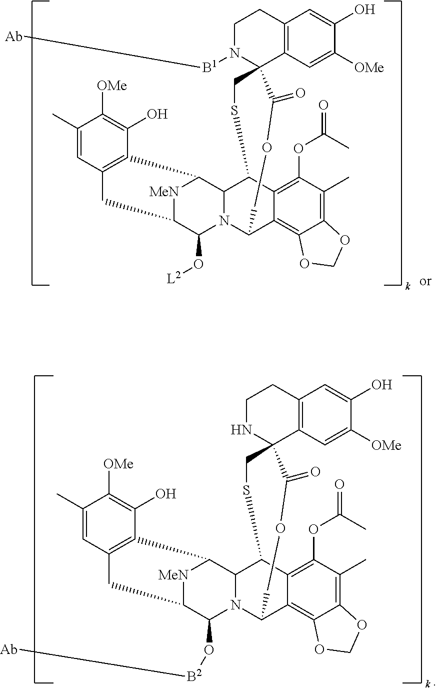

In certain embodiments, the compound (e.g. a compound of Formula (Va) or (Yb)) can be at least one of the compounds listed in Table C.

TABLE C

| | |
|---|---|
| III-A | 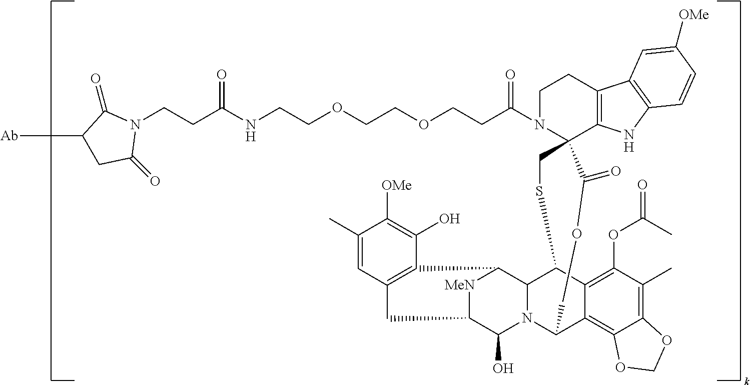 |
| III-B | 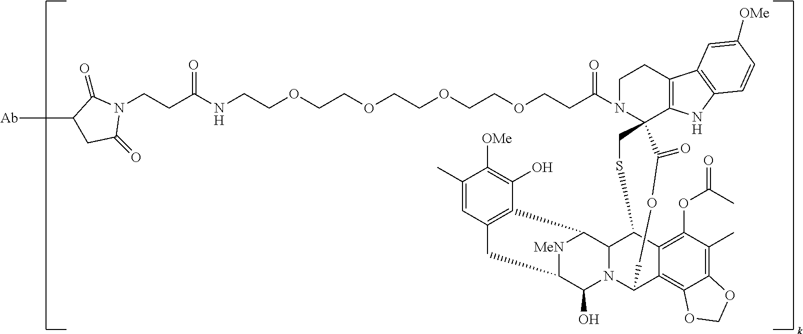 |
| III-C | 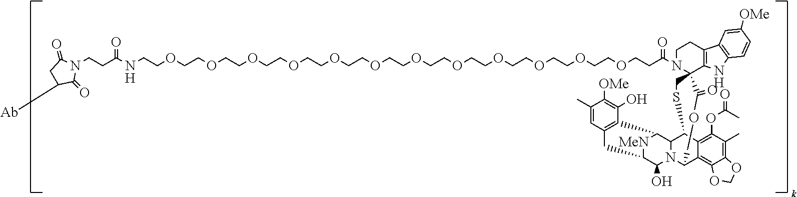 |
| III-D | 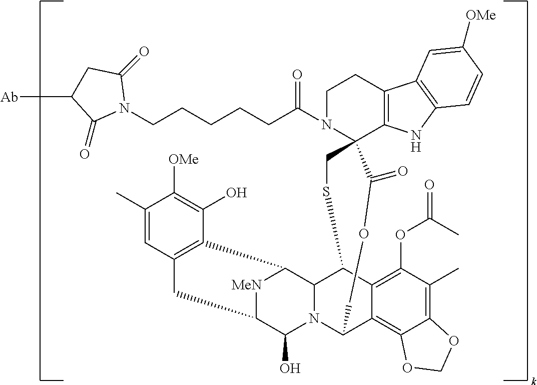 |
| III-E | 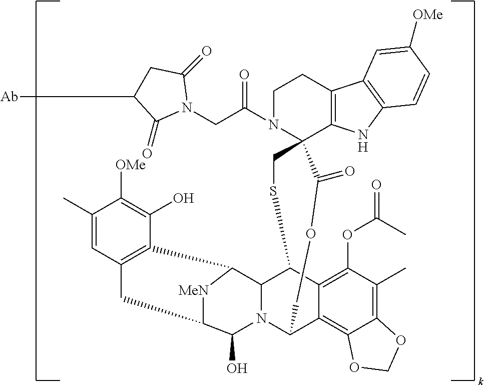 |
| III-F | 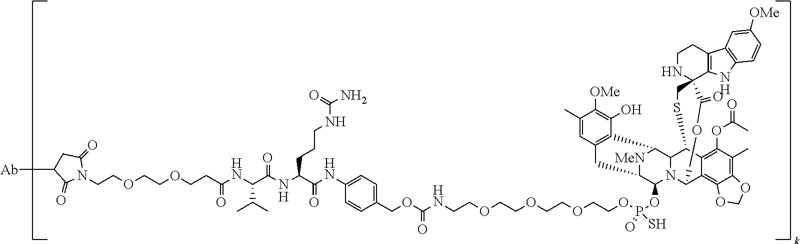 |
| III-G | 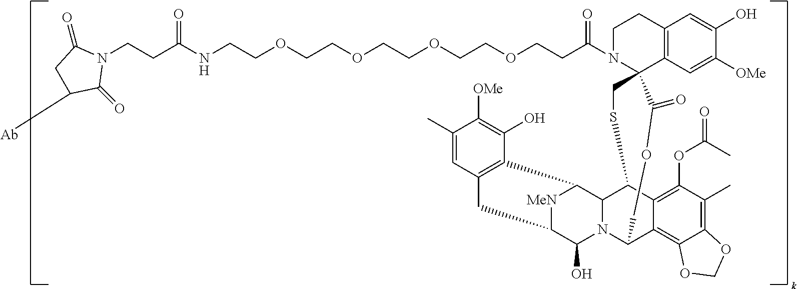 |
| III-H | 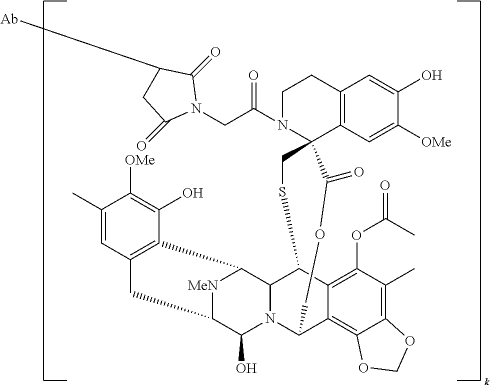 |
| III-I | 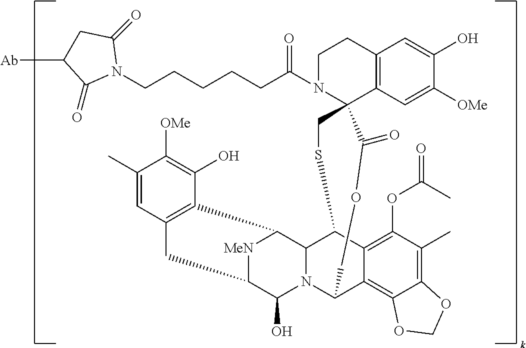 |
| III-J | 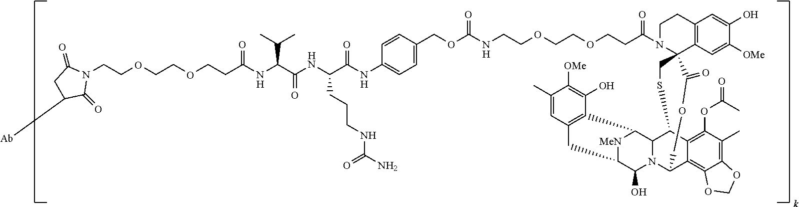 |
| III-K | 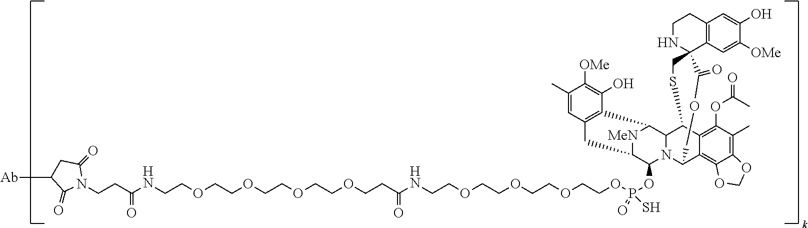 |
| III-L | 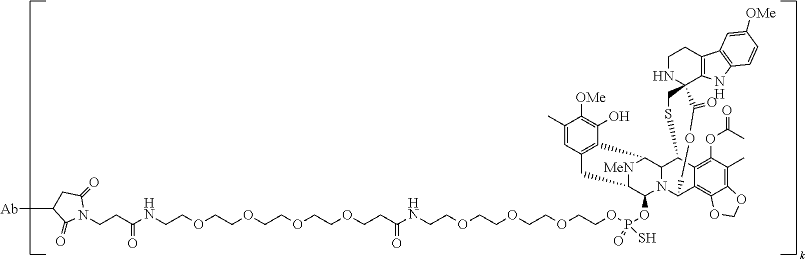 |
| III-M | 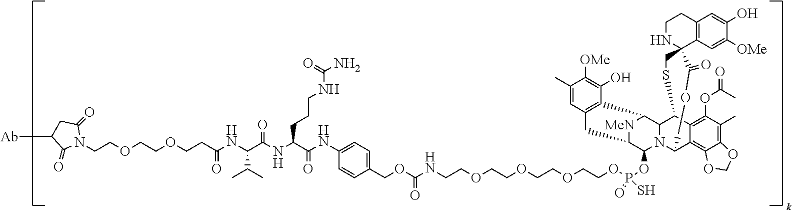 |
| III-N | 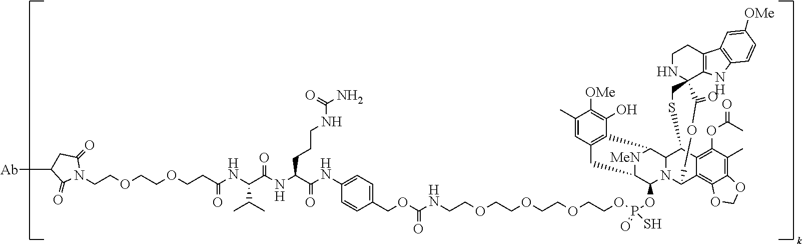 |
| III-O | 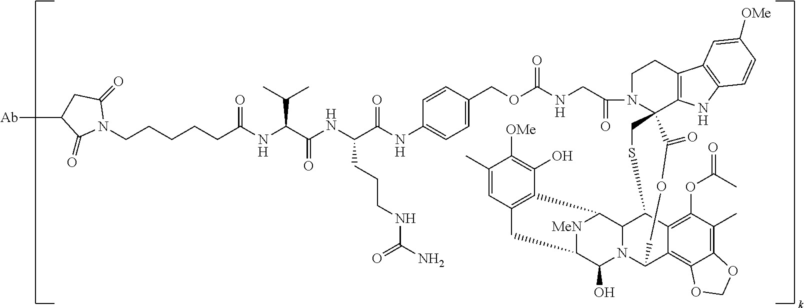 |

In certain embodiments, provided herein are compounds according to Formula (XI), or a pharmaceutically acceptable salt, ester, stereoisomer, or tautomer thereof:

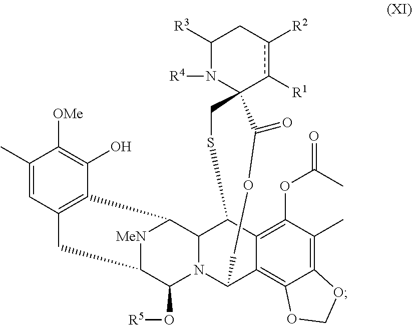

wherein:
R¹ and R² taken together to form an aromatic ring or bicyclic heteroaromatic ring; wherein the aromatic ring and bicyclic heteroaromatic ring are optionally substituted with one to six substituents independently selected from the group consisting of C₁₋₁₀-alkoxy, C₁₋₁₀-alkyl, amino, hydroxyl, halogen, nitro, —P(O)(OH)₂, —SH, and —SO₃H;
R³ is hydrogen or CH₂OR⁶;
R⁴ is H, C₁₋₁₀-alkyl, C₆₋₁₀-aryl, C₃₋₁₀-heterocycle, C(O)R⁸, C(O)OR⁸, C(O)NHR, SO₂R⁸, SO₂NHR⁸; wherein the C₁₋₁₀-alkyl or C₆₋₁₀-aryl is optionally substituted with 1-3 substituents independently selected from the group consisting of —OH, —F, —Cl, —Br, —C₁₋₆-alkyl, —C₃₋₆-cycloalkyl, and —C₃₋₆-heterocyclyl; or L¹
R⁵ and R⁶ are each independently selected from the group consisting of H, C₁₋₁₀-alkyl, C₆₋₁₀-aryl, phosphate, thiophosphate, phosphoramide, C(O)R, C(O)OR⁹, and C(O)NHR⁹, SO₃H, SO₂R⁹, wherein the C₁₋₁₀-alkyl or C₆₋₁₀-aryl is optionally substituted with 1-3 substituents independently selected from the group consisting of —OH, —F, —Cl, —Br, optionally substituted —C₁₋₆-alkyl, optionally substituted —C₃₋₆-cycloalkyl, and optionally substituted —C₃₋₆-heterocyclyl; or L2
wherein the phosphate and thiophosphate of R⁵ or R⁶ are optionally substituted with one to six substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
   wherein the substituent on the phosphate and thiophosphate is optionally terminated with a protecting group;
R⁸ is selected from the group consisting of C₁₋₆-alkyl, C₃₋₁₀-cycloalkyl, [PEG]_{y}, C₆₋₁₀-aryl, and C₆₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
R⁹ is selected from the group consisting of C₁₋₆-alkyl, C₃₋₁₀-cycloalkyl, [PEG]_{y}, C₆₋₁₀-aryl, and C₆₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
wherein one of R⁴, R⁵, and R⁶ is a linker to a reactive group R¹⁰; or is a linker to an antibody Ab;
wherein [symbol] is either a single or a double bond; and
wherein y is an integer selected from 0 to 32.

In certain embodiments of Formula (XI), L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₄-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰; or
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

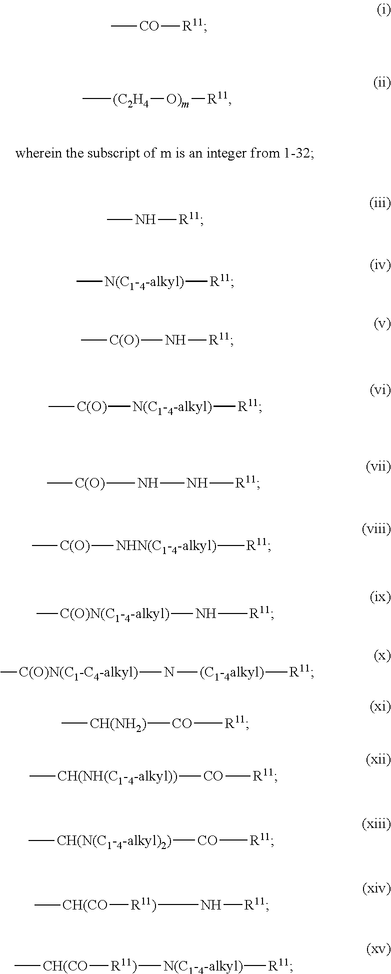

wherein R¹¹ is selected from the group consisting of R^{11a}, R^{11b}, R^{11c}, R^{11d}, and combinations thereof; wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

In certain embodiments of Formula (XI), L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰.
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

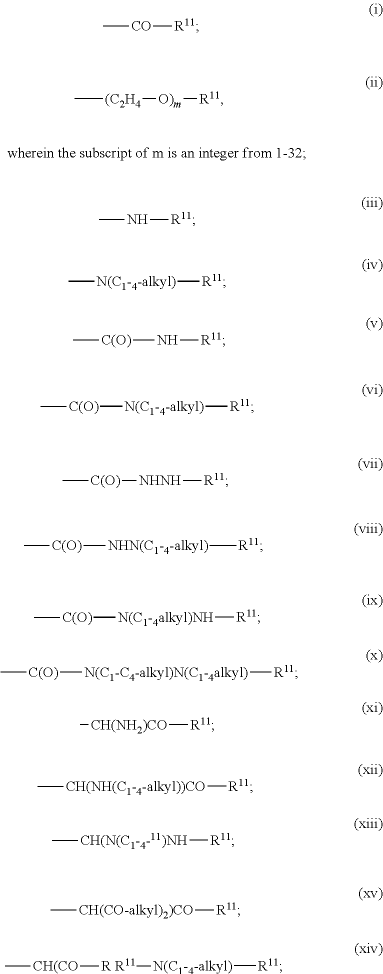

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
   R^{11c} is —(CH₂)ₓ—, wherein the subscript of x is an integer selected from 1 to 10; and
R^{11d} is selected from the group consisting of —O— and —NH.

In certain embodiments, R¹¹ is selected from:
R^{11a}-R^{11b}-R^{11c}-R^{11d};
R^{11b}-R^{11c}-R^{11d};
R^{11c}-R^{11d}; or
R^{11a}-R^{11a}-R^{11d}.

In certain embodiments, R¹¹ is terminated in a reactive group, R⁴⁰.

In certain embodiments, L¹ comprises at least one of the following bivalent structures:

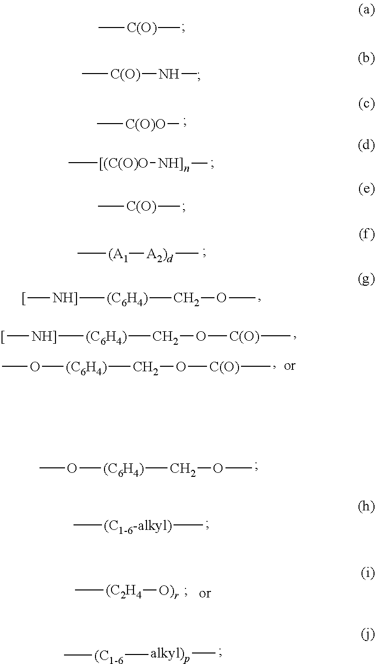

wherein L¹ or L² are terminated in R⁴⁰, wherein R⁴⁰ is a reactive group;
A₁ and A₂ are independent in each instance an amino acid, wherein the amino acid is selected from a natural amino acid or a non-natural amino acid;
wherein subscript:
   d is an integer selected from 0 to 1;
   n is an integer selected from 0 to 4;
   r is an integer selected from 0 to 32; and
   p is an integer 0 or 1.

In certain embodiments of a compound of Formula (XI), L¹ comprises at least one of the following bivalent structures:

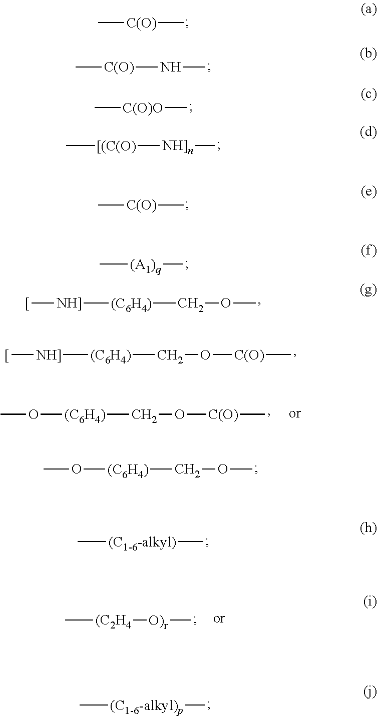

wherein L¹ or L2 are terminated in R⁴⁰, wherein R⁴⁰ is a reactive group;
each A₁ is independently an amino acid, wherein the amino acid is selected from a natural amino acid or a non-natural amino acid;
wherein subscript:
   q is an integer selected from 1 to 6;
   n is an integer selected from 0 to 4;
   r is an integer selected from 0 to 32; and
   p is an integer 0 or 1.

In certain embodiments, L² comprises at least one of the following bivalent structures.

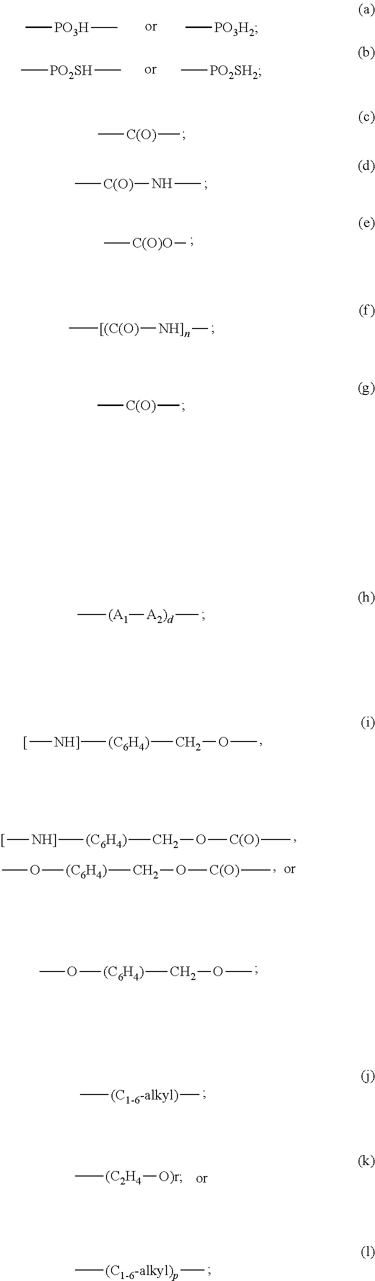

wherein L¹ or L2 are terminated in R⁴⁰, wherein R⁴⁰ is a reactive group;
A₁ and A₂ are independent in each instance an amino acid, wherein the amino acid is selected from a natural amino acid or a non-natural amino acid;
wherein subscript:
   d is an integer selected from 0 to 1;
   n is an integer selected from 0 to 4;
   r is an integer selected from 0 to 32; and
   p is an integer 0 or 1.

In certain embodiments, R⁴ is a C₁-alkyl optionally substituted with at least one C₁₋₆-heterocycle. In certain embodiments, R⁴ is a C₁-alkyl optionally substituted with 1 to 2 pyrrolidinyl substituents. In certain embodiments, R⁴ is

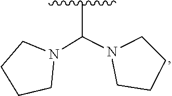 [SMILES: *C(N1CCCC1)N1CCCC1]

wherein

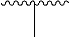

indicates a bond through which the illustrated substituent is bonded. In certain embodiments, R⁵ is phosphate or thiophosphate. In certain embodiments, R⁵ is

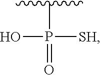 [SMILES: *P(=O)(O)S]

wherein

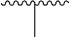

indicates the bond through which the thiophosphate is bonded. In certain embodiments, R⁶ is substituted phosphate or substituted thiophosphate. In certain embodiments, the compound of Formula (XI) is selected from

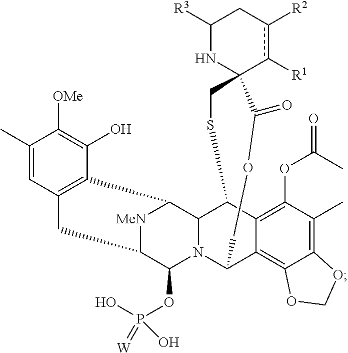

wherein W is O or S. In certain embodiments, W is O. In certain embodiments, W is S.

In certain embodiments, the compound of Formula (XI) is selected from

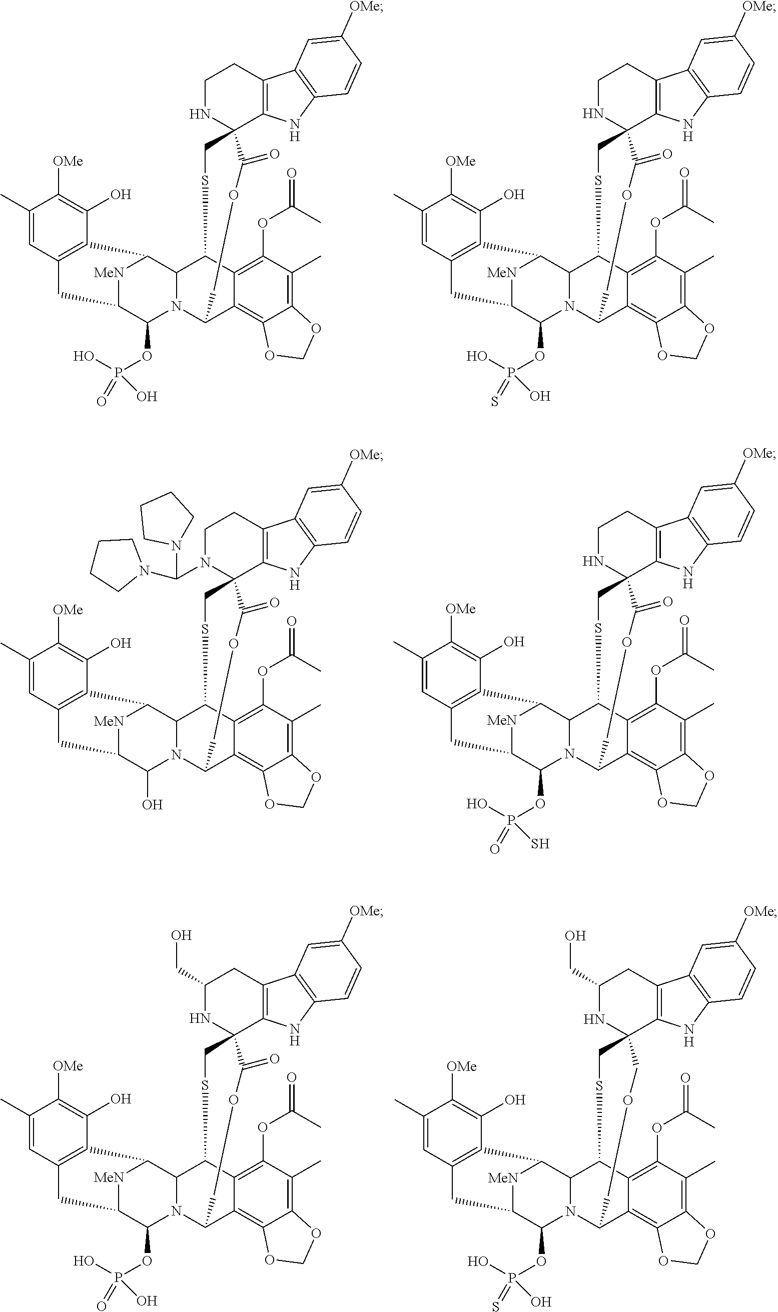

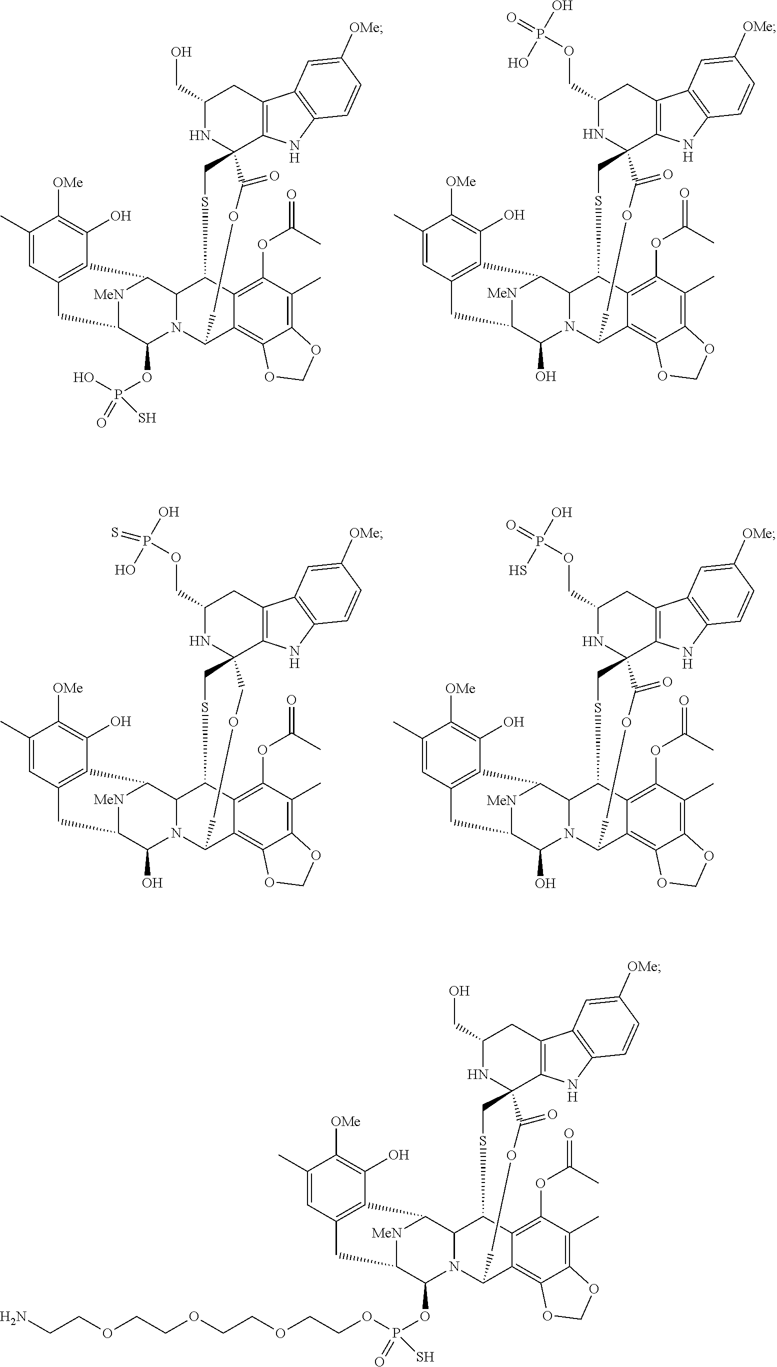

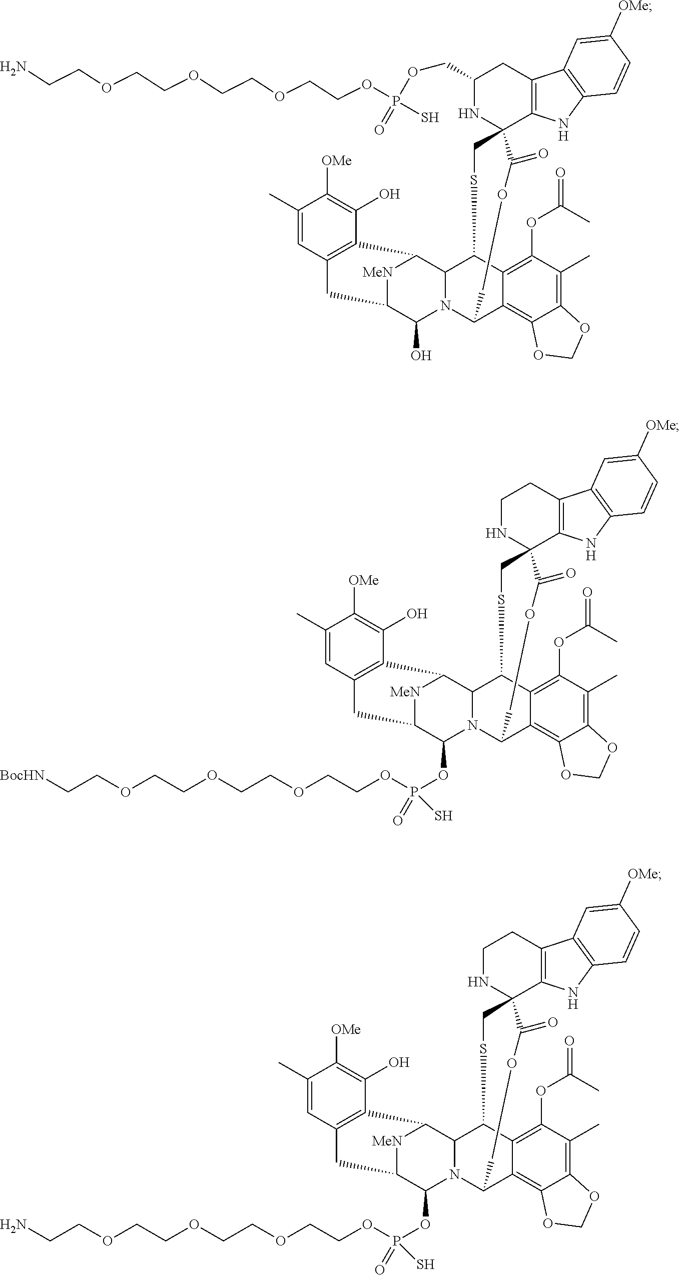 [SMILES: COc1ccc2[nH]c3c(c2c1)C[C@@H](COP(=O)(S)OCCOCCOCCOCCN)N[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCN)N2C)OCO4]

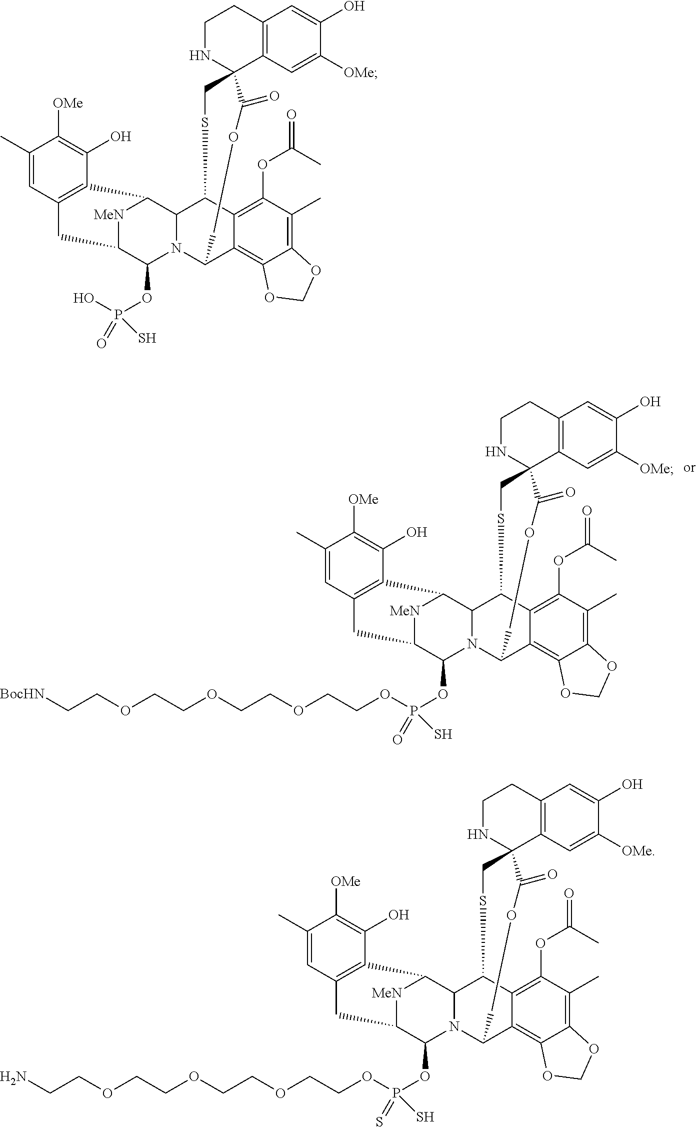 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; "or" ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=S)(S)OCCOCCOCCOCCN)N2C)OCO4]

In certain embodiments, a compound of Formula (XII) or a pharmaceutically acceptable salt, ester, stereoisomer, or tautomer thereof, comprising:

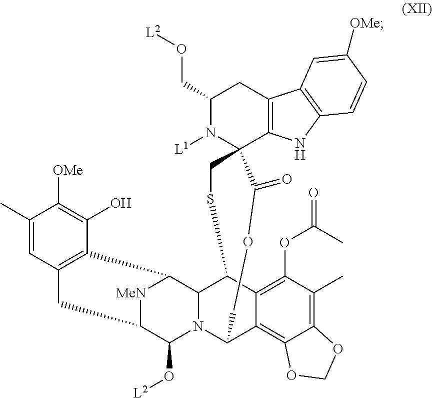

wherein L¹ or one instance of L² is a reactive linker.

In certain embodiments, the compound of Formula (XII), wherein L¹ comprises at least one of the following:
(a) —H, if L² is not H;
(b) —CH₃;
(c) —C₂-s-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of

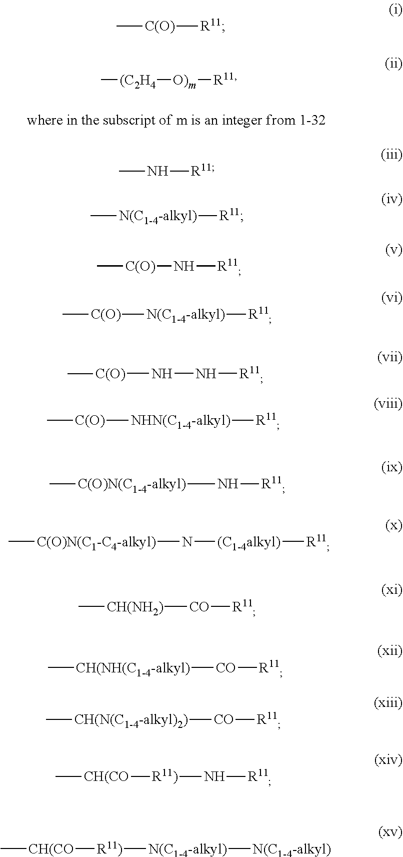

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

In certain embodiments, the compound of Formula (XII), wherein L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

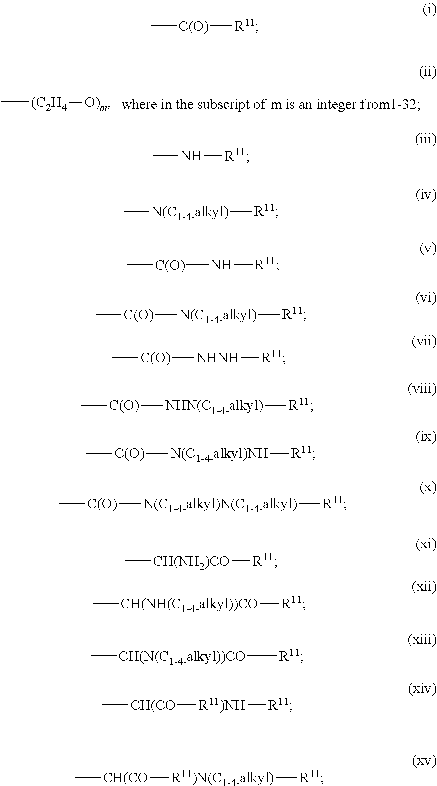

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

In certain embodiments, R¹¹ is terminated in a reactive group, R.

In certain embodiments,
L¹ is selected from the group consisting of H, C₁₋₁₀-alkyl, C₆₋₁₀-aryl, C₃₋₁₀-heterocycle, C(O)R⁸, C(O)OR, C(O)NHR⁸, SO₂R, and SO₂NHR⁸,
wherein the C₅₋₁₀-alkyl and C₆₋₁₀-aryl are each, individually, substituted, or unsubstituted; or
wherein, L¹ is optionally substituted by 1-3 substituents, independently in each instance, selected from the group consisting of OH, F, Cl, Br, C₁₋₅-alkyl, C₃₋₆-cycloalkyl, and C₃₋₆-heterocycle;
wherein R⁸ is selected from the group consisting of C₁₋₆-alkyl, C₃₋₁₀-cycloalkyl, [PEG]_{y}, C₆₋₁₀-aryl, and C₆₋₁₀-heteroaryl,
wherein C₁₋₁₀-alkyl is optionally substituted with one to three substituents independently selected from the group consisting of methyl, ethyl, propyl, fluoro, chloro, bromo, iodo, amino, nitro, —P(O)(OH)₂, thiol, and —SO₃H;
wherein at least one L¹ or L² is not H; and
wherein subscript y is an integer selected from 0 to 32.

In certain embodiments,
L² is selected from the group consisting of H, C₁₋₁₀-alkyl, C₆₋₁₀-aryl, phosphate, thiophosphate, phosphoramide, C(O)R, C(O)OR⁹, and C(O)NHR⁹, SO₃H, SO₂R⁹,
wherein the C₁₋₁₀-alkyl and C₆₋₁₀-aryl are each, individually, substituted, or unsubstituted; or
wherein the phosphate and thiophosphate are optionally substituted with one to two substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
   wherein the substituent on the phosphate and thiophosphate is optionally terminated with a protecting group;
wherein R⁹ is selected from the group consisting of C₁₋₆-alkyl, C₃₋₁₀-cycloalkyl, [PEG]_{y}, C₆₋₁₀-aryl, and C₆₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, and —SO₃H;
wherein at least one L¹ or L² is not H; and
wherein subscript y is an integer selected from 0 to 32.

In certain embodiments, a compound of Formula (XIII), or a pharmaceutically acceptable salt, ester, stereoisomer, or tautomer thereof, comprising:

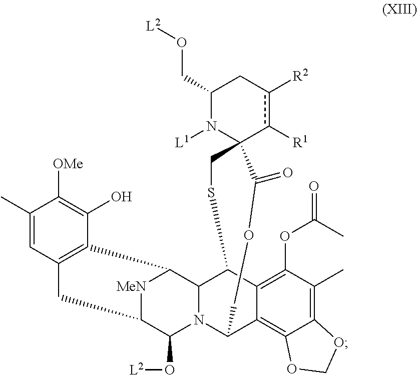

wherein R¹ and R² form at least one aromatic ring or bicyclic heteroaromatic ring, optionally substituted with one to six substituents, independently in each instance, selected from C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰; (e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

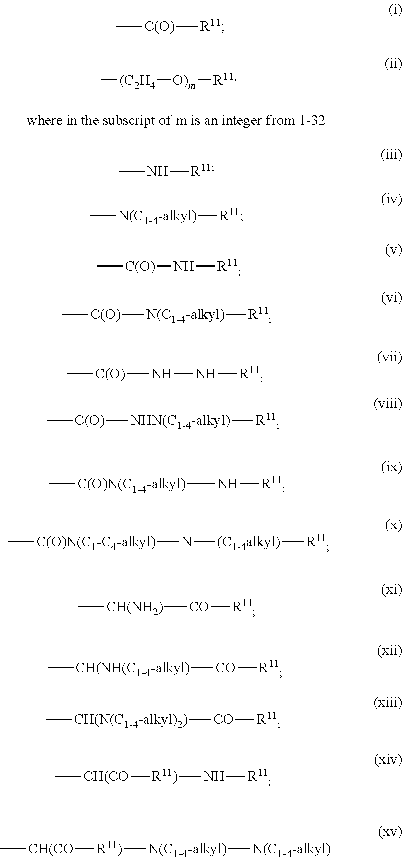

wherein R¹¹ is selected from the group consisting of R^{11a}, R^{11b}, R^{11c}, R^{11d}, and combinations thereof; wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH;

Wherein each L² independently comprises at least one of the following:
(a) —H, if L¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

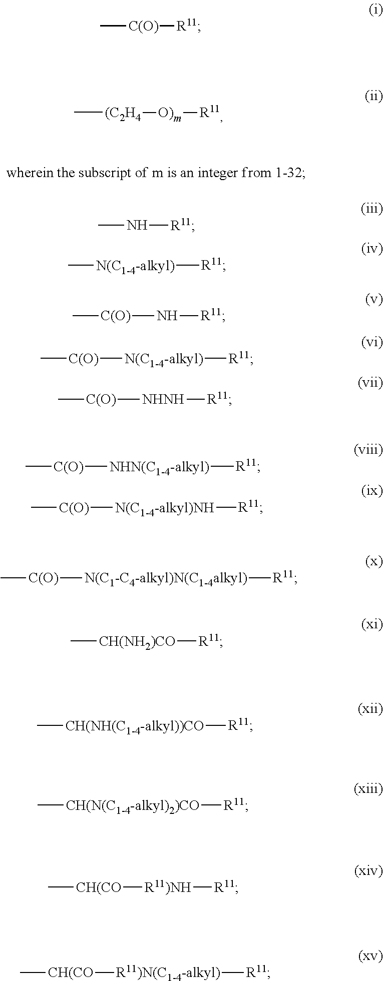

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
R^{11d} is selected from the group consisting of —O— and —NH.
wherein each L² independently comprises at least one of the following:
(a) —H, if L¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

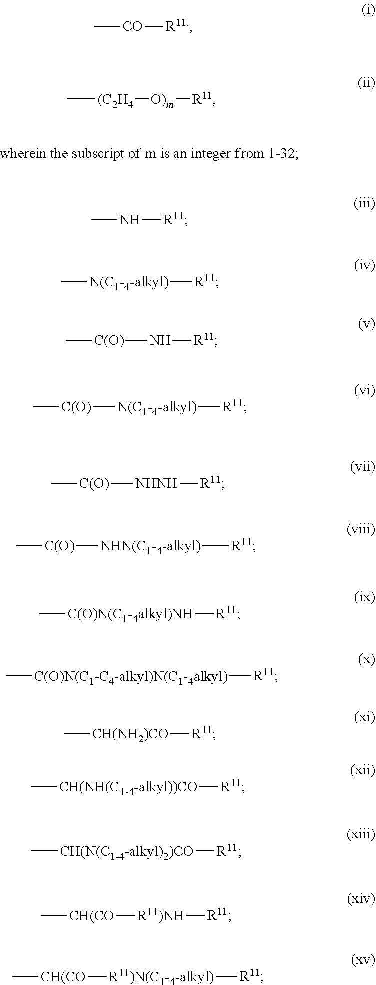

wherein R¹¹ is selected from the group consisting of R^{11a}, R^{11b}, R^{11c}, R^{11d}, and combinations thereof; wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

In certain embodiments, a compound of Formula (XIV) or a pharmaceutically acceptable salt, ester, or tautomer thereof, comprising:

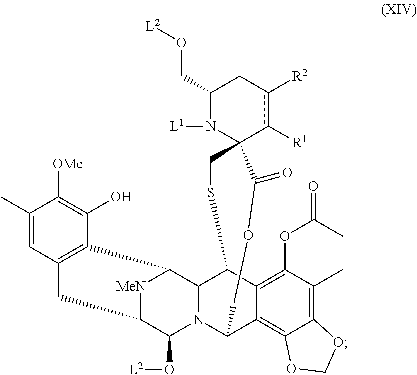

wherein R¹ and R² form at least one aromatic ring or bicyclic heteroaromatic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein one of L¹ or L² is a linker;
wherein L¹ is:

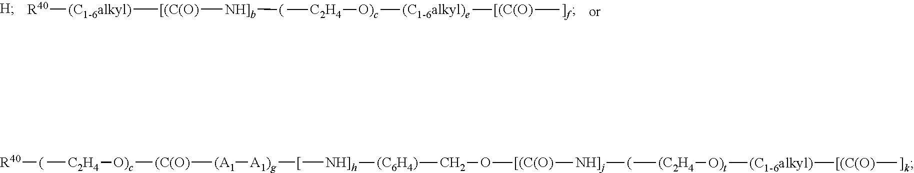

wherein L² is:

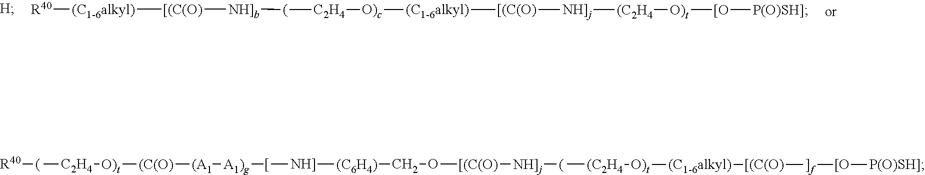

wherein one of L¹ or L2 is not H;
wherein R⁴⁰ is a reactive group;
wherein A₁ and A₂ are independently in each instance an amino acid;
wherein subscript:
   b and j are, independently in each instance, selected from 0 to 4;
   c and t are, independently in each instance, selected from 0 to 32;
   e, f, g, h, and k, are, independently in each instance, 0 or 1.

In certain embodiments, R⁴⁰ is maleimido, —NH₂, —COO-succinimide, halogen or substituted alkyne (e.g., a bicyclooctyne).

In certain embodiments of a compound of Formula (XI), a compound of Formula (XII), a compound of Formula (XIII), a compound of Formula (XIV), or an antibody drug conjugate derived from any one thereof, R^{11b} is -(A₁-A₂)_{q}-. In certain embodiments, A₁-A₂ is: valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

In some embodiments of a compound of Formula (XI), a compound of Formula (XII), a compound of Formula (XIII), a compound of Formula (XIV), or an antibody drug conjugate derived from any one thereof, R^{11b} is -(A₁)_{q}- wherein A₁ is an amino acid, wherein the amino acid is selected from a natural amino acid or a non-natural amino acid, wherein subscript q is 2, 3, or 4. In some embodiments, R¹ b is -(A₁)₂-. In some embodiments, R^{11b} is -(A₁)₄-. In some embodiments, R¹ b is -(A₁)_{q}- is selected from Val-Cit, Val-Ala, Phe-Lys or Gly-Gly-Phe-Gly, wherein q is 2 or 4. In certain embodiments, A₁-A₂ are selected from Val-Cit, Val-Ala, or Phe-Lys. In some embodiments, -(A₁)₄- (or alternatively written, -A₁-A₂-A₃-A₄-) is Gly-Gly-Phe-Gly. In certain embodiments, R^{11b} (e.g., A₁-A₂ or -A₁-A₂-A₃-A₄-) is selected from Val-Ala, or Gly-Gly-Phe-Gly. In certain embodiments, A₁-A₂ are selected from Val-Ala. In certain embodiments, A₁-A₂ are selected from Val-Cit. In certain embodiments, -A₁-A₂-A₃-A₄- are selected from Gly-Gly-Phe-Gly. In some embodiments, -(A₁)_{q}- (e.g., -(A₁)₄-, -A₁-A₂-A₃-A₄-) is selected from SEQ ID NO.: 21.

In some embodiments of a compound of Formula (XI), a compound of Formula (XII), a compound of Formula (XIII), a compound of Formula (XIV), or an antibody drug conjugate derived from any one thereof, A₁ is an amino acid, wherein the amino acid is selected from a natural amino acid or a non-natural amino acid. In some embodiments of -(A₁)_{q}-, subscript q is 2, 3, or 4. In some embodiments, -(A₁)_{q}- is -(A₁)₂-. In some embodiments, -(A₁)_{q}- is -(A₁)₄-. In some embodiments, -(A₁)_{q}- is selected from Val-Cit, Val-Ala, Phe-Lys or Gly-Gly-Phe-Gly, wherein q is 2 or 4. In certain embodiments, -(A₁)_{q}- is -(A₁-A₂)-, wherein A₁-A₂ is selected from Val-Cit, Val-Ala, or Phe-Lys. In some embodiments, -(A₁)₄- (or alternatively written, -A₁-A₂-A₃-A₄-) is Gly-Gly-Phe-Gly. In certain embodiments, -(A₁)_{q}- (e.g., A₁-A₂ or -A₁-A₂-A₃-A₄-) is selected from Val-Ala, or Gly-Gly-Phe-Gly. In certain embodiments, A₁-A₂ are selected from Val-Ala. In certain embodiments, A₁-A₂ are selected from Val-Cit. In certain embodiments, A₁-A₂-A₃-A₄- are selected from Gly-Gly-Phe-Gly. In some embodiments, -(A₁)_{q}- (e.g., -(A₁)₄-, -A₁-A₂-A₃-A₄-) is selected from SEQ ID NO.: 21.

In certain embodiments of a compound of Formula (XI), a compound of Formula (XII), a compound of Formula (XIII), or a compound of Formula (XIV), the compound is:

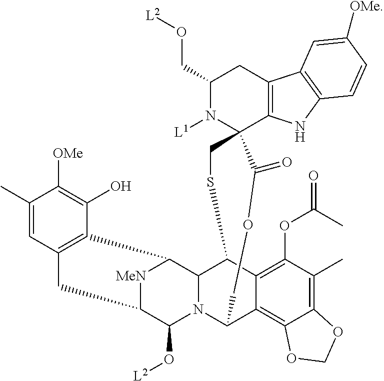

In certain embodiments, at least one of L¹ or L² independently comprises:

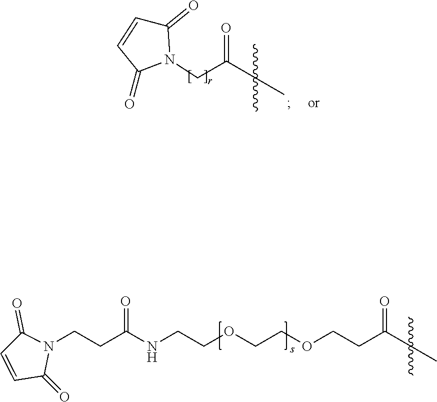

wherein

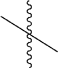

indicates the point of attachment to the compound of Formula (IV) or to a remaining portion of L¹ or L²;
wherein r is an integer select from 1 to 12; and
wherein s is an integer select from 1 to 32.

In certain embodiments, r is an integer selected from 2 to 12; and
s is an integer selected from 2 to 32. In certain embodiments, r is 5. In certain embodiments, s is 3.

In some embodiments of a compound of Formula (XI), a compound of Formula (XII), a compound of Formula (XIII), or a compound of Formula (XIV), L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—; wherein L¹ is terminated in R⁴⁰, wherein R⁴⁰ is a reactive group. In some embodiments, one or more occurrences of -(A₁)_{q}- is Val-Cit or Cit-Val. In some embodiments, L¹ is —C(O)—(C₁₋₆-alkyl)-R⁴⁰. In some embodiments, L¹ is —C(O)—(C₂H₄—O)—(C₁₋₆-alkyl)-NH—C(O)—(C₁₋₆-alkyl)-R⁴⁰. In some embodiments, L¹ is —C(O)—(C₂H₄—O)—(CH₂)₂—NH—C(O)—(CH₂)₂—R⁴⁰. In some embodiments, L¹ is —C(O)—(C₂H₄—O)ₘ—NH—C(O)—O—CH₂—(C₆H₄)NH-(A₁)_{q}-C(O)(C₂H₄—O)ₘ—(C₁₋₆-alkyl)-R⁴⁰. In some embodiments, L¹ is -(A₁)_{q}-C(O)—O—CH₂—(C₆H₄)—NH-(A₁)_{q}-C(O)—(C₁₋₆-alkyl)-R⁴⁰. In some embodiments, L¹ is di(pyrrolidin-1-yl)methyl. In some embodiments, L¹ is —C(O)—(C₂H₄—O)₂—(CH₂)—NH—C(O)—(CH₂)—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L¹ is -(Gly)-C(O)—O—CH₂—(C₆H₄)—NH-(Cit-Val)-C(O)—(CH₂)₅—R⁴⁰. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In some embodiments of a compound of Formula (XI), a compound of Formula (XII), a compound of Formula (XIII), or a compound of Formula (XIV), L² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L² is terminated in R⁴⁰, wherein R⁴⁰ is a reactive group. In some embodiments, L² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)_{M}—; wherein L² is terminated in R⁴⁰, wherein R⁴⁰ is a reactive group. In some embodiments, L² is —PO₂SH—(C₂H₄—O)ₘ—(C₁₋₆-alkyl)-R⁴⁰. In some embodiments, L² is —PO₂SH—(C₂H₄—O)ₘ—(C₁₋₆ alkyl)-NH—C(O)—O—CH₂—(CH)—NH-(A₁)_{q}-C(O)—(C₂H₄—O)ₘ—(C₁₋₆-alkyl)-R⁴⁰. In some embodiments, L² is —PO₂SH—(C₂H₄—O)ₘ—NH—C(O)—(C₂H₄—O)ₘ—NH—C(O)—(C₁₋₆-alkyl)-R⁴⁰. In some embodiments, L² is —PO₂SH₂. In some embodiments, L² is —PO₃H₂. In some embodiments, L² is —PO₂SH—(C₂H₄—O)₃—(CH₂)₂—R⁴⁰. In some embodiments, L² is —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L² is —PO₂SH—(C₂H₄—O)₃—(CH₂)—NH—C(O)—(C₂H₄—O)₄—(CH₂)₂—NH—C(O)—(CH₂)₂—R⁴⁰.

In some embodiments of an antibody drug conjugate derived from a compound of Formula (XI), a compound of Formula (XII), a compound of Formula (XIII), or a compound of Formula (XIV), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, one or more occurrences of -(A₁)_{q}- is Val-Cit or Cit-Val. In some embodiments, B¹ comprises —C(O)—(C₁₋₆-alkyl)-. In some embodiments, B¹ comprises —C(O)—(C₂H₄—O)ₘ—(C₁₋₆-alkyl)-NH—C(O)—(C₁₋₆-alkyl)-. In some embodiments, B¹ comprises —C(O)—(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)₂—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)—(CH₂)₅—.

In some embodiments of an antibody drug conjugate derived from a compound of Formula (XI), a compound of Formula (XII), a compound of Formula (XIII), or a compound of Formula (XIV), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₂H₄)—NH-(A₁)_{q}-C(O)—(C₂H₄—O)ₙ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—.

In certain embodiments of a compound of Formula (XI), a compound of Formula (XII), a compound of Formula (XIII), or a compound of Formula (XIV), L¹ or one instance of L² is terminated in R⁴⁰, wherein R⁴⁰ is a reactive group. In certain embodiments, R¹¹ is terminated in R⁴⁰, wherein R⁴⁰ is a reactive group. In some embodiments, the reactive group is -NHBoc

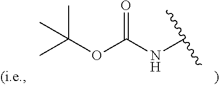 [SMILES: *NC(=O)OC(C)(C)C]

or —NH₂. In some embodiments, the reactive group is maleimide.

In some embodiments of a compound of Formula (XI), a compound of Formula (XII), a compound of Formula (XIII), or a compound of Formula (XIV), when L¹ is H, then at least one occurrence of L² is not H; and when all occurrences of L² are H, then L¹ is not H.

In some embodiments of an antibody drug conjugate derived from a compound of Formula (XI), a compound of Formula (XII), a compound of Formula (XIII), or a compound of Formula (XIV), B¹ comprises the reaction product of R⁴⁰ as found in L with Ab, such that a covalent linkage is formed therebetween. In some embodiments, B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

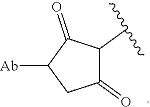

In certain embodiments of a compound of Formula (XI), a compound of Formula (XII), a compound of Formula (XIII), a compound of Formula (XIV), or an antibody drug conjugate derived from any one thereof, A₁ and A₂ are independent in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of a compound of Formula (XI), a compound of Formula (XII), a compound of Formula (XIII), a compound of Formula (XIV), or an antibody drug conjugate derived from any one thereof, subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently an integer selected from 0 to 4; each instance of subscript m is independently an integer selected from 1 to 20; each instance of subscript p is independently an integer 0 or 1; each instance of subscript q is independently an integer selected from 1 to 6; and each instance of subscript n is independently an integer selected from 0 to 10.

In certain embodiments, the compound (e.g., a compound of Formula (XI), a compound of Formula (XII), a compound of Formula (XIII), a compound of Formula (XIV)) is:

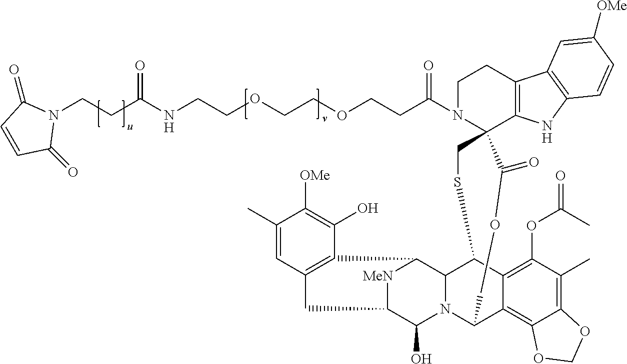

wherein subscript v is an integer from 1 to 32; and wherein subscript u is an integer from 0 to 11. In certain embodiments, subscript v is an integer selected from 1 to 14.

In certain embodiments, the compound (e.g., a compound of Formula (XI), a compound of Formula (XII), a compound of Formula (XIII), a compound of Formula (XIV)) is

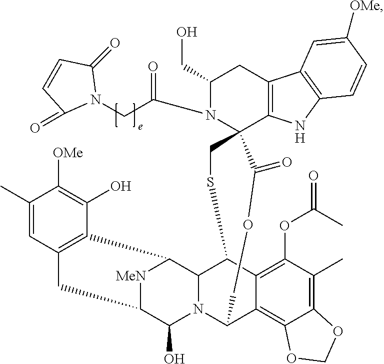

wherein subscript e is an integer selected from 1 to 6. In certain embodiments, e is an integer selected from 1 to 5. In certain embodiments, e is an integer selected from 1 to 4. In certain embodiments, e is an integer selected from 1 to 3.

In certain embodiments, the compound (e.g., a compound of Formula (XI), a compound of Formula (XII), a compound of Formula (XIII), a compound of Formula (XIV)) has the structure:

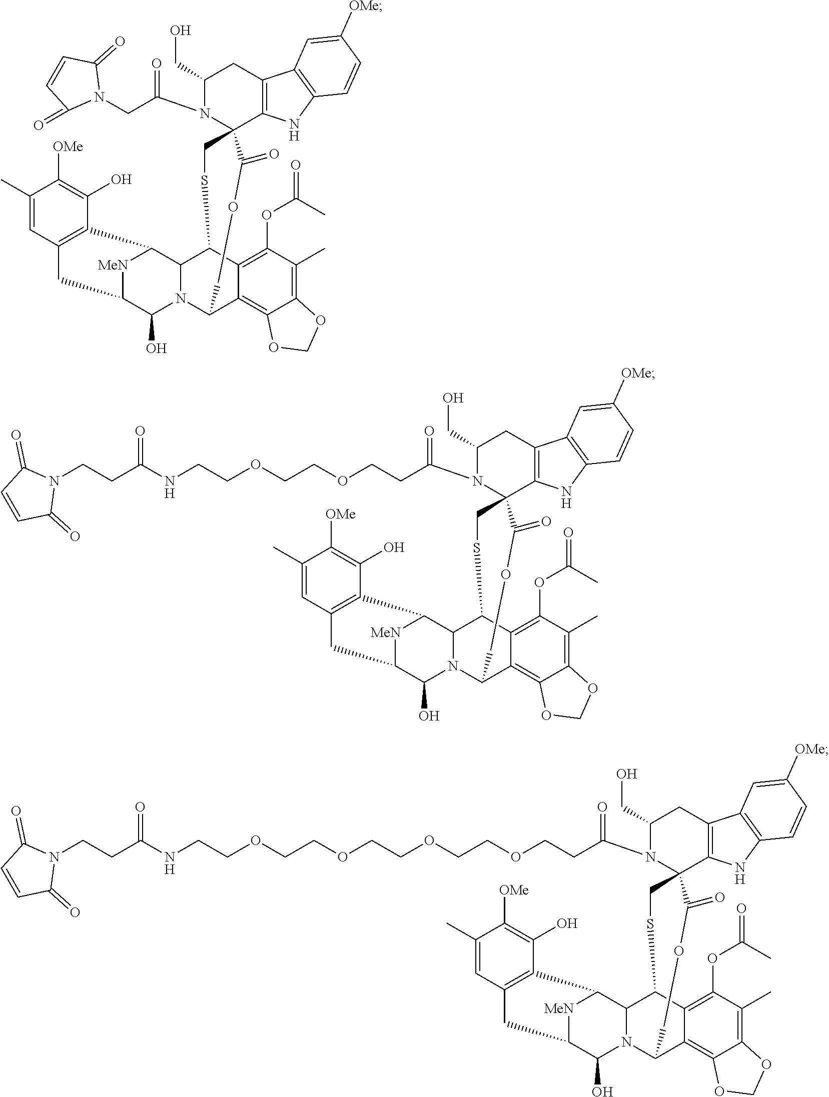

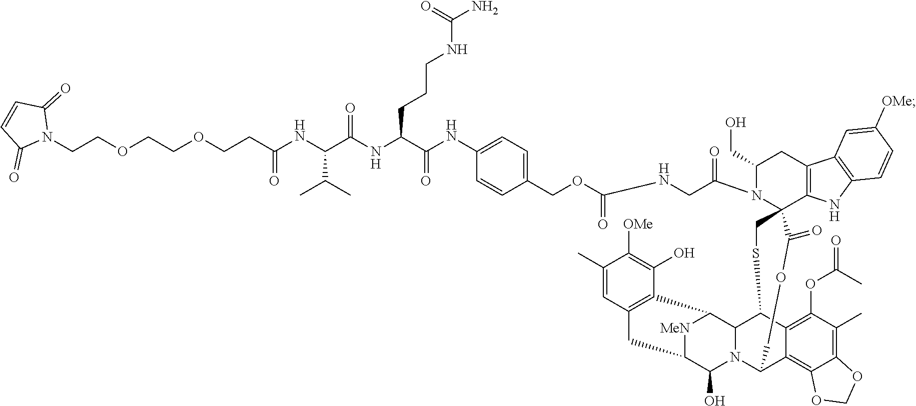 [SMILES: COc1ccc2[nH]c3c(c2c1)C[C@@H](CO)N(C(=O)CNC(=O)OCc1ccc(NC(=O)[C@H](CCCNC(N)=O)NC(=O)[C@@H](NC(=O)CCOCCOCCN2C(=O)C=CC2=O)C(C)C)cc1)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

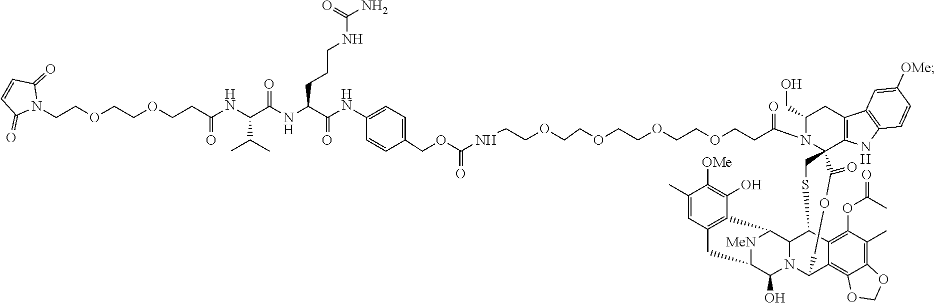 [SMILES: COc1ccc2[nH]c3c(c2c1)C[C@@H](CO)N(C(=O)CCOCCOCCOCCOCCNC(=O)OCc1ccc(NC(=O)[C@H](CCCNC(N)=O)NC(=O)[C@@H](NC(=O)CCOCCOCCN2C(=O)C=CC2=O)C(C)C)cc1)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

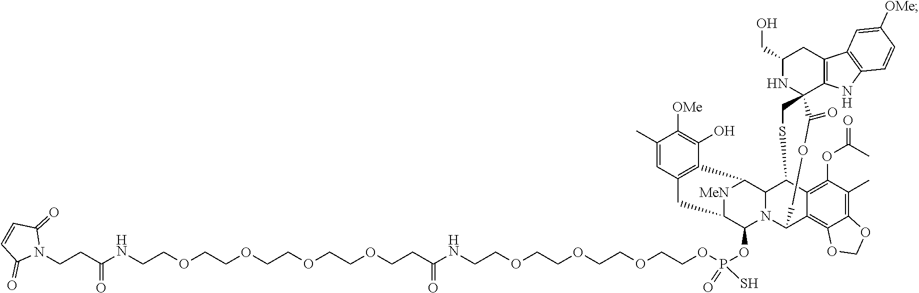 [SMILES: COc1ccc2[nH]c3c(c2c1)C[C@@H](CO)N[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)CCOCCOCCOCCOCCNC(=O)CCN1C(=O)C=CC1=O)N2C)OCO4]

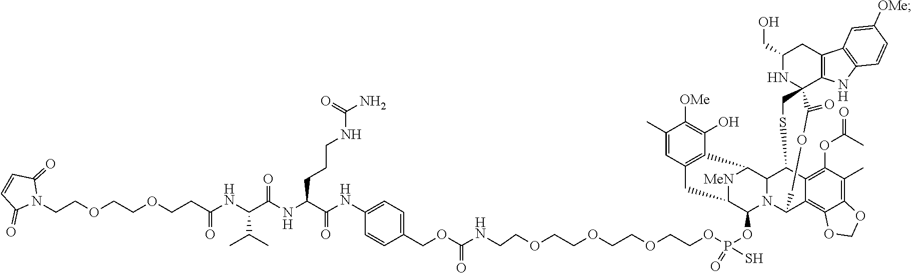 [SMILES: COc1ccc2[nH]c3c(c2c1)C[C@@H](CO)N[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4]

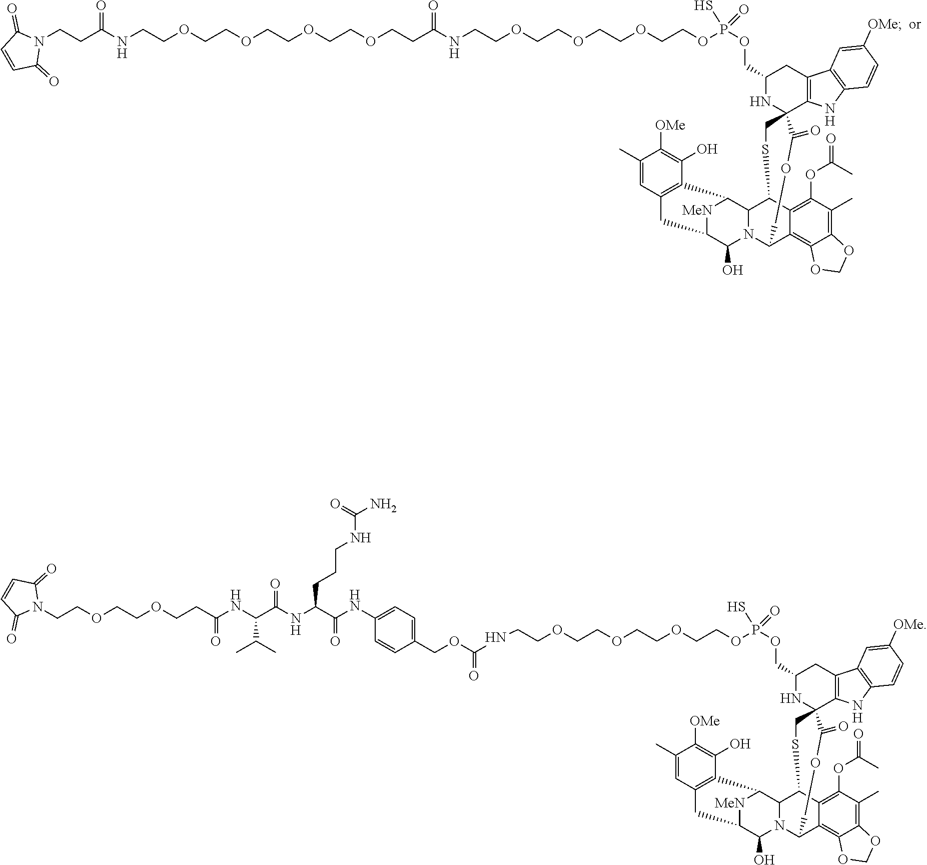

In certain embodiments, provided herein is a compound comprising a linker or reactive linker covalently bonded to lurbinectedin or ecubectedin via a secondary alcohol, or a secondary amine. In certain embodiments, the compound is covalently bonded to a targeting agent. In certain embodiments, compound is covalently bonded to an antibody or antibody fragment thereof. In some embodiments, the antibody of the antibody fragment thereof comprises at least one of SEQ ID NOs. 1-20 as listed in Table 6.

In certain embodiments, provided herein is a compound of Formula (XVa), (XVb), or (XVc), or a pharmaceutically acceptable salt, ester, stereoisomer, or tautomer thereof, comprising:

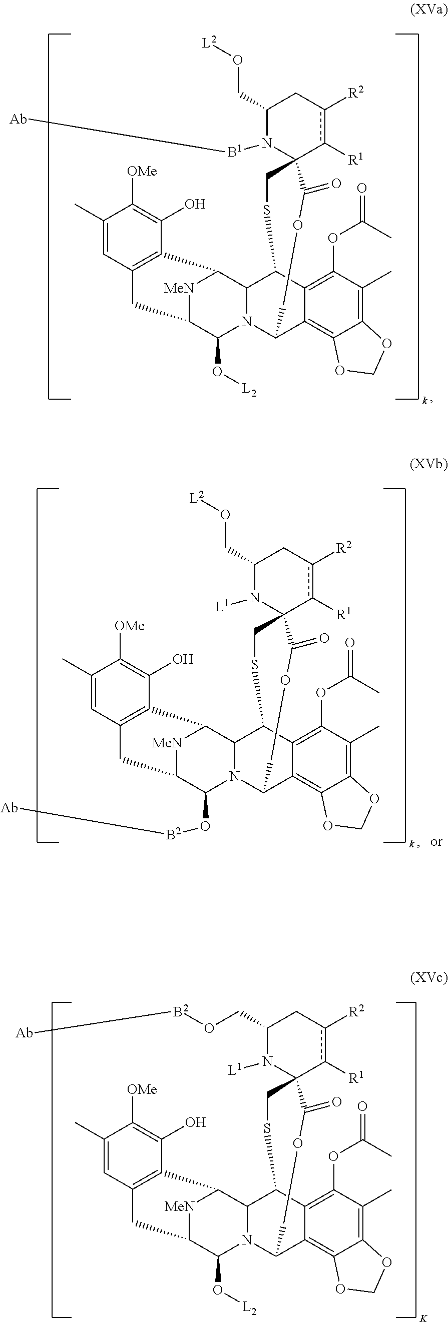

wherein Ab is targeting agent;
wherein subscript k is an integer from 1 to 10;
wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H; and
wherein B¹ is a linker;

In some embodiments, B² is a linker.

In some embodiments of a compound of Formula (XVa), Formula (XVb), or Formula (XVc), Ab is an antibody, antibody fragment, protein, or peptide. In some embodiments, of a compound of Formula (XVa), Formula (XVb), or Formula (XVc), the compound is an antibody drug conjugate derived from a compound of Formula (XI), Formula (XII), Formula (XIII), or Formula (XIV). In some embodiments, the antibody or antibody fragment includes one or more of SEQ ID NOs. 1 to 20 as listed in Table 6. Non-limiting examples of antibodies include trastuzumab and brentuximab. The sequences associated with trastuzumab and brentuximab are listed in Table 6 below:

TABLE 6
Sequence ID NOs.

| SEQ | | |
|---|---|---|
| ID | | |
| NO. | Description | Sequence |
| 1 | Trastuzumab | EVQLVESGGG LVQPGGSLRL SCAASGFNIK |
| | VH | DTYIHWVRQA PGKGLEWVAR IYPTNGYTRY |
| | | ADSVKGRFTI SADTSKNTAY LQMNSLRAED |
| | | TAVYYCSRWG GDGFYAMDYW GQGTLVTVSS |
| 2 | Trastuzumab | DTYIH |
| | HCDR1 | |
| 3 | Trastuzumab | RIYPTNGYTR YADSVKG |
| | HCDR2 | |
| 4 | Trastuzumab | WGGDGFYAMD Y |
| | HCDR3 | |
| 5 | Trastuzumab | DIQMTQSPSS LSASVGDRVT ITCRASQDVN |
| | VL | TAVAWYQQKP GKAPKLLIYS ASFLYSGVPS |
| | | RFSGSRSGTD FTLTISSLQP EDFATYYCQQ |
| | | HYTTPPTFGQ GTKVEIK |
| 6 | Trastuzumab | RASQDVNTAV A |
| | LCDR1 | |
| 7 | Trastuzumab | SASFLYS |
| | LCDR2 | |
| 8 | Trastuzumab | QQHYTTPPT |
| | LCDR3 | |
| 9 | Trastuzumab | EVQLVESGGG LVQPGGSLRL SCAASGFNIK |
| | IgG HC | DTYIHWVRQA PGKGLEWVAR IYPTNGYTRY |
| | | ADSVKGRFTI SADTSKNTAY LQMNSLRAED |
| | | TAVYYCSRWGGDGFYAMDYWGQGTLVTVSS |
| | | ASTKGPSVFP LAPSSKSTSG GTAALGCLVK |
| | | DYFPEPVTVS WNSGALTSGV HTFPAVLQSS |
| | | GLYSLSSVVT VPSSSLGTQT YICNVNHKPS |
| | | NTKVDKKVEP KSCDKTHTCP PCPAPELLGG |
| | | PSVFLFPPKPKDTLMISRTPEVTCVVVDVSHE |
| | | DPEVKFNWYVDGV |
| | | EVHNAKTKPREEQYNSTYRVVSVLTVLHQDWL |
| | | NGKEYKCKVSNKA |
| | | LPAPIEKTISKAKGQPREPQVYTLPPSREEMT |
| | | KNQVSLTCLVKGFY |
| | | PSDIAVEWESNGQPENNYKTTPPVLDSDGSFF |
| | | LYSKLTVDKSRW |
| | | QQGNVFSCSMHEALHNHYTQKSLSLSPGK |
| 10 | Trastuzumab | DIQMTQSPSSLSASVGDRVTITCRASQDVNTA |
| | IgG LC | VAWYQQKPGKAPKLLIYSASFLYSGVPSRFSG |
| | | SRSGTDFTLTISSLQPEDFATYYCQQHYTTPP |
| | | TFGQGTKVEIKRTVAAPSVFIFPPSDEQLKSG |
| | | TASVVCLLNNFYPREAKVQWKVDNALQSGNSQ |
| | | ESVTEQDSKDSTYSLSSTLTLSKADYEKHKVY |
| | | ACEVTHQGLSSPVTKSFNRGEC |
| 11 | Brentuximab | QIQLQQSGPE VVKPGASVKI SCKASGYTFT |
| | VH | DYYITWVKQK PGQGLEWIGW IYPGSGNTKY |
| | | NEKFKGKATL TVDTSSSTAF MQLSSLTSED |
| | | TAVYFCANYG NYWFAYWGQG TQVTVSA |
| 12 | Brentuximab | DYYIT |
| | HCDR1 | |
| 13 | Brentuximab | WIYPGSGNTKYNEKFKG |
| | HCDR2 | |
| 14 | Brentuximab | YGNYWFAY |
| | HCDR3 | |
| 15 | Brentuximab | DIVLTQSPAS LAVSLGQRAT ISCKASQSVD |
| | VL | FDGDSYMNWY QQKPGQPPKV LIYAASNLES |
| | | GIPARFSGSG SGTDFTLNIH PVEEEDAATY |
| | | YCQQSNEDPW TFGGGTKLEI K |
| 16 | Brentuximab | KASQSVDFDGDSYMN |
| | LCDR1 | |
| 17 | Brentuximab | AASNLES |
| | LCDR2 | |
| 18 | Brentuximab | QQSNEDPWT |
| | LCDR3 | |
| 19 | Brentuximab | QIQLQQSGPE VVKPGASVKI SCKASGYTFT |
| | IgG HC | DYYITWVKQK PGQGLEWIGW IYPGSGNTKY |
| | | NEKFKGKATL TVDTSSSTAF MQLSSLTSED |
| | | TAVYFCANYG NYWFAYWGQG TQVTVSAAST |
| | | KGPSVFPLAP SSKSTSGGTA ALGCLVKDYF |
| | | PEPVTVSWNS GALTSGVHTF PAVLQSSGLY |
| | | SLSSVVTVPS SSLGTQTYIC NVNHKPSNTK |
| | | VDKKVEPKSC DKTHTCPPCP APELLGGPSV |
| | | FLFPPKPKDT LMISRTPEVT CVVVDVSHED |
| | | PEVKFNWYVD GVEVHNAKTK PREEQYNSTY |
| | | RVVSVLTVLH QDWLNGKEYK CKVSNKALPA |
| | | PIEKTISKAK GQPREPQVYT LPPSRDELTK |
| | | NQVSLTCLVK GFYPSDIAVE WESNGQPENN |
| | | YKTTPPVLDS DGSFFLYSKL TVDKSRWQQG |
| | | NVFSCSVMHE ALHNHYTQKS LSLSPGK |
| 20 | Brentuximab | DIVLTQSPAS LAVSLGQRAT ISCKASQSVD |
| | IgG LC | FDGDSYMNWY QQKPGQPPKV LIYAASNLES |
| | | GIPARFSGSG SGTDFTLNIH PVEEEDAATY |
| | | YCQQSNEDPW TFGGGTKLEI KRTVAAPSVF |
| | | IFPPSDEQLK SGTASVVCLL NNFYPREAKV |
| | | QWKVDNALQS GNSQESVTEQ DSKDSTYSLS |
| | | STLTLSKADY EKHKVYACEV THQGLSSPVT |
| | | KSFNRGEC |
| 21 | Embodiment | GGFG |
| | of-(A₁)_{q}- | |

In certain embodiments, for a compound of Formula (XVa), (XVb), or (XVc), B¹ comprises at least one of the following:
(a) —H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

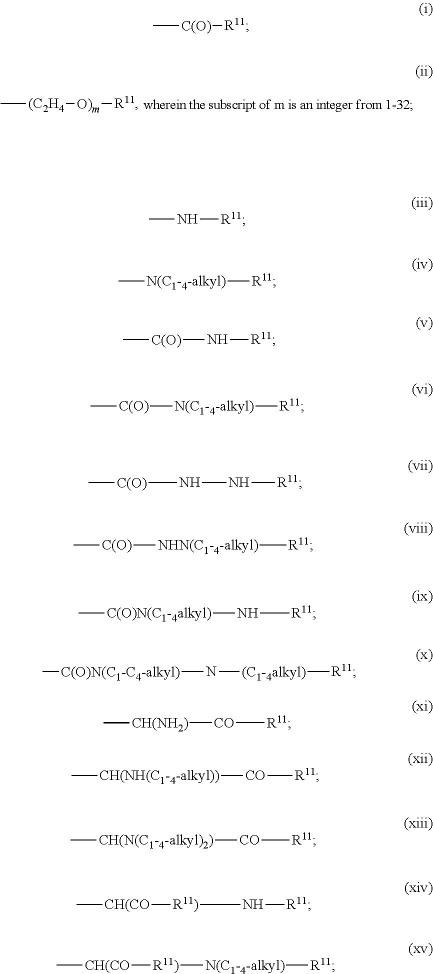

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH;
wherein B² comprises at least one of the following:
(a) —H;
(b) —PO₃H—;
(c) —PO₂SH—;
(d) —CH₃;
(e) —C₂-M-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰; (j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

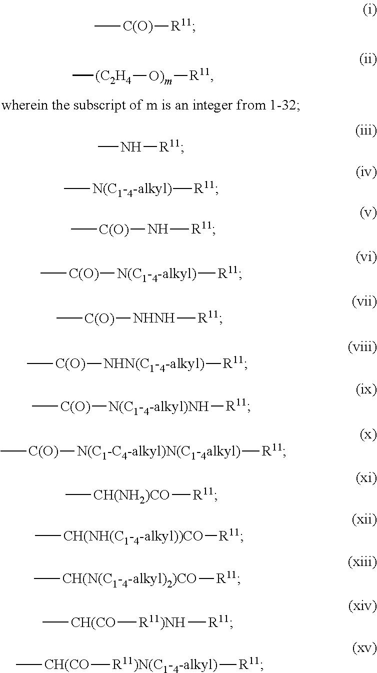

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH; and
wherein one of B¹ or B² is not H; and
wherein one of B¹ or B² is bonded to Ab.

In some embodiments of a compound of Formula (XVa), Formula (XVb), or Formula (XVc), B¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—. In some embodiments, one or more occurrences of -(A₁)_{q}- is Val-Cit or Cit-Val. In some embodiments, B¹ comprises —C(O)—(C₁₋₆-alkyl)-. In some embodiments, B¹ comprises —C(O(C₂H₄—O)ₘ—(C₁₋₆-alkyl)-NH—C(O)—(C₁₋₆-alkyl)-. In some embodiments, B¹ comprises —C(O)—(C₂H₄—O)ₘ—(CH₂)₂—NH—C(O)—(CH₂)₂—. In some embodiments, B¹ comprises —C(O)—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B¹ comprises -(A₁)_{q}-C(O)—O—CH₂—(C₆H₄)—NH-(A₁)_{q}-C(O)—(C₁₋₆-alkyl)-. In some embodiments, B¹ comprises —C(O)—(C₂H₄—O)₂—(CH₂)₂—NH—C(O)—(CH₂)₂—. In some embodiments, B¹ comprises —C(O)—(C₂H₄—O)₄—(CH₂)₂—NH—C(O)—(CH₂)₂—. In some embodiments, B¹ comprises —C(O)—(C₂H₄—O)₁₂—(CH₂)₂—NH—C(O)—(CH₂)₂—. In some embodiments, B¹ comprises —C(O)—(CH₂)₅—. In some embodiments, B¹ comprises —C(O)—(CH₂)—. In some embodiments, B¹ comprises —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B¹ comprises -(Gly)-C(O)—O—CH₂—(C₆H₄)—NH-(Cit-Val)-C(O)(CH₂)₅—. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In some embodiments of a compound of Formula (XVa), Formula (XVb), or Formula (XVc), B² comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—. In some embodiments, B² comprises: i) —PO₂SH—; and ii) 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(CH₄)—NH—, and 0-3 instances of —(C₂H₄—O)ₘ—. In some embodiments, B² comprises —PO₂SH—(C₂H₄—O)ₘ—(C₁₋₆-alkyl)-. In some embodiments, B² comprises —PO₂SH—(C₂H₄—O)ₘ—NH—C(O)—O—CH₂—(C₆H₄)—NH-(A₁)_{q}-C(O)—(C₂H₄—O)ₘ—(C₁₋₆-alkyl)-. In some embodiments, B² comprises —PO₂SH—(C₂H₄—O)ₘ—NH—C(O)—(C₂H₄—O)ₘ—NH—C(O)—(C₁₋₆-alkyl)-. In some embodiments, B² comprises —PO₂SH—(C₂H₄—O)₃—(CH₂)₂—. In some embodiments, B² comprises —PO₂SH—(C₂H₄—O)₃—(CH₂)₂—NH—C(O)—O—CH₂—(C₆H₄)—NH-(Cit-Val)-C(O)—(C₂H₄—O)₂—(CH₂)₂—. In some embodiments, B² comprises —PO₂SH—(C₂H₄—O)₃—(CH₂)₂—NH—C(O)—(C₂H₄—O)₄—(CH₂)₂—NH—C(O)—(CH₂)₂—. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In some embodiments of a compound of Formula (XVa), Formula (XVb), or Formula (XVc), L¹ or L² is H.

In some embodiments of a compound of Formula (XVa), Formula (XVb), or Formula (XVc), B¹ comprises the reaction product of R⁴⁰ as found in L¹ with Ab, such that a covalent linkage is formed therebetween. In some embodiments of a compound of Formula (XVa), Formula (XVb), or Formula (XVc), B² comprises the reaction product of R⁴⁰ as found in L² with Ab, such that a covalent linkage is formed therebetween. Accordingly, when R⁴⁰ is maleimide, then Ab-B¹— or Ab-B²— can independently terminate with

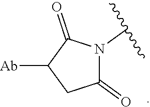

In certain embodiments of a compound of Formula (XVa), Formula (XVb), or Formula (XVc), A₁ and A₂ are independent in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of a compound of Formula (XVa), Formula (XVb), or Formula (XVc), subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently an integer selected from 0 to 4; each instance of subscript m is independently an integer selected from 1 to 20; each instance of subscript p is independently an integer 0 or 1; each instance of subscript q is independently an integer selected from 1 to 6; and each instance of subscript n is independently an integer selected from 0 to 10.

In certain embodiments, the compound (e.g., a compound of Formula (XVa), a compound of Formula (XVb), a compound of Formula (XVc)) is

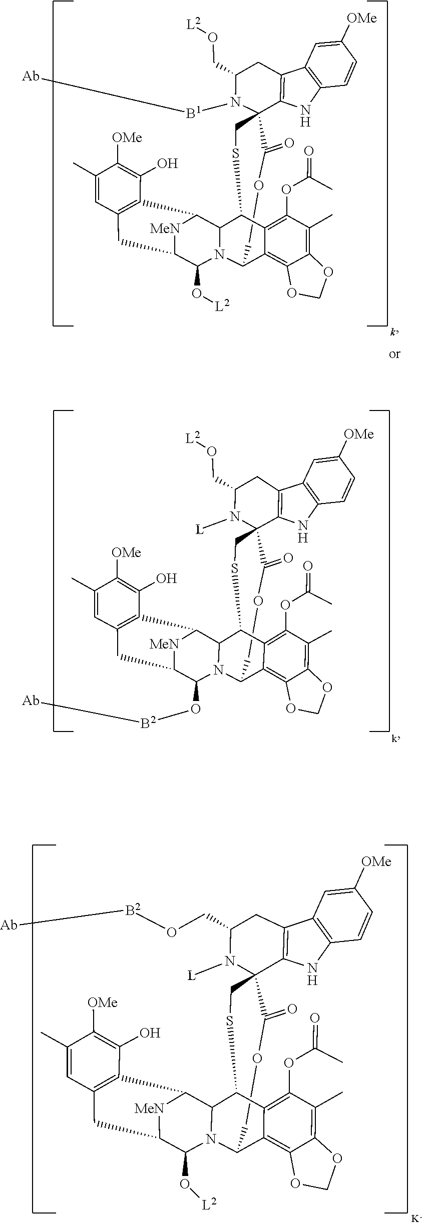

In certain embodiments of Formula (XVa), Formula (XVb), or Formula (XVc), at least one of B¹ or B² independently comprises

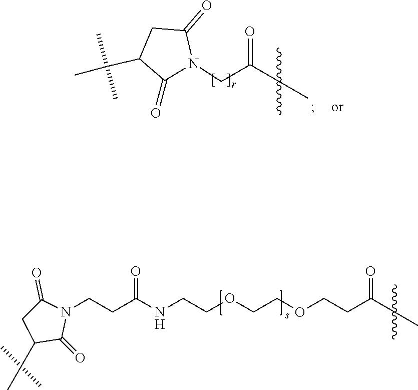

wherein

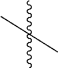

indicates the point of attachment to the compound of Formula (XVa), (XVb) or (XVc) or to a remaining portion of L¹ or L²;
wherein

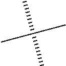

indicates the point of attachment to an antibody or antibody fragment;
wherein subscript r is an integer selected from 1 to 6; and
wherein subscript s is an integer selected from 2 to 14. In certain embodiments, r is an integer selected from 2 to 6; and s is an integer selected from 2 to 14.

In certain embodiments, the compound is

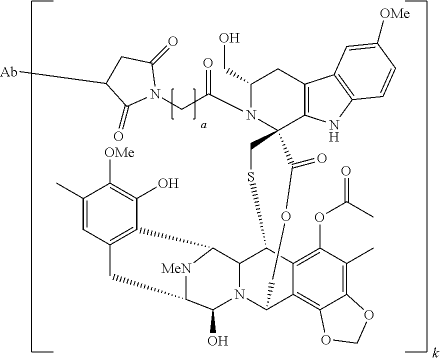

wherein a is an integer selected from 2 to 6. In certain embodiments, a is 4. In certain embodiments, a is 5.

In certain embodiments, the compound (e.g., a compound of Formula (XVa), a compound of Formula (XVb), a compound of Formula (XVc)) is selected from

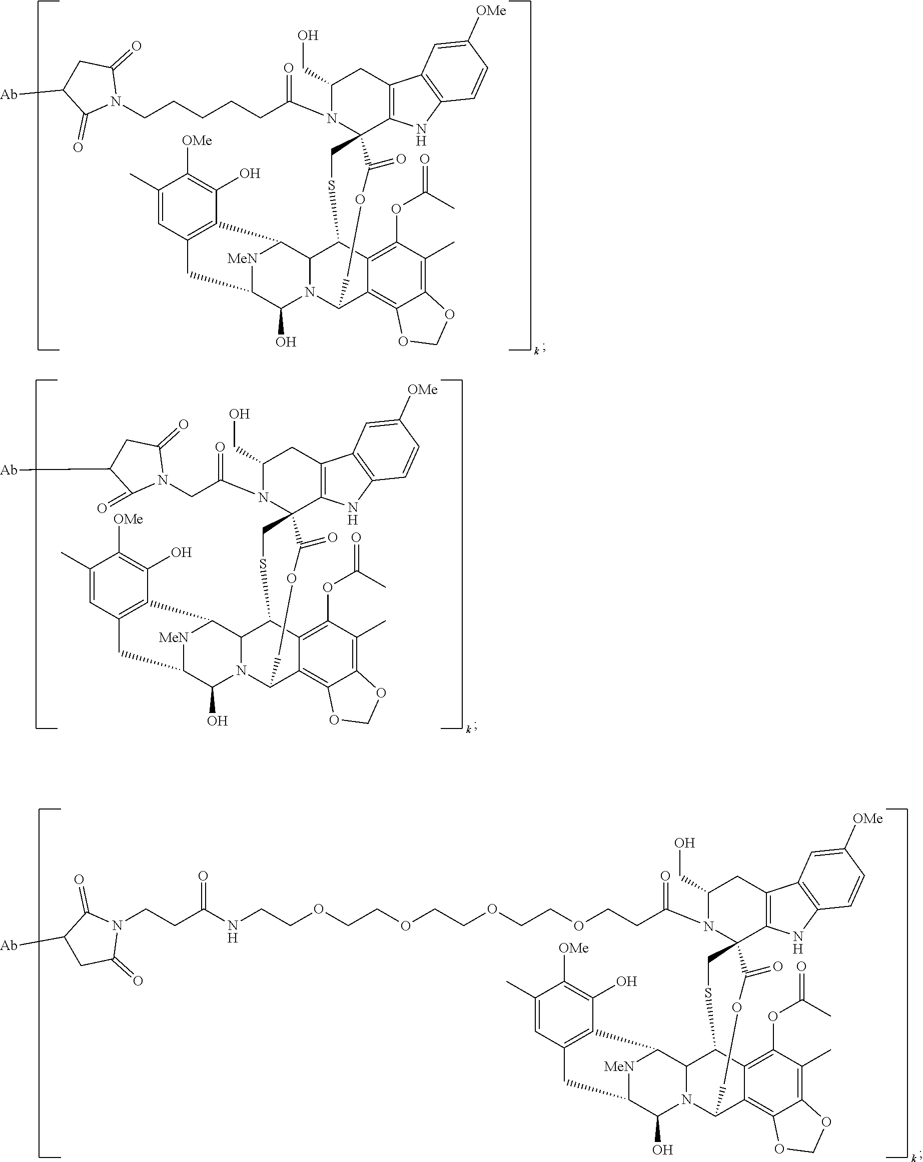

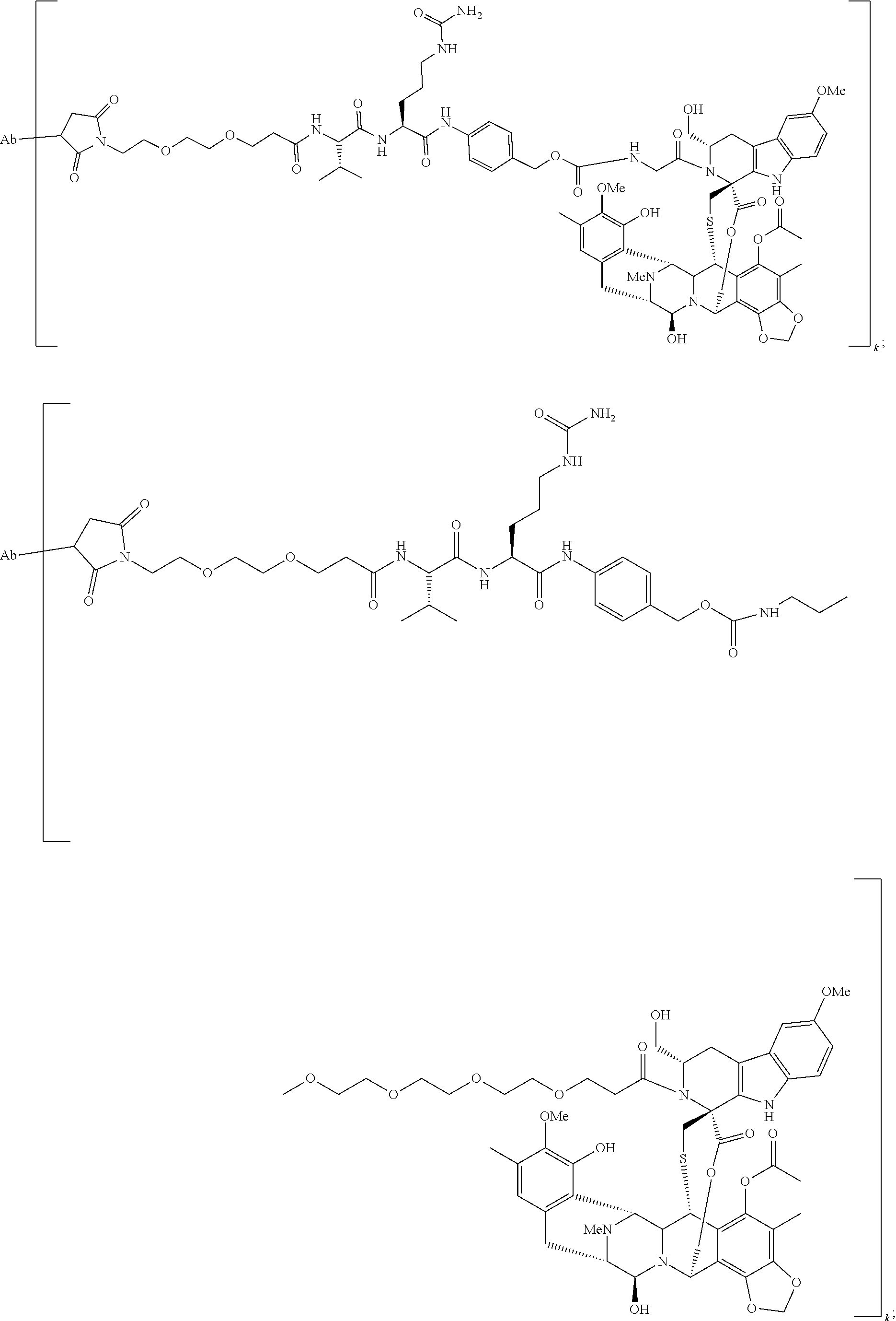

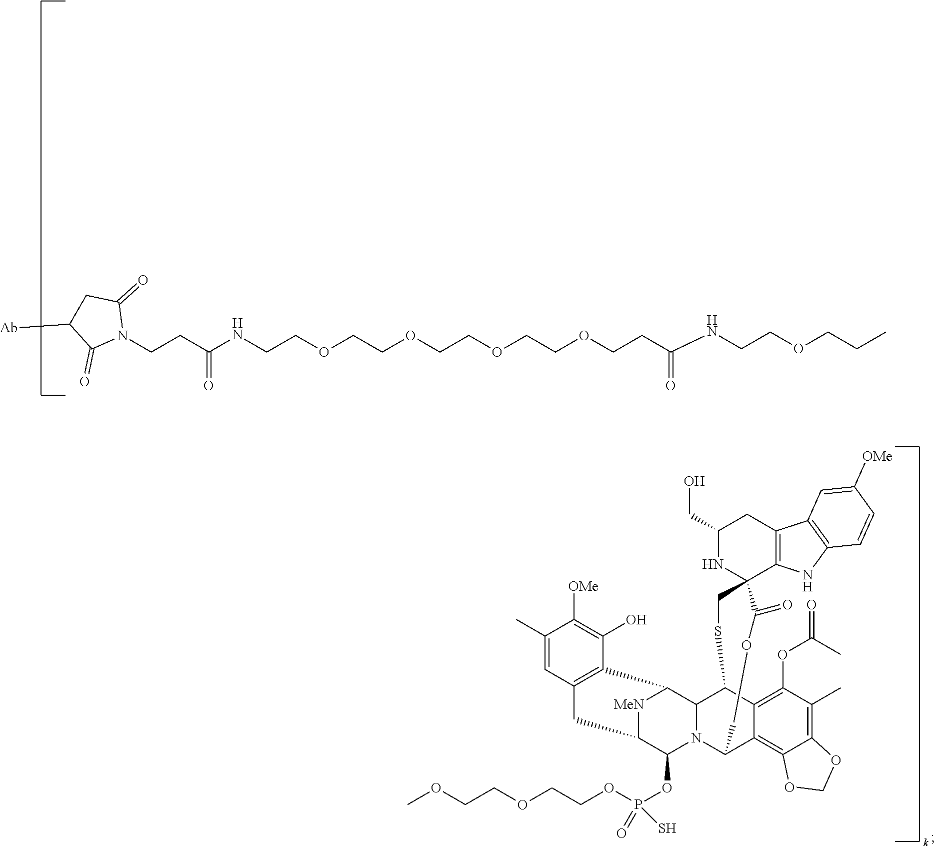

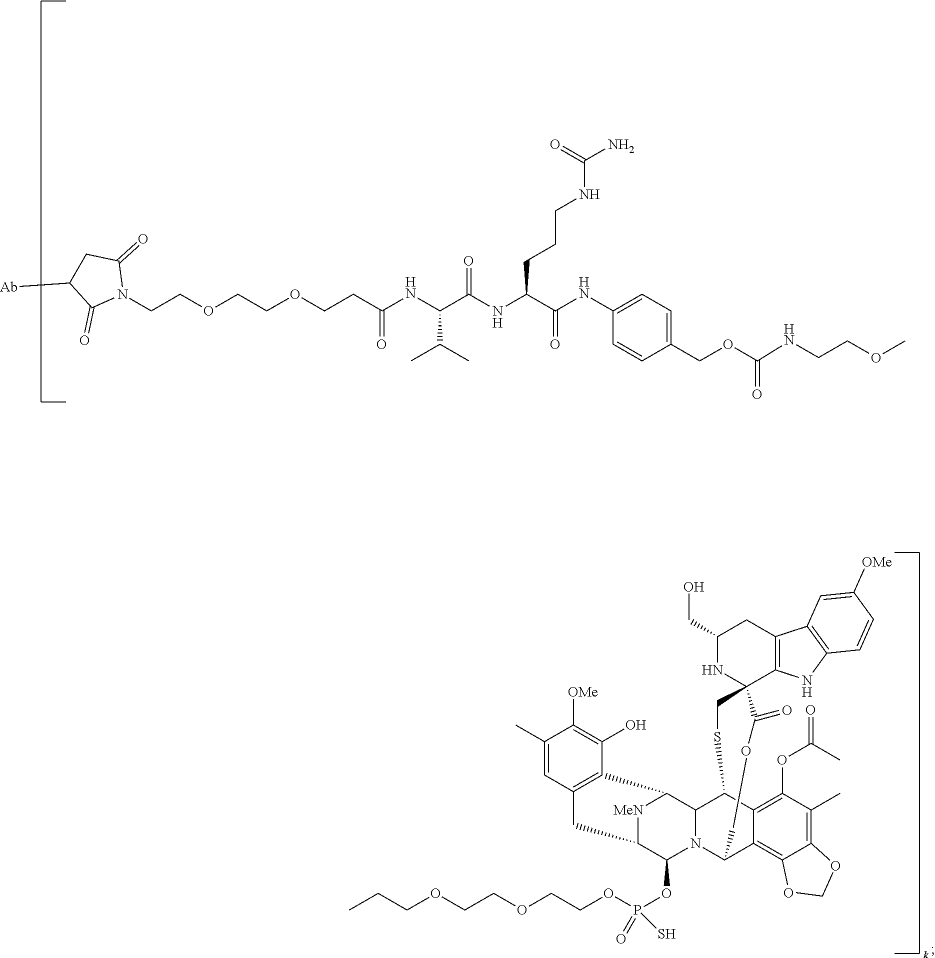

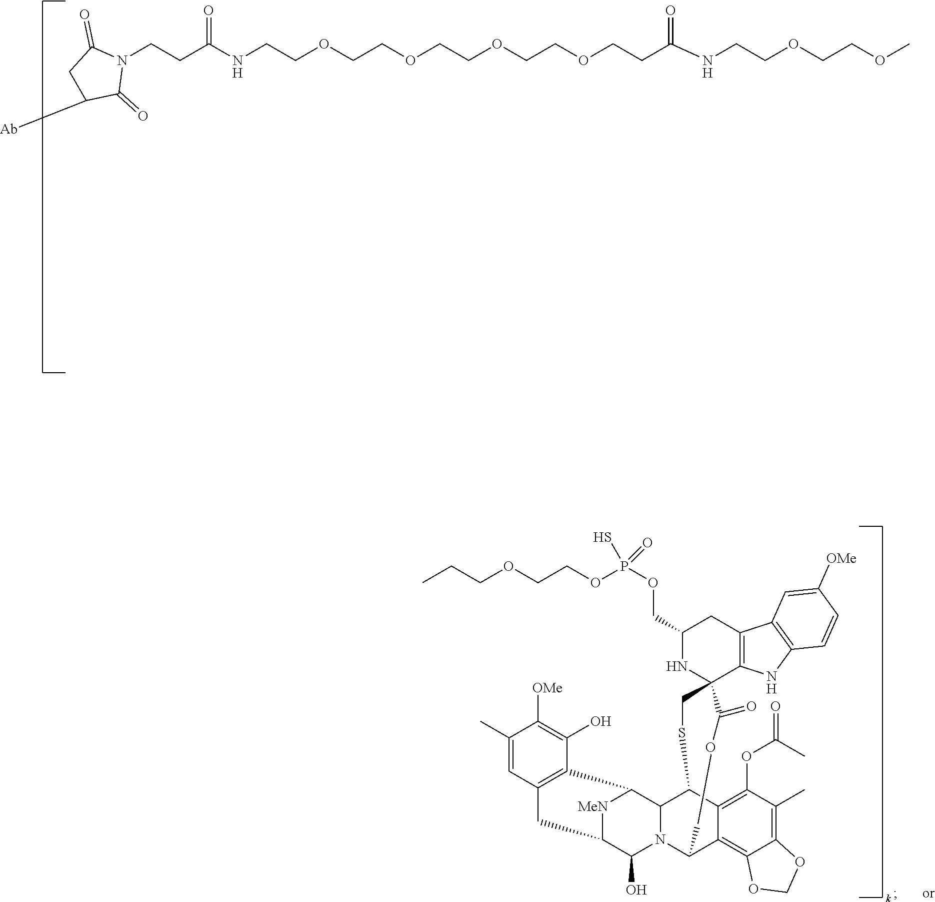

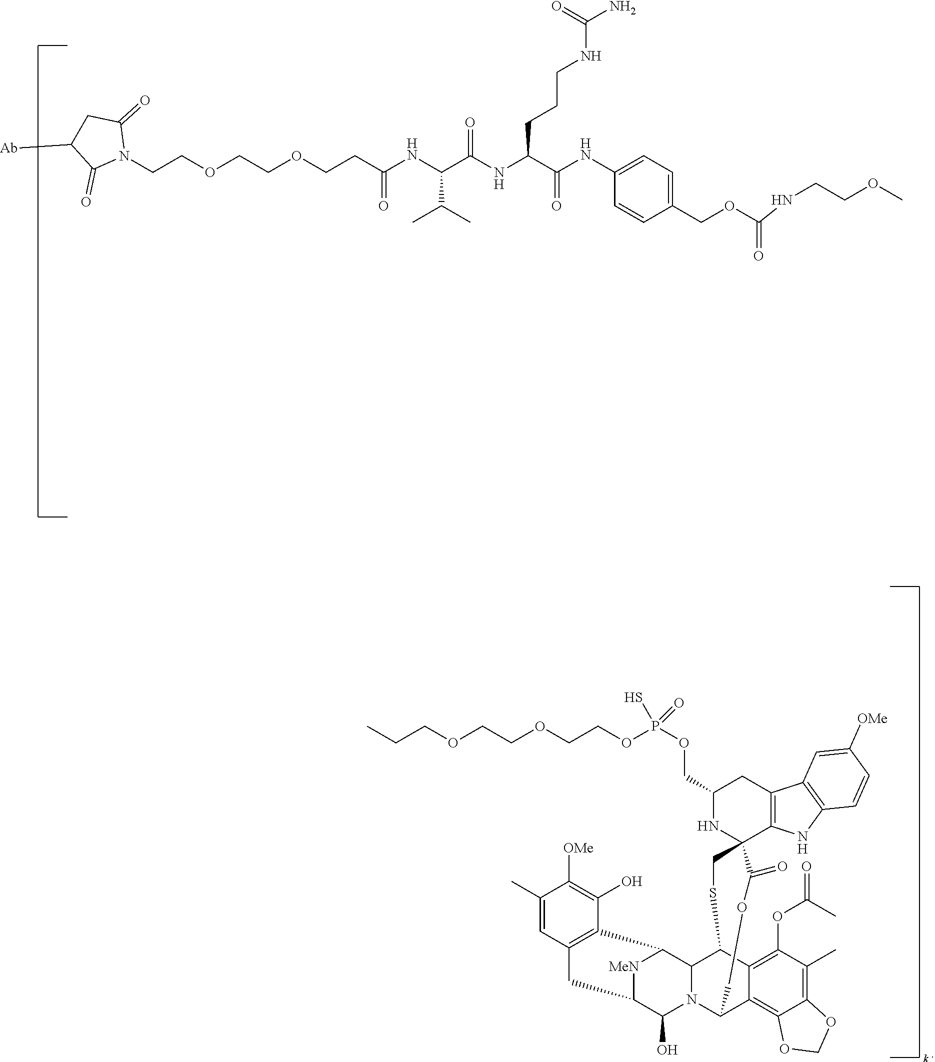

### Methods of Preparing Linker-Drug Conjugates

Disclosed herein are methods for the synthesis of compounds of Formula (XVI) as outlined in Scheme A.

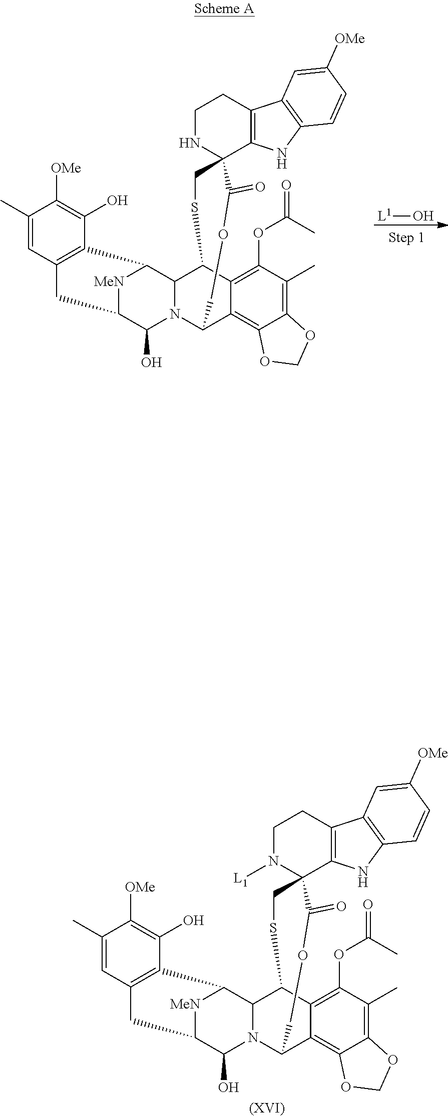

Disclosed herein are methods for the synthesis of compounds of Formula (XVII) as outlined in Scheme B.

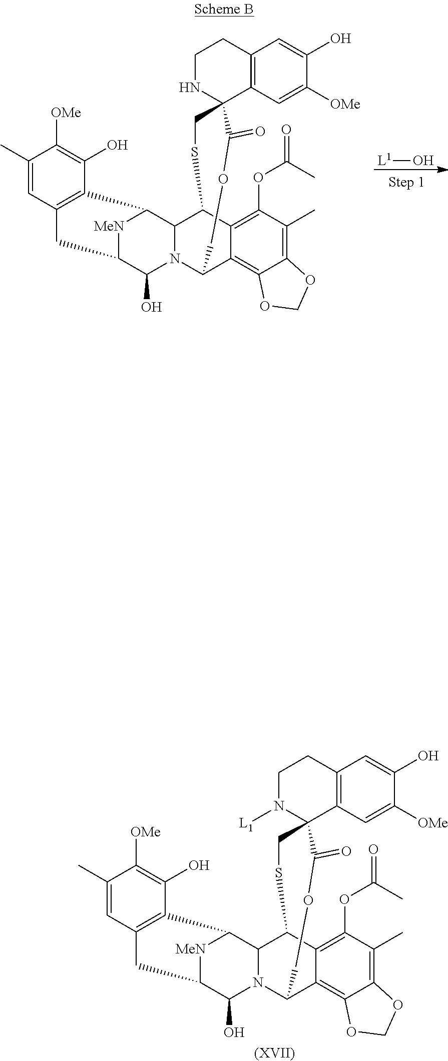

As disclosed herein, variables in Scheme A and Scheme B are defined as follows: L¹ is a linking group terminated with a reactive group. In some embodiments, L¹ is as defined for a compound of Formula (I), Formula (II), Formula (III), Formula (IV), or Formula (XI). In some embodiments, L¹ is a linking group terminated with a reactive group; wherein the reactive group is selected from R⁴⁰. In some embodiments, L¹ is a linking group terminated with a reactive group; wherein the reactive group is selected from maleimide or -NHBoc. In some embodiments, L¹ comprises 0-3 instances of —C(O)—, 0-3 instances of —C(O)—NH— or —NH—C(O)—, 0-3 instances of -(A₁)_{q}-, 0-3 instances of —(C₁₋₆-alkyl)-, 0-2 instances of —O—CH₂—(C₆H₄)—NH—, and 0-3 instances of —(C₂H₄—O)ₘ—; wherein L¹ is terminated in R¹, wherein R⁴⁰ is a reactive group. In some embodiments, at least one occurrence of -(A₁)_{q}- is selected from: Val-Cit or Cit-Val. In some embodiments, L¹ is —C(O)(C₁₋₆-alkyl)-R⁴⁰. In some embodiments, L¹ is —C(O)—(C₂H₄—O)ₘ—(C₁₋₆-alkyl)-NH—C(O)—(C₁₋₆-alkyl)-R⁴⁰. In some embodiments, L¹ is —C(O)—(C₂H₄—O)ₘ—(CH₂)₂—NH—C(O)—(CH₂)₂—R⁴⁰. In some embodiments, L¹ is —C(O)—(C₂H₄—O)ₘ—NH—C(O)—O—CH₂—(C₆H₄—NH-(A₁)-C(O(C₂H₄—O)ₘ—(C₁₋₆-alkyl)-R⁴⁰. In some embodiments, L¹ is -(A₁)-C(O)—O—CH₂—(C₆H₄)—NH-(A₁)_{q}-C(O)—(C₁₋₆-alkyl)-R⁴⁰. In some embodiments, L¹ is —C(O)—(C₂H₄—O)₂—(CH₂)₂—NH—C(O)—(CH₂)₂—R⁴⁰. In some embodiments, L¹ is —C(O)—(C₂H₄—O)₄—(CH₂)₂—NH—C(O)—(CH₂)₂—R⁴⁰. In some embodiments, L¹ is —C(O)—(C₂H₄—O)₁₂—(CH₂)₂—NH—C(O)—(CH₂)₂—R⁴⁰. In some embodiments, L¹ is —C(O)—(CH₂)₅—R⁴⁰. In some embodiments, L¹ is —C(O)—(CH₂)—R⁴⁰. In some embodiments, L¹ is —C(O)—(C₂H₄—O)₃—(CH₂)₂—NH—C(O)—O—CH₂—(C₆H₄)—NH-(Cit-Val)-C(O)—(C₂H₄—O)₂—(CH₂)₂—R⁴⁰. In some embodiments, L¹ is -(Gly)-C(O)—O—CH₂—(CH₄)—NH-(Cit-Val)-C(O)—(CH₂)₅—R⁴⁰. In some embodiments, R⁴⁰ is maleimide or -NHBoc. In some embodiments, R⁴⁰ is maleimide. In some embodiments, R⁴⁰ is -NHBoc.

In certain embodiments of L¹, A₁ and A₂ are independently in each instance an amino acid (e.g., an amino acid residue), wherein the amino acid is selected from a natural amino acid or a non-natural amino acid. In some embodiments, one or more instances of -(A₁)_{q}- is -(A₁-A₂)_{d}- or -(A₁-A₂)_{g}- as defined throughout the present disclosure (e.g., wherein subscript d is 0 or 1, wherein subscript g is 0 or 1).

In certain embodiments of L¹, subscript n is an integer selected from 0 to 4; subscript m is an integer selected from 0 to 20; subscript p is an integer 0 or 1; subscript q is an integer selected from 1 to 6; and subscript t is an integer selected from 0 to 10. In certain embodiments, each instance of subscript n is independently an integer selected from 0 to 4; each instance of subscript m is independently an integer selected from 1 to 20; each instance of subscript p is independently an integer 0 or 1; each instance of subscript q is independently an integer selected from 1 to 6; and each instance of subscript n is independently an integer selected from 0 to 10.

As disclosed herein, in some embodiments of Scheme, A Step 1, a compound of Formula (XVI) is prepared from lurbinectedin. In some embodiments, lurbinectedin is contacted to a suitable coupling reagent and to a compound having the structure L¹-OH in a suitable solvent to yield the compound of Formula (XVI). In some embodiments, lurbinectedin is contacted to a suitable coupling reagent, to a suitable base, and to a compound having the structure L¹-OH in a suitable solvent to yield the compound of Formula (XVI). In some embodiments, the suitable coupling reagent is selected from fluoro-N,N,N′,N′-bis(tetramethylene)formamidinium hexafluorophosphate (BTFFH), tetramethylfluoroformamidinium hexafluorophosphate (TFFH), or a combination thereof. In some embodiments, the suitable solvent comprises N,N-dimethylformamide (DMF). In some embodiments, the suitable base comprises diisopropylethylamine (DIPEA). In some embodiments, lurbinectedin is contacted to (i) BTFFH, TFFH, or a combination thereof, (ii) DIPEA; and (iii) a compound having the structure L¹-OH in DMF to yield the compound of Formula (XVI).

As disclosed herein, in some embodiments of Scheme B, Step 1, a compound of Formula (XVII) is prepared from trabectedin. In some embodiments, trabectedin is contacted to a suitable coupling reagent and to a compound having the structure L¹-OH in a suitable solvent to yield the compound of Formula (XVII). In some embodiments, trabectedin is contacted to a suitable coupling reagent, to a suitable base, and to a compound having the structure L¹-OH in a suitable solvent to yield the compound of Formula (XVII). In some embodiments, the suitable coupling reagent is selected from fluoro-N,N,N′,N′-bis(tetramethylene)formamidinium hexafluorophosphate (BTFFH), tetramethylfluoroformamidinium hexafluorophosphate (TFFH), or a combination thereof. In some embodiments, the suitable solvent comprises N,N-dimethylformamide (DMF). In some embodiments, the suitable base comprises diisopropylethylamine (DIPEA). In some embodiments, trabectedin is contacted to (i) BTFFH, TFFH, or a combination thereof; (ii) DIPEA; and (iii) a compound having the structure L¹-OH in DMF to yield the compound of Formula (XVII).

In some embodiments of Scheme A, Step 1 or Scheme B, Step 1, L-OH is selected from:

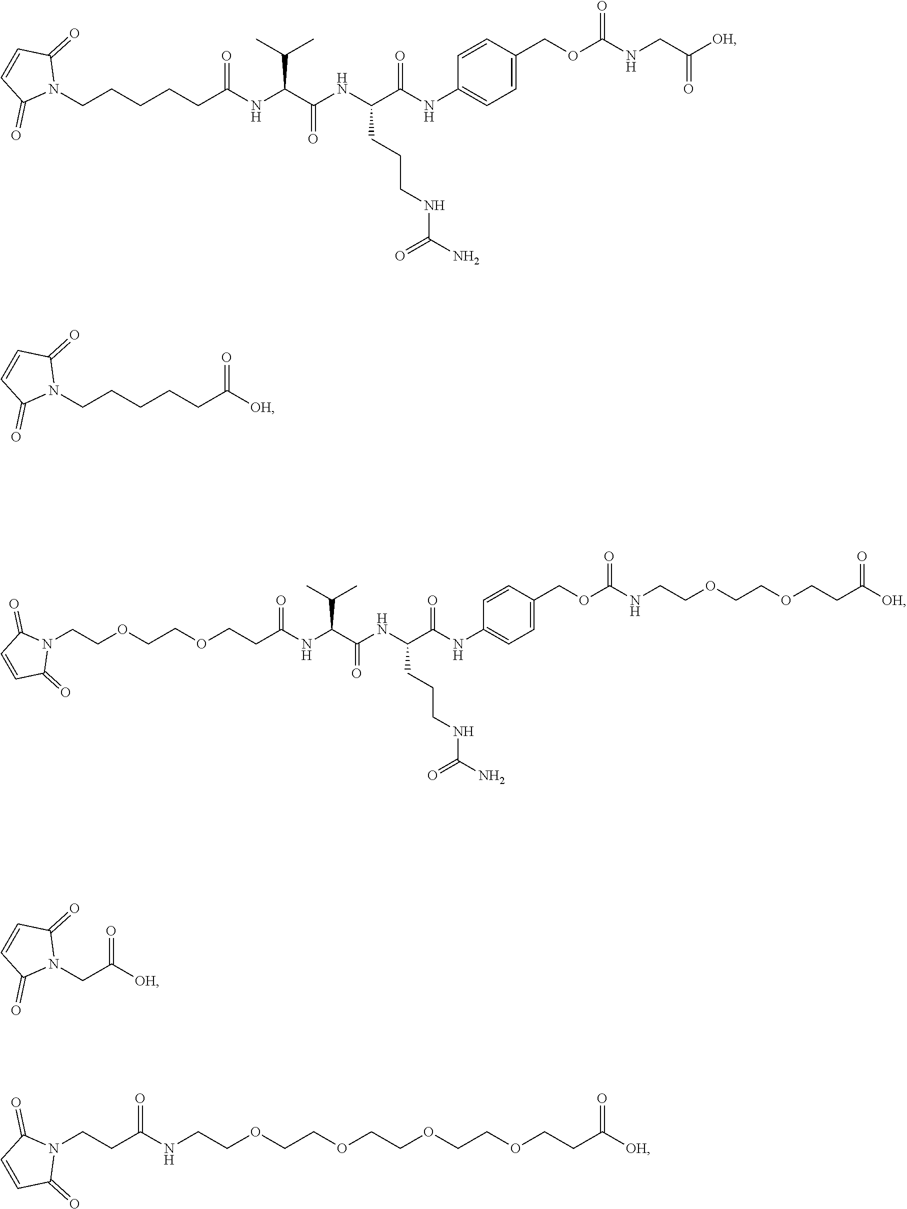

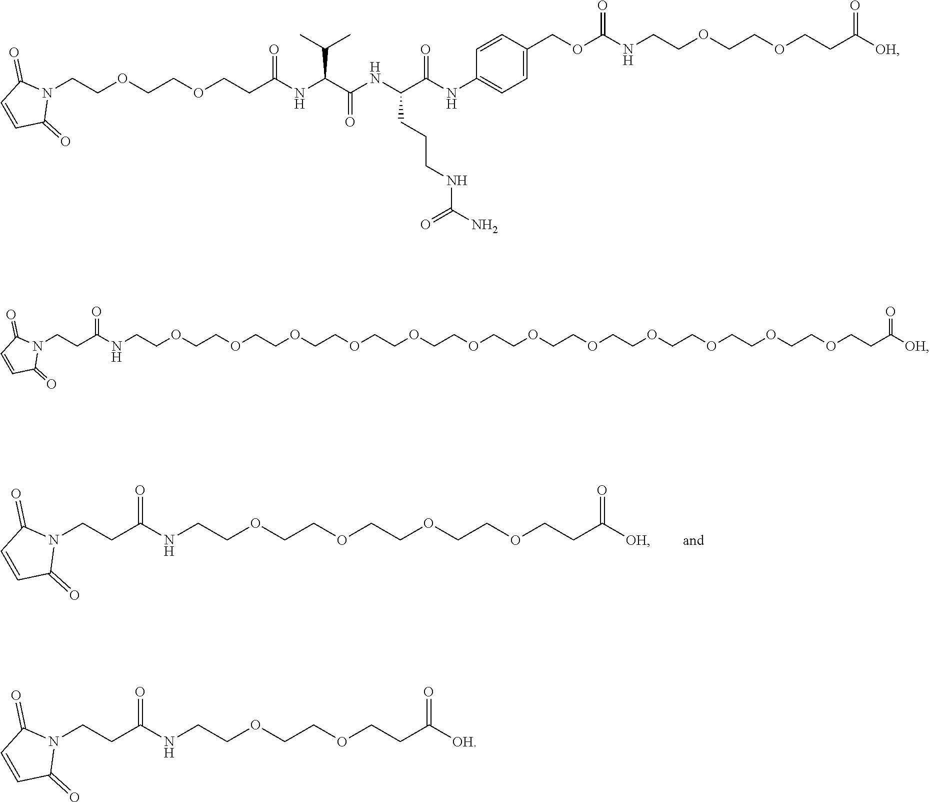 [SMILES: CC(C)[C@H](NC(=O)CCOCCOCCN1C(=O)C=CC1=O)C(=O)N[C@@H](CCCNC(N)=O)C(=O)Nc1ccc(COC(=O)NCCOCCOCCC(=O)O)cc1 ; O=C(O)CCOCCOCCOCCOCCOCCOCCOCCOCCOCCOCCOCCOCCNC(=O)CCN1C(=O)C=CC1=O ; O=C(O)CCOCCOCCOCCOCCNC(=O)CCN1C(=O)C=CC1=O ; O=C(O)CCOCCOCCNC(=O)CCN1C(=O)C=CC1=O]

In some embodiments of a compound of Formula (I), Formula (III), or Formula (IV), or a pharmaceutically acceptable salt, ester, stereoisomer, or tautomer thereof, the compound is a compound of Formula (XVI):

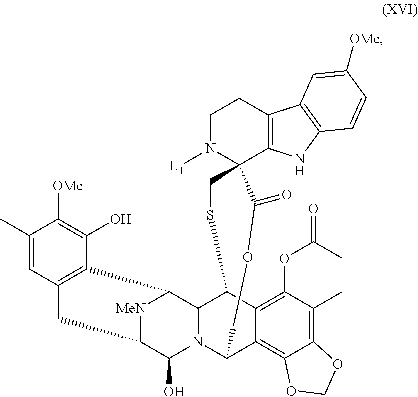

or a pharmaceutically acceptable salt, ester, stereoisomer, or tautomer thereof; wherein L¹ is as defined for a compound of Formula (I), Formula (III), or Formula (IV). Accordingly, in some embodiments of L-OH, L¹ is as defined for a compound of Formula (I), Formula (III), or Formula (IV).

In some embodiments of a compound of Formula (XI), or a pharmaceutically acceptable salt, ester, stereoisomer, or tautomer thereof, the compound is a compound of Formula (XVI):

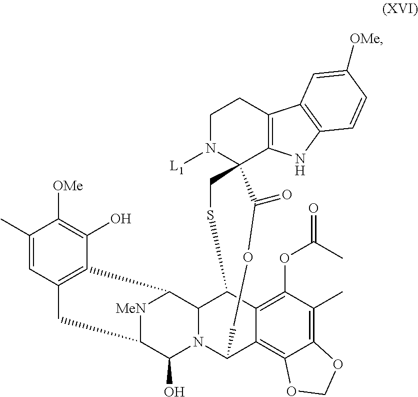

or a pharmaceutically acceptable salt, ester, stereoisomer, or tautomer thereof; wherein L¹ is as defined for a compound of Formula (XI). Accordingly, in some embodiments of L-OH, L¹ is as defined for a compound of Formula (XI).

In some embodiments of a compound of Formula (I), Formula (II), Formula (III), or Formula (IV), or a pharmaceutically acceptable salt, ester, stereoisomer, or tautomer thereof, the compound is a compound of Formula (XVII):

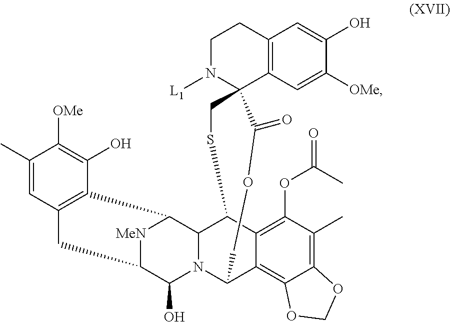

or a pharmaceutically acceptable salt, ester, stereoisomer, or tautomer thereof; wherein L¹ is as defined for a compound of Formula (I), Formula (II), Formula (III), or Formula (IV). Accordingly, in some embodiments of L-OH, L¹ is as defined for a compound of Formula (I), Formula (II), Formula (III), or Formula (IV).

In some embodiments of a compound of Formula (XI), or a pharmaceutically acceptable salt, ester, stereoisomer, or tautomer thereof, the compound is a compound of Formula (XVII):

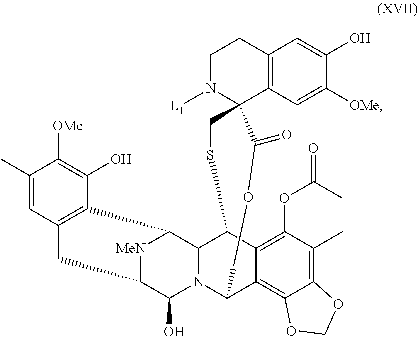

or a pharmaceutically acceptable salt, ester, stereoisomer, or tautomer thereof; wherein L¹ is as defined for a compound of Formula (XI). Accordingly, in some embodiments of L-OH, L¹ is as defined for a compound of Formula (XI).

### Optically Active Compounds

It is appreciated that compounds provided herein can have several chiral centers and may exist in and be isolated in optically active and racemic forms. Some compounds may exhibit polymorphism. It is to be understood that any racemic, optically-active, diastereomeric, polymorphic, or stereoisomeric form, or mixtures thereof, of a compound provided herein, which possess the useful properties described herein is within the scope of the invention. It being well known in the art how to prepare optically active forms (for example, by resolution of the racemic form by recrystallization techniques, by synthesis from optically-active starting materials, by chiral synthesis, or by chromatographic separation using a chiral stationary phase).

Likewise, most amino acids are chiral (designated as L or D, wherein the L enantiomer is the naturally occurring configuration) and can exist as separate enantiomers.

Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

In some embodiments, compositions of the compounds that are substantially free of a designated enantiomer of that compound. In certain embodiments, in the methods and compounds of this invention, the compounds are substantially free of enantiomers. In some embodiments, the composition includes that includes a compound that is at least 85, 90%, 95%, 98%, 99% to 100% by weight, of the compound, the remainder comprising other chemical species or enantiomers.

### Geometric Isomers and Tautomers

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

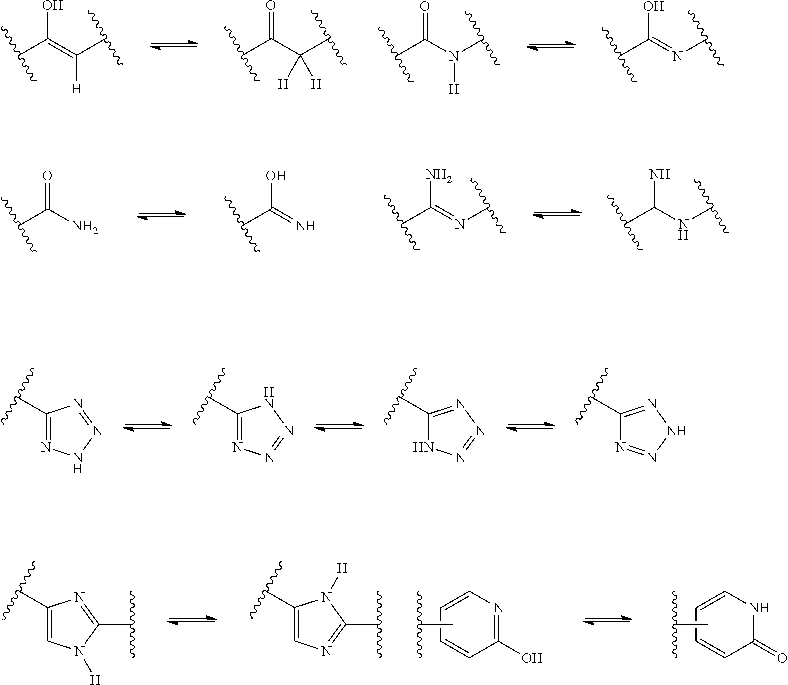

### Isotopically Enriched Compounds

Also provided herein are isotopically enriched compounds, including but not limited to isotopically enriched compounds.

Isotopic enrichment (for example, deuteration) of pharmaceuticals to improve pharmacokinetics (“PK”), pharmacodynamics (“PD”), and toxicity profiles, has been demonstrated previously with some classes of drugs. See, for example, Lijinsky et. al., Food Cosmet. Toxicol., 20: 393 (1982); Lijinsky et. al, J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999).

Isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrees the production of deleterious metabolites in specific tissues and/or create a more effective drug and/or a safer drug for combination therapy, whether the combination therapy is intentional or not.

Replacement of an atom for one of its isotopes often will result in a change in the reaction rate of a chemical reaction. This phenomenon is known as the Kinetic Isotope Effect (“KIE”). For example, if a C—H bond is broken during a rate-determining step in a chemical reaction (i.e., the step with the highest transition state energy), substitution of a deuterium for that hydrogen will cause a decrease in the reaction rate and the process will slow down. This phenomenon is known as the Deuterium Kinetic Isotope Effect (“DKIE”). (See, e.g., Foster et al, Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. High DKIE values may be due in part to a phenomenon known as tunnelling, which is a consequence of the uncertainty principle. Tunnelling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

For example, the DKIE was used to decrease the hepatotoxicity of halothane by presumably limiting the production of reactive species such as trifluoroacetyl chloride. However, this method may not be applicable to all drug classes. For example, deuterium incorporation can lead to metabolic switching. The concept of metabolic switching asserts that xenogens, when sequestered by Phase I enzymes, may bind transiently and re-bind in a variety of conformations prior to the chemical reaction (e.g., oxidation). This hypothesis is supported by the relatively vast size of binding pockets in many Phase I enzymes and the promiscuous nature of many metabolic reactions. Metabolic switching can potentially lead to different proportions of known metabolites as well as altogether new metabolites. This new metabolic profile may impart more or less toxicity.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein will produce a detectable KIE that will affect the pharmacokinetic, pharmacologic, and/or toxicological profiles of a compound provided herein in comparison with a similar compound having a natural isotopic composition.

### Preparation of Compounds & Pharmaceutical Compositions

In certain embodiments, one or more protection or deprotection steps may be included in the methods of preparation described in the Examples.

The present compounds can be formulated into pharmaceutical compositions using methods available in the art and those disclosed herein. Any of the compounds disclosed herein can be provided in the appropriate pharmaceutical composition and be administered by a suitable route of administration.

The methods provided herein encompass administering pharmaceutical compositions containing at least one compound as described herein if appropriate in the salt form, either used alone or in the form of a combination with one or more compatible and pharmaceutically acceptable carriers, such as diluents or adjuvants, or with another agent.

In certain embodiments, the second agent can be formulated or packaged with the compound provided herein. Of course, the second agent will only be formulated with the compound provided herein when, according to the judgment of those of skill in the art, such co-formulation should not interfere with the activity of either agent or the method of administration. In certain embodiments, the compound provided herein and the second agent are formulated separately. They can be packaged together, or packaged separately, for the convenience of the practitioner of skill in the art.

In clinical practice the active agents provided herein may be administered by any conventional route, in particular parenterally, rectally or by inhalation (e.g., in the form of aerosols).

Use may be made, as solid compositions for powders or granules. In these compositions, the active product is mixed with one or more inert diluents or adjuvants, such as sucrose, lactose or starch.

The compositions for parenteral administration can be emulsions or sterile solutions. These compositions can also contain adjuvants, in particular wetting, isotonizing, emulsifying, dispersing and stabilizing agents. Sterilization can be carried out in several ways, for example using a bacteriological filter, by radiation or by heating. They can also be prepared in the form of sterile solid compositions which can be dissolved at the time of use in sterile water or any other injectable sterile medium.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., a compound provided herein, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Whether a particular excipient is suitable for incorporation into a pharmaceutical composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmocopia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further encompassed herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long term storage in order to determine characteristics such as shelf life or the stability of formulations over time. See, e.g., Jens T. Carstensen, Drug Stability: Principles & Practice, 2d. Ed., Marcel Dekker, NY, NY, 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, powders, and the like. Such compositions and dosage forms will contain a prophylactically or therapeutically effective amount of a prophylactic or therapeutic agent, in certain embodiments, in purified form, together with a suitable amount of carrier so as to provide the form for proper administration to the subject. The formulation should suit the mode of administration. In a certain embodiment, the pharmaceutical compositions or single unit dosage forms are sterile and in suitable form for administration to a subject, for example, an animal subject, such as a mammalian subject, for example, a human subject.

A pharmaceutical composition is formulated to be compatible with its intended route of administration. Examples of routes of administration include, but are not limited to, parenteral, e.g., intravenous, intradermal, subcutaneous, intramuscular, subcutaneous, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: dispersions; suppositories; ointments; cataplasms (poultices); pastes; powders; dressings; creams; plasters; solutions; patches; aerosols (e.g., nasal sprays or inhalers); gels; liquid dosage forms suitable for mucosal administration to a subject, including suspensions (e.g., aqueous or non-aqueous liquid suspensions, oil in water emulsions, or a water in oil liquid emulsions), solutions, and elixirs; liquid dosage forms suitable for parenteral administration to a subject; and sterile solids (e.g., crystalline or amorphous solids) that can be reconstituted to provide liquid dosage forms suitable for parenteral administration to a subject.

The composition, shape, and type of dosage forms provided herein will typically vary depending on their use. For example, a dosage form used in the initial treatment of viral infection may contain larger amounts of one or more of the active ingredients it comprises than a dosage form used in the maintenance treatment of the same infection. These and other ways in which specific dosage forms encompassed herein will vary from one another will be readily apparent to those skilled in the art. See, e.g., Remington's Pharmaceutical Sciences, 20th ed., Mack Publishing, Easton PA (2000).

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms comprise a compound provided herein, or a pharmaceutically acceptable salt, solvate or hydrate thereof lie within the range of from about 0.01 mg to about 1000 mg per day, given as a single once-a-day dose in the morning or as divided doses throughout the day taken with food. Particular dosage forms can have about 0.01, 0.02, 0.03, 0.04, 0.05, 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active compound.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defences against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms.

### Dosage and Unit Dosage Forms

In human therapeutics, the doctor will determine the posology which he considers most appropriate according to a preventive or curative treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

In further aspects, provided are methods of treating or preventing cancer in a subject by administering, to a subject in need thereof, an effective amount of a compound provided herein, or a pharmaceutically acceptable salt thereof. The amount of the compound or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the active compound per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). For compositions provided herein, in certain embodiments, the dosage administered to a subject is 0.140 mg/kg to 3 mg/kg of the subject's body weight, based on weight of the active compound. In certain embodiments, the dosage administered to a subject is between 0.20 mg/kg and 2.00 mg/kg, or between 0.30 mg/kg and 1.50 mg/kg of the subject's body weight.

In certain embodiments, the recommended daily dose range of a composition provided herein for the conditions described herein lie within the range of from about 0.1 mg to about 1000 mg per day, given as a single once-a-day dose or as divided doses throughout a day. In certain embodiments, the daily dose is administered twice daily in equally divided doses. In certain embodiments, a daily dose range should be from about 10 mg to about 200 mg per day, in other embodiments, between about 10 mg and about 150 mg per day, in further embodiments, between about 25 and about 100 mg per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above-described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiment, the dosage of the composition provided herein, based on weight of the active compound, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the composition or a composition provided herein administered to prevent, treat, manage, or ameliorate a disorder, or one or more symptoms thereof in a subject is a unit dose of 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, treatment or prevention can be initiated with one or more loading doses of a compound or composition provided herein followed by one or more maintenance doses. In such embodiments, the loading dose can be, for instance, about 60 to about 400 mg per day, or about 100 to about 200 mg per day for one day to five weeks. The loading dose can be followed by one or more maintenance doses. In certain embodiments, each maintenance does is, independently, about from about 10 mg to about 200 mg per day, between about 25 mg and about 150 mg per day, or between about 25 and about 80 mg per day. Maintenance doses can be administered daily and can be administered as single doses, or as divided doses.

In certain embodiments, a dose of a compound or composition provided herein can be administered to achieve a steady-state concentration of the active ingredient in blood or serum of the subject. The steady-state concentration can be determined by measurement according to techniques available to those of skill or can be based on the physical characteristics of the subject such as height, weight and age. In certain embodiments, a sufficient amount of a compound or composition provided herein is administered to achieve a steady-state concentration in blood or serum of the subject of from about 300 to about 4000 ng/mL, from about 400 to about 1600 ng/mL, or from about 600 to about 1200 ng/mL. In some embodiments, loading doses can be administered to achieve steady-state blood or serum concentrations of about 1200 to about 8000 ng/mL, or about 2000 to about 4000 ng/mL for one to five days. In certain embodiments, maintenance doses can be administered to achieve a steady-state concentration in blood or serum of the subject of from about 300 to about 4000 ng/mL, from about 400 to about 1600 ng/mL, or from about 600 to about 1200 ng/mL.

In certain embodiments, administration of the same composition may be repeated, and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated, and the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising a compound, or a pharmaceutically acceptable salt thereof, in a form suitable for administration. Such forms are described in detail above. In certain embodiments, the unit dosage comprises 1 to 1000 mg, 5 to 250 mg or 10 to 50 mg active ingredient. In particular embodiments, the unit dosages comprise about 1, 5, 10, 25, 50, 100, 125, 250, 500 or 1000 mg active ingredient. Such unit dosages can be prepared according to techniques familiar to those of skill in the art.

The dosages of the second agents are to be used in the combination therapies provided herein. In certain embodiments, dosages lower than those which have been or are currently being used to prevent or treat cancer are used in the combination therapies provided herein. The recommended dosages of second agents can be obtained from the knowledge of those of skill. For those second agents that are approved for clinical use, recommended dosages are described in, for example, Hardman et al., eds., 1996, Goodman & Gilman's The Pharmacological Basis of Basis of Therapeutics 9th Ed, Mc-Graw-Hill, New York; Physician's Desk Reference (PDR) 57th Ed., 2003, Medical Economics Co., Inc., Montvale, NJ, which are incorporated herein by reference in its entirety.

In various embodiments, the therapies (e.g., a compound provided herein and the second agent) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In various embodiments, the therapies are administered no more than 24 hours apart or no more than 48 hours apart. In certain embodiments, two or more therapies are administered within the same patient visit. In other embodiments, the compound provided herein and the second agent are administered concurrently.

In other embodiments, the compound provided herein and the second agent are administered at about 2 to 4 days apart, at about 4 to 6 days apart, at about 1 week part, at about 1 to 2 weeks apart, or more than 2 weeks apart.

In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, a compound provided herein and a second agent are administered to a patient, for example, a mammal, such as a human, in a sequence and within a time interval such that the compound provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In certain embodiments, the compound provided herein and the second active agent exert their effect at times which overlap. Each second active agent can be administered separately, in any appropriate form and by any suitable route. In other embodiments, the compound provided herein is administered before, concurrently or after administration of the second active agent.

In certain embodiments, the compound provided herein and the second agent are cyclically administered to a patient. Cycling therapy involves the administration of a first agent (e.g., a first prophylactic or therapeutic agents) for a period of time, followed by the administration of a second agent and/or third agent (e.g., a second and/or third prophylactic or therapeutic agents) for a period of time and repeating this sequential administration. Cycling therapy can reduce the development of resistance to one or more of the therapies, avoid or reduce the side effects of one of the therapies, and/or improve the efficacy of the treatment.

In certain embodiments, the compound provided herein and the second active agent are administered in a cycle of less than about 3 weeks, about once every two weeks, about once every 10 days or about once every week. One cycle can comprise the administration of a compound provided herein and the second agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the compound provided herein. In certain embodiments, the compound provided herein is administered concurrently with one or more second agents in the same pharmaceutical composition. In another embodiment, a compound provided herein is administered concurrently with one or more second agents in separate pharmaceutical compositions. In still another embodiment, a compound provided herein is administered prior to or subsequent to administration of a second agent. Also contemplated are administration of a compound provided herein and a second agent by the same or different routes of administration, e.g., parenteral. In certain embodiments, when the compound provided herein is administered concurrently with a second agent that potentially produces adverse side effects including, but not limited to, toxicity, the second active agent can advantageously be administered at a dose that falls below the threshold that the adverse side effect is elicited.

### Kits

Also provided are kits for use in methods of treatment of cancer. The kits can include a compound or composition provided herein, a second agent or composition, and instructions providing information to a health care provider regarding usage for treating the disorder. Instructions may be provided in printed form or in the form of an electronic medium such as a floppy disc, CD, or DVD, or in the form of a website address where such instructions may be obtained. A unit dose of a compound or composition provided herein, or a second agent or composition, can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition.

In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

### Methods of Use

In certain embodiments, provided herein are methods for the treatment and/or prophylaxis of cancer that includes the administration of an effective amount of a compounds provided herein, or a pharmaceutically acceptable salt thereof. In certain embodiments, provided herein are methods for treating cancer in a subject. In certain embodiments, the methods encompass the step of administering to the subject in need thereof an amount of a compound effective for the treatment or prevention of cancer in combination with a second agent effective for the treatment or prevention of the infection. The compound can be any compound as described herein, and the second agent can be any second agent described in the art or herein. In certain embodiments, the compound is in the form of a pharmaceutical composition or dosage form, as described elsewhere herein.

In certain embodiments, the subject has received a cancer therapy and discontinued that therapy prior to administration of a method provided herein. In further embodiments, the subject has received therapy and continues to receive that therapy along with administration of a method provided herein. The methods can be co-administered with other therapy for cancer according to the judgment of one of skill in the art. In certain embodiments, the methods or compositions provided herein can be co-administered with a reduced dose of the other therapy for cancer.

### Second Therapeutic Agents

In certain embodiments, the compounds and compositions provided herein are useful in methods of treatment of cancer, that comprises further administration of a second agent effective for the treatment of the disorder. The second agent can be any agent known to those of skill in the art to be effective for the treatment of the disorder, including those currently approved by the FDA.

In certain embodiments, a compound provided herein is administered in combination with one second agent. In further embodiments, a second agent is administered in combination with two second agents. In still further embodiments, a second agent is administered in combination with two or more second agents.

As used herein, the term “in combination” includes the use of more than one therapy (e.g., one or more prophylactic and/or therapeutic agents). The use of the term “in combination” does not restrict the order in which therapies (e.g., prophylactic and/or therapeutic agents) are administered to a subject with a disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to a subject with a disorder.

As used herein, the term “synergistic” includes a combination of a compound provided herein and another therapy (e.g., a prophylactic or therapeutic agent) which has been or is currently being used to prevent, manage or treat a disorder, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention or treatment of a disorder). In addition, a synergistic effect can result in improved efficacy of agents in the prevention or treatment of a disorder. Finally, a synergistic effect of a combination of therapies (e.g., a combination of prophylactic or therapeutic agents) may avoid or reduce adverse or unwanted side effects associated with the use of either therapy alone.

The active compounds provided herein can be administered in combination or alternation with another therapeutic agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

### Some Embodiments of the Disclosure

Provided below are some non-limiting embodiments of the present disclosure.

Embodiment 1. A compound of Formula (I), or a pharmaceutically acceptable salt, ester, stereoisomer, or tautomer thereof:

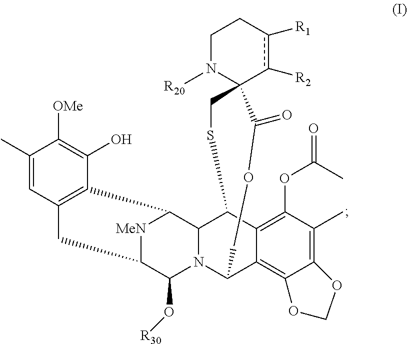

wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H;
wherein R²⁰ is either:
   (a) selected from the group consisting of H, C₁₋₁₀-alkyl, C₆₋₁₀-aryl, C₃₋₁₀-heterocycle, C(O)R₅, C(O)OR₅, C(O)NHR₅, SO₂R₅, and SO₂NHR₅, wherein the C₁₋₁₀-alkyl and C₆₋₁₀-aryl are each, individually, substituted, or unsubstituted; or
   (b) L¹, wherein L¹ is a reactive linker group;
wherein, R²⁰ is optionally substituted by 1-3 substituents, independently in each instance, selected from the group consisting of OH, F, Cl, Br, C₁₋₅-alkyl, C₃₋₆-cycloalkyl, and C₃₋₆-heterocycle;
wherein R³⁰ is either:
   (c) selected from the group consisting of H, C₁₋₁₀-alkyl, C₆₋₁₀-aryl, phosphate, thiophosphate, phosphoramide, C(O)R₆, C(O)OR₆, and C(O)NHR₆, sulfonyl, sulfonylamide,
      wherein the C₁₋₁₀-alkyl and C₆₋₁₀-aryl are each, individually, substituted, or unsubstituted; or
   (d) L², wherein L² is a reactive linker group;
wherein the phosphate and thiophosphate are optionally substituted with one to two substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
   wherein the substituent on the phosphate and thiophosphate is optionally terminated with a protecting group;
wherein R⁵ and R⁶ are each, independently in each instance, selected from the group consisting of C₁₋₆-alkyl, C₃₋₁₀-cycloalkyl, [PEG]_{y}, C₆₋₁₀-aryl, and C₆₋₁₀-heteroaryl,
   wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
wherein at least one of R²⁰ or R³⁰ is not H;
wherein [symbol] is either a single or a double bond; and
wherein subscript y is an integer selected from 0 to 32.

Embodiment 2. The compound of Embodiment 1, wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰; or
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

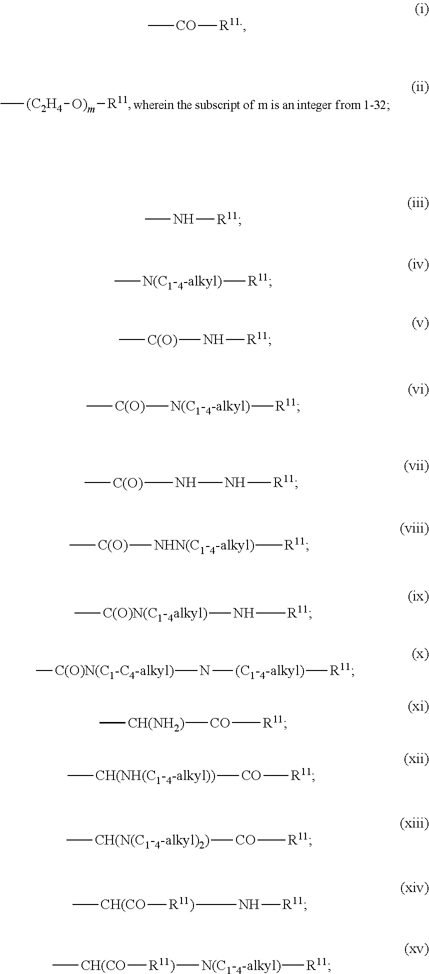

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
   R^{11c} is —(CH₂)ₓ—, wherein the subscript of x is an integer selected from 1 to 10; and R^{11d} is selected from the group consisting of —O— and —NH.

Embodiment 3. The compound of Embodiment 1 or 2, wherein L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of

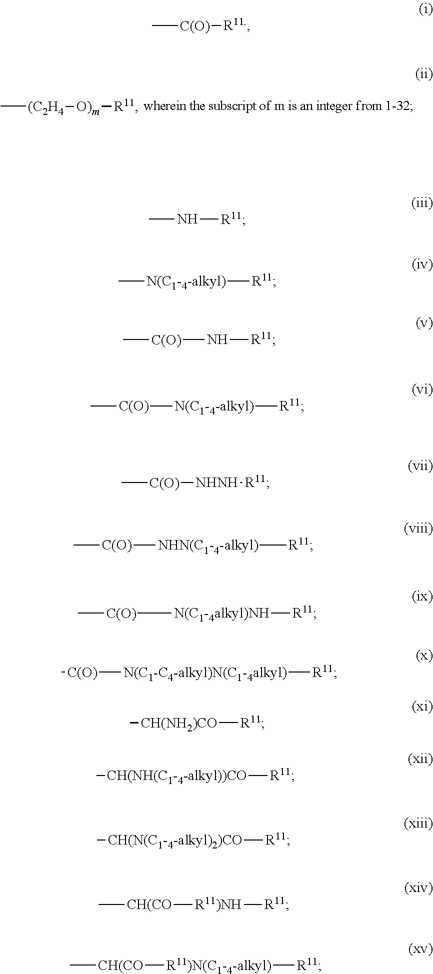

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

Embodiment 4. The compound of Embodiment 2 or 3, wherein R¹¹ is selected from:
R^{11a}-R^{11b}-R^{11c}-R^{11d};
R^{11b}-R^{11c}-R^{11d};
R^{11c}-R^{11d}; or
R^{11a}-R^{11c}-R^{11d}.

Embodiment 5. The compound of any one of Embodiments 2-4, wherein R¹¹ is terminated in a reactive group, R⁴⁰.

Embodiment 6. The compound of any one of Embodiments 1-5, wherein L¹ comprises at least one of the following bivalent structures:

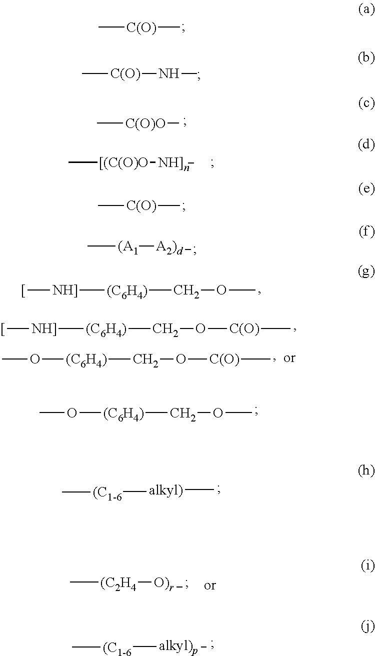

wherein L¹ or L2 are terminated in R⁴⁰, wherein R⁴⁰ is a reactive group;
A₁ and A₂ are independent in each instance an amino acid, wherein the amino acid is selected from a natural amino acid or a non-natural amino acid;
wherein subscript:
   d is an integer selected from 0 to 1;
   n is an integer selected from 0 to 4;
   r is an integer selected from 0 to 32; and
   p is an integer 0 or 1.

Embodiment 7. The compound of any one of Embodiments 1-5, wherein L² comprise at least one of the following bivalent structures:

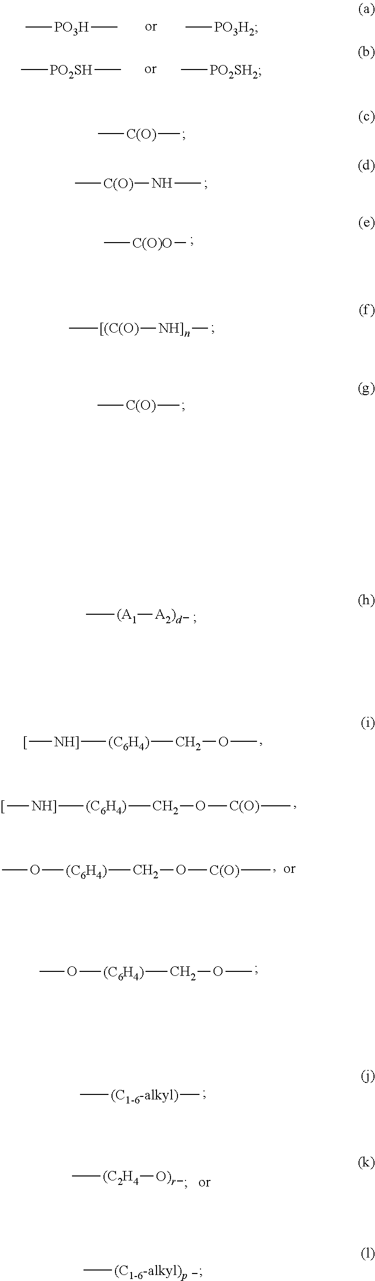

wherein L¹ or L2 are terminated in R⁴⁰, wherein R is a reactive group;
A₁ and A₂ are independent in each instance an amino acid, wherein the amino acid is selected from a natural amino acid or a non-natural amino acid;
wherein subscript:
   d is an integer selected from 0 to 1;
   n is an integer selected from 0 to 4;
   r is an integer selected from 0 to 32; and
   p is an integer 0 or 1.

Embodiment 8. The compound of any one of Embodiments 1-7, wherein R²⁰ is a C₁-alkyl optionally substituted with at least one C₁₋₆-heterocycle.

Embodiment 9. The compound of any one of Embodiments 1-8, wherein R²⁰ is a C₁-alkyl optionally substituted with a bi-pyrrolidinyl substituent.

Embodiment 10. The compound of any one of Embodiments 1-9, wherein R²⁰ is a C₁-alkyl optionally substituted with 1 to 2 pyrrolidinyl substituents.

Embodiment 11. The compound of any one of Embodiments 1-10, wherein R²⁰ is

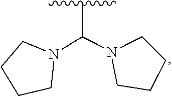 [SMILES: *C(N1CCCC1)N1CCCC1]

wherein

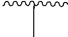

indicates a bond through which the illustrated substituent is bonded.

Embodiment 12. The compound of any one of Embodiments 1-11, wherein R³⁰ is phosphate or thiophosphate.

Embodiment 13. The compound of any one of Embodiments 1-11, wherein R³⁰ is substituted phosphate or substituted thiophosphate.

Embodiment 14. The compound of Embodiment 12, wherein R³⁰ is

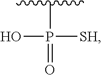 [SMILES: *P(=O)(O)S]

wherein

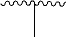

indicates the bond through which the thiophosphate is bonded.

Embodiment 15. A compound of Embodiment 1, wherein the compound is selected from

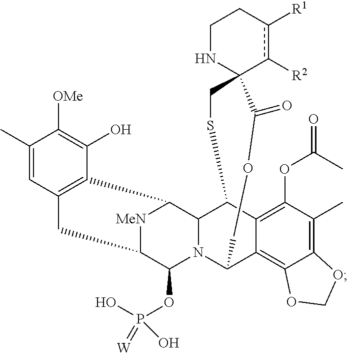

wherein W is O or S.

Embodiment 16. The compound of Embodiment 1, wherein the compound is selected from

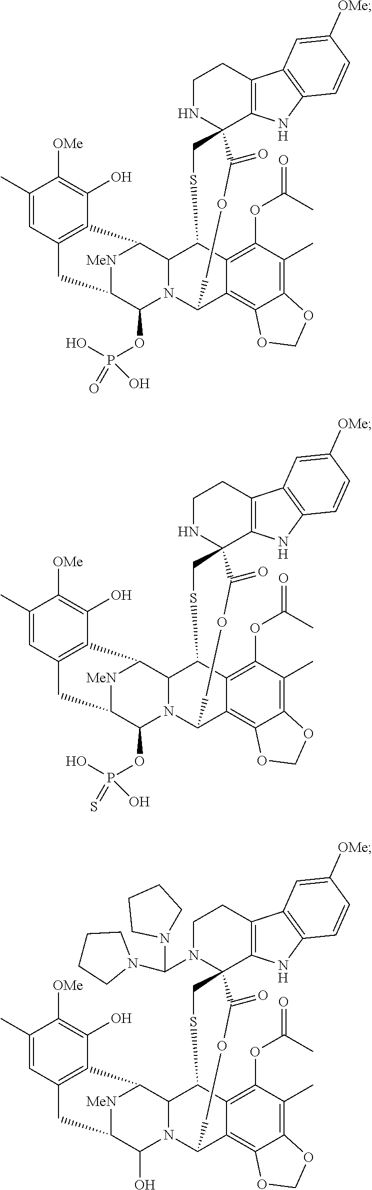 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)O)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(O)(O)=S)N2C)OCO4 ; COc1ccc2[nH]c3c(c2c1)CCN(C(N1CCCC1)N1CCCC1)[C@]31CS[C@@H]2c3c(OC(C)=O)c(C)c4c(c3[C@H](COC1=O)N1C2[C@H]2c3c(cc(C)c(OC)c3O)C[C@@H](C1O)N2C)OCO4]

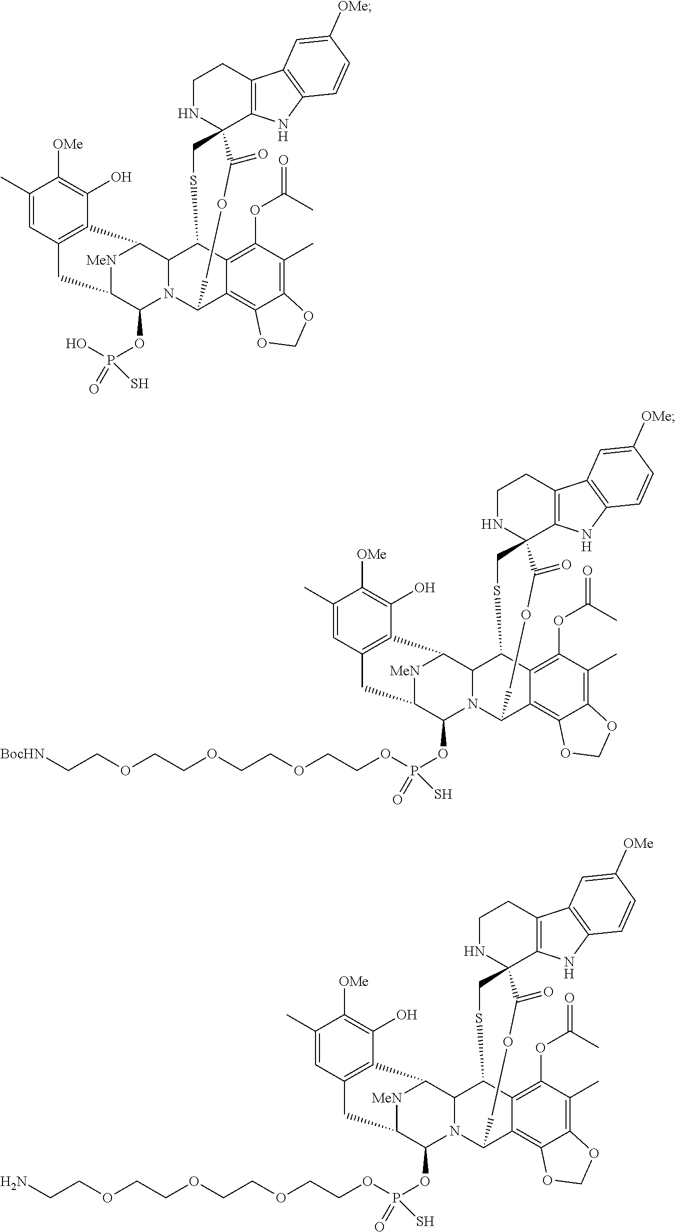 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCN)N2C)OCO4]

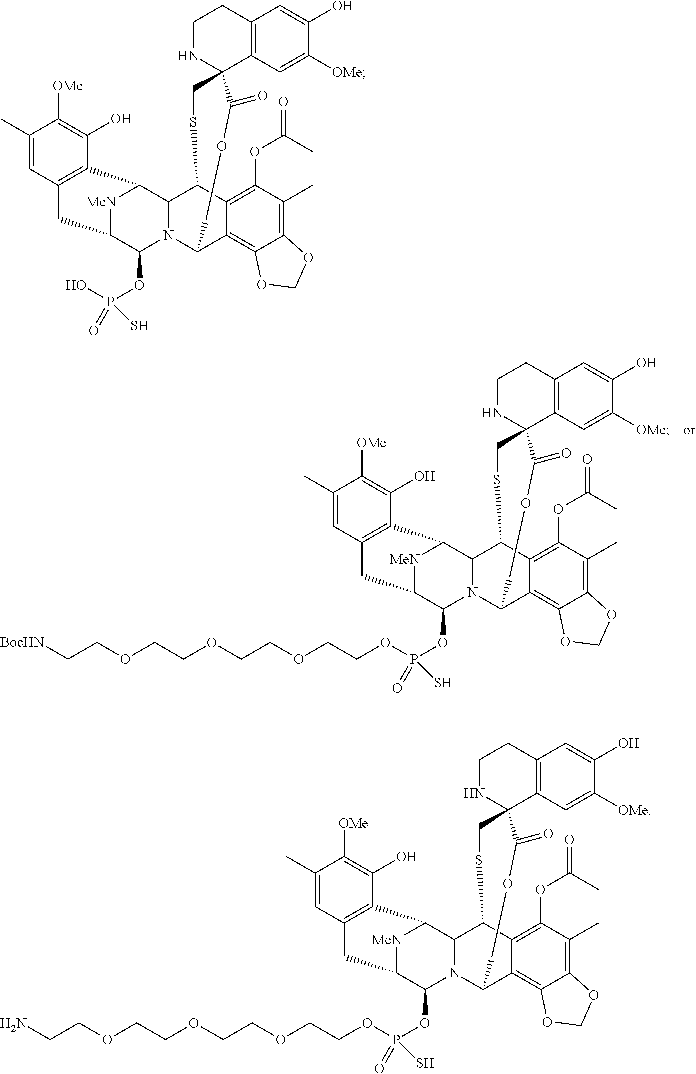 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCN)N2C)OCO4]

Embodiment 17. A compound of Formula (II) or a pharmaceutically acceptable salt, ester, stereoisomer, or tautomer thereof, comprising:

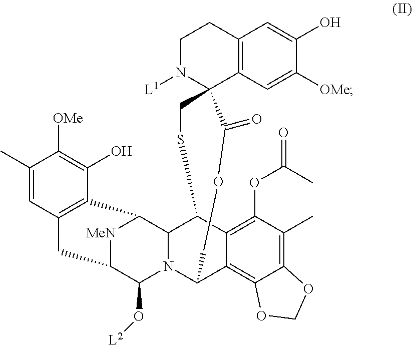

wherein one of L¹ or L² is a reactive linker.

Embodiment 18. The compound of Embodiment 17, wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

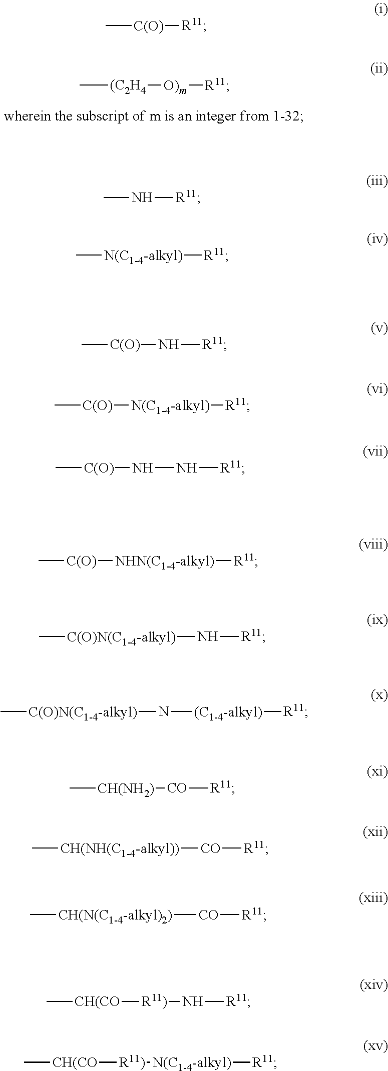

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

Embodiment 19. The compound of Embodiment 17 or 18, wherein L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

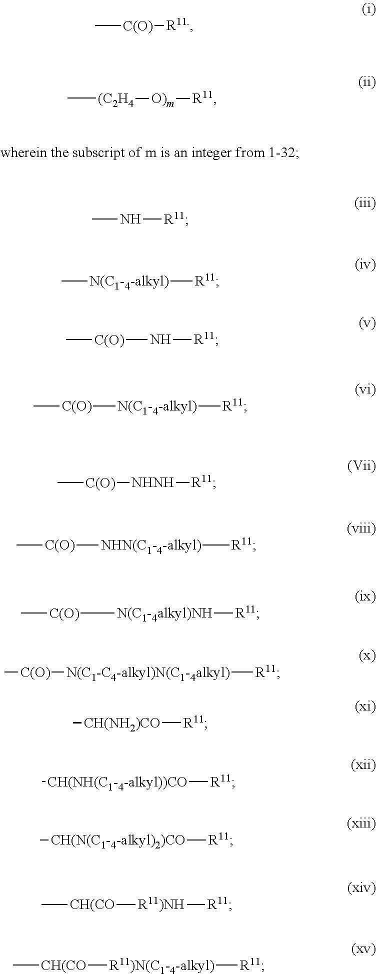

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH.

Embodiment 20. The compound of Embodiment 18 or 19, wherein R¹¹ is selected from:
R^{11a}-R^{11b}-R^{11c}-R^{11d};
R^{11b}-R^{11c}-R^{11d};
R^{11c}-R^{11d}; or
R^{11a}-R^{11c}-R^{11d}.

Embodiment 21. The compound of any one of Embodiments 18-20, wherein R¹¹ is terminated in a reactive group, R⁴⁰.

Embodiment 22. The compound of any one of Embodiments 17-21, wherein L¹ is selected from the group consisting of H, C₁₋₁₀-alkyl, C₆₋₁₀-aryl, C₃₋₁₀-heterocycle, C(O)R₅, C(O)OR₅, C(O)NHR₅, SO₂R₅, and SO₂NHR₅,
wherein the C₁₋₁₀-alkyl and C₆₋₁₀-aryl are each, individually, substituted, or unsubstituted; or
wherein, L¹ is optionally substituted by 1-3 substituents, independently in each instance, selected from the group consisting of OH, F, Cl, Br, C₁₋₅-alkyl, C₃₋₆-cycloalkyl, and C₃₋₆-heterocycle;
wherein R⁵ is selected from the group consisting of C₁₋₆-alkyl, C₃₋₁₀-cycloalkyl, [PEG]_{y}, C₆₋₁₀-aryl, and C₆₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, and —SO₃H;
wherein at least one L¹ or L² is not H; and
wherein subscript y is an integer selected from 0 to 32.

Embodiment 23. The compound of any one of Embodiments 17-21, wherein L² is selected from the group consisting of H, C₁₋₁₀-alkyl, C₆₋₁₀-aryl, phosphate, thiophosphate, phosphoramide, C(O)R₆, C(O)OR₆, and C(O)NHR₆, sulfonyl, sulfonylamide,
wherein the C₁₋₁₀-alkyl and C₆₋₁₀-aryl are each, individually, substituted, or unsubstituted; or
wherein the phosphate and thiophosphate are optionally substituted with one to two substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
   wherein the substituent on the phosphate and thiophosphate is optionally terminated with a protecting group;
wherein R⁶ is selected from the group consisting of C₁₋₆-alkyl, C₃₋₁₀-cycloalkyl, [PEG]_{y}, C₆₋₁₀-aryl, and C₆₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, and —SO₃H;
wherein at least one L¹ or L² is not H; and
wherein subscript y is an integer selected from 0 to 32.

Embodiment 24. A compound of Formula (III), or a pharmaceutically acceptable salt, ester, stereoisomer, or tautomer thereof, comprising:

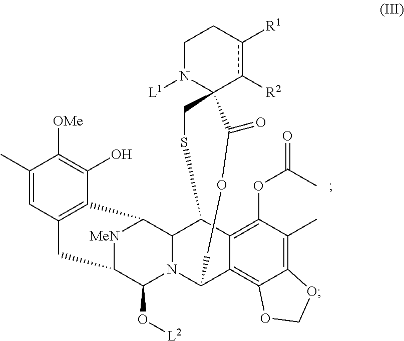

wherein R¹ and R² form at least one aromatic ring or bicyclic heterocyclic ring, optionally substituted with one to six substituents, independently in each instance, selected from C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

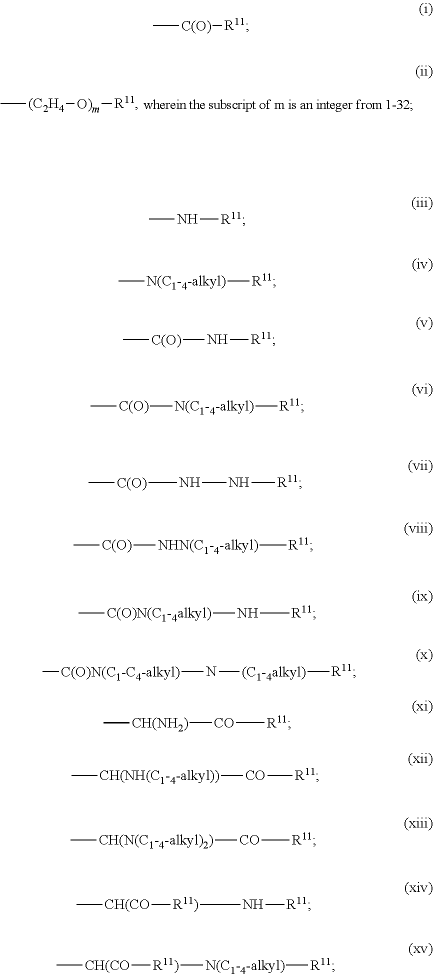

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH;
wherein L² comprises at least one of the following:
(a) —H, if L¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

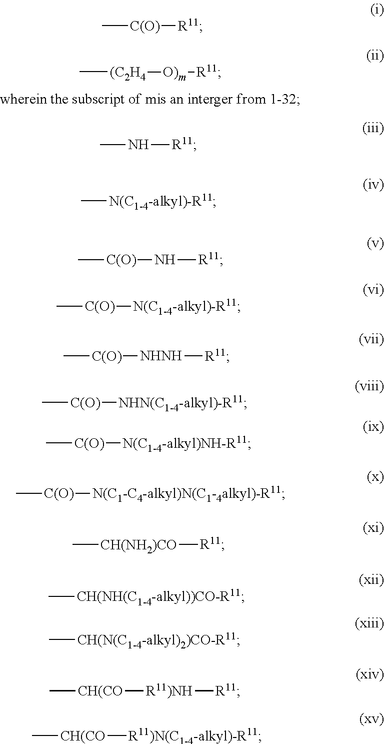

wherein R¹¹ is selected from the group consisting of R^{11a}, R^{11b}, R^{11c}, R^{11d}, and combinations thereof, wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
R^{11d} is selected from the group consisting of —O— and —NH.

Embodiment 25. A compound of Formula (IV) or a pharmaceutically acceptable salt, ester, or tautomer thereof, comprising:

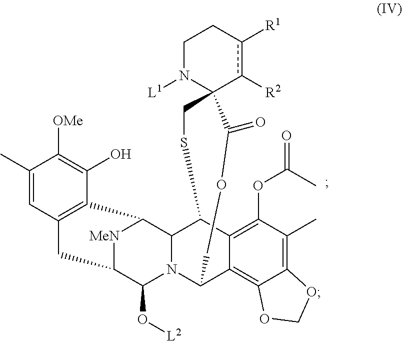

wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H;
wherein [symbol] is either a single or a double bond;
wherein one of L¹ or L² is a linker;
wherein L¹ is:

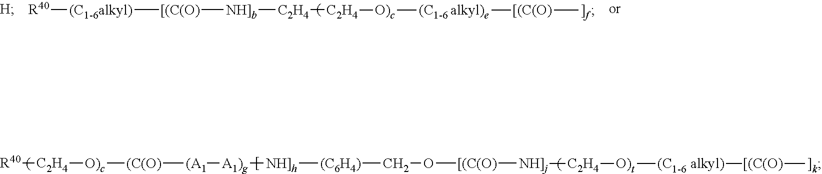

wherein L² is:

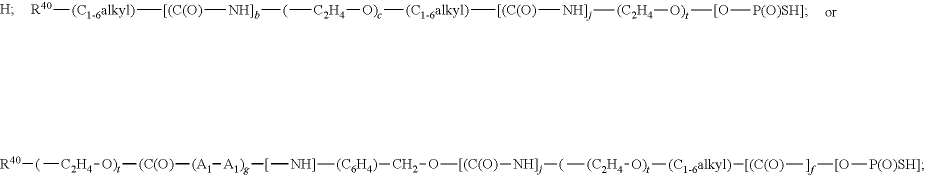

wherein one of L¹ or L² is not H;
wherein R⁴⁰ is a reactive group;
wherein A₁ and A₂ are independently in each instance an amino acid;
wherein subscript:
   b and j are, independently in each instance, selected from 0 to 4;
   c and t are, independently in each instance, selected from 0 to 32;
   e, f, g, h, and k, are, independently in each instance, 0 or 1.

Embodiment 26. The compound of any one of Embodiments 21-25, wherein R⁴⁰ is maleimido.

Embodiment 27. The compound of any one of Embodiments 18-26, wherein R^{11b} is -(A₁-A₂)_{q}-, wherein A₁-A₂ is: valine-citrulline, citrulline-valine, lysine-phenylalanine, phenylalanine-lysine, valine-asparagine, asparagine-valine, threonine-asparagine, asparagine-threonine, serine-asparagine, asparagine-serine, phenylalanine-asparagine, asparagine-phenylalanine, leucine-asparagine, asparagine-leucine, isoleucine-asparagine, asparagine-isoleucine, glycine-asparagine, asparagine-glycine, glutamic acid-asparagine, asparagine-glutamic acid, citrulline-asparagine, asparagine-citrulline, alanine-asparagine, or asparagine-alanine.

Embodiment 28. The compound of Embodiment 27, wherein A₁-A₂ are selected from Val and Cit.

Embodiment 29. The compound of any one of Embodiments 24-28, wherein the compound is:

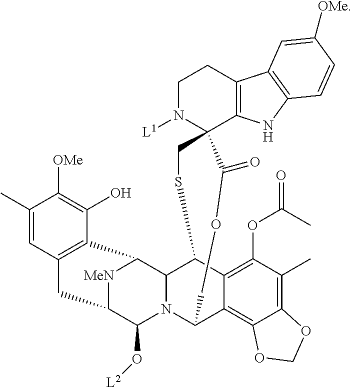

Embodiment 30. The compound of Embodiment 24-28, wherein the compound is:

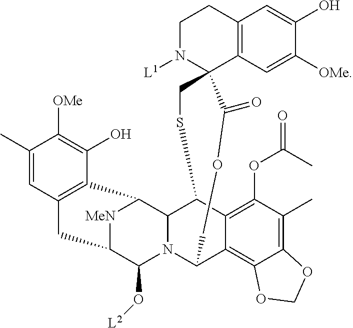

Embodiment 31. The compound of any one of Embodiments 17-30, wherein at least one of L¹ or L² independently comprises:

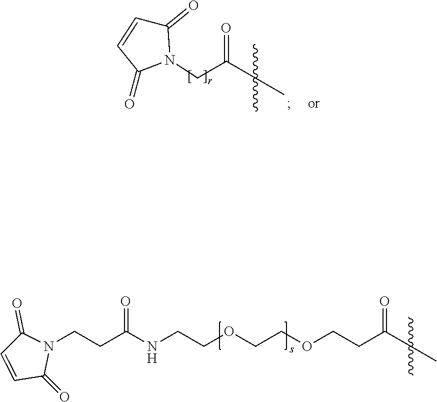

wherein

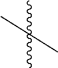

indicates the point of attachment to the compound of Formula (IV) or to a remaining portion of L¹ or L²;
wherein r is an integer select from 1 to 12; and
wherein s is an integer select from 1 to 32.

Embodiment 32. The compound of Embodiment 31, wherein
wherein r is an integer selected from 2 to 12; and
wherein s is an integer selected from 2 to 32.

Embodiment 33. The compound of Embodiment 29, wherein the compound is:

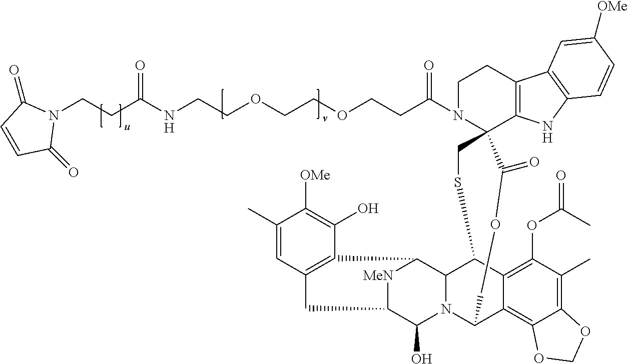

wherein subscript v is an integer from 1 to 32; and
wherein subscript u is an integer from 0 to 11.

Embodiment 34. The compound of Embodiment 33, wherein subscript v is an integer selected from 1 to 14.

Embodiment 35. The compound of Embodiment 33 or 34, wherein the compound is

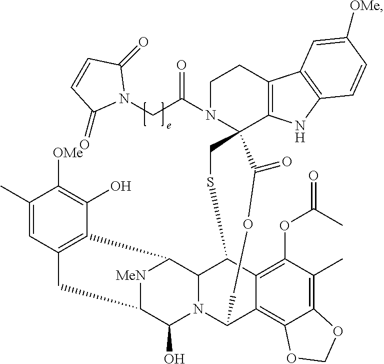

wherein subscript e is an integer selected from 1 to 6.

Embodiment 36. The compound of Embodiment 35, wherein subscript e is an integer from 1 to 4.

Embodiment 37. The compound of Embodiment 17, wherein the compound has the structure:

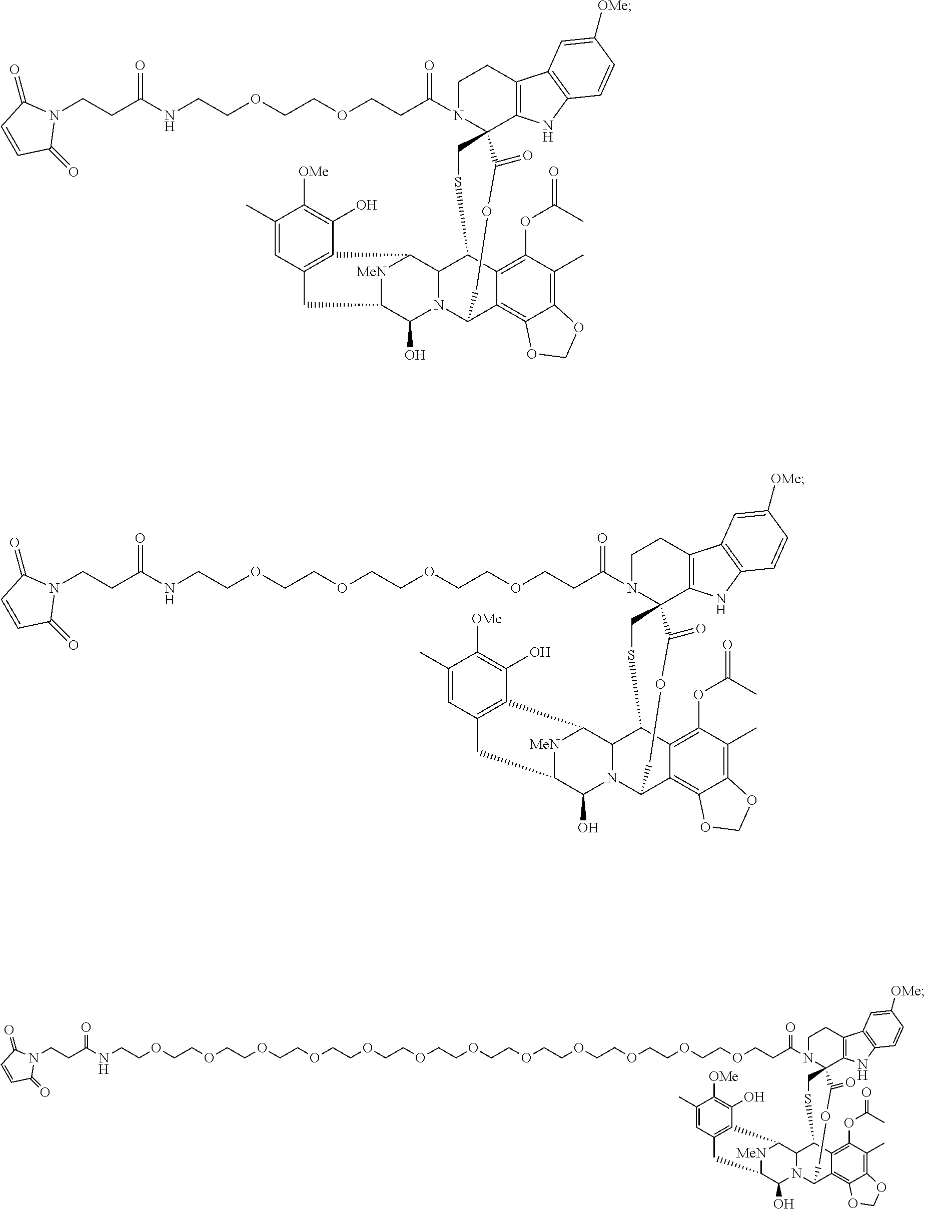

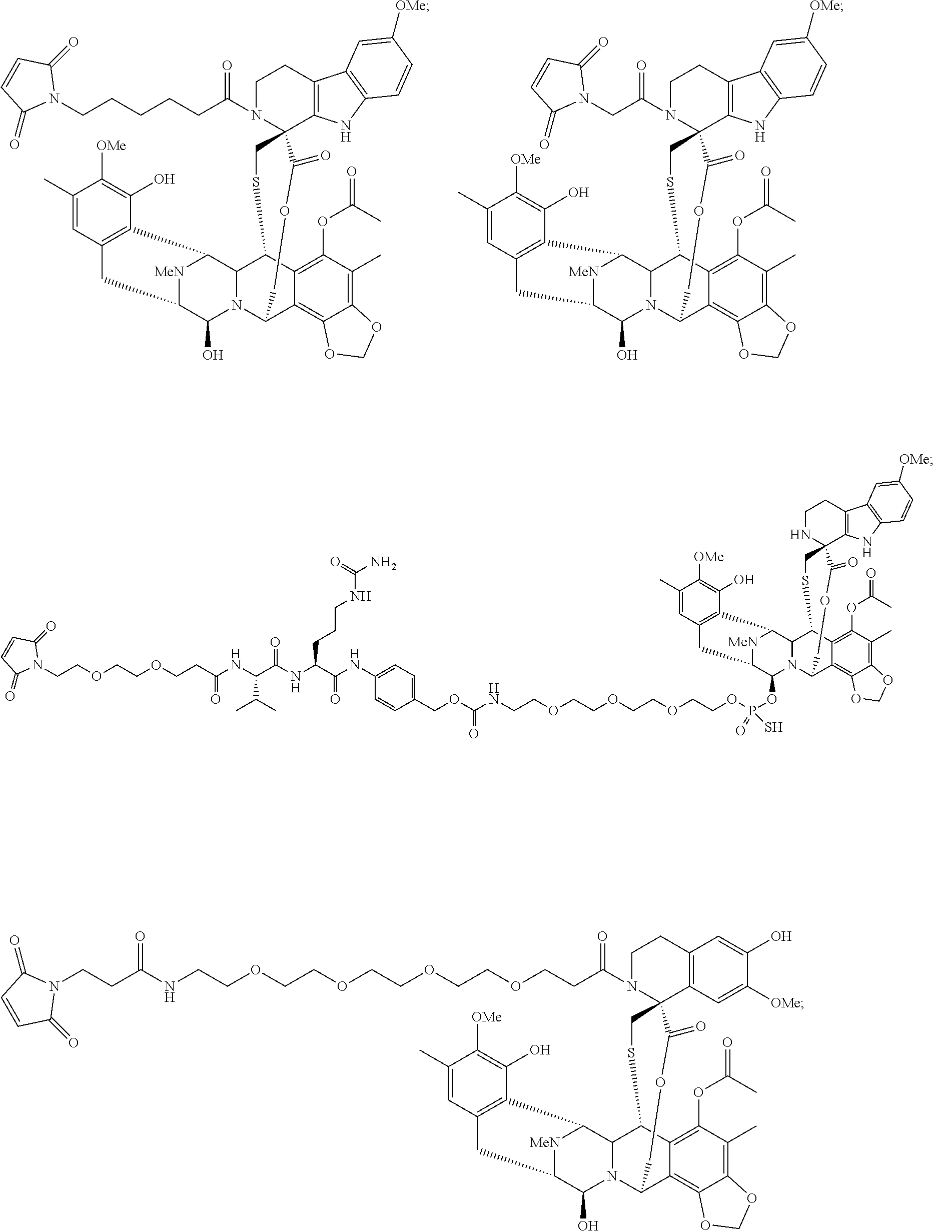

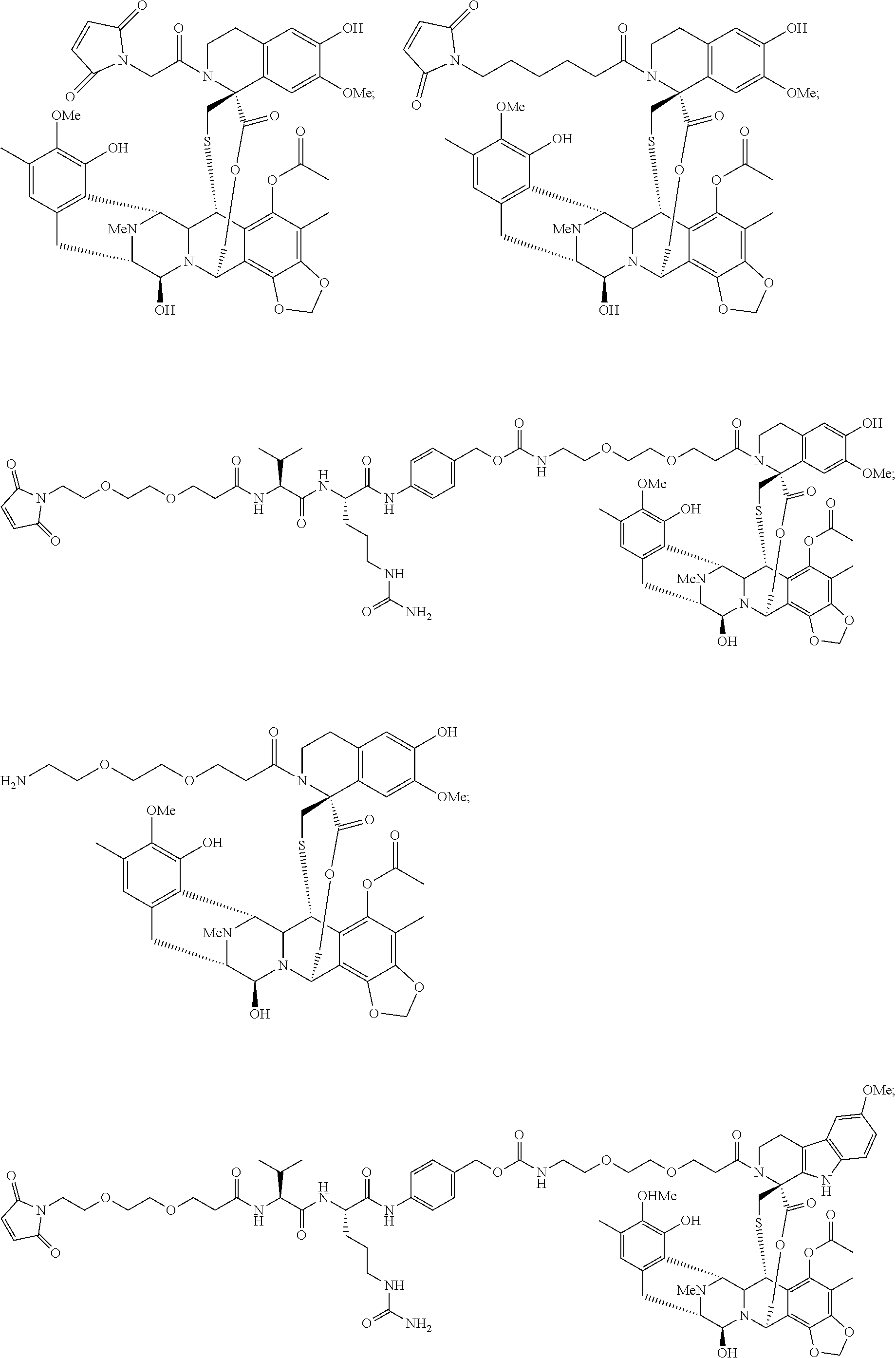

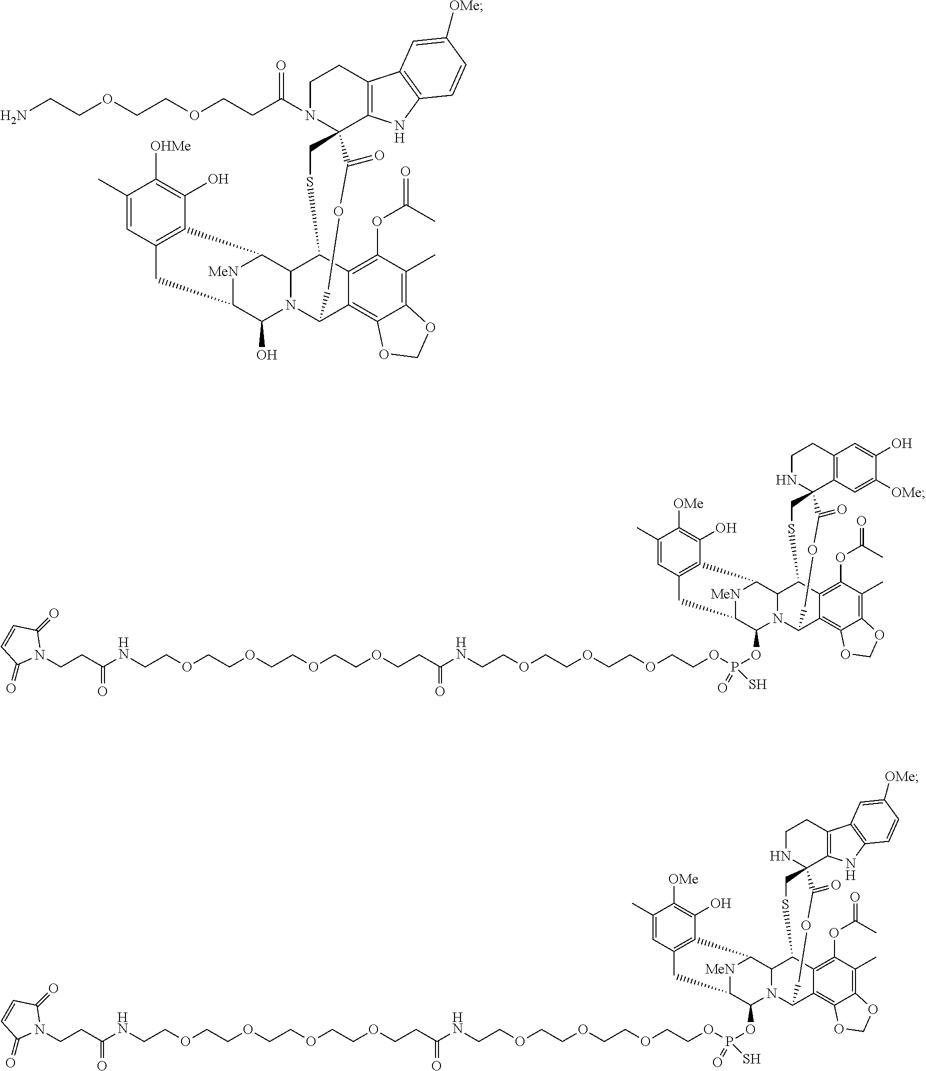

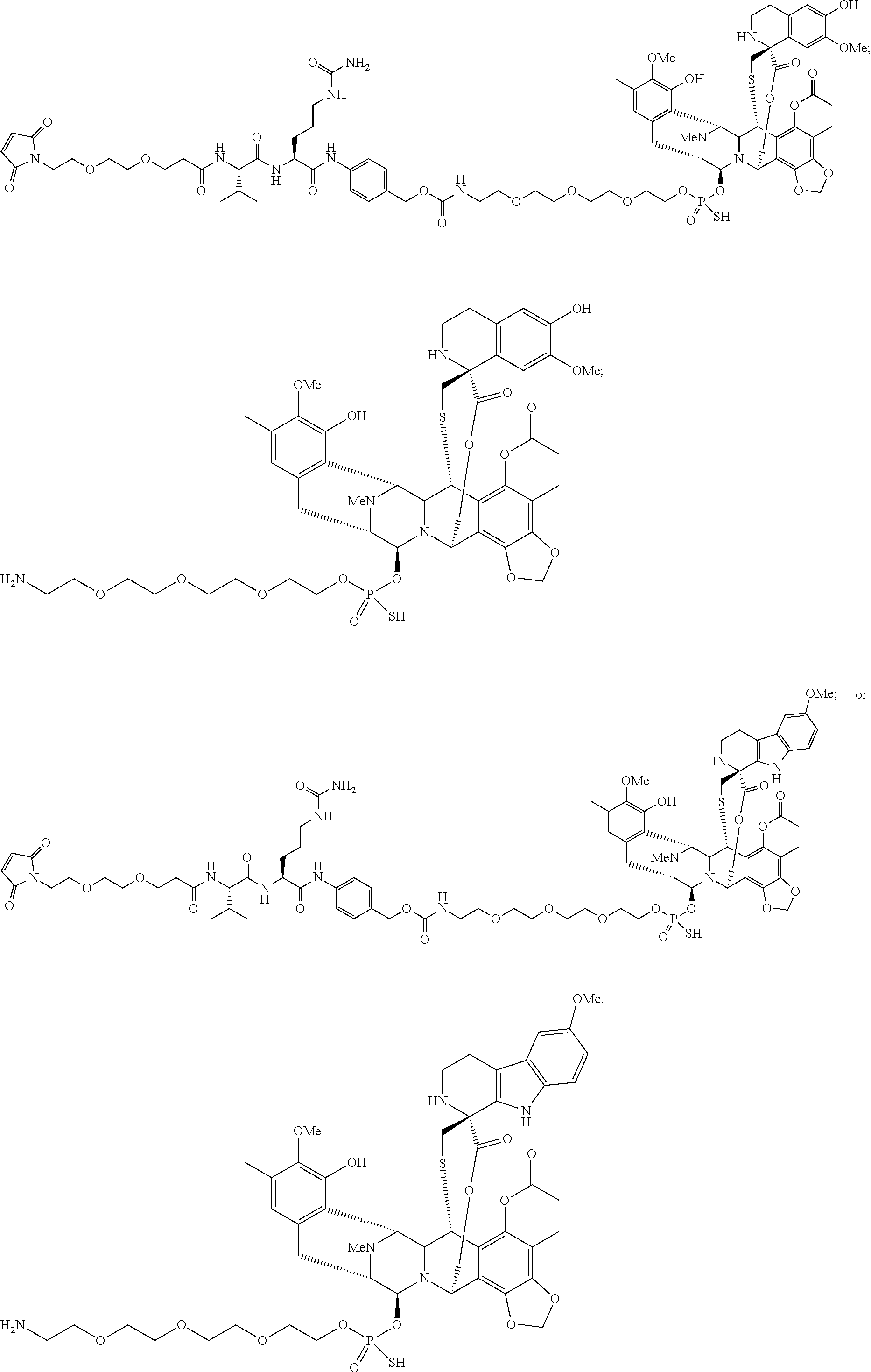

Embodiment 38. A compound comprising a linker or reactive linker covalently bonded to lurbinectedin via a secondary alcohol, or a secondary amine.

Embodiment 39. A compound comprising a linker or reactive linker covalently bonded to trabectedin via a secondary alcohol, or a secondary amine.

Embodiment 40. The compound Embodiments 38 or 39, wherein the compound is covalently bonded to a targeting agent.

Embodiment 41. The compound any one of Embodiments 38-40, wherein the compound is covalently bonded to an antibody or antibody fragment thereof.

Embodiment 42. A compound of Formula (Va) or (Vb), or a pharmaceutically acceptable salt, ester, stereoisomer, or tautomer thereof, comprising:

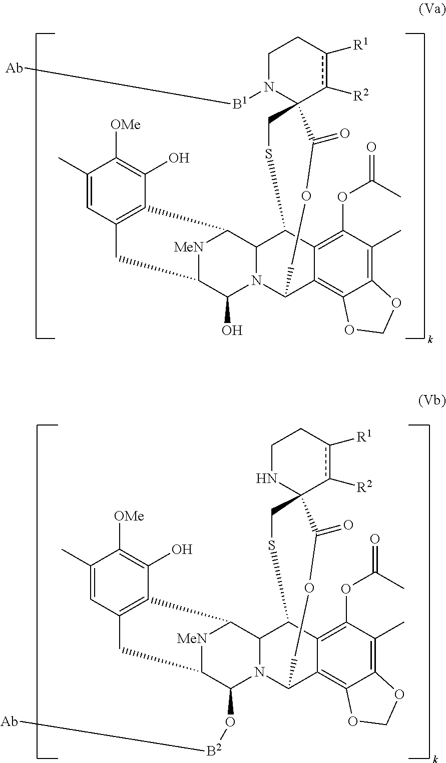

wherein Ab is targeting agent;
wherein subscript k is an integer from 1 to 10;
wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H; and
wherein B¹ is a linker;
wherein B² is a linker.

Embodiment 43. The compound of Embodiment 42, wherein Ab is an antibody, antibody fragment, protein, or peptide.

Embodiment 44. The compound of Embodiment 42 or 43, wherein
wherein B¹ comprises at least one of the following:
(a) —H, if B² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

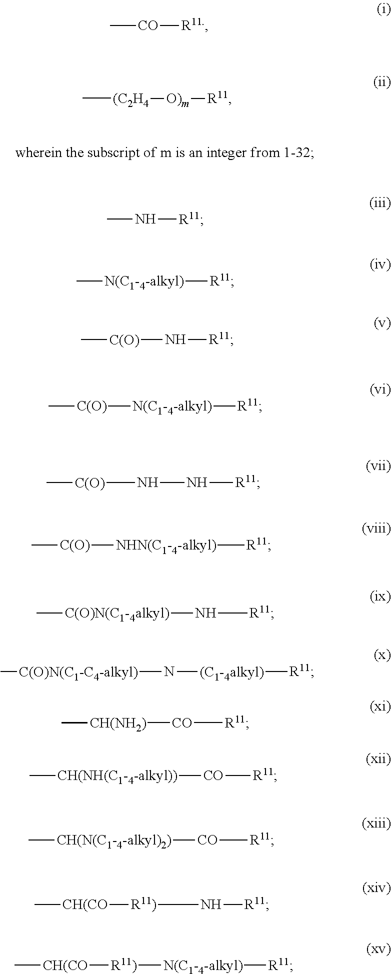

wherein R¹¹ is selected from the group consisting of R^{11a}, R^{11b}, R^{11c}, R^{11d}, and combinations thereof; wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH;
wherein B² comprises at least one of the following:
(a) —H, if B¹ is not H;
(b) —PO₃H—;
(c) —PO₂SH—;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
   R^{10a} is either absent or —(CH₂)ₑ—, wherein e is independently in each instance, selected from 1-4;
   R^{10b} is selected from the group consisting of:

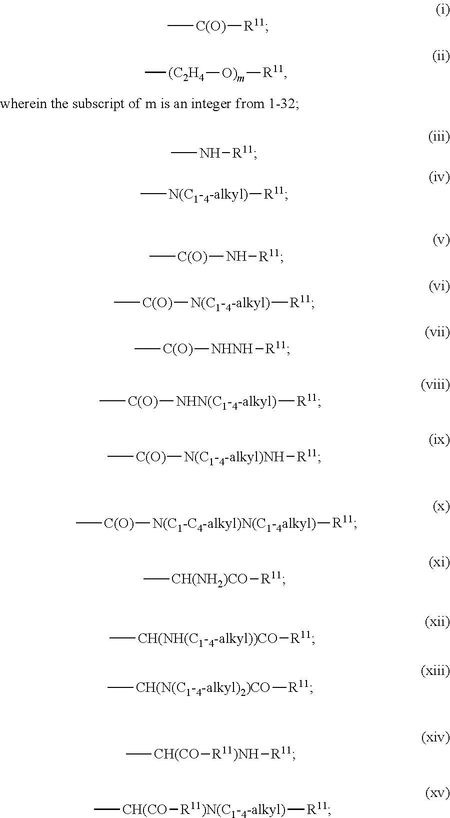

wherein R¹¹ is selected from the group consisting of R^{11a}, R^{11b}, R¹¹, R^{11d}, and combinations thereof; wherein
   R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
   R^{11b} is -(A₁)_{q}-, wherein A₁ is an amino acid, wherein the amino acid is selected from a natural amino acid or a non-natural amino acid, wherein subscript q is an integer selected from 1 to 6;
   R^{11c} is —(CH₂)ₓ—, wherein the subscript of x is an integer selected from 1 to 10; and
   R^{11d} is selected from the group consisting of —O— and —NH; and
wherein one of B¹ or B² is not H; and
wherein one of B¹ or B² is bonded to Ab.

Embodiment 45. The compound of any one of Embodiment 41-43, wherein the compound is

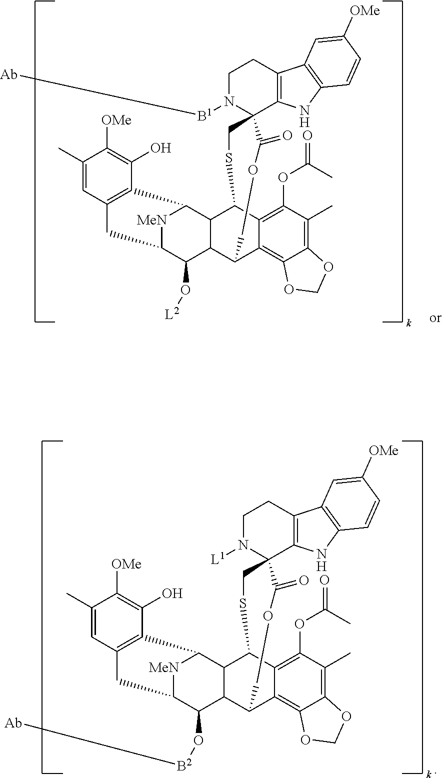

Embodiment 46. The compound of any one of Embodiments 41-43, wherein the compound is

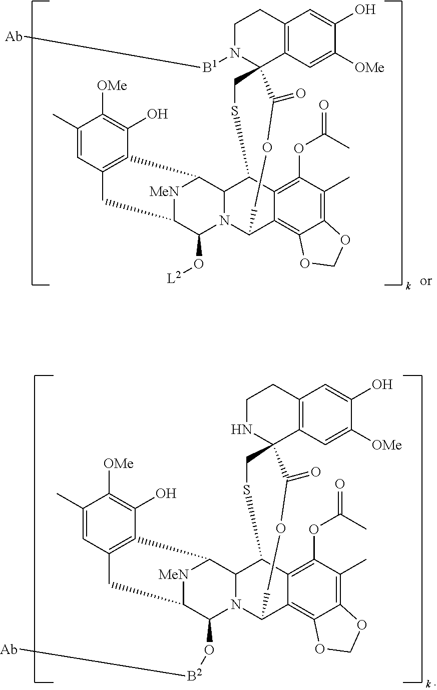

Embodiment 47. The compound of any one of Embodiments 42-46, wherein at least one of B¹ or B² independently comprises

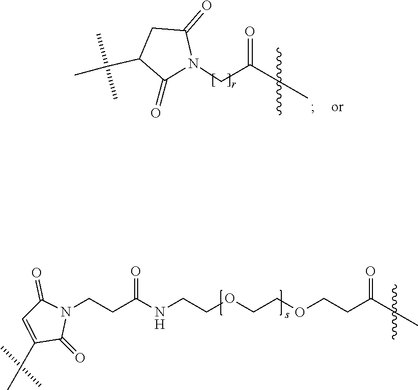

wherein

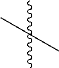

indicates the point of attachment to the compound of Formula (Va) or (Vb) or to a remaining portion of L¹ or L2
wherein

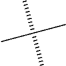

indicates the point of attachment to an antibody or antibody fragment;
wherein subscript r is an integer selected from 1 to 6; and
wherein subscript s is an integer selected from 2 to 14.

Embodiment 48. The compound of Embodiment 47, wherein
wherein subscript r is an integer selected from 2 to 6; and
wherein subscript s is an integer selected from 2 to 14.

Embodiment 49. The compound of Embodiment 47 or 48, wherein the compound is

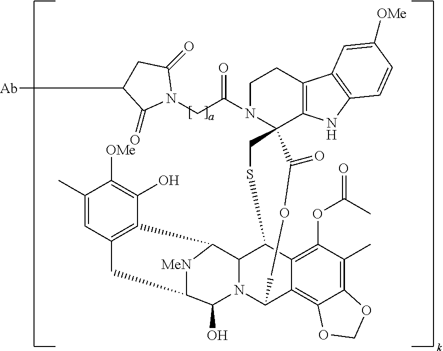

wherein a is an integer selected from 2 to 6.

Embodiment 50. The compound of Embodiment 42, wherein the compound is selected from

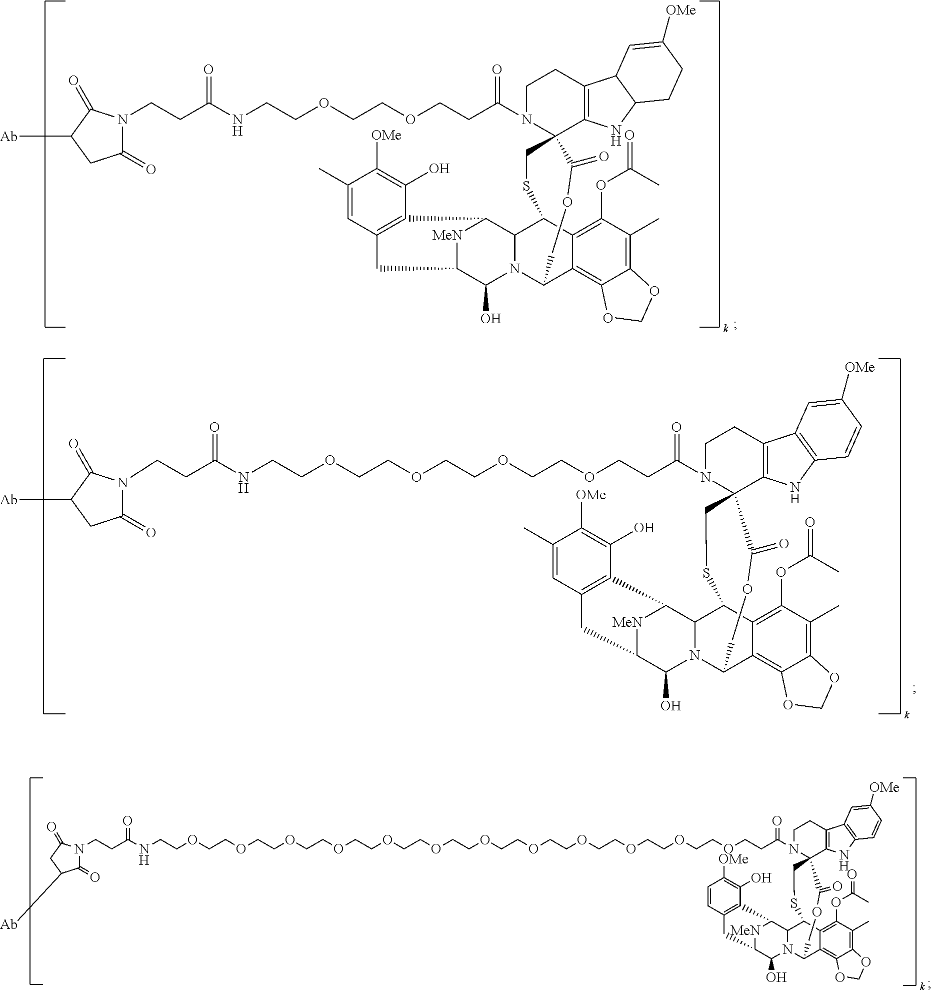

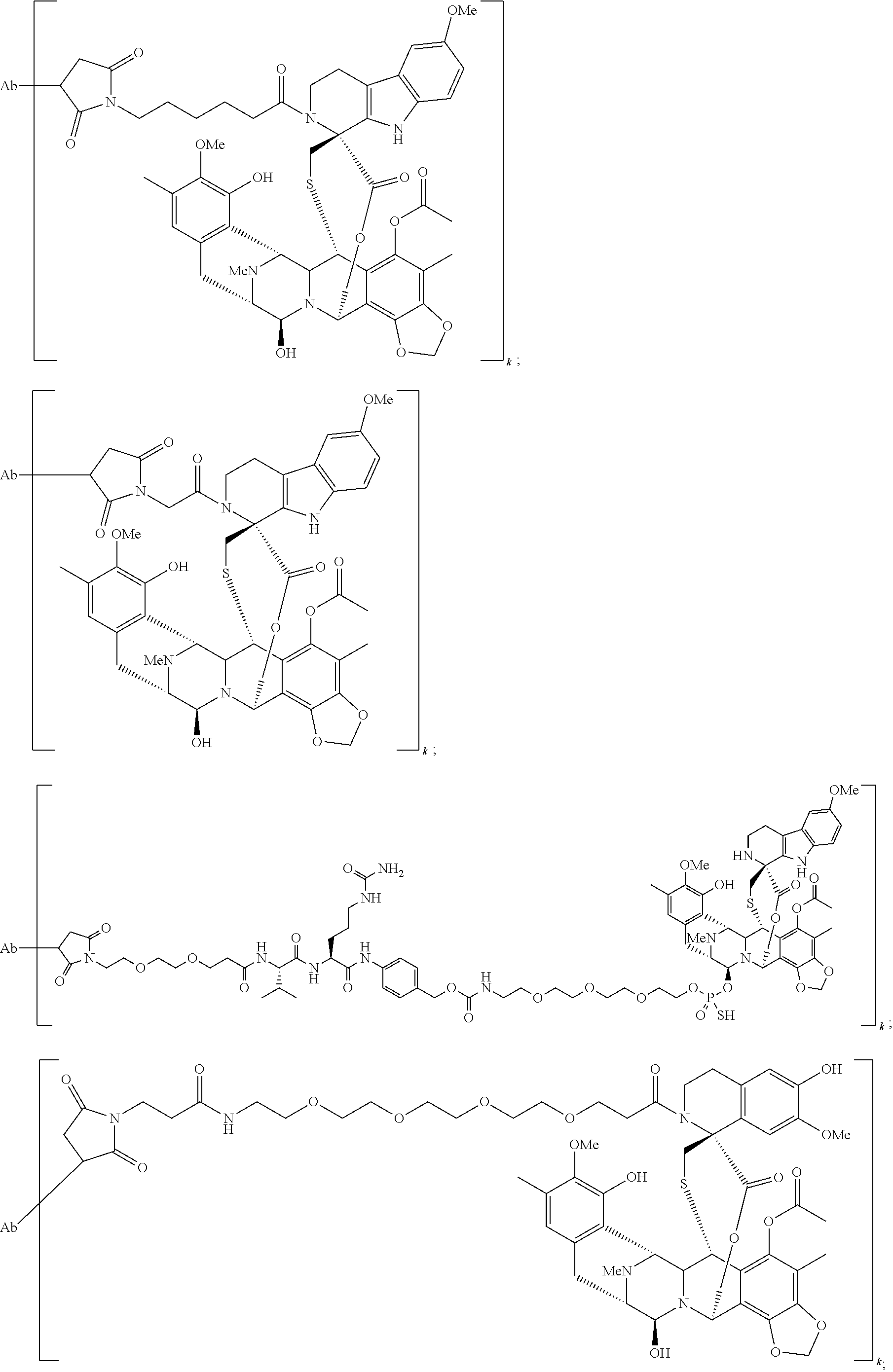

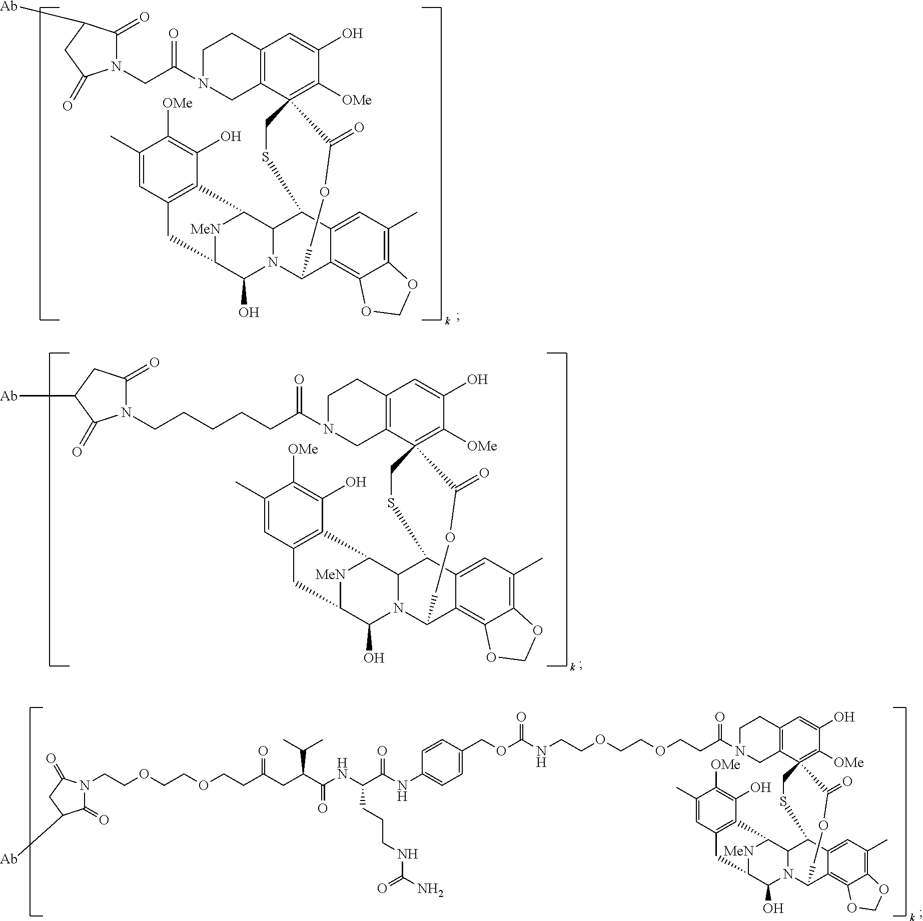

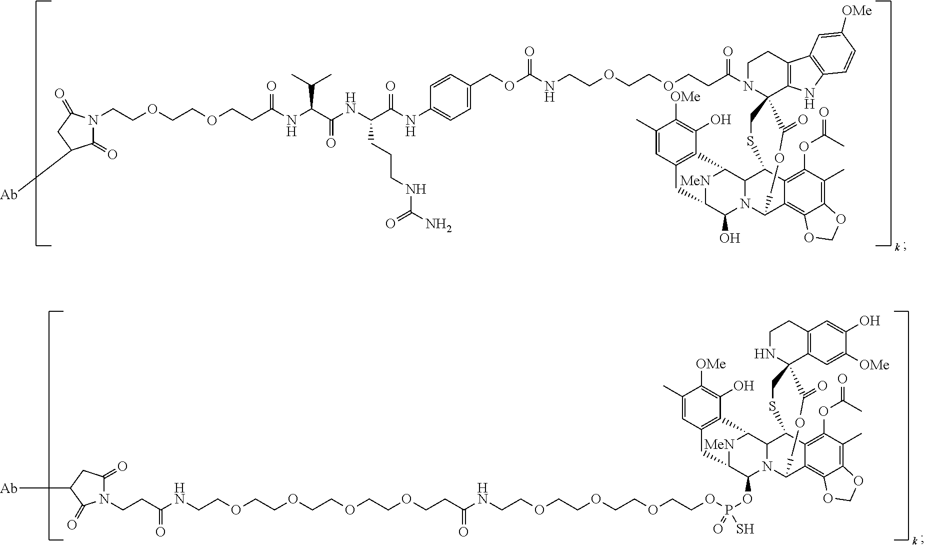

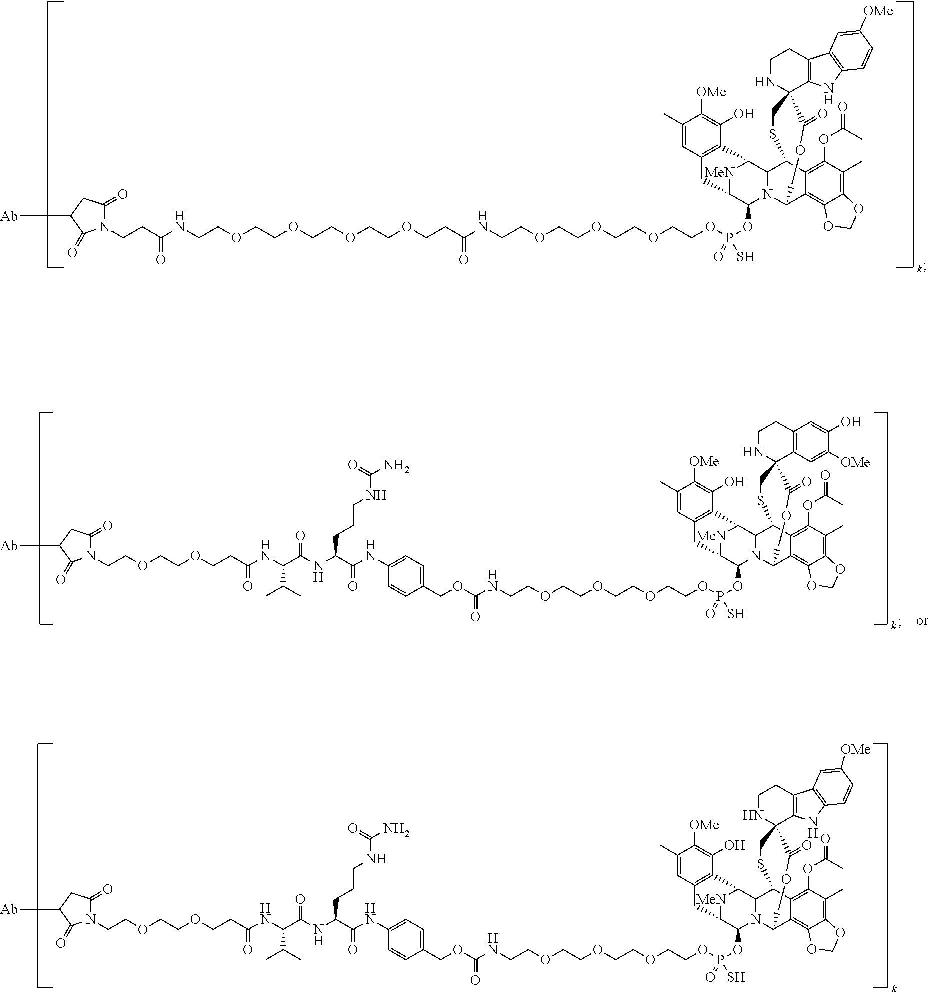

Embodiment 51. A pharmaceutical composition comprising the compound of any of Embodiments 1-16 or 38-49 and a pharmaceutically acceptable excipient.

Embodiment 52. A method of treating a disease or condition comprising administering the compound of Embodiments 1-16 or 38-49.

### EXAMPLES

The following Analytical Methods were used unless stated otherwise.

### Analytical Method A.

Instrument: Agilent 1260 Infinity Lab LC/MSD. Column: Agilent Zorbax C18.5 mm, 4.6×50 mm. Column temperature: 45° C. Mobile Phase A: 0.1% trifluoro acetic acid (TFA) water. Mobile Phase B: 0.1% TFA in acetonitrile. Gradient of 5% to 95% Mobile phase B over 3 min, hold at 95% B for 3 min, column wash for 4 min. UV trace monitoring at 210 nm and 254 nm. Positive and negative ionization modes monitoring molecular ions between 115 Da-1200 Da.

### Analytical Method B.

Instrument: Agilent 6230 TOF LC/MS. Column: Agilent Zorbax 300SB—C8.5 mm, 4.6×50 mm. Column temperature: 45° C. Mobile Phase A: 0.1% FA water. Mobile Phase B: 0.1% TFA in acetonitrile. Gradient of 5% to 95% Mobile phase B over 10 min, hold at 95% B for 5 min, column wash for 5 min. UV trace monitoring at 210 nm and 254 nm.

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hours (hours); min (minutes); MS (mass spectrometry); ESI (electrospray ionization); TLC (thin layer chromatography); HPLC (high pressure liquid chromatography); THF (tetrahydrofuran); CDCl₃ (deuterated chloroform); AcOH (acetic acid); DCM (dichloromethane); DMSO (dimethylsulfoxide); DMSO-d₆ (deuterated dimethylsulfoxide); EtOAc (ethyl acetate); MeOH (methanol); and BOC (t-butyloxycarbonyl).

For all the following examples, standard work-up and purification methods known to those skilled in the art can be utilized. Unless otherwise indicated, all temperatures are expressed in ° C. (degrees Centigrade). All reactions are conducted at room temperature unless otherwise noted. Synthetic methodologies illustrated herein are intended to exemplify the applicable chemistry through the use of specific examples and are not limiting as to the scope of the disclosure.

### Example 1

### Preparation of LD1

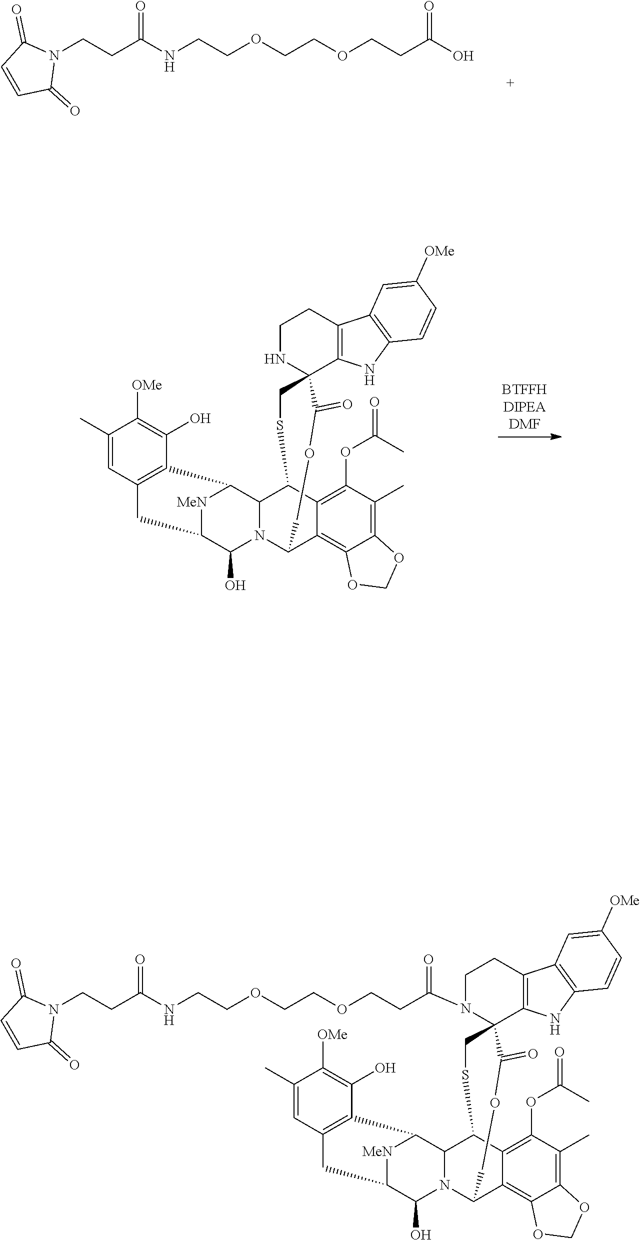 [SMILES: O=C(O)CCOCCOCCNC(=O)CCN1C(=O)C=CC1=O ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD1.

Maleimido-(PEG)₂-carboxylic acid (1.5 equivalents) was dissolved in anhydrous dimethyl formamide (DMF) at 0.1M concentration, followed by the addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 minutes at room temperature before addition of lurbinectedin (1 equivalents). The reaction mixture was stirred for 16 hours (hrs) at room temperature. The crude product was purified using flash reverse phase chromatography using Biotage Isolera chromatography instrument equipped with Biotage Sfar C18 column, gradient of 5%-95% acetonitrile/water with 0.1% formic acid. LCMS analytical method A, Rₜ=4.8 min, Expected [M+H]⁺ 1095.40, found [M+H]⁺ 1095.4, [M+2H]²⁺ 548.5, off-white solid.

### Example 2

### Preparation of LD2

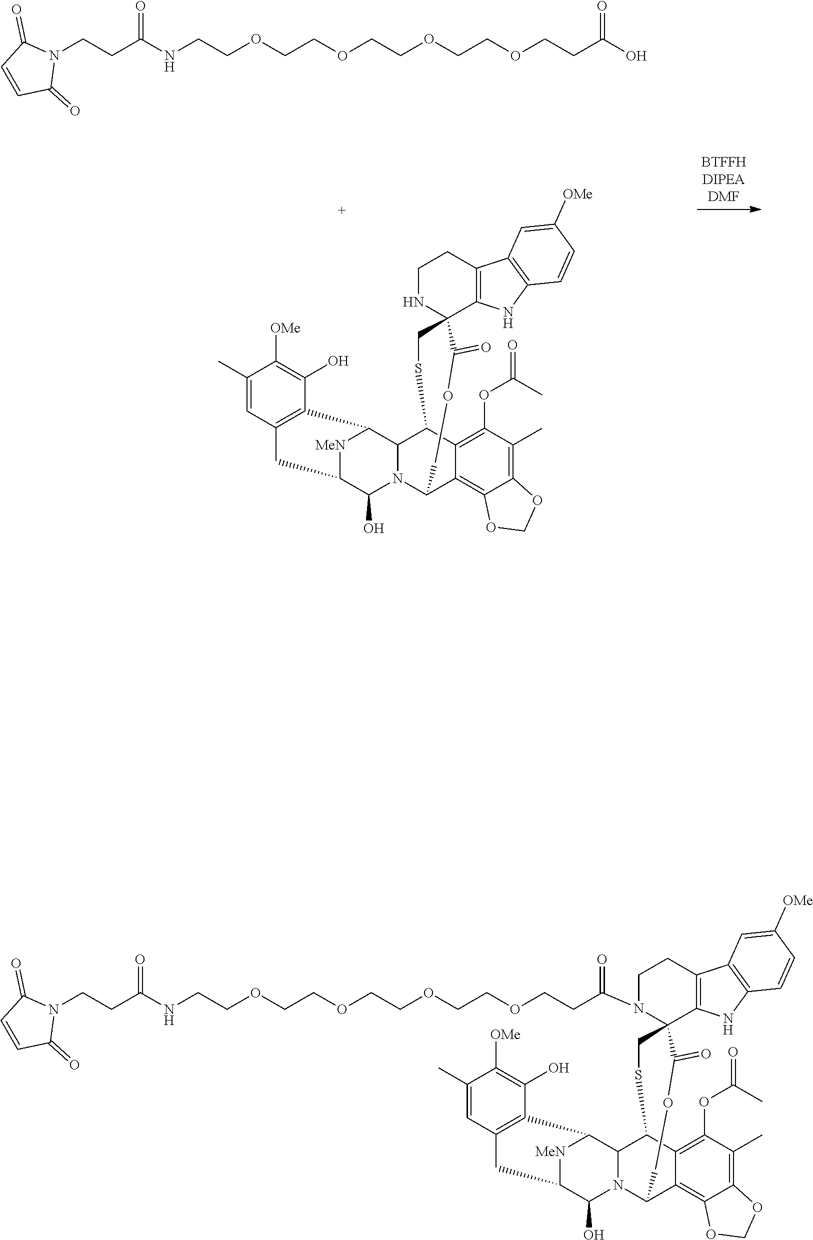 [SMILES: O=C(O)CCOCCOCCOCCOCCNC(=O)CCN1C(=O)C=CC1=O ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CCOCCOCCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD2.

Maleimido-(PEG)₄-carboxylic acid (1.5 equivalents) was dissolved in anhydrous dimethyl formamide (DMF) at 0.1M concentration, followed by the addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of lurbinectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.92 min, Expected [M+H]⁺ 1183.45, found [M+H]⁺ 1184.0, [M+2H]²⁺ 592.5, off-white solid.

### Example 3

### Preparation of LD3

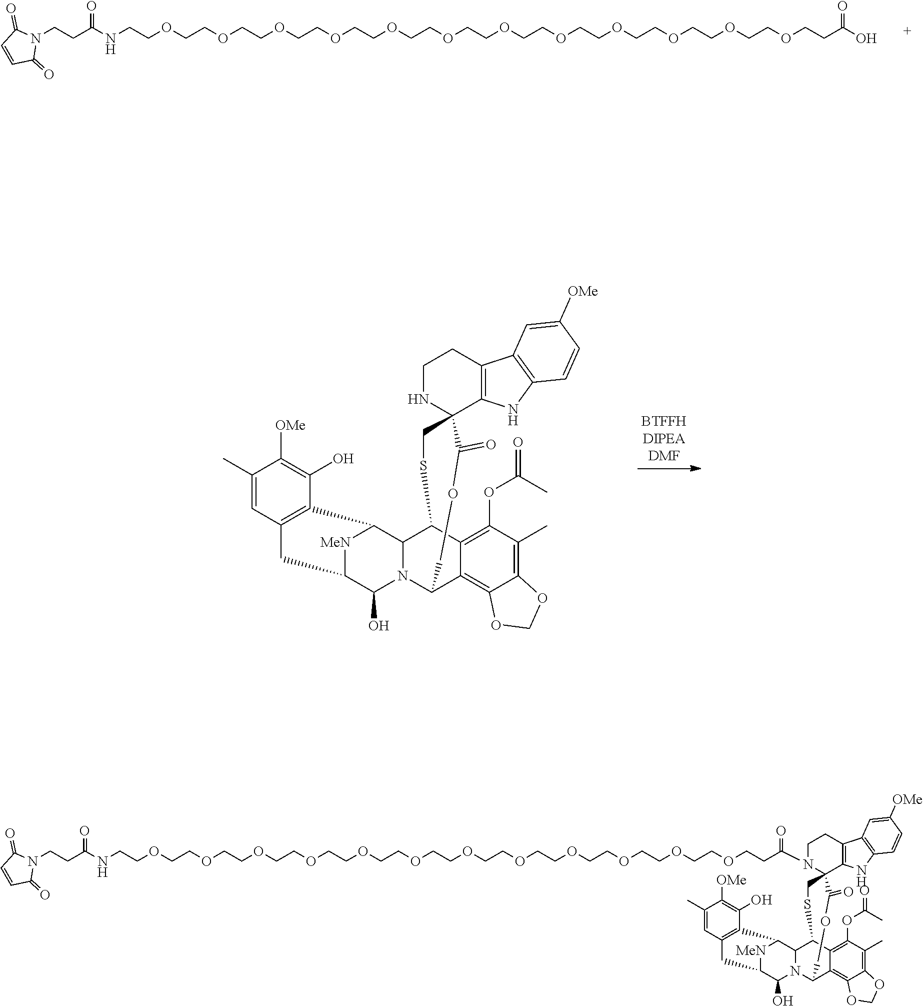 [SMILES: O=C(O)CCOCCOCCOCCOCCOCCOCCOCCOCCOCCOCCOCCOCCNC(=O)CCN1C(=O)C=CC1=O ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CCOCCOCCOCCOCCOCCOCCOCCOCCOCCOCCOCCOCCNC(=O)CCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD3.

Maleimido-(PEG)₁₂-carboxylic acid (1.5 equivalents) was dissolved in anhydrous dimethyl formamide (DMF) at 0.1M concentration, followed by the addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of lurbinectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.63 min, Expected [M+H]⁺ 1535.66, found [M+2H]²⁺ 768.4, off-white solid. High resolution TOF-MS expected [M+H]⁺ 1535.6637, found [M+H]⁺ 1535.7737.

### Example 4

### Preparation of LD4

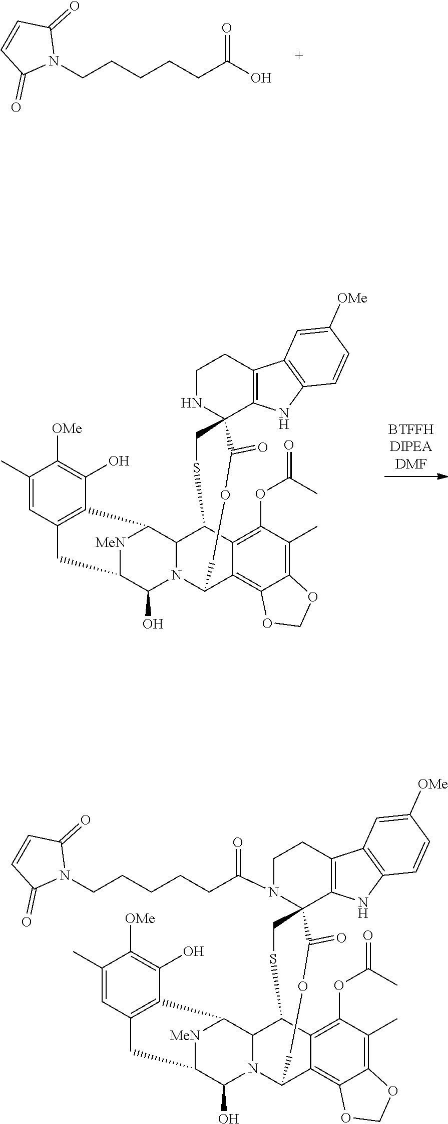 [SMILES: O=C(O)CCCCCN1C(=O)C=CC1=O ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CCCCCN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD4.

Maleimido-hexanoic acid (1.5 equivalents) was dissolved in anhydrous dimethyl formamide (DMF) at 0.1M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of lurbinectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=5.2 min, Expected [M+H]⁺ 978.35, found [M+H]⁺ 978.4, [M+2H]²⁺ 490, off-white solid.

### Example 5

### Preparation of LD5

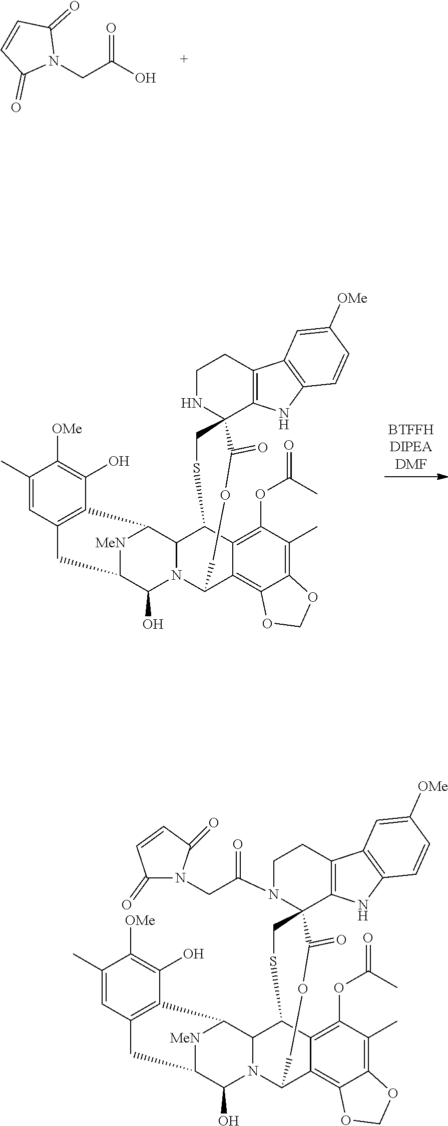 [SMILES: O=C(O)CN1C(=O)C=CC1=O ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CN1C(=O)C=CC1=O)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD5.

Maleimido-glycine acid (1.5 equivalents) was dissolved in anhydrous dimethyl formamide (DMF) at 0.1 M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of lurbinectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.75 min, Expected [M+H]⁺ 922.29, found [M+H]⁺ 922.3, [M+2H]²⁺ 461.6, off-white solid.

### Example 6

### Preparation of LD6

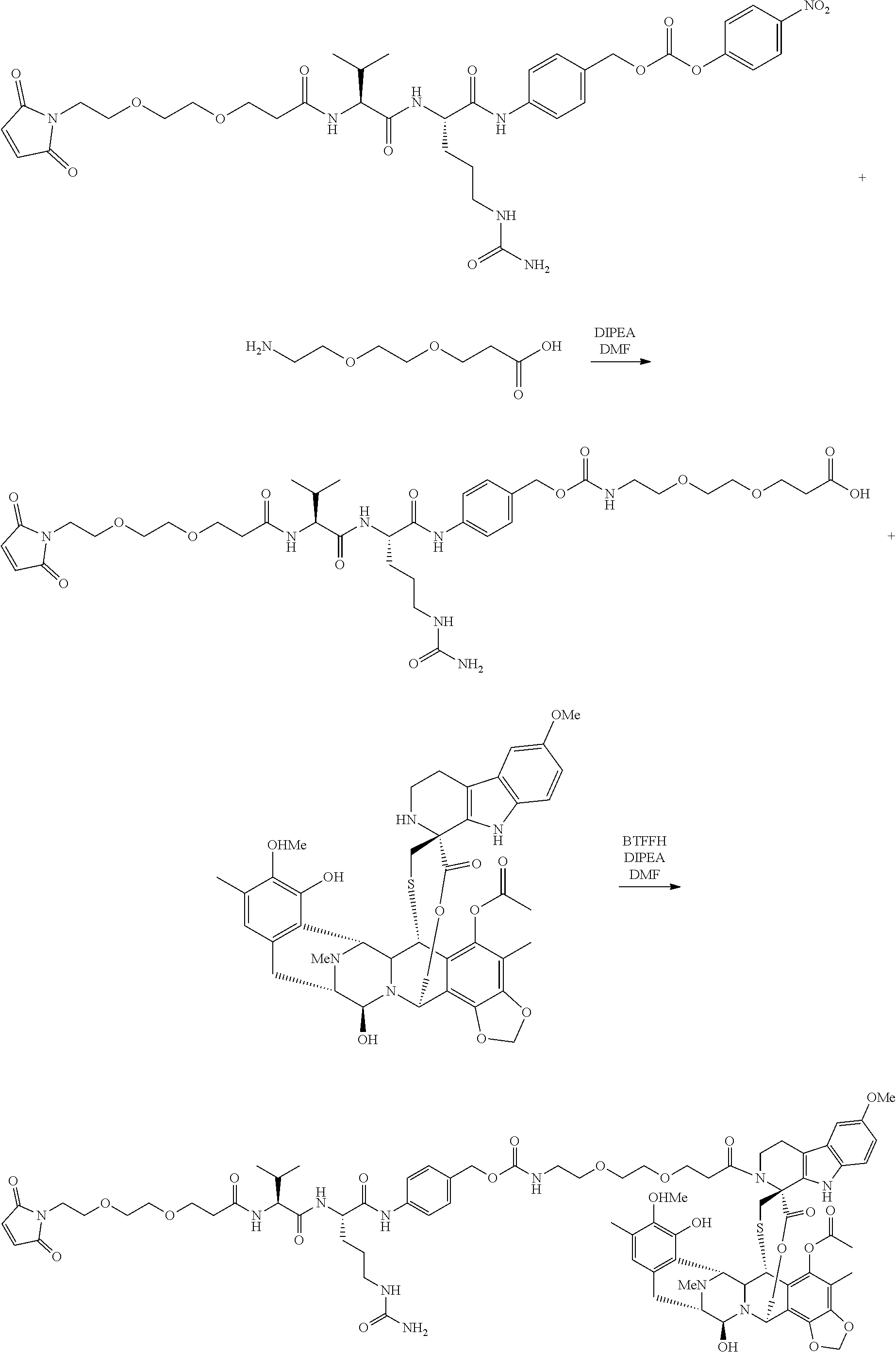

This example provides synthesis of lurbinectedin LD6.

Maleimido-Val-Cit-PAB-PNP (1 equivalents) was dissolved in anhydrous dimethyl formamide (DMF) at 0.1M followed by addition of amino-(PEG)₂-carboxylic acid (1.5 equivalents) and DIPEA (5 equivalents). The reaction was stirred at room temperature for 24 hours followed by purification of the crude product using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.86 min, Expected [M+H]⁺ 1589.65, found [M+2H]²⁺ 795.4, [M−OH+H]². 786.4, off-white solid.

Maleimido-Val-Cit-PAB-amido-(PEG)₂-carboxylic acid (1.5 equivalents) was dissolved in anhydrous dimethyl formamide (DMF) at 0.1 M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of lurbinectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.86 min, Expected [M+H]⁺ 1589.66, found [M+H]⁺ 795.4, [M−OH+H]2⁺ 786.4, off-white solid.

### Example 7

### Preparation of LD7

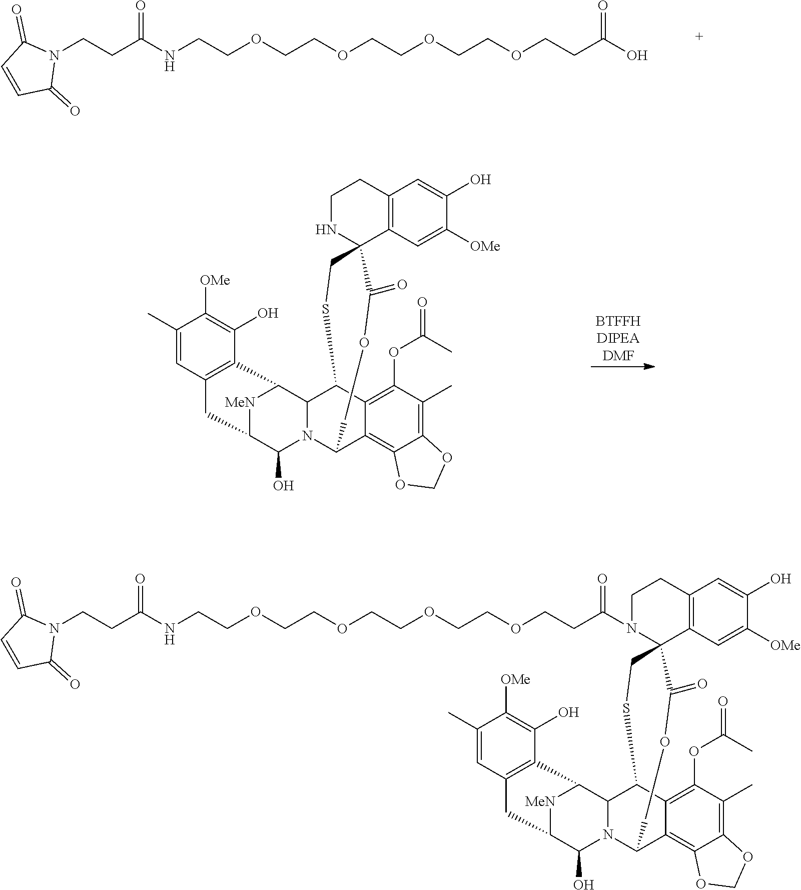 [SMILES: O=C(O)CCOCCOCCOCCOCCNC(=O)CCN1C(=O)C=CC1=O ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1cc2c(cc1O)CCN(C(=O)CCOCCOCCOCCOCCNC(=O)CCN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of trabectedin LD7.

Maleimido-(PEG)₄-carboxylic acid (1.5 equivalents) was dissolved in anhydrous dimethyl formamide (DMF) at 0.1M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of trabectedin (1 equivalents). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.54 min, Expected [M+H]⁺ 1160.44, found [M+H]⁺ 1160.4, [M+2H]²⁺ 580.7, pale yellow solid.

### Example 8

### Preparation of LD8

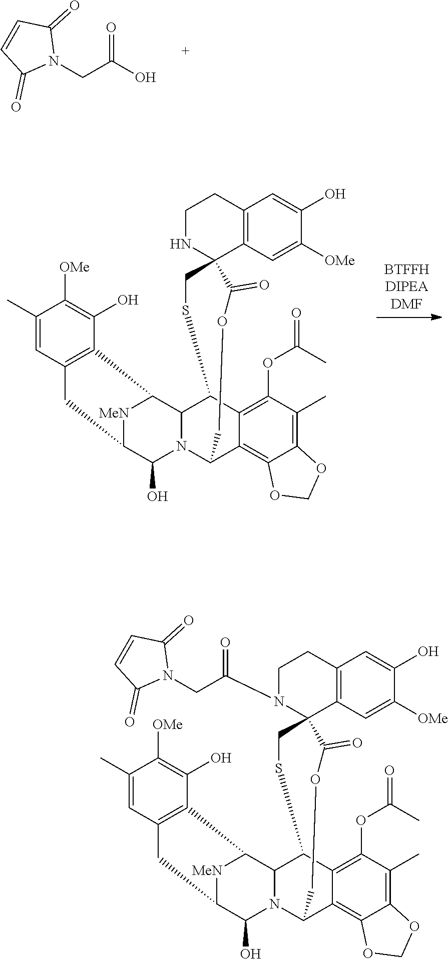 [SMILES: O=C(O)CN1C(=O)C=CC1=O ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1cc2c(cc1O)CCN(C(=O)CN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of trabectedin LD8.

2-(2,5-dioxo-2,5-dihydro-1H-pyrrol-1-yl)acetic acid (1.5 equivalents) was dissolved in anhydrous dimethyl formamide (DMF) at 0.1 M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of trabectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.32 min, Expected [M+H]⁺ 899.28, found [M+H]⁺ 899.3, red solid.

### Example 9

### Preparation of LD9

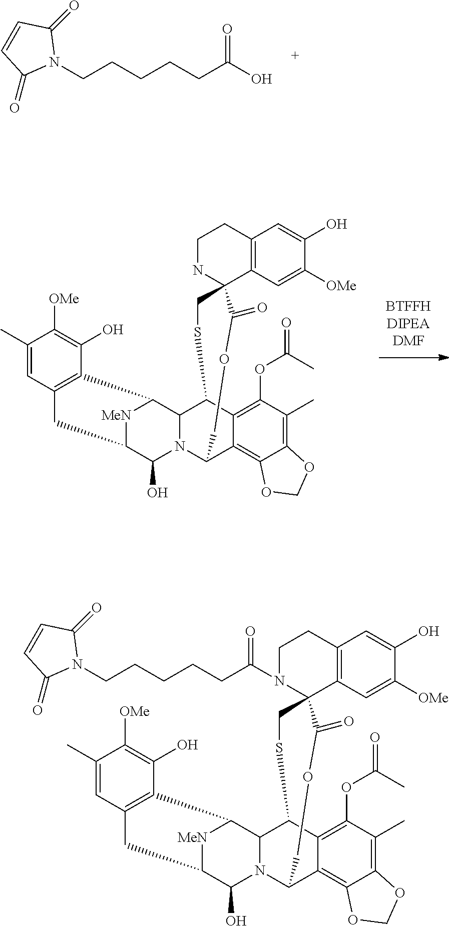 [SMILES: O=C(O)CCCCCN1C(=O)C=CC1=O ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1cc2c(cc1O)CCN(C(=O)CCCCCN1C(=O)C=CC1=O)[C@]21CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of trabectedin LD9.

Maleimidohexanoic acid (1.5 equivalents) was dissolved in anhydrous dimethyl formamide (DMF) at 0.1 M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 min at room temperature before addition of trabectedin (1 equivalent). The reaction mixture was stirred for 16 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.9 min, Expected [M+H]⁺ 955.34, found [M+H]⁺ 955.9, yellow solid.

### Example 10

### Preparation of LD10

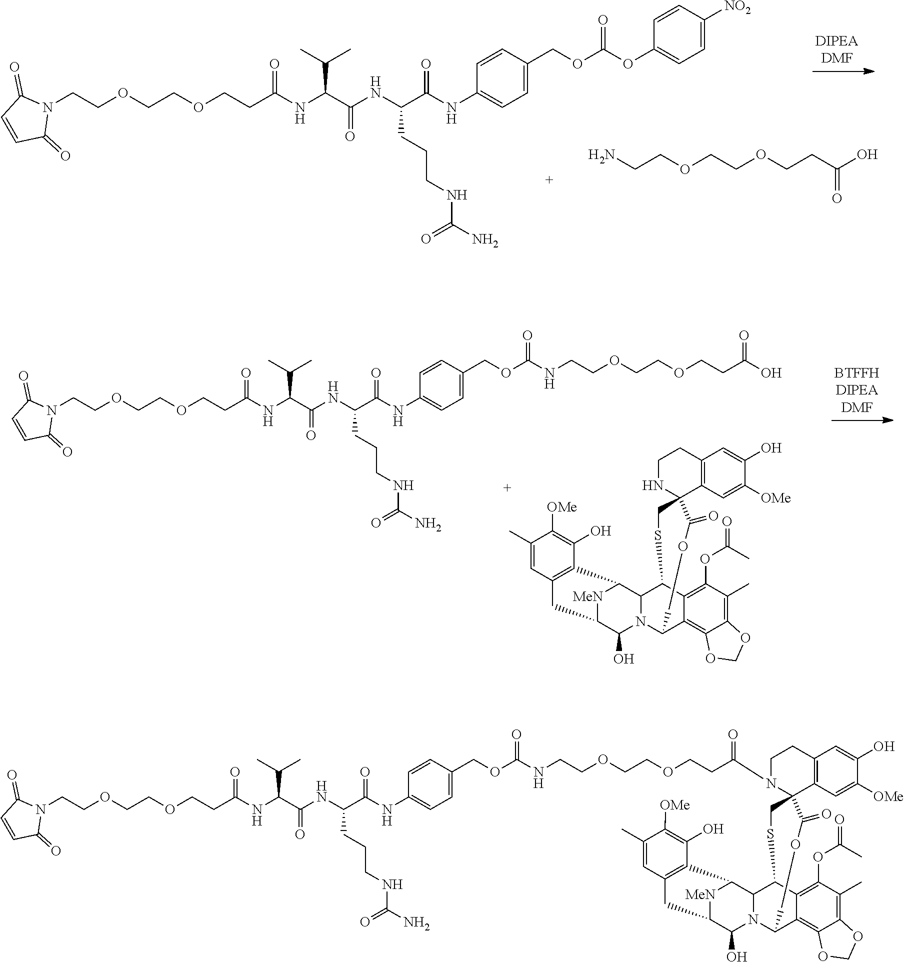

This example provides synthesis of trabectedin LD10.

Maleimido-Val-Cit-PAB-PNP (1 equivalents) was dissolved in anhydrous dimethyl formamide (DMF) at 0.1M followed by addition of amino-(PEG)₂-carboxylic acid (1.5 equivalents) and DIPEA (5 equivalents). The reaction was stirred at room temperature for 24 hours followed by purification of the crude product using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.56 min, Expected [M+H]⁺ 822.39, found [M+H]⁺ 822.4, off-white solid.

Maleimido-Val-Cit-PAB-amido-(PEG)₂-carboxylic acid (1.5 equivalents) was dissolved in anhydrous dimethyl formamide (DMF) at 0.1 M concentration, followed by addition of fluoro-N,N,N′,N′-bis(tetramethylene)formamidinium hexafluorophosphate (BTFFH; 5 equivalents) and diisopropyl ethylamine (DIPEA; 10 equivalents). The reaction mixture was stirred for 10 minutes at room temperature before addition of trabectedin (1 equivalent). The reaction mixture was stirred for 16 hours at room temperature. Crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.96 min, Expected [M+H]⁺ 1565.63, found [M+2H]²⁺ 783.4, [M−OH+H]²⁺ 774.2, pale yellow solid.

### Example 11

### Preparation of Lurbinectedin Derivative 1

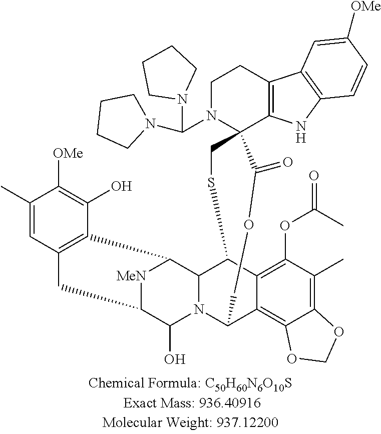 [SMILES: COc1ccc2[nH]c3c(c2c1)CCN(C(N1CCCC1)N1CCCC1)[C@]31CS[C@@H]2c3c(OC(C)=O)c(C)c4c(c3[C@H](COC1=O)N1C2[C@H]2c3c(cc(C)c(OC)c3O)C[C@@H](C1O)N2C)OCO4]

This example provides synthesis of the above compound.

Lurbinectedin was dissolved in anhydrous dimethyl formamide (DMF) at 0.01 M concentration followed by addition of fluoro-N,N,N′,N′-bis(tetramethylene)formamidinium hexafluorophosphate (BTFFH; 10 equivalents) and diisopropyl ethylamine (DIPEA; 20 equivalents). The reaction mixture was stirred at room temperature overnight. Crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.81 min, Expected [M+H]⁺ 937.41, found [M+H]⁺ 937.4, red solid.

### Example 12

### Preparation of Lurbinectedin Derivative 2

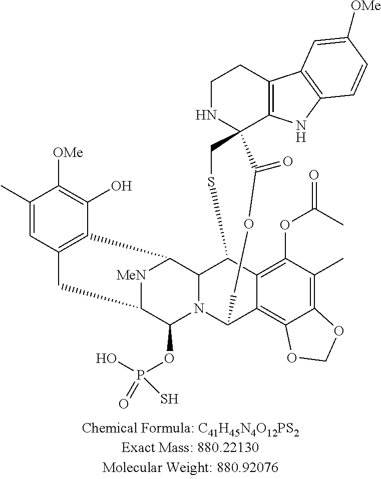 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4]

This example provides synthesis of the above compound.

To the solution of lurbinectedin (1 equivalents) in dimethyl formamide (DMF) was added (−)PSI reagent (4 equivalents) and 1,8-diazabicyclo[5.4.0]undec-7-ene (DBU) (5 equivalents) and reaction was stirred at room temperature for 1 hour followed by addition of water and reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.64 min, Expected [M+H]⁺ 881.22, found [M+H]⁺ 881.2, yellow solid.

### Example 13

### Preparation of Lurbinectedin Derivative 3

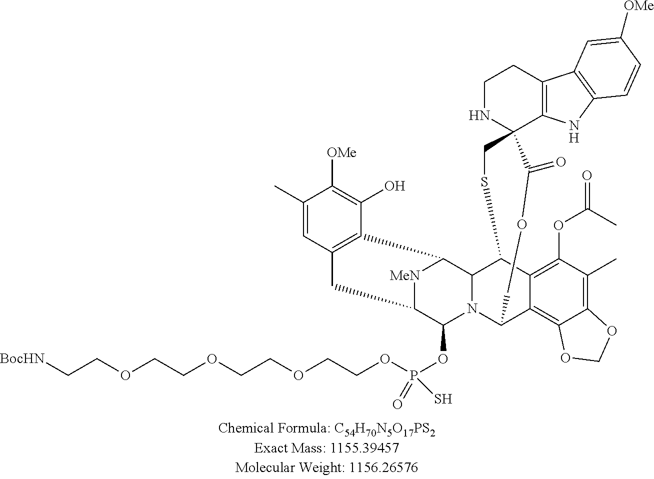 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4]

This example provides synthesis of the above compound.

To the solution of lurbinectedin (1 equivalents) was added (−)PSI reagent (4 equivalents) and 1,8-diazabicyclo[5.4.0]undec-7-ene (DBU) (5 equivalents) and reaction was stirred at room temperature for 1 hour followed by addition of N-Boc amino-(PEG)₄-alcohol (5 equivalents) and 1 hour of stirring. The crude product was purified by reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.52 min, Expected [M+H]⁺ 1156.40, found [M+H]⁺ 1155.4, [M+2H]²⁺ 578.5, yellow solid.

The structure of (−)PSI reagent is shown below:

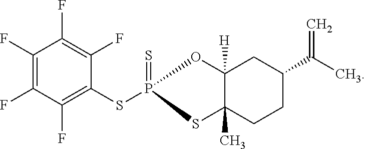 [SMILES: C=C(C)[C@@H]1CC[C@]2(C)S[P@](=S)(Sc3c(F)c(F)c(F)c(F)c3F)O[C@H]2C1]

### Example 14

### Preparation of Trabectedin Derivative 4

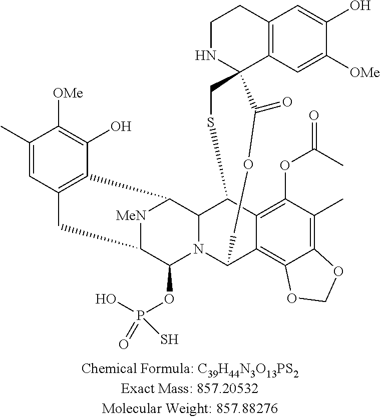 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4]

This example provides synthesis of the above compound.

To the solution of trabectedin (1 equivalents) in dimethyl formamide (DMF) was added (−)PSI reagent (4 equivalents) and diazabicyclo[5.4.0]undec-7-ene (DBU) (5 equivalents) and reaction was stirred at room temperature for 1 hour followed by addition of water and reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.42 min, Expected [M+H]⁺ 858.21, found [M+H]⁺ 858.2, pale yellow solid.

### Example 15

### Preparation of Trabectedin Derivative 5

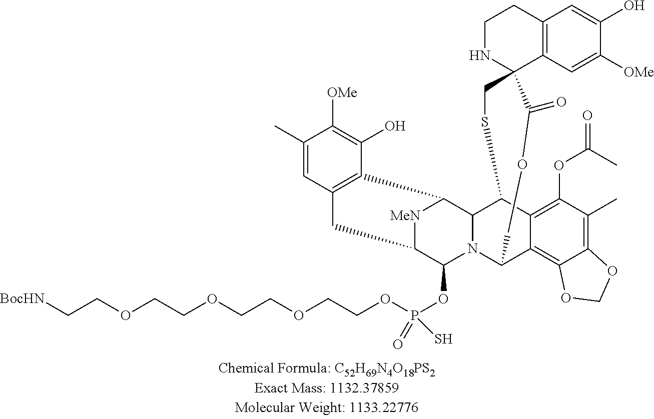 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4]

This example provides synthesis of the above compound.

To the solution of trabectedin (1 equivalents) was added (−)PSI reagent (4 equivalents) and diazabicyclo[5.4.0]undec-7-ene (DBU) (5 equivalents) and reaction was stirred at room temperature for 1 hour followed by addition of N-Boc amino-(PEG)₄-alcohol (5 equivalents) and 1 hour of stirring. The crude product was purified by reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.52 min, Expected [M+H]⁺ 1133.38, found [M+H]⁺ 1133.4, [M+2H]²⁺ 567.0, yellow solid.

### Example 16

### Preparation of LD11

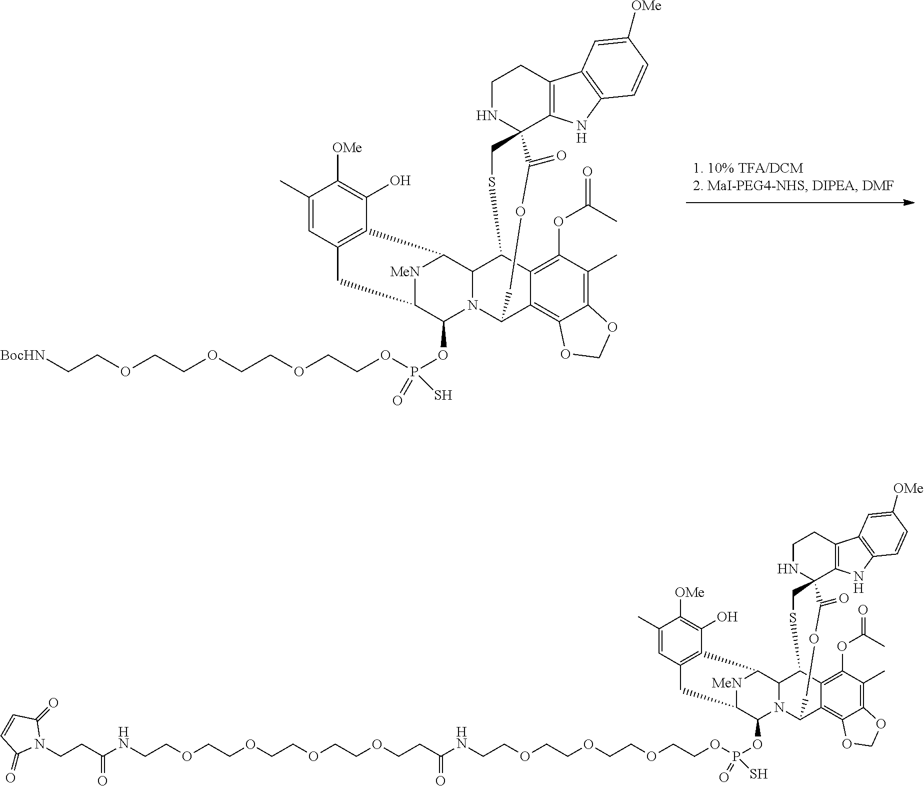 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)CCOCCOCCOCCOCCNC(=O)CCN1C(=O)C=CC1=O)N2C)OCO4]

This example provides synthesis of lurbinectedin LD11.

Lurbinectedin Derivative 3 was dissolved in 10% TFA/DCM and stirred at room temperature for 30 minutes. The reaction mixture was concentrated on a rotary evaporator. The crude lurbinectedin amine product was used for the next step.

Crude lurbinectedin amine was dissolved in anhydrous dimethyl formamide (DMF) followed by addition of DIPEA (10 equivalents) and Mal-(PEG)₄-NHS (2 equivalents) reagents. The reaction mixture was stirred at room temperature for 16 hours. The crude product was purified by reverse phase chromatography using Biotage Isolera chromatography instrument equipped with Biotage Sfar C18 column, gradient of 5%-95% Acetonitrile/Water with 0.1% formic acid.

The structure of Mal-(PEG)₄-NHS (i.e., Mal-PEG4-NHS) as used in the Examples of the present disclosure is shown below:

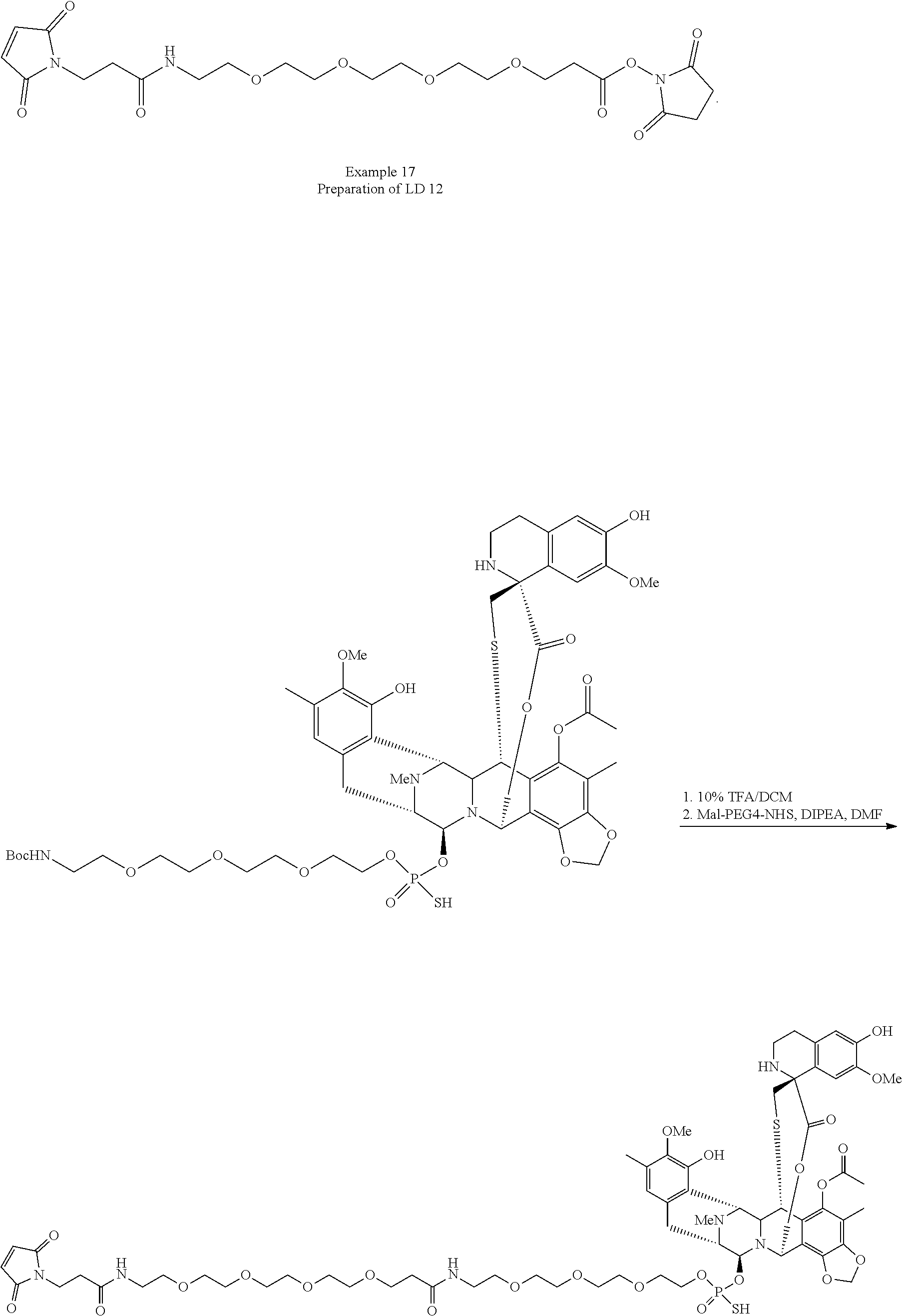 [SMILES: O=C(CCN1C(=O)C=CC1=O)NCCOCCOCCOCCOCCC(=O)ON1C(=O)CCC1=O ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)CCOCCOCCOCCOCCNC(=O)CCN1C(=O)C=CC1=O)N2C)OCO4]

This example provides synthesis of trabectedin LD12.

Trabectedin Derivative 5 was dissolved in 10% TFA/DCM and stirred at room temperature for 30 minutes. The reaction mixture was concentrated on a rotary evaporator and the crude trabectedin amine product was used for the next step.

Crude trabectedin amine was dissolved in anhydrous dimethyl formamide (DMF) followed by addition of DIPEA (10 equivalents) and Mal-(PEG)₄-NHS (2 equivalents) reagent. The reaction mixture was stirred at room temperature for 16 hours. The crude product was purified by reverse phase chromatography using Biotage Isolera chromatography instrument equipped with Biotage Sfar C18 column, gradient of 5%-95% Acetonitrile/Water with 0.1% formic acid.

### Example 18

### Preparation of LD13

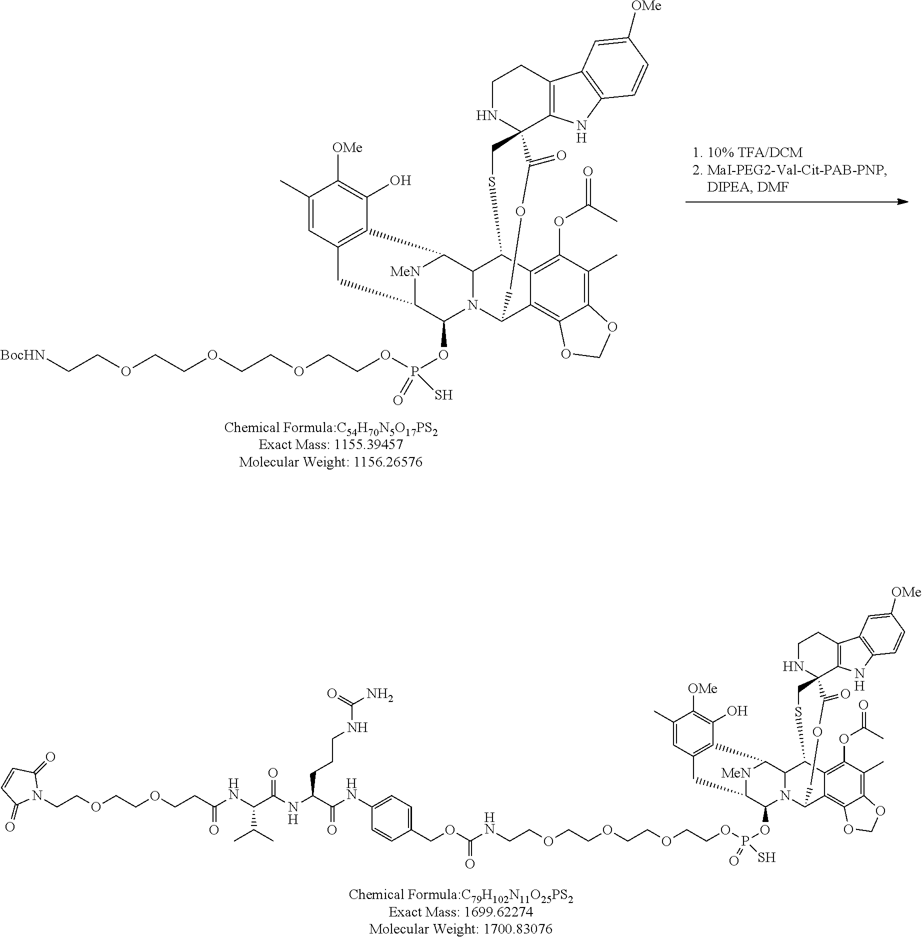 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4]

This example provides synthesis of lurbinectedin LD13.

Lurbinectedin Derivative 3 was dissolved in 10% TFA/DCM (TFA=trifluoroacetic acid) and stirred at room temperature for 30 minutes. The reaction mixture was concentrated on a rotary evaporator and the crude lurbinectedin amine product was used for the next step.

Crude lurbinectedin amine was dissolved in anhydrous dimethyl formamide (DMF) followed by addition of DIPEA (10 equivalents, DIPEA=diisopropylethylamine) and Mal-(PEG)₂-Val-Cit-PAB-PNP (2 equivalents) reagent. Reaction mixture was stirred at room temperature for 16 hours. The crude product was purified by reverse phase chromatography using Biotage Isolera chromatography instrument equipped with Biotage Sfar C18 column, gradient of 5%-95% Acetonitrile/Water with 0.1% formic acid.

The structure of Mal-(PEG)₂-Val-Cit-PAB-PNP (i.e., Mal-PEG2-Val-Cit-PAB-PNP) as used in the Examples of the present disclosure is shown below:

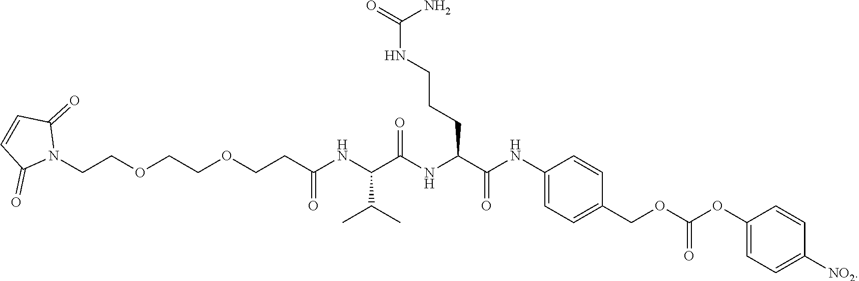 [SMILES: CC(C)[C@H](NC(=O)CCOCCOCCN1C(=O)C=CC1=O)C(=O)N[C@@H](CCCNC(N)=O)C(=O)Nc1ccc(COC(=O)Oc2ccc([N+](=O)[O-])cc2)cc1]

### Example 19

### Preparation of LD14

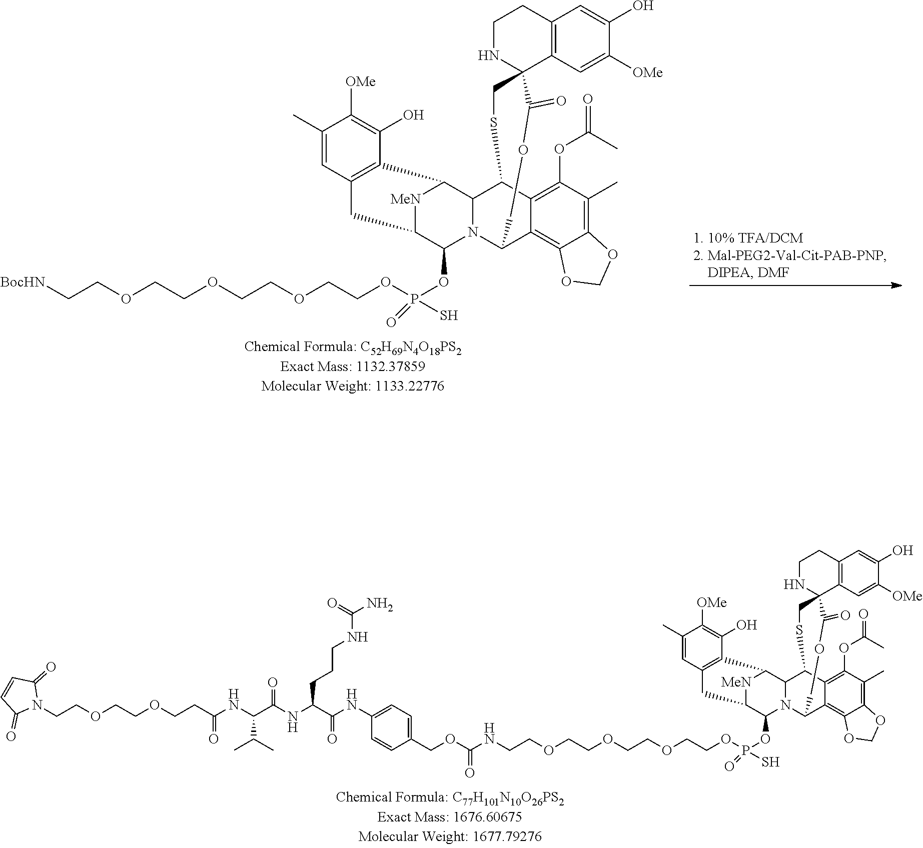 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OCc1ccc(NC(=O)[C@H](CCCNC(N)=O)NC(=O)[C@@H](NC(=O)CCOCCOCCN3C(=O)C=CC3=O)C(C)C)cc1)N2C)OCO4]

This example provides synthesis of trabectedin LD14.

Trabectedin Derivative 5 was dissolved in 10% TFA/DCM and stirred at room temperature for 30 minutes. The reaction mixture was concentrated on a rotary evaporator and the crude trabectedin amine product was used for the next step.

Crude trabectedin amine was dissolved in anhydrous dimethyl formamide (DMF) followed by addition of DIPEA (10 equivalents) and Mal-(PEG)₂-Val-Cit-PAB-PNP (2 equivalents) reagent. Reaction mixture was stirred at room temperature for 16 hours. The crude product was purified by reverse phase chromatography using Biotage Isolera chromatography instrument equipped with Biotage Sfar C18 column, gradient of 5%-95% Acetonitrile/Water with 0.1% formic acid.

### Example 20

### Antibody Drug Conjugate Generation and Characterization

### Conjugation

The mAb was pH adjusted with 200 mM Tris, 5 mM EDTA, pH 8.5 (10% if in PBS, 30% if in proA) then reduced with TCEP 4-10 equivalents at 37° C. for 1 hour. The number of free thiols was quantified using Ellman's test. 10% DMSO and 2 M excess of linker-drug (LD, e.g., LD1) per mol of free thiol were added, the solution was vortexed and let sit for 2 hours at room temperature. Then, 1.2 mol N-acetylcysteine (NAC) per mol linker-drug was added, the solution was vortexed and let sit for 20 minutes at room temperature. The resulting antibody drug conjugate (ADC) was then buffer exchanged 3X into PBS pH 7.4 to remove free linker-drug.

The drug-to-antibody ratio (DAR) was determined by either hydrophobic interaction chromatography (HIC) or using mass spectrometry of intact ADC. Aggregation was determined by size exclusion chromatography (SEC).

Full characterization data is presented in Table 1 below.

### Hydrophobic Interaction Characterization of ADC

Hydrophobic Interaction Characterization (HIC) was conducted using a PolyPropyl A column (PolyLC) with 1.5 M ammonium sulfate and 25 mM potassium phosphate in water as mobile phase A, and 0.25% w/v CHAPS and 25 mM potassium phosphate in water as mobile phase B. Samples were injected directly onto the column, where a gradient of 0-100% mobile phase B was applied over 15 min. UV signal at 280 nm was collected, and the chromatogram analysed for unconjugated antibody and higher DAR species. DAR calculations were performed by integrating the area under the HIC curve of the previously established peaks (DAR=0, DAR=1, DAR=2, DAR=4, etc) and calculating the % of each peak.

### Mass Spectrometry Characterization of Intact ADC

The non-reduced masses of antibody-drug conjugates were determined on Agilent 6230 TOF LC/MS equipped with electrospray ionization (ESI) source, which was directly coupled to high-performance liquid chromatography Agilent 1260 system. Samples were first diluted to 1 mg/mL then analyzed in their non-reduced form. The proteins are separated on a reverse phase column (Zorbax 300SB—C8, 5 mm, 4.6×50 mm) with a denaturing mobile phase system. Mobile Phase A is 0.1% (v/v) formic acid in water. Mobile phase B is 0.1% (v/v) formic acid in 80% (v/v) 2-propanol, 10% (v/v) acetonitrile, 10% (v/v) water (mobile phase B). The MS spectra of each protein are averaged and then deconvoluted to obtain the average mass and monoisotopic mass.

### Size Exclusion Chromatography Characterization of ADC Aggregation

Size-exclusion chromatography (SEC) was used to characterize size heterogeneity of the antibody-drug conjugates. The analysis employed an Acquity 1.7 83, 4.6×300 mm UPLC BEH200 SEC column with 25 mM sodium phosphate, pH 6.5, 500 mM L-arginine, and 10%/isopropanol (IIPA) in water as mobile phase. Samples were injected neat and the mobile phase was applied isocratically at 0.2 mL/min for 22 min. The UV signal at 280 nm was collected and the peak area was used to calculate the extent of aggregation and fragmentation of the ADC.

TABLE 1
Ecteinascidin ADCs Characterization.

| | SEC, % | SEC, % | SEC, % | |
|---|---|---|---|---|
| Sample | Aggregate | ADC | Fragments | DAR |
| Brentuximab-LD1 | 8.3 | 91.7 | 0 | 2 |
| Brentuximab-LD2 | 8.4 | 88.5 | 3.1 | 2 |
| Brentuximab-LD3 | 0.5 | 87.5 | 12 | 2.1 |
| Brentuximab-LD4 | 8.2 | 90 | 1.8 | 1.8 |
| Brentuximab-LD5 | 7.5 | 91.8 | 0.7 | 2.2 |
| Brentuximab-LD6 | 8.4 | 87.8 | 3.8 | 1.9 |
| Brentuximab-LD7 | 8.1 | 91.9 | 0 | 2.1 |
| Brentuximab-LD8 | 7.9 | 89.8 | 2.3 | 1.7 |
| Brentuximab-LD9 | 7.2 | 91.4 | 1.4 | 1.9 |
| Brentuximab-LD10 | 5.4 | 91.5 | 3.1 | 2 |
| Trastuzumab-LD1 | 3.8 | 93.7 | 2.5 | 2.1 |
| Trastuzumab-LD2 | 5.1 | 91.9 | 3 | 2.2 |
| Trastuzumab-LD3 | 0.4 | 86.1 | 13.5 | 2 |
| Trastuzumab-LD4 | 0 | 98.5 | 1.5 | 1.8 |
| Trastuzumab-LD5 | 0 | 92.6 | 7.4 | 2.1 |
| Trastuzumab-LD6 | 8.5 | 89.3 | 2.2 | 1.9 |
| Trastuzumab-LD7 | 4.2 | 95.2 | 0.6 | 2 |
| Trastuzumab-LD8 | 0.5 | 98.4 | 1.1 | 1.8 |
| Trastuzumab-LD9 | 0 | 97.3 | 2.7 | 2.1 |
| Trastuzumab-LD10 | 7.8 | 89.5 | 2.7 | 2 |
| Trastuzumab-LD15 | 4.8 | 95.1 | 0.1 | 4.3 |
| Non-targeting IgG1-LD1 | 1.2 | 95.8 | 3 | 2.1 |
| Non-targeting IgG1-LD2 | 0.4 | 96.2 | 3.4 | 1.9 |
| Non-targeting IgG1-LD3 | 0.6 | 96.8 | 2.6 | 2 |
| Non-targeting IgG1-LD4 | 1.7 | 95.1 | 3.2 | 2.1 |
| Non-targeting IgG1-LD5 | 1 | 95.9 | 3.1 | 2.3 |
| Non-targeting IgG1-LD6 | 8 | 88.8 | 3.2 | 2.1 |
| Non-targeting IgG1-LD7 | 1.1 | 96.3 | 2.6 | 2.2 |
| Non-targeting IgG1-LD8 | 1.7 | 95.8 | 2.5 | 1.8 |
| Non-targeting IgG1-LD9 | 0.6 | 96.1 | 3.3 | 2 |
| Non-targeting IgG1-LD10 | 8.6 | 88.4 | 3 | 1.9 |
| Non-targeting IgG1-LD15 | 4.2 | 94.4 | 1.4 | 4.4 |

### In Vitro Plasma Stability

The Trastuzumab-LD3 ADC was spiked in cyno, rat and mouse plasma at ~50 μg/mL concentrations and incubated at 37° C. for 7 days. Various time points were collected from 0 hour, 24 hours, 72 hours, and 168 hours respectively. Post incubations, all the time points were subjected to two-step immuno-affinity enrichment using Pierce™ MS-Compatible Magnetic IP Kit. The first step involved immobilization of the biotinylated anti-human IgG capture reagent on to the streptavidin magnetic beads followed by the second step of affinity purification of the samples. The eluted samples were injected on a high-performance liquid chromatography Agilent 1260 system connected with an Agilent 6230 TOF LC/MS system. The analysis was conducted using a reverse phase column (Zorbax 300SB—C8, 5 mm, 4.6×50 mm) with a denaturing mobile phase system. Mobile Phase A is 0.1% (v/v) formic acid in water. Mobile phase B is 0.1% (v/v) formic acid in acetonitrile (mobile phase B). The MS spectra of each protein are averaged and then deconvoluted to obtain the average mass and monoisotopic mass. MS is operated under denaturing conditions using m/z range of 300-3500 and capillary voltage of 4.5 kV. The MS data was analysed within the MassHunter software using the maximum entropy deconvolution algorithm. Percent conjugate remaining at each time-point was calculated based on time-0 reference with an assumption of time zero values being 100%. The assay imprecision is +/−25%.

The results are summarized in Table 2.

TABLE 2
In Vitro Plasma Stability Results

| Time, | % ADC in | % ADC in | % ADC in |
|---|---|---|---|
| hours | mouse plasma | rat plasma | cyno plasma |
| 0 | 100 | 100 | 100 |
| 72 | 125.0 | 86.3 | 91.1 |
| 168 | 76.3 | 79.9 | 98.0 |

### In Vitro Ecteinascidin ADC Cytotoxicity—Adherent Cells Cytotoxicity Assay

Adherent cells were cultured in a T75 flask to ~50-80% confluency and harvested with trypsin into a single cell suspension. Three to five thousand (3,000-5,000) cells per well were seeded in tissue culture plates in 50 μL/well culture media and incubated at 37° C. for 18-24 hours. Serial dilutions of ADCs in culture media were then dispensed into the plates at 50 μL/well. After plating and treatment, cells were returned to the incubator for an additional 3 to 5 days. CellTiter-Glo reagent was prepared per manufacturer's instructions and added at 100 μL/well to the cultures. CellTiter-Glo allows for relative enumeration of metabolically active cells by quantifying intracellular ATP concentrations. After 5 minutes of incubation with CellTiter-Glo at ambient room temperature, the clear bottom black assay plates were then read in a luminometer within 30 minutes, or 125 μL/well of the Cell Titer Glo/cell lysate solution was transferred into black assay plates and read. Luminescence readings obtained from cultures that did not receive any treatment (cell culture media only) were set as 100% control and all other luminescence values were normalized to these controls (e.g., Normalized RLU, relative luminescence unit). Luminescence signals were detected using a standard plate reader and IC50s were calculated by logistic nonlinear regression using GraphPad Prism (GraphPad Software, San Diego, CA).

### Suspension Cells Cytotoxicity Assay

Suspension cells were cultured in a T75 flask as a single cell suspension. Three to five thousand (3,000-5,000) cells per well were seeded in tissue culture plates in 50 μL/well culture media and incubated at 37° C. for 18-24 hours.

Serial dilutions of ADCs in culture media were then dispensed into the plates at 50 μL/well. After plating and treatment, cells were returned to the incubator for an additional 3-5 days. CellTiter-Glo reagent was prepared per manufacturer's instructions and added at 100 μL/well to the cultures. CellTiter-Glo allows for relative enumeration of metabolically active cells by quantifying intracellular ATP concentrations. After 5 minutes of incubation with CellTiter-Glo at ambient room temperature, the clear bottom black assay plates were then read in a luminometer within 30 minutes, or 125 μL/well of the Cell Titer Glo/cell lysate solution was transferred into black assay plates and read. Luminescence readings obtained from cultures that did not receive any treatment (cell culture media only) were set as 100% control and all other luminescence values were normalized to these controls (e.g., Normalized RLU, relative luminescence unit). Luminescence signals were detected using a standard plate reader and IC50s were calculated by logistic nonlinear regression using GraphPad Prism (GraphPad Software, San Diego, CA).

### Human Neutrophils Cytotoxicity Assay.

Human CD34+ myeloid progenitor cells were seeded at 3,000 cells per well in in growth media supplemented with 10 ng/ml human recombinant IL-3 and 30 ng/ml human recombinant G-CSF (both Peprotech, Cranbury, NJ) in 96-well plates. Test ADCs or free drugs were added to each well in duplicates or triplicate at final concentrations indicated. After 3-4 days of culture, half of the medium (100 μl) was carefully removed and CellTiter-Glo 2.0 (Promega, Madison, WI) viability assays were performed according to the manufacturer's protocol. Luminescence signals were detected using a standard plate reader and IC₅₀s were calculated by logistic nonlinear regression using GraphPad Prism (GraphPad Software, San Diego, CA).

Results are summarized in Tables 3-5 below. All experiments were repeated at least three times with each datapoint in duplicate. As used in the tables below, n/t indicates “not tested.”

TABLE 3
Ecteinascidin ADCs and Small Molecule Derivative 1 In Vitro
Cytotoxicity in HCC1954 (Her2 positive), Confluent HCC1954 (Her2
positive), SKBR3 (Her2 positive) and SUDHL1 (CD30 positive) cell lines.

| | | Confluent | | |
|---|---|---|---|---|
| | HCC1954 | HCC1954 | SKBR3 | SUDHL1 |
| Sample | IC₅₀, nM | IC₅₀, nM | IC₅₀, nM | IC₅₀, nM |
| Brentuximab-LD1 | 71.41 | 91.81 | 98.83 | 0.08086 |
| Brentuximab-LD2 | 85.83 | 135.2 | 285.9 | 0.09145 |
| Brentuximab-LD3 | >200 | >200 | >200 | 0.07949 |
| Brentuximab-LD4 | >200 | 76.15 | >200 | 0.09435 |
| Brentuximab-LD5 | 86.67 | 77.76 | 117.3 | 0.3885 |
| Brentuximab-LD6 | >200 | >200 | 108.3 | 0.05671 |
| Brentuximab-LD7 | 71.88 | 77.75 | 109.1 | 0.06281 |
| Brentuximab-LD8 | 81.46 | 165.8 | 90.08 | 0.04738 |
| Brentuximab-LD9 | 293.3 | 75.8 | 47.0 | 0.159 |
| Brentuximab-LD10 | 68.87 | 53.65 | >200 | 0.03695 |
| Trastuzumab-LD1 | 11.07 | 3.473 | 2.26 | >200 |
| Trastuzumab-LD2 | 21.54 | 9.922 | 1.321 | 261.8 |
| Trastuzumab-LD3 | 0.919 | 7.889 | 0.5008 | >200 |
| Trastuzumab-LD4 | 5.037 | 3.47 | 0.2983 | 287.1 |
| Trastuzumab-LD5 | 23.31 | 9.04 | 1.347 | >200 |
| Trastuzumab-LD6 | 2.284 | 5.328 | 1.484 | 244.2 |
| Trastuzumab-LD7 | 2.251 | 0.8247 | 1.017 | 444.3 |
| Trastuzumab-LD8 | 1.038 | 1.166 | 0.3442 | >200 |
| Trastuzumab-LD9 | 22.04 | 9.958 | 0.1512 | 249.9 |
| Trastuzumab-LD10 | 3.403 | 4.145 | 1.858 | 167.4 |
| Trastuzumab-LD15 | 0.11 | n/t | 0.01 | n/t |
| Non-targeting IgG1-LD1 | 277.6 | 269.5 | >200 | >200 |
| Non-targeting IgG1-LD2 | >200 | >200 | >200 | >200 |
| Non-targeting IgG1-LD3 | >200 | >200 | 154.4 | 120 |
| Non-targeting IgG1-LD4 | 79.68 | 94.49 | >200 | 33.85 |
| Non-targeting IgG1-LD5 | 82.64 | 45.01 | 210.9 | 34.38 |
| Non-targeting IgG1-LD6 | >200 | 75.17 | 73.34 | 362.5 |
| Non-targeting IgG1-LD7 | 252.6 | 82.72 | 32.32 | 40.53 |
| Non-targeting IgG1-LD8 | 75.3 | 91.74 | 24.3 | 95.16 |
| Non-targeting IgG1-LD9 | >200 | >200 | >200 | >200 |
| Non-targeting IgG1-LD10 | >200 | >200 | >200 | >200 |
| Non-targeting IgG1-LD15 | 13.88 | n/t | 13.68 | n/t |
| Lurbinectedin Derivative 1 | 2.579 | 3.697 | 0.5425 | 0.6246 |

TABLE 4
Ecteinascidin ADCs In Vitro Cytotoxicity in SKOV3(Her2
positive) and NCI-N87 (Her2 positive) cell lines.

| | SKOV3 | NCI-N87 |
|---|---|---|
| Sample | IC₅₀, nM | IC₅₀, nM |
| Brentuximab-LD1 | 66.76 | 120 |
| Brentuximab-LD2 | 150 | >200 |
| Brentuximab-LD3 | >200 | >200 |
| Brentuximab-LD4 | 150 | 78.31 |
| Brentuximab-LD5 | 150 | 150 |
| Brentuximab-LD6 | >200 | >200 |
| Brentuximab-LD7 | >200 | 76.72 |
| Brentuximab-LD8 | >200 | >200 |
| Brentuximab-LD9 | 70.34 | 76.29 |
| Brentuximab-LD10 | >200 | >200 |
| Trastuzumab-LD1 | 1.672 | 3.56 |
| Trastuzumab-LD2 | 1.878 | 0.8814 |
| Trastuzumab-LD3 | 0.3943 | 1.697 |
| Trastuzumab-LD4 | 0.5498 | 0.8411 |
| Trastuzumab-LD5 | 0.3655 | 0.7134 |
| Trastuzumab-LD6 | 0.2381 | 0.7989 |
| Trastuzumab-LD7 | 0.5298 | 0.07439 |
| Trastuzumab-LD8 | 0.06324 | 0.07556 |
| Trastuzumab-LD9 | 0.4113 | 1.045 |
| Trastuzumab-LD10 | 0.05066 | 0.7914 |
| Trastuzumab-LD15 | n/t | 0.08 |
| Non-targeting IgG1-LD1 | 74.05 | >200 |
| Non-targeting IgG1-LD2 | 100 | >200 |
| Non-targeting IgG1-LD3 | >200 | >200 |
| Non-targeting IgG1-LD4 | 150 | 181.2 |
| Non-targeting IgG1-LD5 | 150 | >200 |
| Non-targeting IgG1-LD6 | 66.14 | >200 |
| Non-targeting IgG1-LD7 | 150 | >200 |
| Non-targeting IgG1-LD8 | >200 | 120 |
| Non-targeting IgG1-LD9 | >200 | >200 |
| Non-targeting IgG1-LD10 | >200 | >200 |
| Non-targeting IgG1-LD15 | n/t | 56.72 |

TABLE 5
Ecteinascidin ADCs In Vitro
Cytotoxicity in Primary Human Neutrophils.

| | Human Neutrophils |
|---|---|
| Sample | IC₅₀, nM |
| Trastuzumab-LD1 | 66.32 |
| Trastuzumab-LD2 | 54.05 |
| Trastuzumab-LD3 | 120 |
| Trastuzumab-LD4 | 68.02 |
| Trastuzumab-LD6 | 100 |
| Trastuzumab-LD7 | 100 |
| Trastuzumab-LD8 | 28.01 |
| Trastuzumab-LD10 | 94.48 |
| Trastuzumab-LD15 | >100 |

### Example 21

### Antibody Drug Conjugate Efficacy and Tolerability

### In-Vivo Efficacy Study

6-8 weeks old female B-NDG mice were subcutaneously injected with 5×10⁶ NCI-N87 cells (ATCC CRL-5822) mixed with matrigel. The volume of the subcutaneously growing tumor was measured twice weekly using a caliper and calculated by the modified ellipsoidal formula: tumor volume (TV)=0.5×length×width². Body weight of the animal was also recorded twice per week to monitor for tolerability of the test articles. Five days after cell inculcation, when the average tumor volume was around 150 mm³, mice (n=5 per cohort) received their first dose of either vehicle control (PBS) or 0.5 mg/kg bodyweight Trastuzumab-LD7 antibody drug conjugate (ADC) through intra venous injection into the tail vein (Day 0). On Day 10 mice received a second dose of either vehicle control or Trastuzumab-LD7.

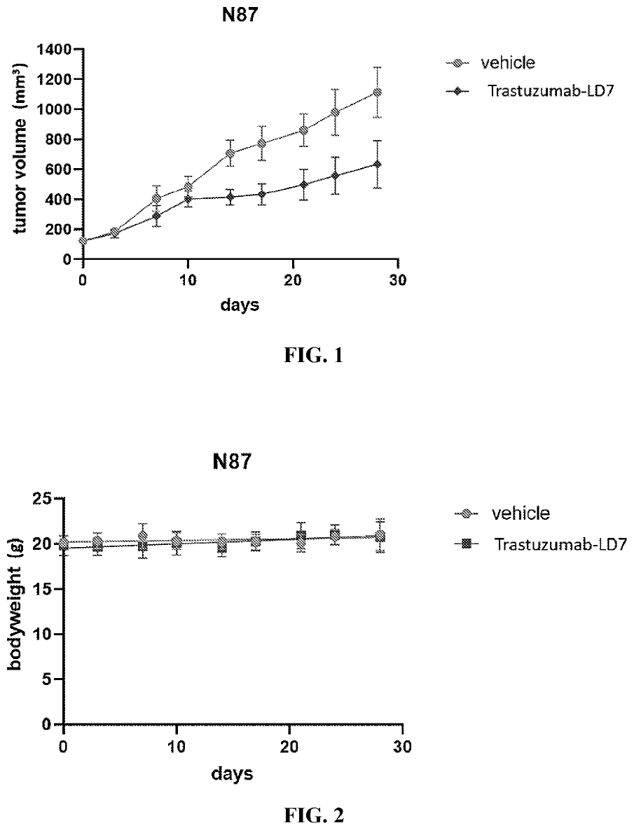

The antibody moiety trastuzumab is expected to bind to HER2 (ERBB2) on the surface of NCI-N87 cells and, if an ADC is functional in vivo, can deliver the LD7 payload after internalization into the cells to elicit cytotoxicity. The significant reduction in tumor volume in comparison to the vehicle control demonstrates that Trastuzumab-LD7 is a functional ADC in vivo without causing any adverse events in dosed animals as evident by the unchanged bodyweight. FIG. 1 provides tumor volume measurements of each cohort of mice throughout this study. FIG. 2 provides bodyweight measurements of each cohort of mice throughout this study.

### In-Vivo Tolerability Study

5-7 weeks old male CD-1 mice were intravenously injected via the tail vein with an isotype human IgG1 antibody conjugated to LD7. Three mice per group were dosed at either 6, 15 or 30 mg/kg body weight (mpk). Body weight of the animal and clinical observations were recorded every other day for 15 days post dosing. Throughout the study all animals continued to gain weight and there was no significant difference between the three dosing levels. Also, no clinical observations were made throughout the study. The gain in body weight and absence of any obvious adverse events suggests that LD7 ADCs are well tolerated in mice at the highest dose tested in this experiment.

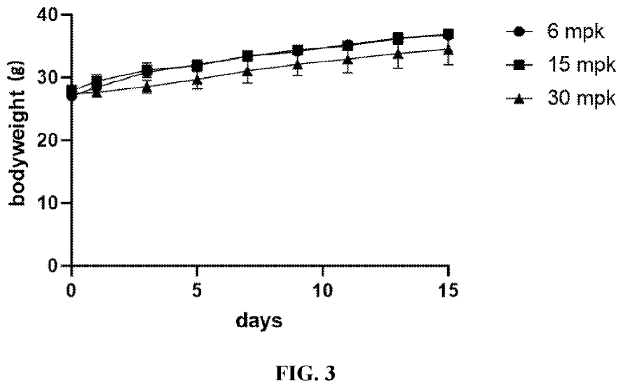

The maximum tolerated dose (MTD) of trabectedin, which is the payload of LD7, has previously been reported to be 0.15 mg/kg in male mice. For an ADC with a drug antibody ratio (DAR) of 2, this would mean that the expected tolerated dose should be 15 mg/kg. The fact that animals were dosed with LD7 at twice the normalized free payload dose without any signs of adverse events suggests that trabectedin delivered as an ADC is better tolerated and can be given at higher drug levels. FIG. 3 provides bodyweight measurements of each cohort of mice throughout this study.

### Example 22

### Synthesis of LD15

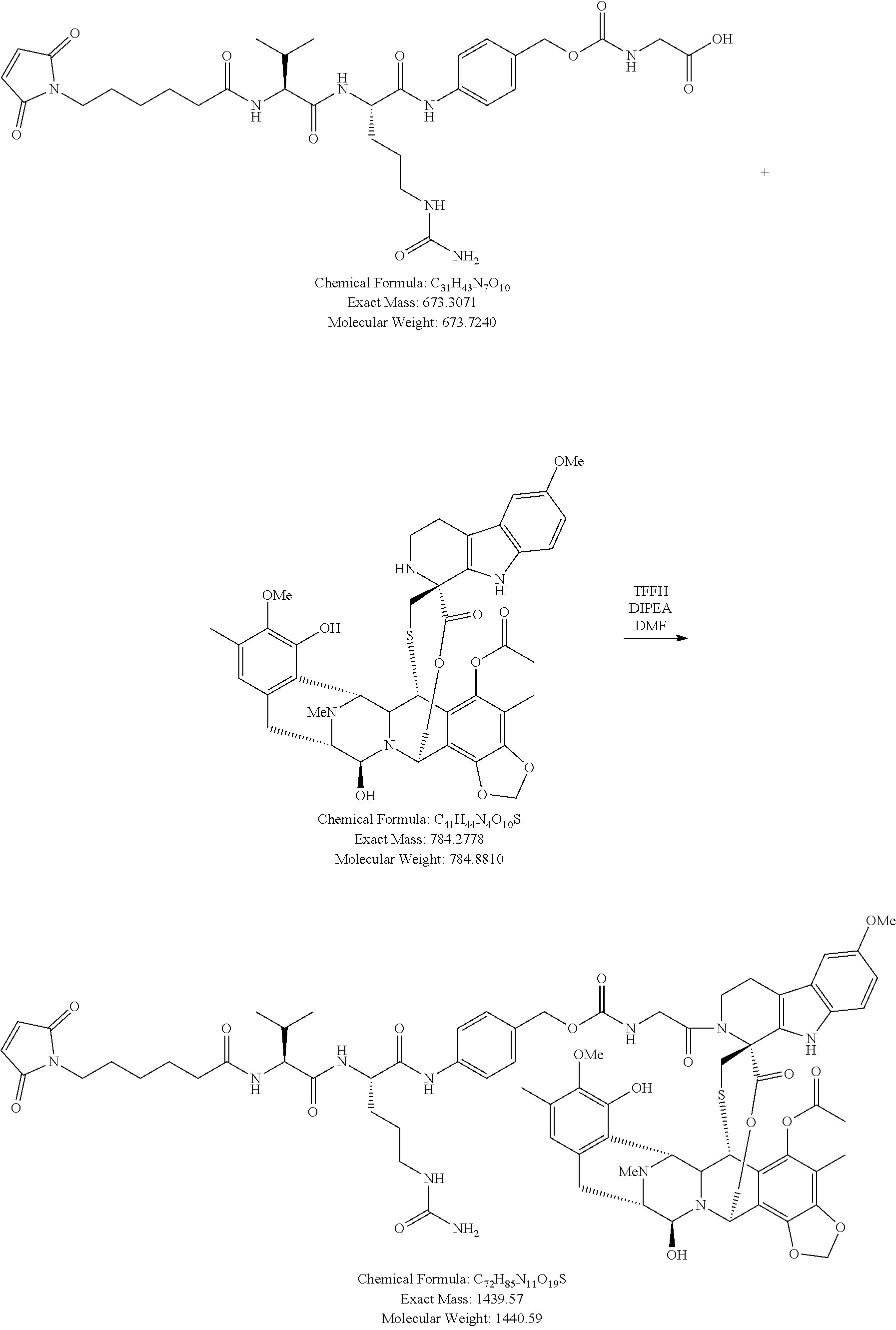 [SMILES: CC(C)[C@H](NC(=O)CCCCCN1C(=O)C=CC1=O)C(=O)N[C@@H](CCCNC(N)=O)C(=O)Nc1ccc(COC(=O)NCC(=O)O)cc1 ; COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4 ; COc1ccc2[nH]c3c(c2c1)CCN(C(=O)CNC(=O)OCc1ccc(NC(=O)[C@H](CCCNC(N)=O)NC(=O)[C@@H](NC(=O)CCCCCN2C(=O)C=CC2=O)C(C)C)cc1)[C@]31CS[C@@H]2c3c(OC(C)=O)c(C)c4c(c3[C@H](COC1=O)N1C2[C@H]2c3c(cc(C)c(OC)c3O)C[C@@H]([C@@H]1O)N2C)OCO4]

This example provides synthesis of lurbinectedin LD15.

MC-vc-PAB-Glycine carboxylic acid (1.3 equivalents) was dissolved in anhydrous dimethyl formamide (DMF) at 0.1M concentration, followed by addition of tetramethylfluoroformaidinium hexafluorophosphate (TFFH; 1.5 equivalents) and diisopropyl ethylamine (DIPEA; 4.5 equivalents). The reaction mixture was stirred for 30 min at room temperature before addition of lurbinectedin (1 equivalents). The reaction mixture was stirred for 2 hours at room temperature. The crude product was purified using flash reverse phase chromatography using Biotage Isolera chromatography instrument equipped with Biotage Sfar C18 column, gradient of 5%-95% Acetonitrile/Water with 0.1% formic acid. LCMS analytical method A, Rₜ=4.79 min, Expected [M+H]⁺ 1440.58, found [M+H−OH]²⁺ 711.9, [M+2H−OH]³⁺ 475, off-white solid.

The structure of MC-vc-PAB-Glycine carboxylic acid as used in the Examples of the present disclosure is shown below:

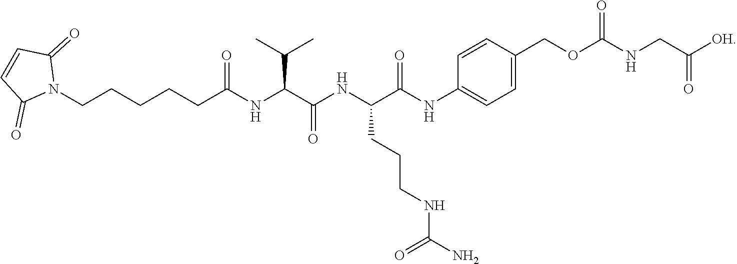 [SMILES: CC(C)[C@H](NC(=O)CCCCCN1C(=O)C=CC1=O)C(=O)N[C@@H](CCCNC(N)=O)C(=O)Nc1ccc(COC(=O)NCC(=O)O)cc1]

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

1. A compound of Formula (I), or a pharmaceutically acceptable salt, ester, stereoisomer, or tautomer thereof: 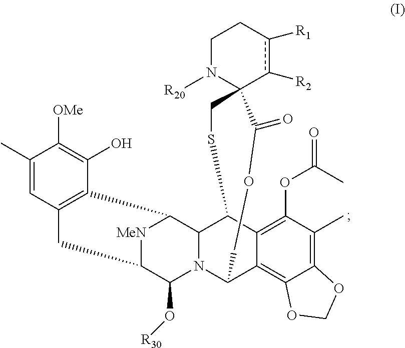
wherein R¹ and R² form at least one aromatic ring or bicyclic heterocyclic ring, optionally substituted with one to six substituents, independently in each instance, selected from the group consisting of C₁₋₁₀-alkoxy, C₁₋₁₀-alkyl, amino, hydroxyl, halogen, nitro, —P(O)(OH)₂, thiol, and —SO₃H;
wherein R²⁰ is either:
(a) selected from the group consisting of H, C₁₋₁₀-alkyl, C₁₋₁₀-aryl, C₃₋₁₀-heterocycle, C(O)R₅, C(O)OR₅, C(O)NHR₅, SO₂R₅, and SO₂NHR₅, wherein the C₁₋₁₀-alkyl and C₆₋₁₀-aryl are each, individually, substituted, or unsubstituted; or
(b) L¹, wherein L¹ is a reactive linker group;
wherein, R²⁰ is optionally substituted by 1-3 substituents, independently in each instance, selected from the group consisting of OH, F, Cl, Br, C₁₋₅-alkyl, C₃₋₆-cycloalkyl, and C₃₋₆-heterocycle;
wherein R³⁰ is either:
(c) selected from the group consisting of H, C₁₋₁₀-alkyl, C₆₋₁₀-aryl, phosphate, thiophosphate, phosphoramide, C(O)R₆, C(O)OR₆, and C(O)NHR₆, sulfonyl, sulfonylamide,
wherein the C₁₋₁₀-alkyl and C₆₋₁₀-aryl are each, individually, substituted, or unsubstituted; or
(d) L², wherein L² is a reactive linker group;
wherein the phosphate and thiophosphate are optionally substituted with one to two substituents independently, in each instance, selected from the group consisting of H, C₁₋₆-alkyl, C₃₋₁₀-cycloalkyl, (poly(ethylene) glycol)_{y} ([PEG]_{y}), C₆₋₁₀-aryl, and C₅₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H; and
wherein the substituent on the phosphate and thiophosphate is optionally terminated with a protecting group;
wherein R⁵ and R⁶ are each, independently in each instance, selected from the group consisting of C₁₋₆-alkyl, C₃₋₁₀-cycloalkyl, [PEG]_{y}, C₆₋₁₀-aryl, and C₆₋₁₀-heteroaryl,
wherein C₁₋₆-alkyl is optionally substituted with one to three substituents independently selected from the group consisting of methyl, ethyl, propyl, fluoro, chloro, bromo, iodo, amino, nitro, —P(O)(OH)₂, thiol, —OH, and —SO₃H;
wherein at least one of R²⁰ or R³⁰ is not H;
wherein [symbol] is either a single or a double bond; and
wherein subscript y is an integer selected from 0 to 32.

2. The compound of claim 1, wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰; or
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
R^{10a} is either absent or —(CH₂)ₑ—, wherein e is independently in each instance, selected from 1-4;
R^{10b} is selected from the group consisting of: 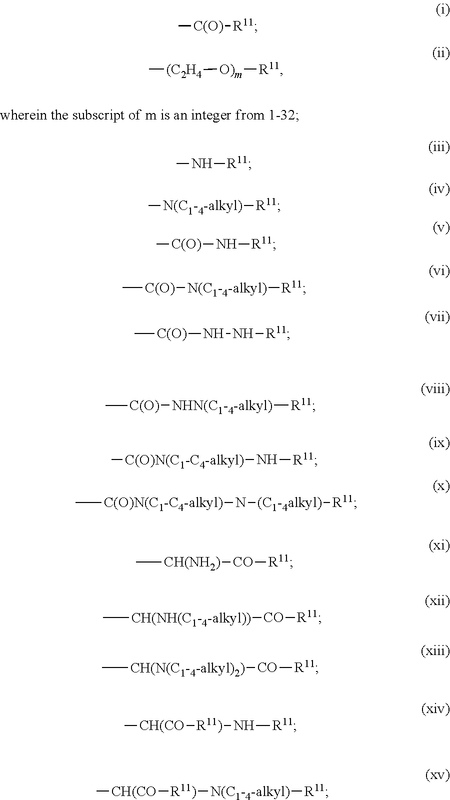
wherein R¹¹ is selected from the group consisting of R^{11a}, R^{11b}, R^{11c}, R^{11d}, and combinations thereof; wherein
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
R^{11c} is —(CH₂)ₓ—, wherein the subscript of x is an integer selected from 1 to 10; and
R^{11d} is selected from the group consisting of —O— and —NH.

3. The compound of claim 1, wherein L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
R^{10a} is either absent or —(CH₂)ₑ—, wherein e is independently in each instance, selected from 1-4;
R^{10b} is selected from the group consisting of: 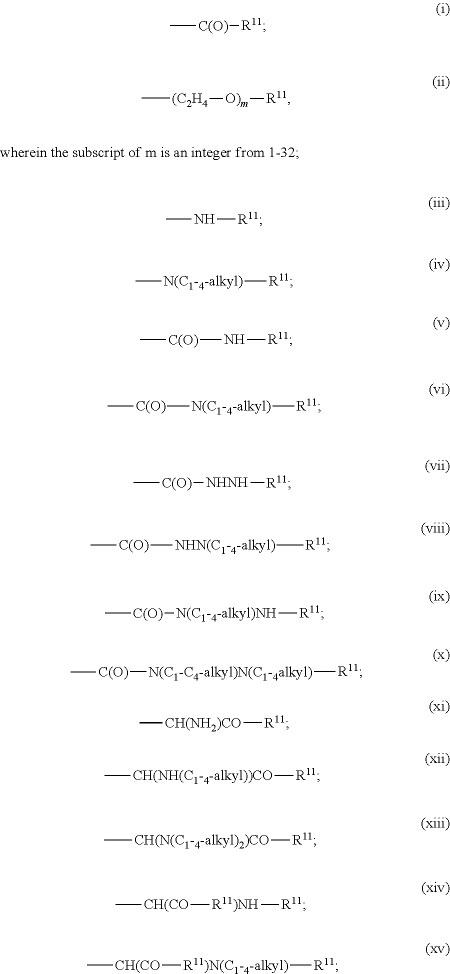
wherein R¹¹ is selected from the group consisting of R^{11a}, R^{11b}, R^{11c}, R^{11d}, and combinations thereof; wherein
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
R^{11b} is -(A₁)_{q}-, wherein A₁ is an amino acid, wherein the amino acid is, independently in each instance, selected from a natural amino acid or a non-natural amino acid, wherein subscript q is an integer selected from 1 to 6;
R^{11c} is —(CH₂)ₓ—, wherein the subscript of x is an integer selected from 1 to 10; and
R^{11d} is selected from the group consisting of —O— and —NH.

4. The compound of claim 2, wherein R¹¹ is selected from:
R^{11a}-R^{11b}-R^{11c}-R^{11d};
R^{11b}-R^{11c}-R^{11d};
R^{11c}-R^{11d}; or
R^{11a}-R^{11c}-R^{11d}.

5. The compound of claim 2, wherein R¹¹ is terminated in a reactive group, R⁴⁰.

6. The compound of claim 1, wherein L¹ comprises at least one of the following bivalent structures: 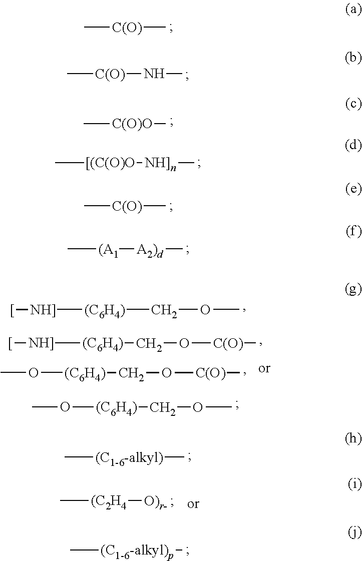
wherein L¹ or L2 are terminated in R⁴⁰, wherein R⁴⁰ is a reactive group;
A₁ and A₂ are independent in each instance an amino acid, wherein the amino acid is selected from a natural amino acid or a non-natural amino acid;
wherein subscript:
d is an integer selected from 0 to 1;
n is an integer selected from 0 to 4;
r is an integer selected from 0 to 32; and
p is an integer 0 or 1.

7. The compound of claim 1, wherein L² comprise at least one of the following bivalent structures: 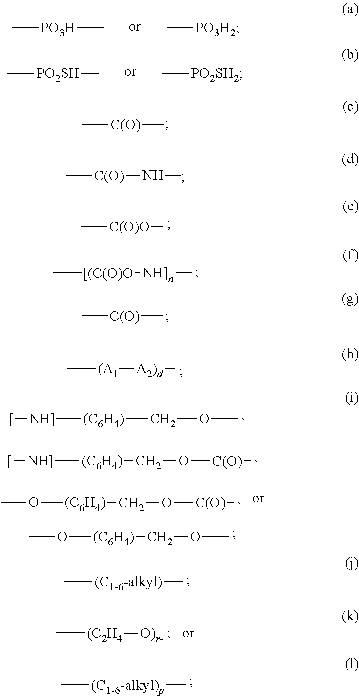
wherein L¹ or L2 are terminated in R⁴⁰, wherein R⁴⁰ is a reactive group;
A₁ and A₂ are independent in each instance an amino acid, wherein the amino acid is selected from a natural amino acid or a non-natural amino acid;
wherein subscript:
d is an integer selected from 0 to 1;
n is an integer selected from 0 to 4;
r is an integer selected from 0 to 32; and
p is an integer 0 or 1.

8. The compound of claim 1, wherein R²⁰ is 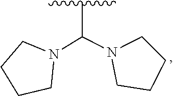 [SMILES: *C(N1CCCC1)N1CCCC1]
wherein 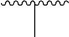
indicates a bond through which the illustrated substituent is bonded.

9. The compound of claim 1, wherein R⁴⁰ is 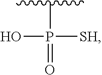 [SMILES: *P(=O)(O)S]
wherein 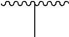
indicates the bond through which the thiophosphate is bonded.

10. The compound of claim 1, wherein the compound is selected from

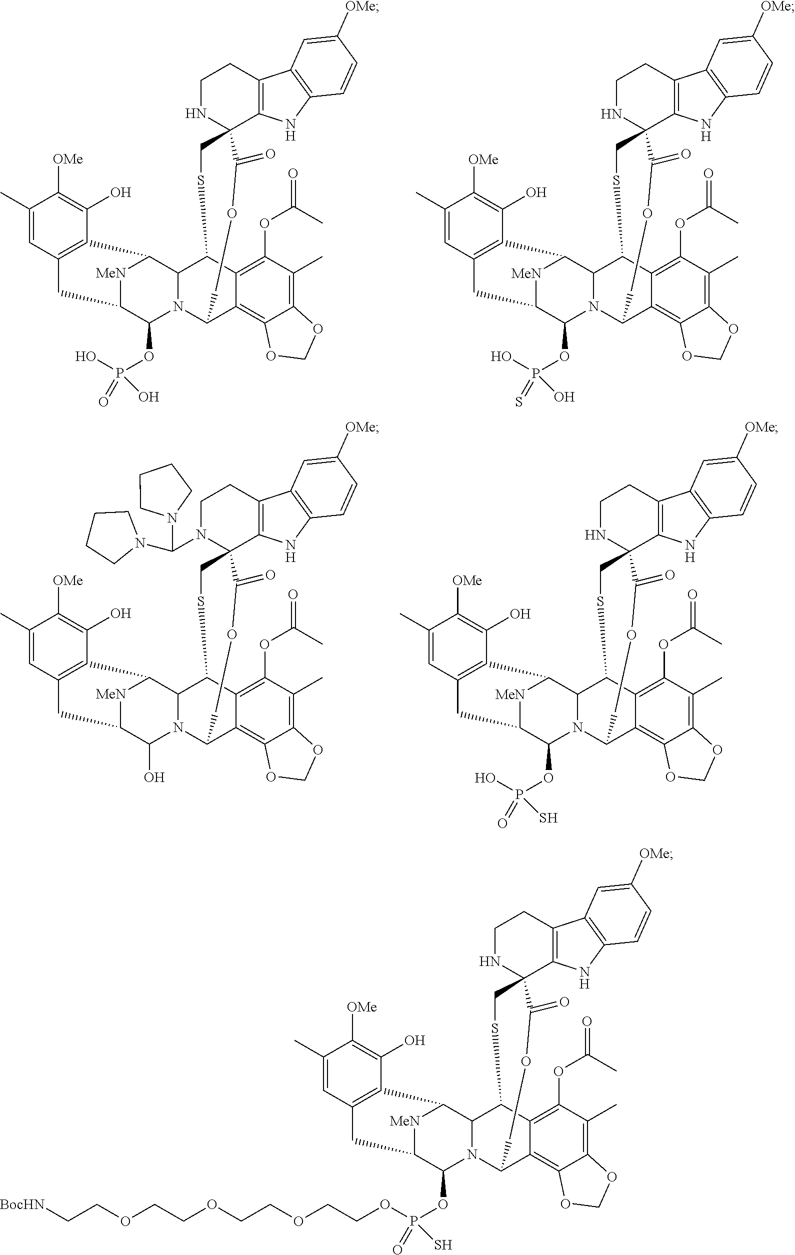

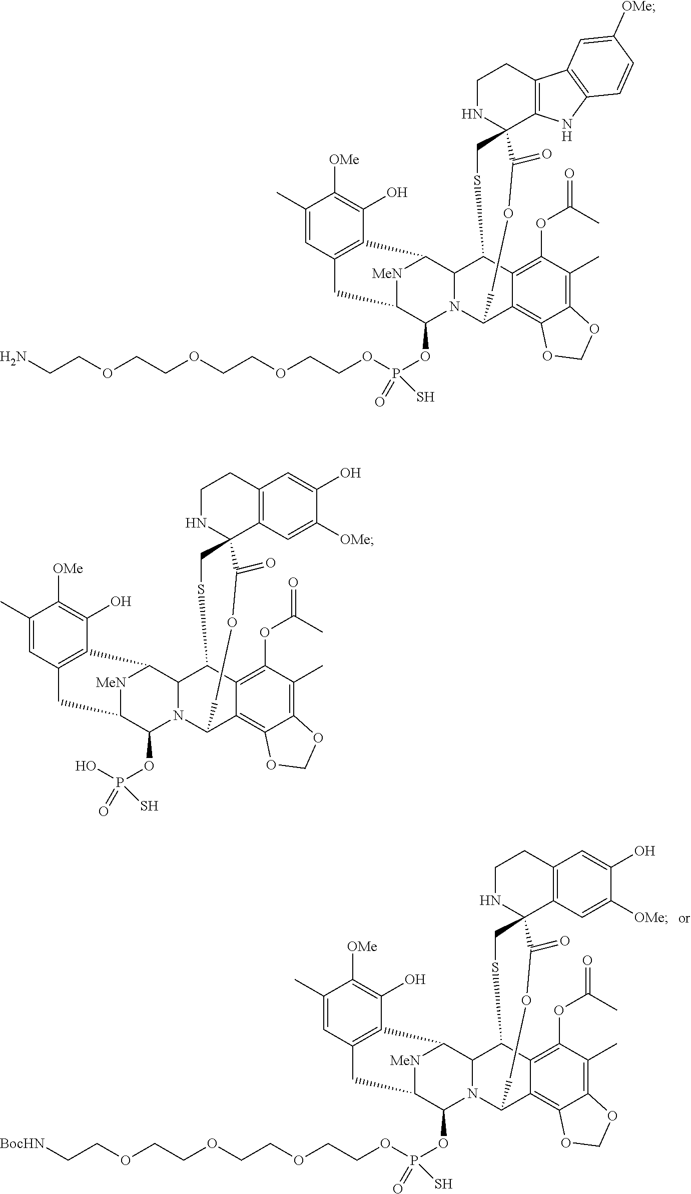 [SMILES: COc1ccc2[nH]c3c(c2c1)CCN[C@]31CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCN)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(O)S)N2C)OCO4 ; COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(=O)(S)OCCOCCOCCOCCNC(=O)OC(C)(C)C)N2C)OCO4]

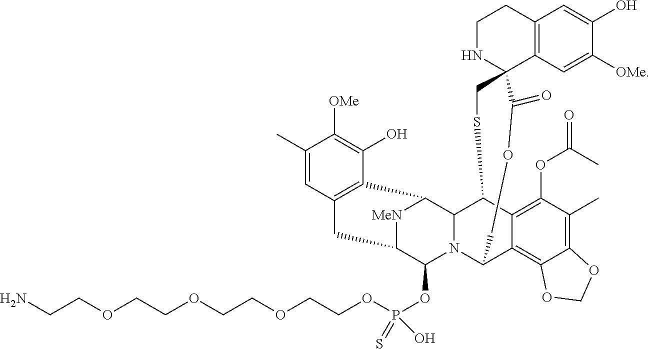 [SMILES: COc1cc2c(cc1O)CCN[C@]21CS[C@@H]2c3c(OC(C)=O)c(C)c4c(c3[C@H](COC1=O)N1C2[C@H]2c3c(cc(C)c(OC)c3O)C[C@@H]([C@@H]1OP(O)(=S)OCCOCCOCCOCCN)N2C)OCO4]

11. A compound of Formula (II) or a pharmaceutically acceptable salt, ester, stereoisomer, or tautomer thereof, comprising: 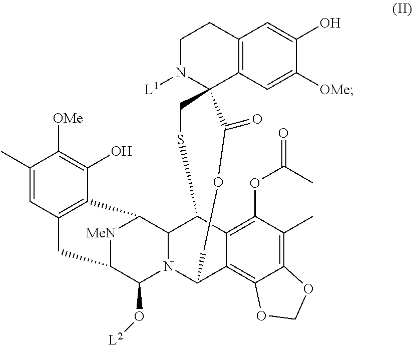
wherein one of L¹ or L² is a reactive linker.

12. The compound of claim 11, wherein L¹ comprises at least one of the following:
(a) —H, if L² is not H;
(b) —CH₃;
(c) —C₂₋₈-alkyl optionally substituted with at least one R¹⁰;
(d) —(C₀₋₆-alkyl)-C₃₋₁₀-cycloalkyl optionally substituted with at least one R¹⁰;
(e) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(f) —(C₀₋₆alkyl)-phenyl optionally substituted with at least one R¹⁰;
(g) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}-R^{10b}, wherein
R^{10a} is either absent or —(CH₂)ₑ—, wherein e is independently in each instance, selected from 1-4;
R^{10b} is selected from the group consisting of: 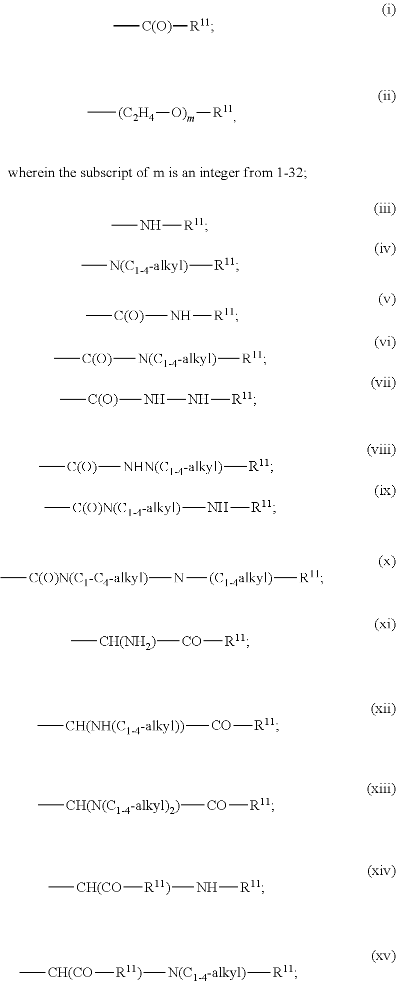
wherein R¹¹ is selected from the group consisting of R^{11a}, R^{11b}, R^{11c}, R^{11d}, and combinations thereof; wherein
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
R^{11c} is —(CH₂)ₓ—, wherein the subscript of x is an integer selected from 1 to 10; and
R^{11d} is selected from the group consisting of —O— and —NH.

13. The compound of claim 11, wherein L² comprises at least one of the following:
(a) —H, if L¹ is not H;
(b) —PO₃H— or —PO₃H₂;
(c) —PO₂SH— or —PO₂SH₂;
(d) —CH₃;
(e) —C₂₋₈-alkyl optionally substituted with at least one R¹⁰;
(f) —(C₀₋₆-alkyl)-C₃₋₁₀-cycloalkyl optionally substituted with at least one R¹⁰;
(g) —(C₀₋₆-alkyl)-C₃₋₁₀-heterocyclyl optionally substituted with at least one R¹⁰, wherein the C₃₋₁₀-heterocyclyl optionally comprises 1-3 heteroatoms independently selected from the group consisting of N, O, and S;
(h) —(C₀₋₆alkyl)-phenyl optionally substituted with at least one R¹⁰;
(j) —(C₀₋₆alkyl)-C₅₋₁₀-heteroaryl optionally substituted with at least one R¹⁰, wherein the C₅₋₁₀-heteroaryl optionally comprises 1-3 heteroatoms independently selected from the group N, O, and S;
wherein R¹⁰ is R^{10a}R^{10b}, wherein
R^{10a} is either absent or —(CH₂)ₑ—, wherein e is independently in each instance, selected from 1-4;
R^{10b} is selected from the group consisting of: 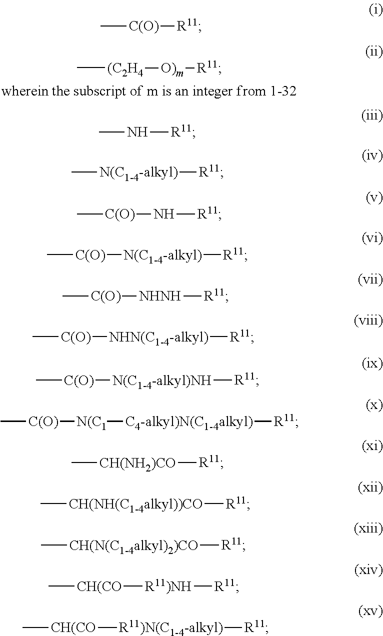
wherein R¹¹ is selected from the group consisting of R^{11a}, R^{11b}, R^{11c}, R^{11d}, and combinations thereof; wherein
R^{11a} is selected from the group consisting of [—NH]—(C₆H₄)—CH₂—O—, [—NH]—(C₆H₄)—CH₂—O—C(O)—, —O—(C₆H₄)—CH₂—O—C(O)—, and —O—(C₆H₄)—CH₂—O—;
R^{11b} is -(A₁-A₂)_{q}-, wherein A₁ and A₂ are independently in each instance an amino acid, wherein the amino acid is selected from a natural amino acid or a non-natural amino acid, wherein subscript q is an integer selected from 1 to 6;
R^{11c} is —(CH₂)ₓ—, wherein the subscript of x is an integer selected from 1 to 10; and
R^{11d} is selected from the group consisting of —O— and —NH.

14. The compound of claim 12, wherein R¹¹ is selected from:
R^{11a}-R^{11b}-R^{11c}-R^{11d};
R^{11b}-R^{11c}-R^{11d};
R^{11c}-R^{11d}; or
R^{11a}-R^{11c}-R^{11d}.

15. The compound of claim 12, wherein R¹¹ is terminated in a reactive group, R⁴⁰.

16-43. (canceled)

44. A pharmaceutical composition comprising the compound of claim 1 and a pharmaceutically acceptable excipient.

45. A method of treating a disease or condition comprising administering the compound of claim 1.

46-91. (canceled)
